SEQUENCE LISTING (150 sequences)

SEQ ID NO: 1 - DNA, 819 bp, Artificial Sequence
  Synthetic Polynucleotide
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggctcaaacctcctacgacaaggtgatatttgggccagggacaagcttatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 2 - protein, 272 aa, Artificial Sequence
  Synthetic Polypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATAQTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 3 - protein, 112 aa, Artificial Sequence
  Synthetic Polypeptide
SQQGEEDPQALSIQEGENATMNCSYKTSINNLQWYRQNSGRGLVHLILIRSNEREKHSGRLRVTLDTSKKSSSLLITASRAADTASYFCATAQTSYDKVIFGPGTSLSVIPN

SEQ ID NO: 4 - protein, 20 aa, Artificial Sequence
  Synthetic Polypeptide
METLLGVSLVILWLQLARVN

SEQ ID NO: 5 - protein, 5 aa, Artificial Sequence
  Synthetic Polypeptide
TSINN

SEQ ID NO: 6 - protein, 7 aa, Artificial Sequence
  Synthetic Polypeptide
IRSNERE

SEQ ID NO: 7 - protein, 11 aa, Artificial Sequence
  Synthetic Polypeptide
ATAQTSYDKVI

SEQ ID NO: 8 - DNA, 828 bp, Artificial Sequence
  Synthetic Polynucleotide
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagtggaggaacctacaaatacatctttggaacaggcaccaggctgaaggttttagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 9 - protein, 275 aa, Artificial Sequence
  Synthetic Polypeptide
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSGGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - protein, 115 aa, Artificial Sequence
  Synthetic Polypeptide
AQTVTQSQPEMSVQEAETVTLSCTYDTSESDYYLFWYKQPPSRQMILVIRQEAYKQQNATENRFSVNFQKAAKSFSLKISDSQLGDAAMYFCAYRSGGTYKYIFGTGTRLKVLAN

SEQ ID NO: 11 - protein, 20 aa, Artificial Sequence
  Synthetic Polypeptide
MACPGFLWALVISTCLEFSM

SEQ ID NO: 12 - protein, 7 aa, Artificial Sequence
  Synthetic Polypeptide
TSESDYY

SEQ ID NO: 13 - protein, 8 aa, Artificial Sequence
  Synthetic Polypeptide
QEAYKQQN

SEQ ID NO: 14 - protein, 11 aa, Artificial Sequence
  Synthetic Polypeptide
AYRSGGTYKYI

SEQ ID NO: 15 - DNA, 933 bp, Artificial Sequence
  Synthetic Polynucleotide
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcactattataatggagaagagagagcaaaaggaaacattcttgaacgattctccgcacaacagttccctgacttgcactctgaactaaacctgagctctctggagctgggggactcagctttgtatttctgtgccagcagcgttgaggggcccacctacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 16 - protein, 310 aa, Artificial Sequence
  Synthetic Polypeptide
MGFRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSVEGPTYNEQFFGPGTRLTVLEDLKNVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 17 - protein, 114 aa, Artificial Sequence
  Synthetic Polypeptide
DSGVTQTPKHLITATGQRVTLRCSPRSGDLSVYWYQQSLDQGLQFLIHYYNGEERAKGNILERFSAQQFPDLHSELNLSSLELGDSALYFCASSVEGPTYNEQFFGPGTRLTVL

SEQ ID NO: 18 - protein, 19 aa, Artificial Sequence
  Synthetic Polypeptide
MGFRLLCCVAFCLLGAGPV

SEQ ID NO: 19 - protein, 5 aa, Artificial Sequence
  Synthetic Polypeptide
SGDLS

SEQ ID NO: 20 - protein, 6 aa, Artificial Sequence
  Synthetic Polypeptide
YYNGEE

SEQ ID NO: 21 - protein, 13 aa, Artificial Sequence
  Synthetic Polypeptide
ASSVEGPTYNEQF

SEQ ID NO: 22 - protein, 9 aa, Homo Sapiens
FVLAAVYRI

SEQ ID NO: 23 - protein, 9 aa, Homo Sapiens
VATSRTLSY

SEQ ID NO: 24 - protein, 9 aa, Homo Sapiens
LSYFIASFR

SEQ ID NO: 25 - protein, 9 aa, Homo Sapiens
LWLLWPVTL

SEQ ID NO: 26 - protein, 9 aa, Homo Sapiens
CVADYSVLY

SEQ ID NO: 27 - protein, 11 aa, Homo Sapiens
SFIEDLLFNKV

SEQ ID NO: 28 - protein, 10 aa, Homo Sapiens
LSSTASALGK

SEQ ID NO: 29 - protein, 9 aa, Homo Sapiens
GTHWFVTQR

SEQ ID NO: 30 - protein, 9 aa, Homo Sapiens
LFVTVYSHL

SEQ ID NO: 31 - protein, 9 aa, Homo Sapiens
FQPTNGVGY

SEQ ID NO: 32 - protein, 9 aa, Homo Sapiens
GVLTESNKK

SEQ ID NO: 33 - protein, 10 aa, Homo Sapiens
VYSSANNCTF

SEQ ID NO: 34 - protein, 10 aa, Homo Sapiens
IVDTVSALVY

SEQ ID NO: 35 - protein, 9 aa, Homo Sapiens
TLVPQEHYV

SEQ ID NO: 36 - protein, 9 aa, Homo Sapiens
KLFAAETLK

SEQ ID NO: 37 - protein, 9 aa, Homo Sapiens
VYIGDPAQL

SEQ ID NO: 38 - protein, 9 aa, Homo Sapiens
VVYRGTTTY

SEQ ID NO: 39 - protein, 10 aa, Homo Sapiens
ELAGIGILTV

SEQ ID NO: 40 - protein, 10 aa, Homo Sapiens
LSELETPGKY

SEQ ID NO: 41 - protein, 10 aa, Homo Sapiens
KVFPCALINK

SEQ ID NO: 42 - protein, 9 aa, Homo Sapiens
NYKRCFPVI

SEQ ID NO: 43 - protein, 9 aa, Homo Sapiens
GLMWLSYFI

SEQ ID NO: 44 - protein, 9 aa, Homo Sapiens
VTDVTQLYL

SEQ ID NO: 45 - protein, 10 aa, Homo Sapiens
KLVLSVNPYV

SEQ ID NO: 46 - protein, 10 aa, Homo Sapiens
VVYRGTTTYK

SEQ ID NO: 47 - protein, 10 aa, Homo Sapiens
FLYIIKLIFL

SEQ ID NO: 48 - protein, 10 aa, Homo Sapiens
TVDSSQGSEY

SEQ ID NO: 49 - protein, 9 aa, Homo Sapiens
KLSYGIATV

SEQ ID NO: 50 - protein, 10 aa, Homo Sapiens
LIFLWLLWPV

SEQ ID NO: 51 - protein, 9 aa, Homo Sapiens
FAIGLALYY

SEQ ID NO: 52 - protein, 9 aa, Homo Sapiens
KLNVGDYFV

SEQ ID NO: 53 - protein, 9 aa, Homo Sapiens
MSYYCKSHK

SEQ ID NO: 54 - protein, 10 aa, Homo Sapiens
DSSQGSEYDY

SEQ ID NO: 55 - protein, 10 aa, Homo Sapiens
SMATNYDLSV

SEQ ID NO: 56 - protein, 10 aa, Homo Sapiens
QLYLGGMSYY

SEQ ID NO: 57 - protein, 10 aa, Homo Sapiens
TLACFVLAAV

SEQ ID NO: 58 - protein, 10 aa, Homo Sapiens
ATEETFKLSY

SEQ ID NO: 59 - protein, 10 aa, Homo Sapiens
TLEQYVFCTV

SEQ ID NO: 60 - protein, 10 aa, Homo Sapiens
SVVNARLRAK

SEQ ID NO: 61 - protein, 9 aa, Homo Sapiens
KLLEQWNLV

SEQ ID NO: 62 - protein, 10 aa, Homo Sapiens
SAQCFKMFYK

SEQ ID NO: 63 - protein, 10 aa, Homo Sapiens
FLWLLWPVTL

SEQ ID NO: 64 - protein, 10 aa, Homo Sapiens
KSAQCFKMFY

SEQ ID NO: 65 - protein, 11 aa, Homo Sapiens
TLVPQEHYVRI

SEQ ID NO: 66 - protein, 10 aa, Homo Sapiens
YLGGMSYYCK

SEQ ID NO: 67 - protein, 11 aa, Homo Sapiens
LLWPVTLACFV

SEQ ID NO: 68 - protein, 10 aa, Homo Sapiens
HFAIGLALYY

SEQ ID NO: 69 - protein, 9 aa, Homo Sapiens
YVFCTVNAL

SEQ ID NO: 70 - protein, 10 aa, Homo Sapiens
RFNVAITRAK

SEQ ID NO: 71 - protein, 10 aa, Homo Sapiens
TCDWTNAGDY

SEQ ID NO: 72 - protein, 9 aa, Homo Sapiens
GLALYYPSA

SEQ ID NO: 73 - protein, 10 aa, Homo Sapiens
YVFTGYRVTK

SEQ ID NO: 74 - protein, 9 aa, Homo Sapiens
LMWLSYFIA

SEQ ID NO: 75 - protein, 9 aa, Homo Sapiens
CTERLKLFA

SEQ ID NO: 76 - protein, 11 aa, Homo Sapiens
FKVNSTLEQYV

SEQ ID NO: 77 - protein, 10 aa, Homo Sapiens
CIRRPFLCCK

SEQ ID NO: 78 - protein, 10 aa, Homo Sapiens
KLPDDFTGCV

SEQ ID NO: 79 - protein, 9 aa, Homo Sapiens
LLFNKVTLA

SEQ ID NO: 80 - protein, 9 aa, Homo Sapiens
RLDKVEAEV

SEQ ID NO: 81 - protein, 10 aa, Homo Sapiens
RLITGRLQSL

SEQ ID NO: 82 - protein, 13 aa, Artificial Sequence
  Synthetic Polypeptide
CATAQTSYDKVIF

SEQ ID NO: 83 - protein, 15 aa, Artificial Sequence
  Synthetic Polypeptide
CASSVEGPTYNEQFF

SEQ ID NO: 84 - protein, 52 aa, Artificial Sequence
  Synthetic Polypeptide
GATTACGCCAAGCTTTGTTGCTCTTGAAGTCCATAGACCTCATGTCTAGCAC

SEQ ID NO: 85 - protein, 43 aa, Artificial Sequence
  Synthetic Polypeptide
GATTACGCCAAGCTTTTCTGATGGCTCAAACACAGCGACCTCG

SEQ ID NO: 86 - DNA, 825 bp, Artificial Sequence
  Synthetic Polynucleotide
atggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagacagaggctttgggaatgtgctgcattgcgggtccggcactcaagtgattgttttaccacatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 87 - protein, 274 aa, Artificial Sequence
  Synthetic Polypeptide
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDRGFGNVLHCGSGTQVIVLPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 88 - protein, 116 aa, Artificial Sequence
  Synthetic Polypeptide
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDRGFGNVLHCGSGTQVIVLPHI

SEQ ID NO: 89 - protein, 19 aa, Artificial Sequence
  Synthetic Polypeptide
MASAPISMLAMLFTLSGLR

SEQ ID NO: 90 - protein, 6 aa, Artificial Sequence
  Synthetic Polypeptide
VSGNPY

SEQ ID NO: 91 - protein, 8 aa, Artificial Sequence
  Synthetic Polypeptide
YITGDNLV

SEQ ID NO: 92 - protein, 12 aa, Artificial Sequence
  Synthetic Polypeptide
AVRDRGFGNVLH

SEQ ID NO: 93 - DNA, 921 bp, Artificial Sequence
  Synthetic Polynucleotide
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgcccggttgaaggactcctacgagcagtacttcgggccgggcaccaggctcacggtcacagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 94 - protein, 306 aa, Artificial Sequence
  Synthetic Polypeptide
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSARLKDSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 95 - protein, 115 aa, Artificial Sequence
  Synthetic Polypeptide
SAVISQKPSRDICQRGTSLTIQCQVDSQVTMMFWYRQQPGQSLTLIATANQGSEATYESGFVIDKFPISRPNLTFSTLTVSNMSPEDSSIYLCSARLKDSYEQYFGPGTRLTVTE

SEQ ID NO: 96 - protein, 15 aa, Artificial Sequence
  Synthetic Polypeptide
MLSLLLLLLGLGSVF

SEQ ID NO: 97 - protein, 5 aa, Artificial Sequence
  Synthetic Polypeptide
SQVTM

SEQ ID NO: 98 - protein, 7 aa, Artificial Sequence
  Synthetic Polypeptide
ANQGSEA

SEQ ID NO: 99 - protein, 11 aa, Artificial Sequence
  Synthetic Polypeptide
SARLKDSYEQY

SEQ ID NO: 100 - DNA, 828 bp, Artificial Sequence
  Synthetic Polynucleotide
atgacacgagttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatgaactccaacaatgccagactcatgtttggagatggaactcagctggtggtgaagcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 101 - protein, 275 aa, Artificial Sequence
  Synthetic Polypeptide
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMNSNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 102 - protein, 115 aa, Artificial Sequence
  Synthetic Polypeptide
AQTVTQSQPEMSVQEAETVTLSCTYDTSENNYYLFWYKQPPSRQMILVIRQEAYKQQNATENRFSVNFQKAAKSFSLKISDSQLGDTAMYFCAFMNSNNARLMFGDGTQLVVKPN

SEQ ID NO: 103 - protein, 20 aa, Artificial Sequence
  Synthetic Polypeptide
MTRVSLLWAVVVSTCLESGM

SEQ ID NO: 104 - protein, 7 aa, Artificial Sequence
  Synthetic Polypeptide
TSENNYY

SEQ ID NO: 105 - protein, 8 aa, Artificial Sequence
  Synthetic Polypeptide
QEAYKQQN

SEQ ID NO: 106 - protein, 11 aa, Artificial Sequence
  Synthetic Polypeptide
AFMNSNNARLM

SEQ ID NO: 107 - DNA, 930 bp, Artificial Sequence
  Synthetic Polynucleotide
atgggcaccaggctcttcttctatgtggccctttgtctgctgtgggcaggacacagggatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctacgttgtctccagatccaagacagagaatttccccctcactctggagtcagctacccgctcccagacatctgtgtatttctgcgccagcagcccggggtccgtcccaggcggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 108 - protein, 309 aa, Artificial Sequence
  Synthetic Polypeptide
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSPGSVPGGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 109 - protein, 113 aa, Artificial Sequence
  Synthetic Polypeptide
DAGITQSPRYKITETGRQVTLMCHQTWSHSYMFWYRQDLGHGLRLIYYSAAADITDKGEVPDGYVVSRSKTENFPLTLESATRSQTSVYFCASSPGSVPGGYTFGSGTRLTVV

SEQ ID NO: 110 - protein, 19 aa, Artificial Sequence
  Synthetic Polypeptide
MGTRLFFYVALCLLWAGHR

SEQ ID NO: 111 - protein, 5 aa, Artificial Sequence
  Synthetic Polypeptide
WSHSY

SEQ ID NO: 112 - protein, 6 aa, Artificial Sequence
  Synthetic Polypeptide
SAAADI

SEQ ID NO: 113 - protein, 12 aa, Artificial Sequence
  Synthetic Polypeptide
ASSPGSVPGGYT

SEQ ID NO: 114 - DNA, 819 bp, Artificial Sequence
  Synthetic Polynucleotide
atgatgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaaagacagctgggggaaattgcagtttggagcagggacccaggttgtggtcaccccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 115 - protein, 272 aa, Artificial Sequence
  Synthetic Polypeptide
MMISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVKDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 116 - protein, 110 aa, Artificial Sequence
  Synthetic Polypeptide
RKEVEQDPGPFNVPEGATVAFNCTYSNSASQSFFWYRQDCRKEPKLLMSVYSSGNEDGRFTAQLNRASQYISLLIRDSKLSDSATYLCVVKDSWGKLQFGAGTQVVVTPD

SEQ ID NO: 117 - protein, 22 aa, Artificial Sequence
  Synthetic Polypeptide
MMISLRVLLVILWLQLSWVWSQ

SEQ ID NO: 118 - protein, 6 aa, Artificial Sequence
  Synthetic Polypeptide
NSASQS

SEQ ID NO: 119 - protein, 6 aa, Artificial Sequence
  Synthetic Polypeptide
VYSSGN

SEQ ID NO: 120 - protein, 10 aa, Artificial Sequence
  Synthetic Polypeptide
VVKDSWGKLQ

SEQ ID NO: 121 - DNA, 933 bp, Artificial Sequence
  Synthetic Polynucleotide
atggccaccaggctcctctgctgtgtggttctttgtctcctgggagaagagcttatagatgctagagtcacccagacaccaaggcacaaggtgacagagatgggacaagaagtaacaatgagatgtcagccaattttaggccacaatactgttttctggtacagacagaccatgatgcaaggactggagttgctggcttacttccgcaaccgggctcctctagatgattcggggatgccgaaggatcgattctcagcagagatgcctgatgcaactttagccactctgaagatccagccctcagaacccagggactcagctgtgtatttttgtgctagtggtccttcagggggctggaacaccatatattttggagagggaagttggctcactgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 122 - protein, 310 aa, Artificial Sequence
  Synthetic Polypeptide
MATRLLCCVVLCLLGEELIDARVTQTPRHKVTEMGQEVTMRCQPILGHNTVFWYRQTMMQGLELLAYFRNRAPLDDSGMPKDRFSAEMPDATLATLKIQPSEPRDSAVYFCASGPSGGWNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 123 - protein, 114 aa, Artificial Sequence
  Synthetic Polypeptide
DARVTQTPRHKVTEMGQEVTMRCQPILGHNTVFWYRQTMMQGLELLAYFRNRAPLDDSGMPKDRFSAEMPDATLATLKIQPSEPRDSAVYFCASGPSGGWNTIYFGEGSWLTVV

SEQ ID NO: 124 - protein, 19 aa, Artificial Sequence
  Synthetic Polypeptide
MATRLLCCVVLCLLGEELI

SEQ ID NO: 125 - protein, 5 aa, Artificial Sequence
  Synthetic Polypeptide
LGHNT

SEQ ID NO: 126 - protein, 6 aa, Artificial Sequence
  Synthetic Polypeptide
FRNRAP

SEQ ID NO: 127 - protein, 12 aa, Artificial Sequence
  Synthetic Polypeptide
ASGPSGGWNTIY

SEQ ID NO: 128 - DNA, 828 bp, Artificial Sequence
  Synthetic Polynucleotide
atgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtaattcaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 129 - protein, 275 aa, Artificial Sequence
  Synthetic Polypeptide
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSNSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 130 - protein, 116 aa, Artificial Sequence
  Synthetic Polypeptide
AQSVTQLGSHVSVSEGALVLLRCNYSSSVPPYLFWYVQYPNQGLQLLLKYTSAATLVKGINGFEAEFKKSETSFHLTKPSAHMSDAAEYFCAVSNSGGGADGLTFGKGTHLIIQPY

SEQ ID NO: 131 - protein, 19 aa, Artificial Sequence
  Synthetic Polypeptide
MLLLLVPVLEVIFTLGGTR

SEQ ID NO: 132 - protein, 6 aa, Artificial Sequence
  Synthetic Polypeptide
SSVPPY

SEQ ID NO: 133 - protein, 8 aa, Artificial Sequence
  Synthetic Polypeptide
YTSAATLV

SEQ ID NO: 134 - protein, 13 aa, Artificial Sequence
  Synthetic Polypeptide
AVSNSGGGADGLT

SEQ ID NO: 135 - DNA, 933 bp, Artificial Sequence
  Synthetic Polynucleotide
atgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtattatggcagggggtcacggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 136 - protein, 310 aa, Artificial Sequence
  Synthetic Polypeptide
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIMAGGHGELFFGEGSRLTVLEDLKNVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 137 - protein, 114 aa, Artificial Sequence
  Synthetic Polypeptide
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIMAGGHGELFFGEGSRLTVL

SEQ ID NO: 138 - protein, 19 aa, Artificial Sequence
  Synthetic Polypeptide
MSNQVLCCVVLCFLGANTV

SEQ ID NO: 139 - protein, 5 aa, Artificial Sequence
  Synthetic Polypeptide
LNHDA

SEQ ID NO: 140 - protein, 6 aa, Artificial Sequence
  Synthetic Polypeptide
SQIVND

SEQ ID NO: 141 - protein, 13 aa, Artificial Sequence
  Synthetic Polypeptide
ASSIMAGGHGELF

SEQ ID NO: 142 - protein, 5 aa, Artificial Sequence
  Synthetic Polypeptide
GSGGS

SEQ ID NO: 143 - protein, 4 aa, Artificial Sequence
  Synthetic Polypeptide
GGGS

SEQ ID NO: 144 - protein, 4 aa, Artificial Sequence
  Synthetic Polypeptide
GGSG

SEQ ID NO: 145 - protein, 5 aa, Artificial Sequence
  Synthetic Polypeptide
GGSGG

SEQ ID NO: 146 - protein, 5 aa, Artificial Sequence
  Synthetic Polypeptide
GSGSG

SEQ ID NO: 147 - protein, 5 aa, Artificial Sequence
  Synthetic Polypeptide
GSGGG

SEQ ID NO: 148 - protein, 5 aa, Artificial Sequence
  Synthetic Polypeptide
GGGSG

SEQ ID NO: 149 - protein, 5 aa, Artificial Sequence
  Synthetic Polypeptide
GSSSG

SEQ ID NO: 150 - protein, 13 aa, Artificial Sequence
  Synthetic Polypeptide
CAYRSGGTYKYIF

(19) United States
(12) Patent Application Publication
(10) Pub. No.: US 2024/0293459 A1
(43) Pub. Date: Sep. 5, 2024

(54) PEPTIDES AND ENGINEERED T CELL RECEPTORS TARGETING SARS-COV-2 ANTIGENS AND METHODS OF USE

(71) Applicants: Cassian YEE, Houston, TX (US); BOARD OF REGENTS, THE UNIVERSITY OF TEXAS SYSTEM, Austin, TX (US)

(72) Inventors: Cassian YEE, Houston, TX (US); Ke PAN, Houston, TX (US); Yulun CHIU, Houston, TX (US)

(73) Assignee: BOARD OF REGENTS, THE UNIVERSITY OF TEXAS SYSTEM, Austin, TX (US)

(21) Appl. No.: 18/552,417

(22) PCT Filed: Mar. 29, 2022
(86) PCT No.: PCT/US2022/022324

§ 371 Date: Sep. 25, 2023

Related U.S. Application Data
(60) Provisional application No. 63/167,433, filed on Mar. 29, 2021.
(60) Provisional application No. 63/210,198, filed on Jun. 14, 2021.
(60) Provisional application No. 63/314,870, filed on Feb. 28, 2022.

(51) Int. Cl. A61K 35/17; A61K 39/00; C07K 14/005; C07K 14/725; C12N 5/0783; C12N 5/0784; C12N 15/86; G01N 33/569

(52) U.S. Cl. CPC A61K 35/17; A61K 39/4611; A61K 39/4615; A61K 39/4622; A61K 39/4632; A61K 39/464838; C07K 14/005; C07K 14/7051; C12N 5/0636; C12N 5/0639; C12N 15/86; G01N 33/56972; A61K 2039/5154; A61K 2039/5158; A61K 2039/572; C07K 2319/02; C12N 2740/15043; C12N 2770/20022; G01N 2800/52

(57) ABSTRACT

This disclosure provides for peptides useful for vaccination and other applications, engineered T cell Receptors (TCRs), cells comprising the peptides and TCRs, and methods of making and using the peptides and TCRs. The current disclosure relates to TCRs that specifically recognize SARS-Cov-2 HLA-A2 restricted peptide from membrane glycol-protein (MGP), MGP-65: FVLAAVYRI (SEQ ID NO:22).

### CROSS REFERENCE TO RELATED APPLICATIONS

This application claims priority to U.S. Provisional Patent Application Ser. No. 63/167,433, filed on Mar. 29, 2021; U.S. Provisional Patent Application Ser. No. 63/210,198, filed Jun. 14, 2021; and U.S. Provisional Patent Application Ser. No. 63/314,870, filed Feb. 28, 2022, which are incorporated by reference herein in their entirety.

### BACKGROUND OF THE INVENTION

### Field of the Invention

This invention relates to the field of immunotherapy and treatment of viral infections.

### Background

Since the emergence of SARS-CoV-2 in December 2019, the World Health Organization has reported spread to over 200 countries with infections surpassing 170 million and deaths surpassing 3.5 million worldwide. Despite this burden, the quest to identify effective vaccines, therapies, and protective biomarkers continues.

SARS-CoV-2 manifests as severe atypical pneumonia associated with high morbidity and mortality in humans. A dysregulated/excessive innate response associated with T-cell lymphopenia are leading contributors to the lung pathology in this syndrome. Decreased numbers of T cells correlate with the severity of acute phase of SARS disease in humans and delayed clearance of the virus. Several lines of evidence from other respiratory virus infections such as influenza A and para-influenza have established that virus-specific CD4 and CD8 T cells generated during primary and memory response can clear the virus and protect the host from severe infections. Indeed, studies from SARS and MERS-infected patients and animals also point to an important role for the T cell-mediated adaptive immune response in protection and clearance of respiratory corona virus infection. In SARS-recovered patients, neutralizing antibody titers and the memory B cell response are short-lived and only last a few months, whereas SARS-CoV-specific memory T cells persist for up to 6 years post-infection.

Direct evidence for the role of virus-specific CD4 and CD8 T cells in SARS-CoV clearance and host protection come from adoptive transfer studies. Adoptively infused SARS-Cov-specific effector CD4 and CD8 T cells in mice susceptible to SARS resulted in rapid virus clearance and improvement in the clinical disease and survival of the animals. There is a need in the art for effective coronavirus treatment and prevention.

### SUMMARY OF THE INVENTION

This disclosure provides for peptides useful for vaccination and other applications, engineered T cell Receptors (TCRs), cells comprising the peptides and TCRs, and methods of making and using the peptides and TCRs. The current disclosure relates to TCRs that specifically recognize coronavirus peptides, such as those represented by SEQ ID NOS:22-81.

Accordingly, aspects of the disclosure relate to a polypeptide comprising an antigen binding variable region comprising the amino acid sequence of a CDR3 of the disclosure or an amino acid sequence with at least 80% sequence identity to a CDR3 of the disclosure. Further aspects relate to an engineered T-cell Receptor (TCR) comprising a TCR-b polypeptide and a TCR-a polypeptide, wherein the TCR-b polypeptide comprises an amino acid sequence of a CDR3 of the disclosure or an amino acid sequence with at least 80% sequence identity to a CDR3 of the disclosure and the TCR-a polypeptide comprises the amino acid sequence of a CDR3 of the disclosure or an amino acid sequence with at least 80% sequence identity to a CDR3 of the disclosure. Further aspects relate to a nucleic acid encoding a TCR-b polypeptide comprising an amino acid sequence of a CDR3 of the disclosure or comprising an amino acid sequence with at least 80% sequence identity to a CDR3 of the disclosure and/or a TCR-a polypeptide comprising an amino acid sequence of a CDR3 of the disclosure or having the amino acid sequence with at least 80% sequence identity to a CDR3 of the disclosure. The CDR3 may be a CDR3 of SEQ ID NO:7, 14, 21, 92, 99, 106, 113, 120, 127, 134, and 141. TCR-a CDR3 aspects include CDRs with an amino acid sequence of SEQ ID NO:7, 14, 92, 106, 120, and 134. TCR-b CDR3 aspects include CDRs with an amino acid sequence of SEQ ID NO:21, 99, 113, 127, and 141.

Yet further aspects relate to nucleic acids encoding the peptides, polypeptides, fusion proteins, and engineered TCRs. The disclosure also describes nucleic acid vectors comprising one or more nucleic acids of the disclosure, and cells comprising the peptides, fusion proteins, polypeptides, engineered TCRs, and/or nucleic acids of the disclosure. Also provided are compositions comprising the peptides, polypeptides, cells, nucleic acids, or engineered TCRs of the disclosure. Further aspects relate to a method of making an engineered cell comprising transferring a nucleic acid or vector of the disclosure into a cell. Further aspects relate to a method for treating or preventing a coronovirus infection in a subject, the method comprising administering a polypeptide, composition, cell, nucleic acid, or engineered TCR to the subject. In some aspects, the method is for treating long covid, the method comprising administering a polypeptide, composition, cell, nucleic acid, or engineered TCR to the subject. Also provided is a method of stimulating an immune response in a subject, the method comprising administering a composition or cell of the disclosure to the subject. Methods also include methods of reducing tumor burden; methods of lysing a cancer cell; methods of killing tumor/cancerous cells; methods of increasing overall survival; methods of reducing the risk of getting cancer or of getting a tumor; methods of increasing recurrent free survival; methods of preventing cancer; and/or methods of reducing, eliminating, or decreasing the spread or metastasis of cancer, the method comprising administering a polypeptide, composition, cell, nucleic acid, or engineered TCR to a subject in need thereof.

Further aspects relate to a fusion protein comprising a TCR of the disclosure, or an antigen-binding fragment thereof, and a CD3 binding region. The CD3 binding region may comprise a CD3-specific fragment antigen binding (Fab), single chain variable fragment (scFv), single domain antibody, or single chain antibody. Exemplary CD3-specific fragment antigen binding (Fab) are known in the art. For example, US20180222981, which is herein incorporated by reference, discloses variable regions that bind specifically to CD3, which may be used in aspects of this disclosure. Anti-CD3 antibodies and variable regions are disclosed in US20180117152, which is also incorporated by reference.

Aspects of the disclosure relate to a peptide comprising at least 60% sequence identity to a peptide of one of SEQ ID NOS:22-81. Also provided is a peptide comprising at least 60% sequence identity to a peptide of one of SEQ ID NOS:22-38. The peptide or polypeptide may have or have at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:22-81. The peptide may have or have at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:22-38. Also provided are polypeptides comprising peptides of the disclosure. Further aspects relate to a molecular complex comprising the peptide or polypeptide of the disclosure and a MHC polypeptide. Other aspects of the disclosure relate to a method of producing peptide/coronavirus-specific immune effector cells comprising: (a) obtaining a starting population of immune effector cells; and (b) contacting the starting population of immune effector cells with a peptide of the disclosure, thereby generating peptide/coronavirus-specific immune effector cells. The disclosure also describes peptide/coronavirus-specific engineered T cells and TCRs produced according to the methods of the disclosure. Further aspects relate to an in vitro isolated dendritic cell comprising a peptide, polypeptide, nucleic acid, or expression vector of the disclosure.

Also provide is a method for prognosing a patient or for detecting T cell responses in a patient, the method comprising: contacting a biological sample from the patient with a composition, peptide, or polypeptide of the disclosure. Yet further aspects relate to a peptide/coronavirus-specific binding molecule that bind to a peptide of the disclosure or that bind to a peptide-MHC complex. Exemplary binding molecules include antibodies, TCR mimic antibodies, scFvs, nanobodies, camellids, aptamers, and DARPINs. Related methods provide for a method comprising contacting a composition comprising at least one MHC polypeptide and a peptide or polypeptide of the disclosure with a composition comprising T cells and detecting T cells with bound peptide and/or MHC polypeptide by detecting a detection tag. Further aspects relate to kits comprising a peptide, polypeptide, nucleic acid, expression vector, or composition of the disclosure. Further method aspects relate to a method of cloning a coronavirus T cell receptor (TCR), the method comprising (a) obtaining a starting population of immune effector cells; (b) contacting the starting population of immune effector cells with the coronavirus peptide of the disclosure, thereby generating coronavirus-specific immune effector cells; (c) purifying immune effector cells specific to the coronavirus peptide, and (d) isolating a TCR sequence from the purified immune effector cells. Further aspects relate to a method of making a cell comprising transferring a nucleic acid or expression vector of the disclosure into the cell. Further aspects relate to an in vitro method for making a therapeutic T cell vaccine comprising co-culturing T cells with a peptide of the disclosure. In some aspects, the method is for treating or preventing SARS. In some aspects, the method is for treating or preventing COVID-19. In some aspect, COVID-19 is further defined as long COVID.

Nucleic acids of the disclosure include those that encode for CDR regions, variable regions, engineered TCRs, polypeptides, TCR-a polypeptides, TCR-b polypeptides, peptides, polypeptides, and fusion proteins described herein. The nucleic acid may be RNA. The nucleic acid may also be DNA or a cDNA encoding the peptide or polypeptide, or a complement of the peptide or polypeptide. The nucleic acid may comprise one of SEQ ID NOS:1, 8, 15, 86, 93, 100, 107, 114, 121, 128, or 135, or a fragment thereof. In some aspects, the nucleic acid comprises a nucleotide having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:1, 8, 15, 86, 93, 100, 107, 114, 121, 128, or 135, or a fragment thereof.

The polypeptide or the TCR-a polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:7. In some aspects, the polypeptide or the TCR-a polypeptide comprises a CDR3 having the amino acid sequence of SEQ ID NO:7. The polypeptide or the TCR-a polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:14. In some aspects, the polypeptide or the TCR-a polypeptide comprises a CDR3 having the amino acid sequence of SEQ ID NO:14. The polypeptide may comprise a CDR3 comprising the amino acid sequence of one of SEQ ID NOS:7, 14, or 21. The polypeptide or the TCR-b polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:21. The polypeptide or the TCR-b polypeptide may comprise a CDR3 comprising the amino acid sequence of SEQ ID NO:21. The engineered TCR may comprise a TCR-a polypeptide comprising: (i) a CDR3 with the amino acid sequence of SEQ ID NO:7 or an amino acid sequence with at least 80% sequence identity to SEQ ID NO:7; or (ii) a CDR3 with the amino acid sequence of SEQ ID NO:14 or an amino acid sequence with at least 80% sequence identity to SEQ ID NO:14; and a TCR-b polypeptide comprising a CDR3 with the amino acid sequence of SEQ ID NO:21 or an amino acid sequence with at least 80% sequence identity to SEQ ID NO:21. The engineered TCR may comprise a TCR-a polypeptide comprising: (i) a CDR3 with the amino acid sequence of SEQ ID NO:7 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:7; or (ii) a CDR3 with the amino acid sequence of SEQ ID NO:14 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:14; and a TCR-b polypeptide comprising a CDR3 with the amino acid sequence of SEQ ID NO:21 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:21.

The polypeptide or TCR may comprise a variable region comprising a CDR1, CDR2, and/or CDR3 from a TCR-a polypeptide and/or a TCR-b polypeptide. The variable region may comprise a CDR1 with an amino acid sequence of one of SEQ ID NOS:5, 12, or 19, or with at least 80% sequence identity to one of SEQ ID NOS:5, 12, or 19. The variable region may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to sequence identity to a one of SEQ ID NOS:5, 12, or 19.

The polypeptide, TCR, or the TCR-a polypeptide may comprise a CDR1 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:5 and 12. The polypeptide, TCR, or the TCR-a polypeptide may comprise a CDR1 comprising the amino acid sequence of one of SEQ ID NOS:5 and 12. The polypeptide, TCR, or the TCR-b polypeptide may comprise a CDR1 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOS:19. The polypeptide, TCR, or the TCR-b polypeptide may comprise a CDR1 comprising the amino acid sequence of SEQ ID NO:19.

The engineered TCR may comprise a TCR-a polypeptide comprising a CDR1 with an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:5 and 12 and a TCR-b polypeptide comprising a CDR1 having an amino acid having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:19. The engineered TCR may comprise a TCR-a polypeptide comprising a CDR1 having the amino acid sequence of SEQ ID NO:5 or 12 and a TCR-b polypeptide comprising a CDR1 having the amino acid sequence of SEQ ID NO:19.

The polypeptides, TCR-a or TCR-b may comprise a variable region. The variable region may comprise a CDR2 with an amino acid sequence of one of SEQ ID NOS:6, 13, and 20, or with at least 80% sequence identity to one of SEQ ID NOS:6, 13, and 20. The variable region may comprise a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to sequence identity to a one of SEQ ID NOS:6, 13, and 20.

The polypeptide or the TCR-a polypeptide may comprise a CDR2 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:6 and 13. The polypeptide or the TCR-a polypeptide may comprise a CDR2 comprising the amino acid sequence of one of SEQ ID NOS:6 and 13. The polypeptide or the TCR-b polypeptide may comprise a CDR2 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:20. The polypeptide or the TCR-b polypeptide may comprise a CDR2 comprising the amino acid sequence of SEQ ID NO:20.

The engineered TCR may comprise a TCR-a polypeptide comprising a CDR2 with an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:6 and 13, and a TCR-b polypeptide comprising a CDR2 with an amino acid sequence that has or has at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:20. The engineered TCR may comprise a TCR-a polypeptide comprising a CDR2 having the amino acid sequence of one of SEQ ID NOS:6 and 13, and a TCR-b polypeptide comprising a CDR2 having the amino acid sequence of SEQ ID NO:20.

The variable region may comprise an amino acid sequence with at least 70% sequence identity to one of SEQ ID NOS:3, 10, and 17. The variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:3, 10, and 17. The variable region may comprise the amino acid sequence of one of SEQ ID NOS:3, 10, and 17. The TCR-a variable region may comprise an amino acid sequence with at least 70% sequence identity to one of SEQ ID NOS:3 and 10. The TCR-a variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:3 and 10. The TCR-a variable region may comprise the amino acid sequence of one of SEQ ID NOS:3 and 10. The TCR-b variable region may comprise an amino acid sequence with at least 70% sequence identity to SEQ ID NO:17. The TCR-b variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:17. The TCR-b variable region may comprise the amino acid sequence of SEQ ID NO:17.

The polypeptide may comprise a T cell receptor alpha (TCR-a) variable region. The variable region may comprise a CDR1, CDR2, and/or CDR3. The polypeptide may comprise a TCR-a variable and constant region. The polypeptide may comprise or further comprises a signal peptide. The signal peptide may comprise an amino acid sequence with at least 80% identity to one of SEQ ID NOS:4, 11, and 18. The signal peptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:4, 11, and 18. The signal peptide may comprise an amino acid sequence of one of SEQ ID NOS:4, 11, and 18.

TCR aspects of the disclosure relate to a TCR comprising a TCR-a polypeptide and a TCR-b polypeptide, wherein the CDR1, CDR2, and CDR3 of the TCR-a polypeptide comprise: (i) the amino acid sequence of SEQ ID NOS:5, 6, and 7, respectively; or (ii) the amino acid sequence of SEQ ID NOS:12, 13, and 14, respectively; and wherein the CDR1, CDR3, and CDR3 of the TCR-b polypeptide comprise the amino acid sequence of SEQ ID NO:19, 20, and 21, respectively.

The TCR-a polypeptide may comprise the amino acid sequence of SEQ ID NO:2 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:2 and the TCR-b polypeptide may comprise the amino acid sequence of SEQ ID NO:16 or an amino acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:16. The TCR-a polypeptide may comprise the amino acid sequence of SEQ ID NO:9 or an amino acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:9 and the TCR-b polypeptide comprises the amino acid sequence of SEQ ID NO:16 or an amino acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:16.

The TCR-a polypeptide may be encoded by the nucleic acid sequence of SEQ ID NO:1 or a nucleic acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:1 and the TCR-b polypeptide may be encoded by the nucleic acid sequence of SEQ ID NO:15 or a nucleic acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:15. The TCR-a polypeptide may be encoded by the nucleic acid sequence of SEQ ID NO:8 or a nucleic acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:8 and the TCR-b polypeptide may be encoded by the nucleic acid sequence of SEQ ID NO:15 or a nucleic acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:15.

The polypeptide or the TCR-a polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:92. In some aspects, the polypeptide or the TCR-a polypeptide comprises a CDR3 having the amino acid sequence of SEQ ID NO:92. The polypeptide or the TCR-b polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:99. The polypeptide or the TCR-b polypeptide may comprise a CDR3 comprising the amino acid sequence of SEQ ID NO:99. The engineered TCR may comprise a TCR-a polypeptide comprising a CDR3 with the amino acid sequence of SEQ ID NO:92 or an amino acid sequence with at least 80% sequence identity to SEQ ID NO:92 and a TCR-b polypeptide comprising a CDR3 with the amino acid sequence of SEQ ID NO:99 or an amino acid sequence with at least 80% sequence identity to SEQ ID NO:99. The engineered TCR may comprise a TCR-a polypeptide comprising a CDR3 with the amino acid sequence of SEQ ID NO:92 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:92 and a TCR-b polypeptide comprising a CDR3 with the amino acid sequence of SEQ ID NO:99 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:99.

The polypeptide or TCR may comprise a variable region comprising a CDR1, CDR2, and/or CDR3 from a TCR-a polypeptide and/or a TCR-b polypeptide. The variable region may comprise a CDR1 with an amino acid sequence of SEQ ID NOS:90 or 97, or with at least 80% sequence identity to SEQ ID NOS:90 or 97. The variable region may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to sequence identity to a one of SEQ ID NOS:90 or 97.

The polypeptide, TCR, or the TCR-a polypeptide may comprise a CDR1 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:90. The polypeptide, TCR, or the TCR-a polypeptide may comprise a CDR1 comprising the amino acid sequence of SEQ ID NO:90. The polypeptide, TCR, or the TCR-b polypeptide may comprise a CDR1 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:97. The polypeptide, TCR, or the TCR-b polypeptide may comprise a CDR1 comprising the amino acid sequence of SEQ ID NO:97.

The engineered TCR may comprise a TCR-a polypeptide comprising a CDR1 with an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:90 and a TCR-b polypeptide comprising a CDR1 having an amino acid having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:97. The engineered TCR may comprise a TCR-a polypeptide comprising a CDR1 having the amino acid sequence of SEQ ID NO:90 and a TCR-b polypeptide comprising a CDR1 having the amino acid sequence of SEQ ID NO:97.

The polypeptides, TCR-a or TCR-b may comprise a variable region. The variable region may comprise a CDR2 with an amino acid sequence of one of SEQ ID NOS:91 or 98, or with at least 80% sequence identity to one of SEQ ID NOS:91 or 98. The variable region may comprise a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to sequence identity to a one of SEQ ID NOS:91 or 98.

The polypeptide or the TCR-a polypeptide may comprise a CDR2 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:91. The polypeptide or the TCR-a polypeptide may comprise a CDR2 comprising the amino acid sequence of SEQ ID NOS:91. The polypeptide or the TCR-b polypeptide may comprise a CDR2 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:98. The polypeptide or the TCR-b polypeptide may comprise a CDR2 comprising the amino acid sequence of SEQ ID NO:98.

The engineered TCR may comprise a TCR-a polypeptide comprising a CDR2 with an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:91, and a TCR-b polypeptide comprising a CDR2 with an amino acid sequence that has or has at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:98. The engineered TCR may comprise a TCR-a polypeptide comprising a CDR2 having the amino acid sequence of SEQ ID NO:91, and a TCR-b polypeptide comprising a CDR2 having the amino acid sequence of SEQ ID NO:98.

The variable region may comprise an amino acid sequence with at least 70% sequence identity to SEQ ID NO:88 or 95. The variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:88 or 95. The variable region may comprise the amino acid sequence of SEQ ID NO:88 or 95. The TCR-a variable region may comprise an amino acid sequence with at least 70% sequence identity to SEQ ID NO:88. The TCR-a variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:88. The TCR-a variable region may comprise the amino acid sequence of SEQ ID NO:88. The TCR-b variable region may comprise an amino acid sequence with at least 70% sequence identity to SEQ ID NO:95. The TCR-b variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:95. The TCR-b variable region may comprise the amino acid sequence of SEQ ID NO:95.

The polypeptide may comprise a T cell receptor alpha (TCR-a) variable region. The variable region may comprise a CDR1, CDR2, and/or CDR3. The polypeptide may comprise a TCR-a variable and constant region. The polypeptide may comprise or further comprises a signal peptide. The signal peptide may comprise an amino acid sequence with at least 80% identity to one of SEQ ID NOS:89 and 96. The signal peptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:89 and 96. The signal peptide may comprise an amino acid sequence of one of SEQ ID NOS:89 and 96.

TCR aspects of the disclosure relate to a TCR comprising a TCR-a polypeptide and a TCR-b polypeptide, wherein the CDR1, CDR2, and CDR3 of the TCR-a polypeptide comprise the amino acid sequences of SEQ ID NOS:90, 91, and 92, respectively; and wherein the CDR1, CDR3, and CDR3 of the TCR-b polypeptide comprise the amino acid sequences of SEQ ID NOS:97, 98, and 99, respectively.

The TCR-a polypeptide may comprise the amino acid sequence of SEQ ID NO:87 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:87 and the TCR-b polypeptide may comprise the amino acid sequence of SEQ ID NO:94 or an amino acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:94.

The TCR-a polypeptide may be encoded by the nucleic acid sequence of SEQ ID NO:86 or a nucleic acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:86 and the TCR-b polypeptide may be encoded by the nucleic acid sequence of SEQ ID NO:93 or a nucleic acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:93.

The polypeptide or the TCR-a polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:106. In some aspects, the polypeptide or the TCR-a polypeptide comprises a CDR3 having the amino acid sequence of SEQ ID NO:106. The polypeptide or the TCR-b polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:113. The polypeptide or the TCR-b polypeptide may comprise a CDR3 comprising the amino acid sequence of SEQ ID NO:113. The engineered TCR may comprise a TCR-a polypeptide comprising a CDR3 with the amino acid sequence of SEQ ID NO:106 or an amino acid sequence with at least 80% sequence identity to SEQ ID NO:106 and a TCR-b polypeptide comprising a CDR3 with the amino acid sequence of SEQ ID NO:113 or an amino acid sequence with at least 80% sequence identity to SEQ ID NO:113. The engineered TCR may comprise a TCR-a polypeptide comprising a CDR3 with the amino acid sequence of SEQ ID NO:106 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:106 and a TCR-b polypeptide comprising a CDR3 with the amino acid sequence of SEQ ID NO:113 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:113.

The polypeptide or TCR may comprise a variable region comprising a CDR1, CDR2, and/or CDR3 from a TCR-a polypeptide and/or a TCR-b polypeptide. The variable region may comprise a CDR1 with an amino acid sequence of SEQ ID NO:104 or 111, or with at least 80% sequence identity to SEQ ID NO:104 or 111. The variable region may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to sequence identity to SEQ ID NO:104 or 111.

The polypeptide, TCR, or the TCR-a polypeptide may comprise a CDR1 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:104. The polypeptide, TCR, or the TCR-a polypeptide may comprise a CDR1 comprising the amino acid sequence of SEQ ID NO:104. The polypeptide, TCR, or the TCR-b polypeptide may comprise a CDR1 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:111. The polypeptide, TCR, or the TCR-b polypeptide may comprise a CDR1 comprising the amino acid sequence of SEQ ID NO:111.

The engineered TCR may comprise a TCR-a polypeptide comprising a CDR1 with an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:104 and a TCR-b polypeptide comprising a CDR1 having an amino acid having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:111. The engineered TCR may comprise a TCR-a polypeptide comprising a CDR1 having the amino acid sequence of SEQ ID NO:104 and a TCR-b polypeptide comprising a CDR1 having the amino acid sequence of SEQ ID NO:111.

The polypeptides, TCR-a or TCR-b may comprise a variable region. The variable region may comprise a CDR2 with an amino acid sequence of SEQ ID NO:105 or 112, or with at least 80% sequence identity to SEQ ID NO:105 or 112. The variable region may comprise a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to sequence identity to SEQ ID NO:105 or 112.

The polypeptide or the TCR-a polypeptide may comprise a CDR2 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:105. The polypeptide or the TCR-a polypeptide may comprise a CDR2 comprising the amino acid sequence of SEQ ID NOS:105. The polypeptide or the TCR-b polypeptide may comprise a CDR2 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:112. The polypeptide or the TCR-b polypeptide may comprise a CDR2 comprising the amino acid sequence of SEQ ID NO:112.

The engineered TCR may comprise a TCR-a polypeptide comprising a CDR2 with an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:105, and a TCR-b polypeptide comprising a CDR2 with an amino acid sequence that has or has at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:112. The engineered TCR may comprise a TCR-a polypeptide comprising a CDR2 having the amino acid sequence of SEQ ID NO:105, and a TCR-b polypeptide comprising a CDR2 having the amino acid sequence of SEQ ID NO:112.

The variable region may comprise an amino acid sequence with at least 70% sequence identity to SEQ ID NO:102 or 109. The variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:102 or 109. The variable region may comprise the amino acid sequence of SEQ ID NO:102 or 109. The TCR-a variable region may comprise an amino acid sequence with at least 70% sequence identity to SEQ ID NO:102. The TCR-a variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:102. The TCR-a variable region may comprise the amino acid sequence of SEQ ID NO:102. The TCR-b variable region may comprise an amino acid sequence with at least 70% sequence identity to SEQ ID NO:109. The TCR-b variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:109. The TCR-b variable region may comprise the amino acid sequence of SEQ ID NO:109.

The polypeptide may comprise a T cell receptor alpha (TCR-a) variable region. The variable region may comprise a CDR1, CDR2, and/or CDR3. The polypeptide may comprise a TCR-a variable and constant region. The polypeptide may comprise or further comprises a signal peptide. The signal peptide may comprise an amino acid sequence with at least 80% identity to one of SEQ ID NOS:103 and 110. The signal peptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:103 and 110. The signal peptide may comprise an amino acid sequence of one of SEQ ID NOS:103 and 110.

TCR aspects of the disclosure relate to a TCR comprising a TCR-a polypeptide and a TCR-b polypeptide, wherein the CDR1, CDR2, and CDR3 of the TCR-a polypeptide comprise the amino acid sequences of SEQ ID NOS:104, 105, and 106, respectively; and wherein the CDR1, CDR3, and CDR3 of the TCR-b polypeptide comprise the amino acid sequences of SEQ ID NOS:111, 112, and 113, respectively.

The TCR-a polypeptide may comprise the amino acid sequence of SEQ ID NO:101 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:101 and the TCR-b polypeptide may comprise the amino acid sequence of SEQ ID NO:108 or an amino acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:108.

The TCR-a polypeptide may be encoded by the nucleic acid sequence of SEQ ID NO:100 or a nucleic acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:100 and the TCR-b polypeptide may be encoded by the nucleic acid sequence of SEQ ID NO:107 or a nucleic acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:107.

The polypeptide or the TCR-a polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:120. In some aspects, the polypeptide or the TCR-a polypeptide comprises a CDR3 having the amino acid sequence of SEQ ID NO:120. The polypeptide or the TCR-b polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:127. The polypeptide or the TCR-b polypeptide may comprise a CDR3 comprising the amino acid sequence of SEQ ID NO:127. The engineered TCR may comprise a TCR-a polypeptide comprising a CDR3 with the amino acid sequence of SEQ ID NO:120 or an amino acid sequence with at least 80% sequence identity to SEQ ID NO:120 and a TCR-b polypeptide comprising a CDR3 with the amino acid sequence of SEQ ID NO:127 or an amino acid sequence with at least 80% sequence identity to SEQ ID NO:127. The engineered TCR may comprise a TCR-a polypeptide comprising a CDR3 with the amino acid sequence of SEQ ID NO:120 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:120 and a TCR-b polypeptide comprising a CDR3 with the amino acid sequence of SEQ ID NO:127 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:127.

The polypeptide or TCR may comprise a variable region comprising a CDR1, CDR2, and/or CDR3 from a TCR-a polypeptide and/or a TCR-b polypeptide. The variable region may comprise a CDR1 with an amino acid sequence of SEQ ID NO:118 or 125, or with at least 80% sequence identity to SEQ ID NO:118 or 125. The variable region may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to sequence identity to SEQ ID NO:118 or 125.

The polypeptide, TCR, or the TCR-a polypeptide may comprise a CDR1 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:118. The polypeptide, TCR, or the TCR-a polypeptide may comprise a CDR1 comprising the amino acid sequence of SEQ ID NO:118. The polypeptide, TCR, or the TCR-b polypeptide may comprise a CDR1 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:125. The polypeptide, TCR, or the TCR-b polypeptide may comprise a CDR1 comprising the amino acid sequence of SEQ ID NO:125.

The engineered TCR may comprise a TCR-a polypeptide comprising a CDR1 with an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:118 and a TCR-b polypeptide comprising a CDR1 having an amino acid having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:125. The engineered TCR may comprise a TCR-a polypeptide comprising a CDR1 having the amino acid sequence of SEQ ID NO:118 and a TCR-b polypeptide comprising a CDR1 having the amino acid sequence of SEQ ID NO:125.

The polypeptides, TCR-a or TCR-b may comprise a variable region. The variable region may comprise a CDR2 with an amino acid sequence of SEQ ID NO:119 or 126, or with at least 80% sequence identity to SEQ ID NO:119 or 126. The variable region may comprise a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to sequence identity to SEQ ID NO:119 or 126.

The polypeptide or the TCR-a polypeptide may comprise a CDR2 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:119. The polypeptide or the TCR-a polypeptide may comprise a CDR2 comprising the amino acid sequence of SEQ ID NOS:119. The polypeptide or the TCR-b polypeptide may comprise a CDR2 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:126. The polypeptide or the TCR-b polypeptide may comprise a CDR2 comprising the amino acid sequence of SEQ ID NO:126.

The engineered TCR may comprise a TCR-a polypeptide comprising a CDR2 with an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:119, and a TCR-b polypeptide comprising a CDR2 with an amino acid sequence that has or has at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:126. The engineered TCR may comprise a TCR-a polypeptide comprising a CDR2 having the amino acid sequence of SEQ ID NO:119, and a TCR-b polypeptide comprising a CDR2 having the amino acid sequence of SEQ ID NO:126.

The variable region may comprise an amino acid sequence with at least 70% sequence identity to SEQ ID NO:116 or 123. The variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:116 or 123. The variable region may comprise the amino acid sequence of SEQ ID NO:116 or 123. The TCR-a variable region may comprise an amino acid sequence with at least 70% sequence identity to SEQ ID NO:116. The TCR-a variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:116. The TCR-a variable region may comprise the amino acid sequence of SEQ ID NO:116. The TCR-b variable region may comprise an amino acid sequence with at least 70% sequence identity to SEQ ID NO:123. The TCR-b variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:123. The TCR-b variable region may comprise the amino acid sequence of SEQ ID NO:123.

The polypeptide may comprise a T cell receptor alpha (TCR-a) variable region. The variable region may comprise a CDR1, CDR2, and/or CDR3. The polypeptide may comprise a TCR-a variable and constant region. The polypeptide may comprise or further comprises a signal peptide. The signal peptide may comprise an amino acid sequence with at least 80% identity to one of SEQ ID NOS:117 and 124. The signal peptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:117 and 124. The signal peptide may comprise an amino acid sequence of one of SEQ ID NOS:117 and 124.

TCR aspects of the disclosure relate to a TCR comprising a TCR-a polypeptide and a TCR-b polypeptide, wherein the CDR1, CDR2, and CDR3 of the TCR-a polypeptide comprise the amino acid sequences of SEQ ID NOS:118, 119, and 120, respectively; and wherein the CDR1, CDR3, and CDR3 of the TCR-b polypeptide comprise the amino acid sequences of SEQ ID NOS:125, 126, and 127, respectively.

The TCR-a polypeptide may comprise the amino acid sequence of SEQ ID NO:115 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:115 and the TCR-b polypeptide may comprise the amino acid sequence of SEQ ID NO:122 or an amino acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:122.

The TCR-a polypeptide may be encoded by the nucleic acid sequence of SEQ ID NO:114 or a nucleic acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:114 and the TCR-b polypeptide may be encoded by the nucleic acid sequence of SEQ ID NO:121 or a nucleic acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:121.

The polypeptide or the TCR-a polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:134. In some aspects, the polypeptide or the TCR-a polypeptide comprises a CDR3 having the amino acid sequence of SEQ ID NO:134. The polypeptide or the TCR-b polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:141. The polypeptide or the TCR-b polypeptide may comprise a CDR3 comprising the amino acid sequence of SEQ ID NO:141. The engineered TCR may comprise a TCR-a polypeptide comprising a CDR3 with the amino acid sequence of SEQ ID NO:134 or an amino acid sequence with at least 80% sequence identity to SEQ ID NO:134 and a TCR-b polypeptide comprising a CDR3 with the amino acid sequence of SEQ ID NO:141 or an amino acid sequence with at least 80% sequence identity to SEQ ID NO:141. The engineered TCR may comprise a TCR-a polypeptide comprising a CDR3 with the amino acid sequence of SEQ ID NO:134 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:134 and a TCR-b polypeptide comprising a CDR3 with the amino acid sequence of SEQ ID NO:141 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:141.

The polypeptide or TCR may comprise a variable region comprising a CDR1, CDR2, and/or CDR3 from a TCR-a polypeptide and/or a TCR-b polypeptide. The variable region may comprise a CDR1 with an amino acid sequence of SEQ ID NO:132 or 139, or with at least 80% sequence identity to SEQ ID NO:132 or 139. The variable region may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to sequence identity to SEQ ID NO:132 or 139.

The polypeptide, TCR, or the TCR-a polypeptide may comprise a CDR1 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:132. The polypeptide, TCR, or the TCR-a polypeptide may comprise a CDR1 comprising the amino acid sequence of SEQ ID NO:132. The polypeptide, TCR, or the TCR-b polypeptide may comprise a CDR1 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:139. The polypeptide, TCR, or the TCR-b polypeptide may comprise a CDR1 comprising the amino acid sequence of SEQ ID NO:139.

The engineered TCR may comprise a TCR-a polypeptide comprising a CDR1 with an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:132 and a TCR-b polypeptide comprising a CDR1 having an amino acid having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:139. The engineered TCR may comprise a TCR-a polypeptide comprising a CDR1 having the amino acid sequence of SEQ ID NO:132 and a TCR-b polypeptide comprising a CDR1 having the amino acid sequence of SEQ ID NO:139.

The polypeptides, TCR-a or TCR-b may comprise a variable region. The variable region may comprise a CDR2 with an amino acid sequence of SEQ ID NO:133 or 140, or with at least 80% sequence identity to SEQ ID NO:133 or 140. The variable region may comprise a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to sequence identity to SEQ ID NO:133 or 140.

The polypeptide or the TCR-a polypeptide may comprise a CDR2 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:133. The polypeptide or the TCR-a polypeptide may comprise a CDR2 comprising the amino acid sequence of SEQ ID NOS:133. The polypeptide or the TCR-b polypeptide may comprise a CDR2 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:140. The polypeptide or the TCR-b polypeptide may comprise a CDR2 comprising the amino acid sequence of SEQ ID NO:140.

The engineered TCR may comprise a TCR-a polypeptide comprising a CDR2 with an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:133, and a TCR-b polypeptide comprising a CDR2 with an amino acid sequence that has or has at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:140. The engineered TCR may comprise a TCR-a polypeptide comprising a CDR2 having the amino acid sequence of SEQ ID NO:133, and a TCR-b polypeptide comprising a CDR2 having the amino acid sequence of SEQ ID NO:140.

The variable region may comprise an amino acid sequence with at least 70% sequence identity to SEQ ID NO:130 or 137. The variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:130 or 137. The variable region may comprise the amino acid sequence of SEQ ID NO:130 or 137. The TCR-a variable region may comprise an amino acid sequence with at least 70% sequence identity to SEQ ID NO:130. The TCR-a variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:130. The TCR-a variable region may comprise the amino acid sequence of SEQ ID NO:130. The TCR-b variable region may comprise an amino acid sequence with at least 70% sequence identity to SEQ ID NO:137. The TCR-b variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:137. The TCR-b variable region may comprise the amino acid sequence of SEQ ID NO:137.

The polypeptide may comprise a T cell receptor alpha (TCR-a) variable region. The variable region may comprise a CDR1, CDR2, and/or CDR3. The polypeptide may comprise a TCR-a variable and constant region. The polypeptide may comprise or further comprises a signal peptide. The signal peptide may comprise an amino acid sequence with at least 80% identity to one of SEQ ID NOS:131 and 138. The signal peptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:131 and 138. The signal peptide may comprise an amino acid sequence of one of SEQ ID NOS:131 and 138.

TCR aspects of the disclosure relate to a TCR comprising a TCR-a polypeptide and a TCR-b polypeptide, wherein the CDR1, CDR2, and CDR3 of the TCR-a polypeptide comprise the amino acid sequences of SEQ ID NOS:132, 133, and 134, respectively; and wherein the CDR1, CDR3, and CDR3 of the TCR-b polypeptide comprise the amino acid sequences of SEQ ID NOS:1139, 140, and 141, respectively.

The TCR-a polypeptide may comprise the amino acid sequence of SEQ ID NO:129 or an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:129 and the TCR-b polypeptide may comprise the amino acid sequence of SEQ ID NO:136 or an amino acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:136.

The TCR-a polypeptide may be encoded by the nucleic acid sequence of SEQ ID NO:128 or a nucleic acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:128 and the TCR-b polypeptide may be encoded by the nucleic acid sequence of SEQ ID NO:135 or a nucleic acid sequence with or with at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:135.

The TCR may comprise a modification or may be chimeric. The variable region of the TCR may be fused to a TCR constant region that is different from the constant region of the cloned TCR that specifically binds to a peptide of the disclosure.

The TCR-a polypeptide and TCR-b polypeptide may be operably linked. The term “operably linked” can refer to a covalent linkage, such as a peptide bond (e.g., the two elements are polypeptides and are on the same polypeptide), or a non-covalent linkage, such as Van der Waals force (e.g. two polypeptides that have a certain degree of specific binding affinity for each other). The TCR-a polypeptide and TCR-b polypeptide may be operably linked through a peptide bond. The TCR-a polypeptide and TCR-b polypeptide may be on the same polypeptide and wherein the TCR-b is amino-proximal to the TCR-a. The TCR-a polypeptide and TCR-b polypeptide may be on the same polypeptide and wherein the TCR-a is amino-proximal to the TCR-b. According, the TCR may be a single chain TCR. The single chain TCR may comprise or further comprise a linker between the TCR-a and TCR-b polypeptide. The linker may be a linker described herein or known in the art. The linker may also comprise or consist of glycine and serine residues. In some aspects, the linker is composed of only glycine and serine residues (a glycine-serine linker). The linker may be a flexible linker. Exemplary flexible linkers include glycine polymers (G)n, glycine-serine polymers (including, for example, (GS)n, (GSGGS)n, (G₄S)n and (GGGS)n, where n is an integer of at least one. In some aspects, n is at least, at most, or exactly 1, 2, 3, 4, 5, 6, 7, 8, 9, or 10 (or any derivable range therein). Glycine-alanine polymers, alanine-serine polymers, and other flexible linkers known in the art and may be used as a linker in the polypeptides of the disclosure. Exemplary linkers can comprise or consist of GGSG, GGSGG, GSGSG, GSGGG, GGGSG, GSSSG, and the like. A first region is carboxy-proximal to a second region when the first region is attached to the carboxy terminus of the second region. There may be further intervening amino acid residues between the first and second regions. Thus, the regions need not be immediately adjacent, unless specifically specified as not having intervening amino acid residues. The term “amino-proximal” is similarly defined in that a first region is amino-proximal to a second region when the first region is attached to the amino terminus of the second region. Similarly, there may be further intervening amino acid residues between the first and second regions unless stated otherwise.

A CDR may also comprise 1, 2, 3, 4, 5, 6, 7, 8, 9, 10, 11, 12, 13, 14, 15, 16, 16, 18, 19, 20, 21, 22, 23, or more contiguous amino acid residues (or any range derivable therein) flanking one or both sides of a particular CDR sequence in the context of the variable region of the TCR-a or TCR-b polypeptide; therefore, there may be one or more additional amino acids at the N-terminal or C-terminal end of a particular CDR sequence, such as those shown in the variable regions of SEQ ID NOS:3, 10, and 17. Alternatively, or in combination, a CDR may also be a fragment of a CDR described herein and may lack at least 1, 2, 3, 4, or 5 amino acids from the C-terminal or N-terminal end of a particular CDR sequence.

The TCR or fusion protein may be conjugated to a detection or therapeutic agent. The agent may comprise a fluorescent molecule, radiative molecule, or toxin. The TCR or fusion protein may be conjugated to an agent described herein.

The peptide may comprise at least 6 contiguous amino acids of a peptide of one of SEQ ID NOS:22-81. The peptide may comprise at least 4, 5, 6, 7, 8, 9, 10, 11, 12, or 13 contiguous amino acids of a peptide of one of SEQ ID NOS:22-81. The peptide may comprise or consist of one of SEQ ID NOS:22-81. The peptide may comprise or consist of one of SEQ ID NOS:22-38. In some aspects, the peptide is 13 amino acids in length or shorter. The peptide may have at least, at most, exactly, or consist of 7, 8, 9, 10, 11, 12, 13, 14, or 15 amino acids (or any range derivable therein). In a specific aspect, the peptide consists of 9 amino acids. The peptide may consist of 10 amino acids. The peptide may consist of 11 amino acids. The peptide may consist of 12 amino acids. The peptide may consist of 13 amino acids. In some aspects, the peptide is immunogenic. The term immunogenic may refer to the production of an immune response, such as a protective immune response. The peptide may be modified. The modification may comprise conjugation to a molecule. The molecule may be an antibody, a lipid, an adjuvant, or a detection moiety (tag). The peptide may comprise 100% sequence identity to a peptide of one of SEQ ID NOS:22-81. Peptides of the disclosure also include those that have or have at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to a peptide of one of SEQ ID NOS:22-81. The peptide may comprise at least 63% identity to a peptide of one of SEQ ID NOS:22-81. The peptide may comprise at least 66% identity to a peptide of one of SEQ ID NOS:22-81. The peptide may comprise at least 70% identity to a peptide of one of SEQ ID NOS:22-81. The peptide may comprise at least 72% identity to a peptide of one of SEQ ID NOS:22-81. The peptide may comprise at least 77% identity to a peptide of one of SEQ ID NOS:22-81. The peptide may comprise at least 80% identity to a peptide of one of SEQ ID NOS:22-81. The peptide may comprise at least 80% identity to a peptide of one of SEQ ID NOS:22-81. The peptide may comprise at least 81% identity to a peptide of one of SEQ ID NOS:22-81. The peptide may comprise at least 88% identity to a peptide of one of SEQ ID NOS:22-81. The peptide may comprise at least 90% identity to a peptide of one of SEQ ID NOS:22-81. The peptides of the disclosure may have 1, 2, or 3 substitutions relative to a peptide of one of SEQ ID NOS:18-21. The peptide may have at least or have at most 1, 2, 3, 4, or 5 substitutions relative to a peptide of one of SEQ ID NOS:22-81.

The pharmaceutical composition may be formulated for parenteral administration, intravenous injection, intramuscular injection, inhalation, or subcutaneous injection. The peptide may be comprised in a liposome, lipid-containing nanoparticle, or in a lipid-based carrier. The pharmaceutical preparation may be formulated for injection or inhalation as a nasal spray. In some aspects, the compositions of the disclosure are formulated as a vaccine. The composition may further comprise an adjuvant.

In some aspects regarding the dendritic cells of the disclosure, the dendritic cell comprises a mature dendritic cell. The cell may be a cell with an HLA-A type. The HLA may be a HLA-A, HLA-B, or HLA-C. In some aspects, the cell is an HLA-A24 type. In some aspects, the cell is an HLA-A11 type. In some aspects, the cell is an HLA-A01, HLA-A02, HLA-A11, HLA-A24, HLA-B07, HLA-B08, HLA-B15, or HLA-B40.

The method may further comprise isolating the expressed peptide or polypeptide. In some aspects, the T cell comprises a CD8+ T cell. In some aspects, the T cell is a CD4+ T cell, a Th1, Th2, Th17, Th9, or Tfh T cell, a cytotoxic T cell, a memory T cell, a central memory T cell, or an effector memory T cell.

In method aspects of the disclosure, contacting is further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface. In some aspects, the APCs are dendritic cells.

In aspects of the disclosure, the immune effector cells are T cells, peripheral blood lymphocytes, natural killer (NK) cells, invariant NK cells, or NKT cells. The immune effector cells may be ones that have been differentiated from mesenchymal stem cell (MSC) or induced pluripotent stem (iPS) cells. The T cell aspects include T cells that are further defined as CD8⁺ T cells, CD4⁺ T cells, or γδ T cells. In certain aspects, the T cells are cytotoxic T lymphocytes (CTLs). In some aspects, obtaining comprises isolating the starting population of immune effector cells from peripheral blood mononuclear cells (PBMCs).

The nucleic acid may comprise a TCR-a (TRA) and TCR-b (TRB) gene. The nucleic acid may be polycistronic. The nucleic acid may comprise an internal ribosome entry site (IRES) or a 2A cleavable linker, such as a P2A linker. The nucleic acid may comprise a cDNA encoding the TCR-a and/or TCR-b genes. The nucleic acid may further encode for a polypeptide comprising a CD3 binding region. The CD3 binding region may comprise a CD3-specific fragment antigen binding (Fab), single chain variable fragment (scFv), single domain antibody, or single chain antibody.

The vector may comprise both of the TCR-a and TCR-b genes. The vector may comprise a promoter that directs the expression of the nucleic acid. The promoter may comprise a murine stem cell virus (MSCV) promoter. The cell may comprise a stem cell, a progenitor cell, an immune cell, or a natural killer (NK) cell. The cell may comprise a hematopoietic stem or progenitor cell, a T cell, a cell differentiated from mesenchymal stem cells (MSCs) or an induced pluripotent stem cell (iPSC). The cell may be isolated or derived from peripheral blood mononuclear cell (PBMCs). The T cell may comprise a cytotoxic T lymphocyte (CTL), a CD8+ T cell, a CD4+ T cell, an invariant NK T (iNKT) cell, a gamma-delta T cell, a NKT cell, or a regulatory T cell. The cell may be isolated from a patient that has a current or prior coronavirus infection. In some aspects, the cell is isolated from a healthy subject not having a current or past coronavirus infection. In some aspects, the cell is isolated from a healthy patient. The cell may be frozen or may have never been frozen. The cell may be a cell that is in cell culture. In some aspects, the cell lacks endogenous expression of TCR genes. The cell may further comprise a chimeric antigen receptor (CAR).

The composition may be one that has been determined to be serum-free, mycoplasma-free, endotoxin-free, and/or sterile. The method may further comprise culturing the cell in media, incubating the cell at conditions that allow for the division of the cell, screening the cell, and/or freezing the cell.

In aspects of the disclosure, the coronavirus may refer to a coronavirus isolated from bats. In some aspects of the disclosure, the coronavirus is SARS-CoV, which is the virus that causes SARS in humans. In some aspects, the coronavirus is SARS-CoV-2, wherein is the virus that causes COVID-19 in humans. In some aspects, the method is for treating or preventing SARS. In some aspects, the method is for treating or preventing COVID-19. In some aspects, the coronavirus is a coronavirus that expresses a polypeptide comprising one of SEQ ID NO:22-81. The subject may comprise a laboratory test animal, such as a mouse, rat, rabbit, dog, cat, horse, or pig. In some aspects, the subject is a human. The subject may be one that has one or more symptoms of a coronavirus infection. In some aspects, the subject does not have any symptoms of a coronavirus infection. The subject may be one that has and/or has been diagnosed with a coronavirus infection. In some aspects, the subject does not have and/or has not been diagnosed with a coronavirus infection. In some aspects, the subject has been previously treated for a coronavirus infection. The subject may be one that has been determined to be resistant or non-responsive to the previous treatment. In some aspects, the subject is administered an additional therapeutic. In some aspects, the additional therapeutic comprises a steroid or an anti-viral therapeutic. In some aspects, the additional therapeutic comprises dexamethasone, monoclonal antibodies, remdesivir, Paxlovid, Molnupiravir, convalescent plasma, or combinations thereof.

In some aspects, the subject has and/or has been diagnosed with SARS. In some aspects, the subject has been diagnosed with complications relating to a coronavirus infection. In some aspects, the subject has been diagnosed with complications relating to COVID-19 or SARS. The complication may include pneumonia, difficulty breathing or shortness of breath, chest pain or chest pressure, acute respiratory failure, acute respiratory distress syndrome, acute cardiac injury, secondary infection, acute kidney injury, septic shock, blood clots, multisystem inflammatory syndrome, chronic fatigue, rhabdomyolysis, disseminated intravascular coagulation, or acute liver injury. In some aspects, the subject is vaccinated against a coronavirus. In some aspects, the subject is vaccinated against SARS-CoV-2 or SARS-CoV. The subject may also be one that is unvaccinated for a coronavirus. In some aspects, the subject has been diagnosed with long covid. In some aspects, the patient is immunocompromised or immunosuppressed.

The compositions of the disclosure may be formulated as a vaccine. The compositions and methods of the disclosure provide for prophylactic therapies to prevent COVID-19 or a SARS-Cov-2 infection. The composition may further comprise an adjuvant. Adjuvants are known in the art and include, for example, TLR agonists and aluminum salts.

The methods of the disclosure may further comprise screening the cell for one or more cellular properties, such as for TCR expression, incorporation of nucleic acids encoding TCR genes, or for immunogenic properties, such as binding of the TCR to an antigen.

The method may comprise administering a cell or a composition comprising a cell and wherein the cell comprises an autologous cell. In some aspects, the cell comprises a non-autologous cell. The cell may also be defined as an allogenic or xenogenic cell.

The biological sample in methods of the disclosure may comprise a blood sample or a fraction thereof. In some aspects, the biological sample comprises lymphocytes. In some aspects, the biological sample comprises a fractionated sample comprising lymphocytes. The biological sample may also be one described herein.

The compositions may comprise a MHC polypeptide and a peptide of the disclosure and wherein the MHC polypeptide and/or peptide is conjugated to a detection tag. As such, suitable detection tags include, but are not limited to radioisotopes, fluorochromes, chemiluminescent compounds, dyes, and proteins, including enzymes. The tag may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent tags that produce signals include, but are not limited to bioluminescence and chemiluminescence. Examples of suitable fluorescent tags include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th). Detection tags also include streptavidin or its binding partner, biotin.

The MHC polypeptide and peptide may be operatively linked. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. Accordingly, aspects of the disclosure relate to wherein the MHC polypeptide and peptide are operatively linked through a peptide bond. Further aspects relate to wherein the MHC polypeptide and peptide are operatively linked through van der Waals forces. The peptide-MHC may be operatively linked to form a pMHC complex. In some aspects, at least two pMHC complexes are operatively linked together. Other aspects include, include at least, or include at most 2, 3, 4, 5, 6, 7, 8, 9, or 10 pMHC complexes operatively linked to each other. In some aspects, at least two MHC polypeptides are linked to one peptide. In other aspects, the average ratio of MHC polypeptides to peptides is 1:1 to 4:1. In some aspects, the ratio or average ratio is at least, at most, or about 1, 2, 3, 4, 5, or 6 to about 1, 2, 3, 4, 5, or 6 (or any derivable range therein).

In some of the aspects of the disclosure, the peptide is complexed with MHC In some aspects, the MHC comprises HLA-A type. The MHC may be further defined as HLA-A3 or HLA-A11 type. The peptides may be loaded onto dendritic cells, lymphoblastoid cells, peripheral blood mononuclear cells (PBMCs), artificial antigen presentation cells (aAPC) or artificial antigen presenting surfaces. In some aspects, the artificial antigen presenting surface comprises a MHC polypeptide conjugated or linked to a surface. Exemplary surfaces include a bead, microplate, glass slide, or cell culture plate.

Method of the disclosure may further comprise counting the number of T cells bound with peptide and/or MHC. The composition comprising T cells may be isolated from a subject. The subject may be one, as defined herein, such as a human subject. The method may further comprise sorting the number of T cells bound with peptide and/or MHC. Methods of the disclosure may also comprise or further comprise sequencing one or more TCR genes from T cells bound with peptide and/or MHC. In some aspects, the method comprises or further comprises sequencing the TCR alpha and/or beta gene(s) from a TCR, such as a TCR that binds to a peptide of the disclosure. Methods may also comprise or further comprise grouping of lymphocyte interactions by paratope hotspots (GLIPH) analysis. This is further described in Glanville et al., Nature. 2017 Jul. 6; 547(7661): 94-98, which is herein incorporated by reference.

The compositions of the disclosure may be serum-free, mycoplasma-free, endotoxin-free, and/or sterile. The methods may further comprise culturing cells of the disclosure in media, incubating the cells at conditions that allow for the division of the cell, screening the cells, and/or freezing the cells. The methods may also further comprise isolating the expressed peptide or polypeptide from a cell of the disclosure.

Methods of the disclosure may comprise or further comprise screening the dendritic cell for one or more cellular properties. In some aspects, the method further comprises contacting the cell with one or more cytokines or growth factors. The one or more cytokines or growth factors may comprise GM-CSF. In some aspects, the cellular property comprises cell surface expression of one or more of CD86, HLA, and CD14. The dendritic cell may be derived from a CD34+ hematopoietic stem or progenitor cell.

The contacting in the methods of the disclosure may be further defined as co-culturing the starting population of immune effector cells with antigen presenting cells (APCs), wherein the APCs present the peptide on their surface. In a particular aspect, the APCs are dendritic cells. In some aspects, the dendritic cell is derived from a peripheral blood monocyte (PBMC). In some aspects, the dendritic cells are isolated from PBMCs The dendritic cells or the cells in which the DCs are derived from are isolated by leukaphereses.

Peptide-MHC (pMHC) complexes in the aspects of the disclosure may be made by contacting a peptide of the disclosure with a MHC complex. In some aspects, the peptide is expressed in the cell and binds to endogenous MHC complex to form a pMHC. In some aspects, peptide exchange is used to make the pMHC complex. For example, cleavable peptides, such as photocleavable peptides may be designed that bind to and stabilize the MHC. Cleavage of the peptide (eg. by irradiation for photocleavable peptides) dissociates the peptide from the HLA complex and results in an empty HLA complex that disintegrates rapidly, unless UV exposure is performed in the presence of a “rescue peptide.” Thus, the peptides of the disclosure may be used as “rescue peptides” in the peptide exchange procedure. Further aspects of the disclosure relate to pMHC complexes comprising a peptide of the disclosure. The pMHC complex may be operatively linked to a solid support or may be attached to a detectable moiety, such as a fluorescent molecule, a radioisotope, or an antibody. Further aspects of the disclosure relate to peptide-MHC multimeric complexes that include, include at least or include at most 1, 2, 3, 4, 5, or 6 peptide-MHC molecules operatively linked together. The linkage may be covalent, such as through a peptide bond, or non-covalent. In some aspects, pMHC molecules may be bound to a biotin molecule. Such pMHC molecules may be multimerized through binding to a streptavidin molecule. pMHC multimers may be used to detect antigen-specific T cells or TCR molecules that are in a composition or in a tissue. The multimers may be used to detect peptide- or coronavirus-specific T cells in situ or in a biopsy sample. Multimers may be bound to a solid support or deposited on a solid support, such as an array or slide. Cells may then be added to the slide, and detection of the binding between the pMHC multimer and cell may be conducted. Accordingly, the pMHC molecules and multimers of the disclosure may be used to detect and diagnose a SARS-Cov-2 infection in subjects or to determine immune responses in individuals with COVID-19.

Obtaining, as defined in the methods described herein, may comprise or further comprise isolating the starting population of immune effector cells from peripheral blood mononuclear cells (PBMCs). In some aspects, the starting population of immune effector cells is obtained from a subject. The methods of the disclosure may comprise or further comprise introducing the peptides or a nucleic acid encoding the peptide into the dendritic cells prior to the co-culturing. The introduction of the peptide may be done by transfecting or infecting dendritic cells with a nucleic acid encoding the peptide or by incubating the peptide with the dendritic cells. The peptide or nucleic acids encoding the peptide may be introduced by electroporation. Other methods of transfer of nucleic acids are known in the art, such as lipofection, calcium phosphate transfection, transfection with DEAE-dextran, microinjection, and virus-mediated transduction, and are useful in methods of the disclosure for transferring nucleic acids of the disclosure into cells. In some aspects, the peptide or nucleic acids encoding the peptide are introduced by adding the peptide or nucleic acid encoding the peptide to the dendritic cell culture media. In some aspects, the immune effector cells are co-cultured with a second population of dendritic cells into which the peptide or the nucleic acid encoding the peptide has been introduced. In some aspects, a population of CD4-positive or CD8-positive and peptide MHC tetramer-positive T cells are purified from the immune effector cells following the co-culturing. In some aspects, the population of CD4-positive or CD8-positive and peptide MHC tetramer-positive T cells are purified by fluorescence activated cell sorting (FACS). In some aspects, a clonal population of coronavirus-specific immune effector cells are generated by limiting or serial dilution followed by expansion of individual clones by a rapid expansion protocol.

In some aspects, purifying further comprises generation of a clonal population of coronavirus-specific immune effector cells by limiting or serial dilution of sorted cells followed by expansion of individual clones by a rapid expansion protocol. Methods of the disclosure may comprise or further comprise cloning of a T cell receptor (TCR) from the clonal population of coronavirus-specific immune effector cells. The term isolating in the methods of the disclosure may be defined or further defined as cloning of a T cell receptor (TCR) from the clonal population of coronavirus-specific immune effector cells. In some aspects, cloning of the TCR is cloning of a TCR alpha and a beta chain. In some aspects, the TCR is cloned using a 5′-Rapid amplification of cDNA ends (RACE) method. In some aspects, the TCR alpha and beta chains are cloned using a 5′-Rapid amplification of cDNA ends (RACE) method. In some aspects, the cloned TCR is subcloned into an expression vector. In some aspects, the expression vector comprises a linker domain between the TCR alpha sequence and TCR beta sequence. The expression vector may be a retroviral or lentiviral vector. The vector may also be an expression vector described herein. The linker domain may comprise a sequence encoding one or more peptide cleavage sites. The one or more cleavage sites may be a Furin cleavage site and/or a P2A cleavage site. In some aspects, the TCR alpha sequence and TCR beta sequence are linked by an IRES sequence.

A host cell of the disclosure may be transduced with an expression vector to generate an engineered cell that expresses the TCR alpha and/or beta chains. In some aspects, the host cell is an immune cell. The immune cell may be a T cell and the engineered cell may be referred to as an engineered T cell. The T cell may be type of T cell described herein, such as a CD8+ T cell, CD4+ T cell, or γδ T cell. The starting population of immune effector cells may be obtained from a subject having a SARS-Cov-2 infection and the host cell is allogeneic or autologous to the subject. The coronavirus-specific T cells may be autologous or allogeneic. A population of CD4-positive or CD8-positive and peptide MHC tetramer-positive engineered T cells may be purified from the transduced host cells. In some aspects, a clonal population of coronavirus-specific engineered T cells are generated by limiting or serial dilution followed by expansion of individual clones by a rapid expansion protocol. In some aspects, purifying in the methods of the disclosure is defined as purifying a population of CD4-positive or CD8-positive and peptide MHC tetramer-positive T cells from the immune effector cells following the co-culturing.

The peptide may be linked to a solid support. In some aspects, the peptide is conjugated to the solid support or is bound to an antibody that is conjugated to the solid support. The solid support may comprise a microplate, a bead, a glass surface, a slide, or a cell culture dish. In some aspects, the solid support comprises a nanofluidic chip. Detecting T cell responses may comprise or further comprise detecting the binding of the peptide to the T cell or TCR. In some aspects, detecting T cell responses comprises an ELISA, ELISPOT, or a tetramer assay.

Methods of the disclosure may also be used for determining the efficacy of a vaccine, such as a coronavirus, SARS, or SARS-CoV2 vaccine.

Kit aspects of the disclosure may comprise a peptide of the disclosure in a container. The peptide may be comprised in a pharmaceutical preparation. The pharmaceutical preparation may be formulated for parenteral administration or inhalation. In some aspects, the peptide is comprised in a cell culture media.

The term “subject” and “patient” may be used interchangeably and may refer to a human subject. The subject may be defined as a mammalian subject. The subject may also be a mouse, rat, pig, horse, non-human primate, cat, dog, cow, and the like.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment discussed with respect to one aspect of the disclosure applies to other aspects of the disclosure as well and vice versa.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. SARS-Cov-2 membrane glyco-protein (MGP) HLA-A0201 restricted peptide identification from MHC immuno-precipitation (IP) method.

FIG. 2. SARS-Cov-2 HLA-A0201 restricted peptide (MGP-65, FVLAAVYRI—SEQ ID NO:22) CTL generation.

FIG. 3A-C. Functional validation of MGP-65 peptide specific CTL cell lines.

FIG. 4. MGP-65 CTL cell lines recognizing specificity detection with cold target inhibition assay.

FIG. 5. Functional validation of MGP-65 specific CTL cell lines using intracellular cytokine staining (ICS) assay.

FIG. 6. SARS CoV-2 specific TCR-T generation.

FIG. 7A-C. Functional validation of SARS-CoV-2 specific TCR-T. (A) The ability of SARS-CoV-2 specific TCR-T to lyse target cells is shown (Cr51 release assay, CRA). T2 cells pulsed with various concentrations of SARS-CoV-2 peptide were used as targets. The effector to target (E:T) ratio is 20:1. (B, C) The ability of SARS-CoV-2 TCR-T to lyse different targets is shown (CRA). A375, Mel624 cell lines overexpressing eGFP or SARS-Cov-2 full length protein were used as targets.

FIG. 8A-L. SARS-CoV-2 derived HLA Class-I peptide identification with Mass Spectrometry. (A) Schematic representation of HLA-I peptide identification. MS/MS spectra of immunopeptidome and proteome analysis were searched against with Swiss-Prot human and virus protein database and filtered at 1% FDR. (B) MS/MS annotation for NSP13-400 peptide (VYIGDPAQL—SEQ ID NO:37). (C-E) PRM analysis for NSP13-448 peptide (IVDTVSALVY—SEQ ID NO:34), NSP13-242 peptide (TLVPQEHYV—SEQ ID NO:35) and MGP-65 peptide (FVLAAVYRI—SEQ ID NO:22). MS1 XIC areas and MS/MS for each targeted peptide were plotted using Skyline software. (F-J) The multiple sequence alignment of the five candidate peptide sequences to all coronavirus known to infect humans. (K) Adapted from Nextstrain project (http://nextstrain.org). The five candidate SARS-CoV-2 peptide sequences were located in conserved region of SARS-CoV-2 genome. (L) Four mutations were reported from NSP13 protein: E341D, A368V (P.1 lineage), K460R (B.1.1.7 lineage) and T588I (B.1.351 lineage). The following abbreviations are used: VAR: variants, a dot (.) indicates the same amino acid in that position.

FIG. 9A-G. T cell generation and functional validation of membrane glycol-protein (MGP) derived HLA-A0201 peptide MGP-65. (A) Mature dendritic cells (DCs) derived from HLA-A0201 healthy donor were pulsed with MGP-65 peptide (FVLAAVYRI—SEQ ID NO:22) and co-cultured with autologous PBMC. After two rounds of stimulation, small CD8+ and MGP-65 Tetramer+ population were observed (left side). CD8+ and MGP-65 tetramer-positive cells were then sorted and expanded using a standard rapid expansion protocol (REP). After expansion for two weeks, high purity CTLs (tetramer+ population over 90%) were generated (right side). (B) ⁵¹Cr labeled T2 cells pulsed with various concentrations of MGP-65 peptide were co-cultured with MGP-65 specific CTLs at 20:1 of effector to target (E:T) ratio. The lysis ability of MGP-65 specific CTLs was detected with standard ⁵¹Cr release assay (CRA). The data were show as average of triplicate. (C, D)⁵¹Cr labeled MGP or GFP force expressing HLA-A0201 cell lines A375 (A375-MGP, A375-GFP) and Mel624 (Mel624-MGP, Mel624-GFP) were co-cultured with MGP-65 specific CTLs at various E:T ratio (from 40:1 to 1.25:1). The lysis ability of MGP-65 specific CTLs to different targets were detected with standard CRA. The data were show as average of triplicate. (E, F) Cold target inhibition assay. ⁵¹Cr labeled A375-MGP and Mel624-MGP were as hot targets. Non-radiolabeled T2 cells pulsed with MGP-65 peptide or M26 irrelevant peptide were as cold targets. Cold target: Hot target ratio was 10:1 or 20:1. MGP-65 specific CTLs were co-cultured with hot targets alone or hot targets together with cold targets at 20:1 E: hot T ratio. The lysis ability of MGP-65 specific CTLs were detected with standard CRA. The data were show as average of triplicate. (G) Intracellular cytokine staining (ICS) assay. MGP-65 specific CTLs were co-culture with T2 pulsed with MGP-65 peptide or M26 irrelevant peptide, as well as A375-MGP, A375-GFP, Mel624-MGP, Mel624-GFP at 10:1 E:T ratio in the presence of Brefeldin A (BFA) for overnight. After incubation, the levels of IFN-7 and TNF-α, as well as TCR pathway down-stream activated marker CD137 and CD69 were detected using flow cytometry.

FIG. 10A-G. T cell generation and functional validation of non-structure protein NSP13 derived HLA-A0201 peptide NSP13-242. (A) PBMC from HLA-A0201 healthy donor were co-cultured with NSP13-242 peptide (TLVPQEHYV—SEQ ID NO:35) pulsed autologous DCs. After two rounds of stimulation, CD8+ and NSP13-242 tetramer-positive T cells were induced (left side). CD8+ and Tetramer+ T cells were sorted and then expanded with REP for two weeks to generate high purity of NSP13-242 specific CTLs (right side). (B)⁵¹Cr labeled T2 cells pulsed with various concentrations of NSP13-242 peptide were co-cultured with NSP13-242 specific CTLs at 20:1 E:T ratio. The lysis ability of NSP13-242 specific CTLs was detected with standard CRA. The data were show as average of triplicate. (C, D)⁵′Cr labeled NSP13 or GFP force expressing HLA-A0201 cell lines A375 (A375-NSP13, A375-GFP) and Mel624 (Mel624-NSP13, Mel624-GFP) were co-cultured with NSP13-242 specific CTLs at various E:T ratio (from 40:1 to 1.25:1). The lysis ability of NSP13-242 specific CTLs to different targets were detected with standard CRA. The data were show as average of triplicate. (E, F) Cold target inhibition assay. ⁵¹Cr labeled A375-NSP13 and Mel624-NSP13 were as hot targets. Non-radiolabeled T2 cells pulsed with NSP13-242 peptide or M26 irrelevant peptide were as cold targets. Cold target: Hot target ratio was 10:1 or 20:1. NSP13-242 specific CTLs were co-cultured with hot targets alone or hot targets together with cold targets at 20:1 E: hot T ratio. The lysis ability of NSP13-242 specific CTLs were detected with standard CRA. The data were show as average of triplicate. (G) Intracellular cytokine staining (ICS) assay. NSP13-242 specific CTLs were co-culture with T2 pulsed with NSP13-242 peptide or M26 irrelevant peptide, as well as A375-NSP13, A375-GFP, Mel624-NSP13, Mel624-GFP at 10:1 E:T ratio in the presence of BFA for overnight. After incubation, the levels of IFN-γ and TNF-α, as well as TCR pathway down-stream activated marker CD137 and CD69 were detected using flow cytometry.

FIG. 11A-G. T cell generation and functional validation of non-structure protein NSP13 derived HLA-A0101 peptide NSP13-448. (A) PBMC from HLA-A0101 healthy donor were co-cultured with NSP13-448 peptide (VYIGDPAQL—SEQ ID NO:37) pulsed autologous DCs. After two rounds of stimulation, CD8+ and NSP13-448 tetramer-positive T cells were induced (left side). CD8+ and Tetramer+ T cells were sorted and then expanded with REP for two weeks to generate high purity of NSP13-448 specific CTLs (right side). (B) ⁵¹Cr labeled A375 cells pulsed with various concentrations of NSP13-448 peptide were co-cultured with NSP13-448 specific CTLs at 20:1 E:T ratio. The lysis ability of NSP13-448 specific CTLs was detected with standard CRA. The data were show as average of triplicate. (C, D)⁵′Cr labeled NSP13 or GFP force expressing HLA-A0101 cell lines A375 (A375-NSP13, A375-GFP) and RPMI-7951 (RPMI-7951-NSP13, RPMI-7951-GFP) were co-cultured with NSP13-448 specific CTLs at various E:T ratio (from 40:1 to 1.25:1). The lysis ability of NSP13-448 specific CTLs to different targets were detected with standard CRA. The data were show as average of triplicate. (E, F) Cold target inhibition assay. ⁵¹Cr labeled A375-NSP13 and RPMI-7951-NSP13 were as hot targets. Non-radiolabeled A375 cells pulsed with NSP13-448 peptide or irrelevant HLA-A0101 peptide were as cold targets. Cold target: Hot target ratio was 10:1 or 20:1. NSP13-448 specific CTLs were co-cultured with hot targets alone or hot targets together with cold targets at 20:1 E: hot T ratio. The lysis ability of NSP13-448 specific CTLs were detected with standard CRA. The data were show as average of triplicate. (G) Intracellular cytokine staining (ICS) assay. NSP13-448 specific CTLs were co-culture with A375 pulsed with NSP13-448 peptide or irrelevant HLA-A0101 peptide, as well as A375-NSP13, A375-GFP, RPMI-7951-NSP13, RPMI-7951-GFP at 10:1 E:T ratio in the presence of BFA for overnight. After incubation, the levels of IFN-7 and TNF-α, as well as TCR pathway down-stream activated marker CD137 and CD69 were detected using flow cytometry.

FIG. 12A-E. T cell generation and functional validation of non-structure protein NSP13 derived HLA-A0301 peptide NSP13-134. (A) NSP13-134 peptide (KLFAAETLK—SEQ ID NO:36) pulsed DCs were co-cultured with autologous PBMC of HLA-A0301 healthy donor. After two rounds of stimulation, CD8+ and NSP13-134 tetramer-positive T cells were induced (left side). CD8+ and Tetramer+ T cells were sorted and then expanded with REP for two weeks to generate high purity of NSP13-134 specific CTLs (right side). (B)⁵′Cr labeled HLA-A0301 cell lines Hs-578T pulsed with various concentrations of NSP13-134 peptide were co-cultured with NSP13-134 specific CTLs at 20:1 E:T ratio. The lysis ability of NSP13-134 specific CTLs was detected with standard CRA. The data were show as average of triplicate. (C)⁵¹Cr labeled NSP13 or GFP force expressing HLA-A0301 cell lines Hs-578T (Hs-578T-NSP13, Hs-578T-GFP) were co-cultured with NSP13-134 specific CTLs at various E:T ratio (from 40:1 to 1.25:1). The lysis ability of NSP13-134 specific CTLs to different targets were detected with standard CRA. The data were show as average of triplicate. (D) Cold target inhibition assay. ⁵¹Cr labeled Hs-578T-NSP13 cells were as hot targets. Non-radiolabeled Hs-578T cells pulsed with NSP13-134 peptide or irrelevant HLA-A0301 peptide were as cold targets. Cold target: Hot target ratio was 10:1 or 20:1. NSP13-134 specific CTLs were co-cultured with hot targets alone or hot targets together with cold targets at 20:1 E: hot T ratio. The lysis ability of NSP13-134 specific CTLs were detected with standard CRA. The data were show as average of triplicate. (E) Intracellular cytokine staining (ICS) assay. NSP13-134 specific CTLs were co-culture with Hs-578T pulsed with NSP13-134 peptide or irrelevant HLA-A0301 peptide, as well as Hs-578T-NSP13, Hs-578T-GFP at 10:1 E:T ratio in the presence of BFA for overnight. After incubation, the levels of IFN-7 and TNF-α, as well as TCR pathway down-stream activated marker CD137 and CD69 were detected using flow cytometry.

FIG. 13A-G. T cell generation and functional validation of non-structure protein NSP13 derived HLA-A2402 peptide NSP13-400. (A) PBMC from HLA-A2402 healthy donor were co-cultured with NSP13-400 peptide (VYIGDPAQL—SEQ ID NO:37) pulsed autologous DCs. After two rounds of stimulation, CD8+ and NSP13-400 tetramer-positive T cells were induced (left side). After sorting and REP for CD8+ and Tetramer+ T cells with two weeks, high purity of NSP13-400 specific CTLs were expanded (right side). (B)⁵¹Cr labeled HLA-A2402 cell lines M14 pulsed with various concentrations of NSP13-400 peptide were co-cultured with NSP13-400 specific CTLs at 20:1 E:T ratio. The lysis ability of NSP13-400 specific CTLs was detected with standard CRA. The data were show as average of triplicate. (C, D)⁵¹Cr labeled NSP13 or GFP force expressing HLA-A2402 cell lines Hs-578T (Hs-578T-NSP13, Hs-578T-GFP) and M14 (M14-NSP13, M14-GFP) were co-cultured with NSP13-400 specific CTLs at various E:T ratio (from 40:1 to 1.25:1). The lysis ability of NSP13-400 specific CTLs to different targets were detected with standard CRA. The data were show as average of triplicate. (E, F) Cold target inhibition assay. ⁵¹Cr labeled Hs-578T-NSP13 and M14-NSP13 were as hot targets. Non-radiolabeled M14 cells pulsed with NSP13-400 peptide or irrelevant HLA-A2402 peptide were as cold targets. Cold target: Hot target ratio was 10:1 or 20:1. NSP13-400 specific CTLs were co-cultured with hot targets alone or hot targets together with cold targets at 20:1 E: hot T ratio. The lysis ability of NSP13-242 specific CTLs were detected with standard CRA. The data were show as average of triplicate. (G) Intracellular cytokine staining (ICS) assay. NSP13-400 specific CTLs were co-culture with M14 pulsed with NSP13-400 peptide or irrelevant HLA-A2402 peptide, as well as Hs-578T-NSP13, Hs-578T-GFP, M14-NSP13, M14-GFP at 10:1 E:T ratio in the presence of BFA for overnight. After incubation, the levels of IFN-7 and TNF-α, as well as TCR pathway down-stream activated marker CD137 and CD69 were detected using flow cytometry.

FIG. 14A-E. MGP-65 peptide specific T cell receptor engineered T cells (TCR-T) generation and functional validation. (A) The whole length MGP-65 TCR alpha chain and beta chain linked with FP2A peptide were inserted into retrovirus vector pMSGV1 and then the recombinant retrovirus vector were used to infect the OKT3 activated HLA-A0201 allo-PBMC. After infection about 5 days, CD8+ and MGP-65-Tetramer+ T cell population were analyzed by flow cytometry (middle). CD8+ and MGP-65 tetramer-positive cells were then sorted and expanded with REP. After expansion for two weeks, high purity CTLs (tetramer+ population over 90%) were generated (right side). (B) Peptide titration assay for MGP-65 specific TCR-T. ⁵¹Cr labeled T2 cells pulsed with various concentrations of MGP-65 peptide were co-cultured with MGP-65 specific TCR-T. The lysis level of MGP-65 specific TCR-T were analyzed with standard CRA. The data were show as average of triplicate. (C, D) Antigen-specific cytolysis analysis for MGP-65 specific TCR-T. ⁵¹Cr labeled A375-MGP, A375-GFP, Mel624-MGP or Mel624-GFP were co-cultured with MGP-65 specific TCR-T at various E:T ratio. The lysis level of MGP-65 specific TCR-T to different targets were analyzed with standard CRA. The data were show as average of triplicate. (E) Intracellular cytokine staining (ICS) assay. MGP-65 specific TCR-T were co-culture with T2 pulsed with MGP-65 peptide or M26 irrelevant peptide, as well as A375-MGP, A375-GFP, Mel624-MGP, Mel624-GFP at 10:1 E:T ratio in the presence of BFA for overnight. After incubation, the levels of IFN-7 and TNF-α, as well as TCR pathway down-stream activated marker CD137 and CD69 were detected using flow cytometry.

FIG. 15. SARS-CoV-2 MHC bound peptide identification and specific T generation workflow. The schematic representation of the antigen discovery platform for SARS-CoV-2. (1) SARS-CoV-2 structure protein membrane glycol-protein (MGP) and Non-structure protein helicase (NSP13) gene force expressing cell lines with high frequency of HLA allele were established. For immunopeptidome measurements, the expanded MGP or NSP13 force expressing cells were lysed and MHC peptide complex were immunoprecipitated with anti-MHC antibody (W6/32) and eluted with acid. The eluted MHC bound peptides were then analyzed by LC-MS/MS. (2) Endogenous T cell (ETC) generation workflow was deployed to generate peptide-specific CTL. (3) Validation of generated SARS-CoV-2 specific CTL with SARS-CoV-2 target recognition. (4) Development of SARS-CoV-2 functional T cell receptor (TCR) engineered T cells (TCR-T) based on specific CTL validation.

FIG. 16. T cell generation and functional validation for predicted SARS-CoV-2 HLA-A0201 restricted peptide. 3 predicted membrane glycol-protein (MGP) HLA-A0201 peptides MGP-53 (FLWLLWPVTL—SEQ ID NO:63), MGP-56 (LLWPVTLACFV—SEQ ID NO:67), MGP-89 (GLMWLSYFI—SEQ ID NO:43), and 4 predicted spike protein (SP) HLA-A0201 peptide SP-424 (KLPDDFTGCV—SEQ ID NO:78), SP-821 (LLFNKVTLA—SEQ ID NO:79), SP-983 (RLDKVEAEV—SEQ ID NO:80), SP-995 (RLITGRLQSL—SEQ ID NO:81) were selected for antigen specific T cell generation using endogenous T cell (ETC) generation workflow (left side). The antigen specific-cytolysis were analyzed with standard ⁵¹Cr release assay (CRA) using MGP, SP or GFP force expressing HLA-A0201 cell lines (A375-MGP, A375-SP, A375-GFP, Mel624-MGP, Mel624-GFP, SK-MEL-5-SP, SK-MEL-5-GFP) as targets (right side).

FIG. 17. The PRM screen for NSP13 predicted HLA-A0301 peptide. The ten predicted high potential HLA-A0301 binding peptides from NSP13 were selected and analyzed by PRM methods.

FIG. 18. MGP-65 specific T cell generation using ETC workflow. Mature dendritic cells (DCs) derived from HLA-A0201 healthy donor were pulsed with MGP-65 peptide (FVLAAVYRI—SEQ ID NO:22) and co-cultured with autologous PBMC in one 48 well plate. After two rounds of stimulation, CD8+ and MGP-65 Tetramer+ population in individual well were detected using flow cytometry.

FIG. 19. NSP13-242 specific T cell generation using ETC workflow. Mature dendritic cells (DCs) derived from HLA-A0201 healthy donor were pulsed with NSP13-242 peptide (TLVPQEHYV—SEQ ID NO:35) and co-cultured with autologous PBMC in one 48 well plate. After two rounds of stimulation, CD8+ and NSP13-242 Tetramer+ population in individual well were detected using flow cytometry.

FIG. 20. NSP13-448 specific T cell generation using ETC workflow. Mature dendritic cells (DCs) derived from HLA-A0101 healthy donor were pulsed with NSP13-448 peptide (IVDTVSALVY—SEQ ID NO:34) and co-cultured with autologous PBMC in one 48 well plate. After two rounds of stimulation, CD8+ and NSP13-134 Tetramer+ population in individual well were detected using flow cytometry.

FIG. 21. NSP13-134 specific T cell generation using ETC workflow. Mature dendritic cells (DCs) derived from HLA-A0301 healthy donor were pulsed with NSP13-134 peptide (KLFAAETLK—SEQ ID NO:36) and co-cultured with autologous PBMC in one 48 well plate. After two rounds of stimulation, CD8+ and NSP13-134 Tetramer+ population in individual well were detected using flow cytometry.

FIG. 22. NSP13-400 specific T cell generation using ETC workflow. Mature dendritic cells (DCs) derived from HLA-A0201 healthy donor were pulsed with NSP13-400 peptide (VYIGDPAQL—SEQ ID NO:37) and co-cultured with autologous PBMC in one 48 well plate. After two rounds of stimulation, CD8+ and NSP13-400 Tetramer+ population in individual well were detected using flow cytometry.

FIG. 23. NSP13-134 peptide specific TCR-T cell generation. The whole length TCR alpha chain and beta chain were inserted into retrovirus vector pMSGV3 and then the recombinant retrovirus vector was used to infect PBMCs. After infection, the CD8+/Tetramer+ population was detected by flow cytometry. After tetramer guided sorting and expansion, the highly pure TCR-T cells were generated.

FIG. 24A-C. Target killing assay of NSP13-134 peptide specific TCR-T cells. (A) T2 cells pulsed with various concentrations of NSP13-134 peptide were used as targets. The lysis ability of NSP13-134 specific TCR-T cells was detected with a Cr51 release assay (CRA). The effector to target (E:T) ratio used was 20:1. (B, C) SK-MES-1 (HLA-A0301+), Hs-578T (HLA-A0301+) cell lines with forced expression of GFP or SARS-Cov-2 NSP13 peptide were used as the targets and co-cultured with NSP13-134 specific TCR-T cells. The effector to target (E:T) ratio used was from 40:1 to 1.25:1. The lysis ability of NSP13-134 TCR-T cells to different targets was detected with Cr51 release assay (CRA). NSP13-134 TCR-T cells show significantly higher killing levels to positive targets than to negative control.

FIG. 25. Functional validation of NSP13-134 TCR-T cells with intracellular cytokine staining (ICS) assay. NSP13-134 specific TCR-T cells were co-cultured with SK-MES-1+control A0301 peptide, SK-MES-1+NSP13-134, SK-MES-1-GFP, SK-MES-1-NSP13, Hs-578T-GFP and Hs-578T-NSP13 (E:T=10:1). After overnight co-culturing, the TCR pathway down-stream activated markers, CD137, CD69, IFN-7 and TNF-α, were detected with ICS assay. the level of CD137, CD69, IFN-7 and TNF-α of NSP13-134 specific TCR-T was significantly enhanced when they were co-cultured with positive targets compared to negative control targets.

FIG. 26. NSP13-242 peptide specific TCR-T cell generation. The whole length TCR alpha chain and beta chain were inserted into retrovirus vector pMSGV3 and then the recombinant retrovirus vector was used to infect PBMCs. After infection, the CD8+/Tetramer+ population was detected by flow cytometry. After tetramer guided sorting and expansion, the highly pure TCR-T cells were generated.

FIG. 27A-C. Target killing assay of NSP13-242 peptide specific TCR-T cells. (A) T2 cells pulsed with various concentrations of NSP13-242 peptide were used as targets. The lysis ability of NSP13-242 specific TCR-T cells was detected with Cr51 release assay (CRA). The effector to target (E:T) ratio used was 20:1. (B, C) A375 (HLA-A0201+), RPMI-7951 (HLA-A0201+) cell lines with forced expression of GFP or SARS-Cov-2 NSP13 were used as the targets and co-cultured with NSP13-242 specific TCR-T cells. The effector to target (E:T) ratio used was from 40:1 to 1.25:1. The lysis ability of NSP13-242 TCR-T to different targets was detected with Cr51 release assay (CRA). NSP13-242 TCR-T showed significantly higher killing levels to positive targets than to negative control.

FIG. 28. Functional validation of NSP13-242 TCR-T with intracellular cytokine staining (ICS) assay. NSP13-242 specific TCR-T cells were co-cultured with T2+M26, T2+NSP13-242, A375-GFP, A375-NSP13, RPMI-7951-GFP and RPMI-7951-NSP13 (E:T=10:1). After overnight co-culturing, the TCR pathway down-stream activated markers, CD137, CD69, IFN-γ and TNF-α, were detected with ICS assay. The level of CD137, CD69, IFN-γ and TNF-α of NSP13-242 specific TCR-T cells was significantly enhanced when they were co-cultured with positive targets compared to negative control targets.

FIG. 29. NSP13-400 antigen specific TCR-T cell generation. The alpha chain and beta chain TCR genes were cloned out from one of NSP13-400 specific CTL cell lines. The whole length TCR alpha chain and beta chain were inserted into retrovirus vector pMSGV3 and then the recombinant retrovirus vector was used to infect the PBMC. After infection, both CD8+/Tetramer+ and CD4+/Tetramer+ population were observed. After tetramer guided sorting and expansion, high purity of CD8+ and CD4+ TCR-T cells were generated.

FIG. 30A-F. Validation of specificity of MGP-65 specific TCR-T cells with killing assay. (A, D) M14 cells pulsed with various concentrations of NSP13-400 peptide were used as targets. The lysis ability of NGP13-400 CD8+ and CD4+ specific TCR-T cells was detected with Cr51 release assay (CRA). The effector to target (E:T) ratio is 20:1. (B, C, E, F) Hs-578T (HLA-A2402+), M14 (HLA-A2402+) cell lines with forced expression of GFP or SARS-Cov-2 NSP13 were used as the targets and co-cultured with NSP13-400 specific TCR-T cells. The effector to target (E:T) ratio used was from 40:1 to 1.25:1. The lysis ability of NSP13-400 specific TCR-T cells to different targets were detected with Cr51 release assay (CRA).

FIG. 31. Functional validation of NSP13-400 TCR-T with intracellular cytokine staining (ICS) assay. NSP13-400 specific CD8+ and CD4+ TCR-T were co-cultured with M14+control peptide, M14+NSP13-400, Hs-578T-GFP, Hs-578T-NSP13, M14-GFP and M14-NSP13 (E:T=10:1). After overnight co-culturing, the TCR pathway down-stream activated markers, CD137, CD69, IFN-7 and TNF-α, were detected with an ICS assay. The level of CD137, CD69, IFN-7 and TNF-α of NSP13-400 specific CTL cell lines were significantly enhanced when they were co-cultured with positive targets compared to negative control targets.

FIG. 32. NSP13-448 peptide specific TCR-T cell generation. The whole length TCR alpha chain and beta chain was inserted into retrovirus vector pMSGV3 and then the recombinant retrovirus vector were used to infect the PBMC. After infection, the CD8+/Tetramer+ population was detected by flow cytometry. After tetramer guided sorting and expansion, high purity of TCR-T cells was generated.

FIG. 33A-C. Target killing assay of NSP13-448 peptide specific TCR-T cells. (A) A375 cells pulsed with various concentrations of NSP13-448 peptide were used as targets. The lysis ability of NSP13-448 specific TCR-T was detected with Cr51 release assay (CRA). The effector to target (E:T) ratio is 20:1. (B, C) A375 (HLA-A0101+), RPMI-7951 (HLA-A0101+) cell lines forced expressing GFP or SARS-Cov-2 NSP13 were as the targets and co-cultured with NSP13-448 specific TCR-T. The effector to target (E:T) ratio is from 40:1 to 1.25:1. The lysis ability of NSP13-448 TCR-T to different targets were detected with Cr51 release assay (CRA). NSP13-448 TCR-T show significantly higher killing lever to positive targets than negative control.

FIG. 34. Functional validation of NSP13-448 TCR-T cells with intracellular cytokine staining (ICS) assay. NSP13-448 specific TCR-T were co-cultured with A375+control A0101 peptide, A375+NSP13-448, A375-GFP, A375-NSP13, RPMI-7951-GFP and RPMI-7951-NSP13 (E:T=10:1). After overnight co-culturing, the TCR pathway down-stream activated markers, CD137, CD69, IFN-7 and TNF-α, were detected with an ICS assay. The level of CD137, CD69, IFN-7 and TNF-α of NSP13-448 specific TCR-T were significantly enhanced when they were co-cultured with positive targets compared to negative control targets.

### DETAILED DESCRIPTION OF THE INVENTION

SARS-CoV-2 infections elicit both humoral and cellular immune responses. For the prevention and treatment of COVID19, the disease caused by SARS-CoV-2, it has become increasingly apparent that T cell responses are equally, if not more important than humoral responses in mediating recovery and immune-protection. One of the major challenges in developing T cell-based therapies for infectious and malignant diseases has been the identification of immunogenic epitopes that can elicit a meaningful T cell response. Traditionally, this has been achieved using sophisticated in silico methods to predict putative epitopes deduced from binding affinities and consensus data. The inventors found that ‘immunodominant’ SARS-CoV-2 peptides defined in this manner can fail to elicit T cell responses that recognize naturally presented SARS-CoV-2 epitopes. They postulated that immunogenic epitopes for SARS-CoV-2 are best defined empirically by directly analyzing peptides eluted from the naturally-processed peptide-MHC complex and then validating immunogenicity by determining if such peptides can elicit T cells recognizing SARS-CoV-2 antigen-expressing cells. Using a tandem mass spectrometry approach, the inventors identified epitopes of SARS-CoV-2 derived not only from structural but also non-structural genes in regions highly conserved among SARS-CoV-2 strains including recently recognized variants. Finally, there are no reported TCR sequences that when engineered into recombinant vectors, can redirect T cell specificity to recognize and kill SARS-CoV-2 target cells. The inventors report here, for the first time, several novel SARS-CoV-2 epitopes defined by mass-spectrometric analysis of MHC-eluted peptides, provide empiric evidence for their immunogenicity and demonstrate engineered TCR-redirected killing.

The peptides described herein can be used to detect the immunoresponse of patients with a SARS-Cov-2 infection (COVID19) or for vaccination. This will lead to a better understanding of COVID19 immunity and will directly impact the management of patients with COVID19 and will allow for the evaluation of the immunoresponse of the vaccine. Furthermore, this peptide cytotoxic T cell and the engineered TCRs can be used to generate SARS-Cov-2 specific T cells against HLA-matched targets, thus providing off-the shelf T cell therapy for patients with COVID19 disease.

### I. Immunotherapies Using Peptides of the Disclosure

A peptide as described herein (e.g., a peptide of SEQ ID NO:22-81) may be used for immunotherapy for treating a viral infection. For example, a peptide of one of SEQ ID NOS:22-81 may be contacted with or used to stimulate a population of T cells to induce proliferation of the T cells that recognize or bind said peptide. In other aspects, a peptide of the disclosure may be administered to a subject, such as a human patient, to enhance the immune response of the subject against a SARS-Cov-2 infection.

A peptide of the disclosure may be included in an active immunotherapy (e.g., a vaccine) or a passive immunotherapy (e.g., an adoptive immunotherapy). Active immunotherapies include immunizing a subject with a purified peptide antigen or an immunodominant peptide (native or modified); alternatively, antigen presenting cells pulsed with a peptide of the disclosure (or transfected with genes encoding an antigen comprising the peptide) may be administered to a subject. The peptide may be modified or contain one or more mutations such as, e.g., a substitution mutation. Passive immunotherapies include adoptive immunotherapies. Adoptive immunotherapies generally involve administering cells to a subject, wherein the cells (e.g., cytotoxic T cells) have been sensitized in vitro to a peptide of the disclosure (see, e.g., U.S. Pat. No. 7,910,109).

In some aspects, flow cytometry may be used in the adoptive immunotherapy for rapid isolation of human tumor antigen-specific T-cell clones by using, e.g., T-cell receptor (TCR) Vβ antibodies in combination with carboxyfluorescein succinimidyl ester (CFSE)-based proliferation assay. See, e.g., Lee et al., J. Immunol. Methods, 331:13-26, 2008, which is incorporated by reference for all purposes. In some aspects, tetramer-guided cell sorting may be used such as, e.g., the methods described in Pollack, et al., J Immunother Cancer. 2014; 2: 36, which is herein incorporated by reference for all purposes. Various culture protocols are also known for adoptive immunotherapy and may be used in aspects of the disclosure. In some aspects, cells may be cultured in conditions which do not require the use of antigen presenting cells (e.g., Hida et al., Cancer Immunol. Immunotherapy, 51:219-228, 2002, which is incorporated by reference). In other aspects, T cells may be expanded under culture conditions that utilize antigen presenting cells, such as dendritic cells (Nestle et al., 1998, incorporated by reference), and in some aspects artificial antigen presenting cells may be used for this purpose (Maus et al., 2002 incorporated by reference). Additional methods for adoptive immunotherapy are disclosed in Dudley et al. (2003), which is incorporated by reference, that may be used with aspects of the current disclosure. Various methods are known and may be used for cloning and expanding human antigen-specific T cells (see, e.g., Riddell et al., 1990, which is herein incorporated by reference).

In certain aspects, the following protocol may be used to generate T cells that selectively recognize peptides of the disclosure. Peptide-specific T-cell lines may be generated from normal donors or HLA-restricted normal donors and patients using methods previously reported (Hida et al., 2002). ENREF 32 Briefly, PBMCs (1×10⁵ cells/well) can be stimulated with about 10 g/ml of each peptide in quadruplicate in a 96-well, U-bottom-microculture plate (Corning Incorporated, Lowell, MA) in about 200 μl of culture medium. The culture medium may consist of 50% AIM-V medium (Invitrogen), 50% RPMI1640 medium (Invitrogen), 10% human AB serum (Valley Biomedical, Winchester, VA), and 100 IU/ml of interleukin-2 (IL-2). Cells may be restimulated with the corresponding peptide about every 3 days. After 5 stimulations, T cells from each well may be washed and incubated with T2 cells in the presence or absence of the corresponding peptide. After about 18 hours, the production of interferon (IFN)-γ may be determined in the supernatants by ELISA. T cells that secret large amounts of IFN-γ may be further expanded by a rapid expansion protocol (Riddell et al., 1990; Yee et al., 2002b).

In some aspects, an immunotherapy may utilize a peptide of the disclosure that is associated with a cell penetrator, such as a liposome or a cell penetrating peptide (CPP). Antigen presenting cells (such as dendritic cells) pulsed with peptides may be used to enhance antitumour immunity (Celluzzi et al., 1996; Young et al., 1996). Liposomes and CPPs are described in further detail below. In some aspects, an immunotherapy may utilize a nucleic acid encoding a peptide of the disclosure, wherein the nucleic acid is delivered, e.g., in a viral vector or non-viral vector.

### II. Cell Penetrating Peptides

A peptide of the disclosure may also be associated with or covalently bound to a cell penetrating peptide (CPP). Cell penetrating peptides that may be covalently bound to a peptide of the disclosure include, e.g., HIV Tat, herpes virus VP22, the Drosophila Antennapedia homeobox gene product, signal sequences, fusion sequences, or protegrin I. Covalently binding a peptide to a CPP can prolong the presentation of a peptide by dendritic cells, thus enhancing antitumour immunity (Wang and Wang, 2002). In some aspects, a peptide of the disclosure (e.g., comprised within a peptide or polyepitope string) may be covalently bound (e.g., via a peptide bond) to a CPP to generate a fusion protein. In other aspects, a peptide or nucleic acid encoding a peptide, according to the current disclosure, may be encapsulated within or associated with a liposome, such as a mulitlamellar, vesicular, or multivesicular liposome.

As used herein, “association” means a physical association, a chemical association or both. For example, an association can involve a covalent bond, a hydrophobic interaction, encapsulation, surface adsorption, or the like.

As used herein, “cell penetrator” refers to a composition or compound which enhances the intracellular delivery of the peptide/polyepitope string to the antigen presenting cell. For example, the cell penetrator may be a lipid which, when associated with the peptide, enhances its capacity to cross the plasma membrane. Alternatively, the cell penetrator may be a peptide. Cell penetrating peptides (CPPs) are known in the art, and include, e.g., the Tat protein of HIV (Frankel and Pabo, 1988), the VP22 protein of HSV (Elliott and O'Hare, 1997) and fibroblast growth factor (Lin et al., 1995).

Cell-penetrating peptides (or “protein transduction domains”) have been identified from the third helix of the Drosophila Antennapedia homeobox gene (Antp), the HIV Tat, and the herpes virus VP22, all of which contain positively charged domains enriched for arginine and lysine residues (Schwarze et al., 2000; Schwarze et al., 1999). Also, hydrophobic peptides derived from signal sequences have been identified as cell-penetrating peptides. (Rojas et al., 1996; Rojas et al., 1998; Du et al., 1998). Coupling these peptides to marker proteins such as β-galactosidase has been shown to confer efficient internalization of the marker protein into cells, and chimeric, in-frame fusion proteins containing these peptides have been used to deliver proteins to a wide spectrum of cell types both in vitro and in vivo (Drin et al., 2002). Fusion of these cell penetrating peptides to a peptide of the disclosure may enhance cellular uptake of the polypeptides.

In some aspects, cellular uptake is facilitated by the attachment of a lipid, such as stearate or myristilate, to the polypeptide. Lipidation has been shown to enhance the passage of peptides into cells. The attachment of a lipid moiety is another way that the present invention increases polypeptide uptake by the cell.

A peptide of the disclosure may be included in a liposomal vaccine composition. For example, the liposomal composition may be or comprise a proteoliposomal composition. Methods for producing proteoliposomal compositions that may be used with the present invention are described, e.g., in Neelapu et al. (2007) and Popescu et al. (2007). In some aspects, proteoliposomal compositions may be used to treat a melanoma.

By enhancing the uptake of a polypeptide of the disclosure, it may be possible to reduce the amount of protein or peptide required for treatment. This in turn can significantly reduce the cost of treatment and increase the supply of therapeutic agent. Lower dosages can also minimize the potential immunogencity of peptides and limit toxic side effects.

In some aspects, a peptide of the disclosure may be associated with a nanoparticle to form nanoparticle-polypeptide complex. In some aspects, the nanoparticle is a liposomes or other lipid-based nanoparticle such as a lipid-based vesicle (e.g., a DOTAP:cholesterol vesicle). In other aspects, the nanoparticle is an iron-oxide based superparamagnetic nanoparticles. Superparamagnetic nanoparticles ranging in diameter from about 10 to 100 nm are small enough to avoid sequestering by the spleen, but large enough to avoid clearance by the liver. Particles this size can penetrate very small capillaries and can be effectively distributed in body tissues. Superparamagnetic nanoparticles-polypeptide complexes can be used as MRI contrast agents to identify and follow those cells that take up the peptide. In some aspects, the nanoparticle is a semiconductor nanocrystal or a semiconductor quantum dot, both of which can be used in optical imaging. In further aspects, the nanoparticle can be a nanoshell, which comprises a gold layer over a core of silica. One advantage of nanoshells is that polypeptides can be conjugated to the gold layer using standard chemistry. In other aspects, the nanoparticle can be a fullerene or a nanotube (Gupta et al., 2005).

Peptides are rapidly removed from the circulation by the kidney and are sensitive to degradation by proteases in serum. By associating a peptide with a nanoparticle, the nanoparticle-polypeptide complexes of the present invention may protect against degradation and/or reduce clearance by the kidney. This may increase the serum half-life of polypeptides, thereby reducing the polypeptide dose need for effective therapy. Further, this may decrease the costs of treatment, and minimizes immunological problems and toxic reactions of therapy.

### III. Polyepitope Strings

In some aspects, a peptide is included or comprised in a polyepitope string. A polyepitope string is a peptide or polypeptide containing a plurality of antigenic epitopes from one or more antigens linked together. A polyepitope string may be used to induce an immune response in a subject, such as a human subject. Polyepitope strings have been previously used to target malaria and other pathogens (Baraldo et al., 2005; Moorthy et al., 2004; Baird et al., 2004). A polyepitope string may refer to a nucleic acid (e.g., a nucleic acid encoding a plurality of antigens including a peptide of the disclosure) or a peptide or polypeptide (e.g., containing a plurality of antigens including a peptide of the disclosure). A polyepitope string may be included in a vaccine composition.

### IV. Applications of Antigenic Peptides

Various aspects are directed to development of and use of antigenic peptides that that are useful for treating and preventing certain viral infections. In many aspects, antigenic peptides are produced by chemical synthesis or by molecular expression in a host cell. Peptides can be purified and utilized in a variety of applications including (but not limited to) assays to determine peptide immunogenicity, assays to determine recognition by T cells, peptide vaccines for treatment of viral infections, development of modified TCRs of T cells, and development of antibodies.

Peptides can be synthesized chemically by a number of methods. One common method is to use solid-phase peptide synthesis (SPPS). Generally, SPPS is performed by repeating cycles of alternate N-terminal deprotection and coupling reactions, building peptides from the c-terminus to the n-terminus. The c-terminus of the first amino acid is coupled the resin, wherein then the amine is deprecated and then coupled with the free acid of the second amino acid. This cycle repeats until the peptide is synthesized.

Peptides can also be synthesized utilizing molecular tools and a host cell. Nucleic acid sequences corresponding with antigenic peptides can be synthesized. In some aspects, synthetic nucleic acids synthesized in in vitro synthesizers (e.g., phosphoramidite synthesizer), bacterial recombination system, or other suitable methods. Furthermore, synthesized nucleic acids can be purified and lyophilized, or kept stored in a biological system (e.g., bacteria, yeast). For use in a biological system, synthetic nucleic acid molecules can be inserted into a plasmid vector, or similar. A plasmid vector can also be an expression vector, wherein a suitable promoter and a suitable 3′-polyA tail is combined with the transcript sequence.

Aspects are also directed to expression vectors and expression systems that produce antigenic peptides or proteins. These expression systems can incorporate an expression vector to express transcripts and proteins in a suitable expression system. Typical expression systems include bacterial (e.g., E. coli), insect (e.g., SF9), yeast (e.g., S. cerevisiae), animal (e.g., CHO), or human (e.g., HEK 293) cell lines. RNA and/or protein molecules can be purified from these systems using standard biotechnology production procedures.

Assays to determine immunogenicity and/or TCR binding can be performed. One such as is the dextramer flow cytometry assay. Generally, custom-made HLA-matched MHC Class I dextramer:peptide (pMHC) complexes are developed or purchased (Immudex, Copenhagen, Denmark). T cells from peripheral blood mononuclear cells (PBMCs) or tumor-infiltrating lymphocytes (TILs) are incubated the pMHC complexes and stained, which are then run through a flow cytometer to determine if the peptide is capable of binding a TCR of a T cell.

The peptides of the disclosure can also be used to isolate and/or identify T-cell receptors that bind to the peptide. T-cell receptors comprise two different polypeptide chains, termed the T-cell receptor α (TCRα) and β (TCRβ) chains, linked by a disulfide bond. These α:β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated γ and δ. Both types of T-cell receptor differ from the membrane-bound immunoglobulin that serves as the B-cell receptor: a T-cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

The three-dimensional structure of the T-cell receptor has been determined. The structure is indeed similar to that of an antibody Fab fragment, as was suspected from earlier studies on the genes that encoded it. The T-cell receptor chains fold in much the same way as those of a Fab fragment, although the final structure appears a little shorter and wider. There are, however, some distinct differences between T-cell receptors and Fab fragments. The most striking difference is in the Ca domain, where the fold is unlike that of any other immunoglobulin-like domain. The half of the domain that is juxtaposed with the C3 domain forms a R sheet similar to that found in other immunoglobulin-like domains, but the other half of the domain is formed of loosely packed strands and a short segment of a helix. The intramolecular disulfide bond, which in immunoglobulin-like domains normally joins two R strands, in a Ca domain joins a R strand to this segment of a helix.

There are also differences in the way in which the domains interact. The interface between the V and C domains of both T-cell receptor chains is more extensive than in antibodies, which may make the hinge joint between the domains less flexible. And the interaction between the Ca and C3 domains is distinctive in being assisted by carbohydrate, with a sugar group from the Ca domain making a number of hydrogen bonds to the C3 domain. Finally, a comparison of the variable binding sites shows that, although the complementarity-determining region (CDR) loops align fairly closely with those of antibody molecules, there is some displacement relative to those of the antibody molecule. This displacement is particularly marked in the Vα CDR2 loop, which is oriented at roughly right angles to the equivalent loop in antibody V domains, as a result of a shift in the β strand that anchors one end of the loop from one face of the domain to the other. A strand displacement also causes a change in the orientation of the Vβ CDR2 loop in two of the seven Vβ domains whose structures are known. As yet, the crystallographic structures of seven T-cell receptors have been solved to this level of resolution.

Aspects of the disclosure relate to engineered T cell receptors that bind a peptide of the disclosure, such as a peptide of one of SEQ ID NOS:22-81. The term “engineered” refers to T cell receptors that have TCR variable regions grafted onto TCR constant regions to make a chimeric polypeptide that binds to peptides and antigens of the disclosure. In certain aspects, the TCR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

In some aspects, the TCR comprises non-TCR sequences. Accordingly, certain aspects relate to TCRs with sequences that are not from a TCR gene. In some aspects, the TCR is chimeric, in that it contains sequences normally found in a TCR gene, but contains sequences from at least two TCR genes that are not necessarily found together in nature.

### V. Antibodies

Aspects of the disclosure relate to antibodies that target the peptides of the disclosure, or fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COH end. The isotype of an antibody can be IgM, IgD, IgG, IgA, or IgE and is defined by the heavy chains present of which there are five classifications: mu (μ), delta (δ), gamma (γ), alpha (α), or epsilon (F) chains, respectively. IgG has several subtypes, including, but not limited to, IgG1, IgG2, IgG3, and IgG4. IgM subtypes include IgM1 and IgM2. IgA subtypes include IgA1 and IgA2.

### VI. Antibody Conjugates

Aspects of the disclosure relate to antibodies against a peptide of the disclosure, generally of the monoclonal type, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium⁸⁶, rhenium¹⁸⁸, ⁷⁵selenium, ³⁵sulphur, technicium^{99m} and/or yttrium⁹⁰. ¹²⁵I is often being preferred for use in certain aspects, and technicium^{99m} and/or indium¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other aspects, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

In another aspect of the disclosure, the antibody may be linked to semiconductor nanocrystals such as those described in U.S. Pat. Nos. 6,048,616; 5,990,479; 5,690,807; 5,505,928; 5,262,357 (all of which are incorporated herein in their entireties); as well as PCT Publication No. 99/26299 (published May 27, 1999). In particular, exemplary materials for use as semiconductor nanocrystals in the biological and chemical assays of the present invention include, but are not limited to those described above, including group II-VI, III-V and group IV semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe, Ge and Si and ternary and quaternary mixtures thereof. Methods for linking semiconductor nanocrystals to antibodies are described in U.S. Pat. Nos. 6,630,307 and 6,274,323.

In still further aspects, the present invention concerns immunodetection methods for binding, purifying, removing, quantifying and/or otherwise generally detecting biological components such as T cells or that selectively bind or recognize a peptide of the disclosure. In some aspects, a tetramer assay may be used with the present invention. Tetramer assays generally involve generating soluble peptide-MHC tetramers that may bind antigen specific T lymphocytes, and methods for tetramer assays are described, e.g., in Altman et al. (1996). Some immunodetection methods that may be used include, e.g., enzyme linked immunosorbent assay (ELISA), radioimmunoassay (RIA), immunoradiometric assay, fluoroimmunoassay, chemiluminescent assay, bioluminescent assay, tetramer assay, and Western blot. The steps of various useful immunodetection methods have been described in the scientific literature, such as, e.g., Doolittle and Ben-Zeev, 1999; Gulbis and Galand, 1993; De Jager et al., 1993; and Nakamura et al., 1987, each incorporated herein by reference.

### VII. MHC Polypeptides

Aspects of the disclosure relate to compositions comprising MHC polypeptides. In some aspects, the MHC polypeptide comprises at least 2, 3, or 4 MHC polypeptides that may be expressed as separate polypeptides or as a fusion protein. Presentation of antigens to T cells is mediated by two distinct classes of molecules MHC class I (MHC-I) and MHC class II (MHC-II) (also identified as “pMHC” herein), which utilize distinct antigen processing pathways. Peptides derived from intracellular antigens are presented to CD8+ T cells by MHC class I molecules, which are expressed on virtually all cells, while extracellular antigen-derived peptides are presented to CD4+ T cells by MHC-II molecules. In certain aspects, a particular antigen is identified and presented in the antigen-MHC complex in the context of an appropriate MHC class I or II polypeptide. In certain aspects, the genetic makeup of a subject may be assessed to determine which MHC polypeptide is to be used for a particular patient and a particular set of peptides. In certain aspects, the MHC class 1 polypeptide comprises all or part of a HLA-A, HLA-B, HLA-C, HLA-E, HLA-F, HLA-G or CD-1 molecule. In aspects wherein the MHC polypeptide is a MHC class II polypeptide, the MHC class II polypeptide can comprise all or a part of a HLA-DR, HLA-DQ, or HLA-DP.

Non-classical MHC polypeptides are also contemplated for use in MHC complexes of the invention. Non-classical MHC polypeptides are non-polymorphic, conserved among species, and possess narrow, deep, hydrophobic ligand binding pockets. These binding pockets are capable of presenting glycolipids and phospholipids to Natural Killer T (NKT) cells or certain subsets of CD8+ T-cells such as Qa1, HLA-E-restricted CD8+ T-cells, or MAIT cells. NKT cells represent a unique lymphocyte population that co-express NK cell markers and a semi-invariant T cell receptor (TCR). They are implicated in the regulation of immune responses associated with a broad range of diseases.

### VIII. Additional Agents

In some aspects, the method further comprises administration of an additional agent. In some aspects, the additional agent is an immunostimulator. The term “immunostimulator” as used herein refers to a compound that can stimulate an immune response in a subject, and may include an adjuvant. In some aspects, an immunostimulator is an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherichia coli, Salmonella minnesota, Salmonella typhimurium, or Shigella flexneri or specifically with MPL® (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

In some aspects, the additional agent comprises an agonist for pattern recognition receptors (PRR), including, but not limited to Toll-Like Receptors (TLRs), specifically TLRs 2, 3, 4, 5, 7, 8, 9 and/or combinations thereof. In some aspects, additional agents comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. In some aspects, the additional agents also may comprise immunostimulatory RNA molecules, such as but not limited to dsRNA, poly I:C or poly I:poly C12U (available as Ampligen®, both poly I:C and poly I:polyC12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some aspects, an additional agent may be a TLR-4 agonist, such as bacterial lipopolysaccharide (LPS), VSV-G, and/or HMGB-1. In some aspects, additional agents may comprise TLR-5 agonists, such as flagellin, or portions or derivatives thereof, including but not limited to those disclosed in U.S. Pat. Nos. 6,130,082, 6,585,980, and 7,192,725.

In some aspects, additional agents may be proinflammatory stimuli released from necrotic cells (e.g., urate crystals). In some aspects, additional agents may be activated components of the complement cascade (e.g., CD21, CD35, etc.). In some aspects, additional agents may be activated components of immune complexes. Additional agents also include complement receptor agonists, such as a molecule that binds to CD21 or CD35. In some aspects, the complement receptor agonist induces endogenous complement opsonization of the synthetic nanocarrier. In some aspects, immunostimulators are cytokines, which are small proteins or biological factors (in the range of 5 kD-20 kD) that are released by cells and have specific effects on cell-cell interaction, communication and behavior of other cells. In some aspects, the cytokine receptor agonist is a small molecule, antibody, fusion protein, or aptamer.

### IX. Engineered T Cell Receptors

T-cell receptors comprise two different polypeptide chains, termed the T-cell receptor α (TCRα) and β (TCRβ) chains, linked by a disulfide bond. These α:β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated 7 and 6. Both types of T-cell receptor differ from the membrane-bound immunoglobulin that serves as the B-cell receptor: a T-cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

The three-dimensional structure of the T-cell receptor has been determined. The structure is indeed similar to that of an antibody Fab fragment, as was suspected from earlier studies on the genes that encoded it. The T-cell receptor chains fold in much the same way as those of a Fab fragment, although the final structure appears a little shorter and wider. There are, however, some distinct differences between T-cell receptors and Fab fragments. The most striking difference is in the Cα domain, where the fold is unlike that of any other immunoglobulin-like domain. The half of the domain that is juxtaposed with the Cβ domain forms a β sheet similar to that found in other immunoglobulin-like domains, but the other half of the domain is formed of loosely packed strands and a short segment of α helix. The intramolecular disulfide bond, which in immunoglobulin-like domains normally joins two β strands, in a Cα domain joins a β strand to this segment of α helix.

There are also differences in the way in which the domains interact. The interface between the V and C domains of both T-cell receptor chains is more extensive than in antibodies, which may make the hinge joint between the domains less flexible. And the interaction between the Cα and Cβ domains is distinctive in being assisted by carbohydrate, with a sugar group from the Cα domain making a number of hydrogen bonds to the Cβ domain. Finally, a comparison of the variable binding sites shows that, although the complementarity-determining region (CDR) loops align fairly closely with those of antibody molecules, there is some displacement relative to those of the antibody molecule. This displacement is particularly marked in the Vα CDR2 loop, which is oriented at roughly right angles to the equivalent loop in antibody V domains, as a result of a shift in the β strand that anchors one end of the loop from one face of the domain to the other. A strand displacement also causes a change in the orientation of the Vβ CDR2 loop in two of the seven Vβ domains whose structures are known. As yet, the crystallographic structures of seven T-cell receptors have been solved to this level of resolution.

Aspects of the disclosure relate to engineered T cell receptors. The term “engineered” refers to T cell receptors that have TCR variable regions grafted onto TCR constant regions to make a chimeric polypeptide that binds to peptides and antigens of the disclosure. In certain aspects, the TCR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

In some aspects, the TCR comprises non-TCR sequences. Accordingly, certain aspects relate to TCRs with sequences that are not from a TCR gene. In some aspects, the TCR is chimeric, in that it contains sequences normally found in a TCR gene, but contains sequences from at least two TCR genes that are not necessarily found together in nature.

In some aspects the engineered TCRs of the disclosure comprise an aspect as shown below

TABLE 1
TCR Aspects

| | | SEQ |
|---|---|---|
| | | ID |
| Description | Sequence | NO: |
| DNA sequence: | ATGGAAACTCTCCTGGGAGTGTCTTTGG | 1 |
| Alpha Chain | TGATTCTATGGCTTCAACTGGCTAGGGT | |
| (TRAV17*01 F) | GAACAGTCAACAGGGAGAAGAGGATCCT | |
| | CAGGCCTTGAGCATCCAGGAGGGTGAAA | |
| | ATGCCACCATGAACTGCAGTTACAAAAC | |
| | TAGTATAAACAATTTACAGTGGTATAGA | |
| | CAAAATTCAGGTAGAGGCCTTGTCCACC | |
| | TAATTTTAATACGTTCAAATGAAAGAGA | |
| | GAAACACAGTGGAAGATTAAGAGTCACG | |
| | CTTGACACTTCCAAGAAAAGCAGTTCCT | |
| | TGTTGATCACGGCTTCCCGGGCAGCAGA | |
| | CACTGCTTCTTACTTCTGTGCTACGGCT | |
| | CAAACCTCCTACGACAAGGTGATATTTG | |
| | GGCCAGGGACAAGCTTATCAGTCATTCC | |
| | AAATATCCAGAACCCTGACCCTGCCGTG | |
| | TACCAGCTGAGAGACTCTAAATCCAGTG | |
| | ACAAGTCTGTCTGCCTATTCACCGATTT | |
| | TGATTCTCAAACAAATGTGTCACAAAGT | |
| | AAGGATTCTGATGTGTATATCACAGACA | |
| | AAACTGTGCTAGACATGAGGTCTATGGA | |
| | CTTCAAGAGCAACAGTGCTGTGGCCTGG | |
| | AGCAACAAATCTGACTTTGCATGTGCAA | |
| | ACGCCTTCAACAACAGCATTATTCCAGA | |
| | AGACACCTTCTTCCCCAGCCCAGAAAGT | |
| | TCCTGTGATGTCAAGCTGGTCGAGAAAA | |
| | GCTTTGAAACAGATACGAACCTAAACTT | |
| | TCAAAACCTGTCAGTGATTGGGTTCCGA | |
| | ATCCTCCTCCTGAAAGTGGCCGGGTTTA | |
| | ATCTGCTCATGACGCTGCGGCTGTGGTC | |
| | CAGCTAA | |
| Protein sequence | METLLGVSLVILWLQLARVNSQQGEEDP | 2 |
| (272aa): Alpha | QALSIQEGENATMNCSYKTSINNLQWYR | |
| Chain | QNSGRGLVHLILIRSNEREKHSGRLRVT | |
| (TRAV17*01 F) | LDTSKKSSSLLITASRAADTASYFCATA | |
| | QTSYDKVIFGPGTSLSVIPNIQNPDPAV | |
| | YQLRDSKSSDKSVCLFTDFDSQTNVSQS | |
| | KDSDVYITDKTVLDMRSMDFKSNSAVAW | |
| | SNKSDFACANAFNNSIIPEDTFFPSPES | |
| | SCDVKLVEKSFETDTNLNFQNLSVIGFR | |
| | ILLLKVAGFNLLMTLRLWSS | |
| Variable region: | SQQGEEDPQALSIQEGENATMNCSYKTS | 3 |
| Alpha Chain | INNLQWYRQNSGRGLVHLILIRSNEREK | |
| (TRAV17*01 F) | HSGRLRVTLDTSKKSSSLLITASRAADT | |
| | ASYFCATAQTSYDKVIFGPGTSLSVIPN | |
| Leader peptide: | METLLGVSLVILWLQLARVN | 4 |
| Alpha Chain | | |
| (TRAV17*01 F) | | |
| Alpha Chain | TSINN | 5 |
| (TRAV17*01 F) | | |
| CDR1 | | |
| Alpha Chain | IRSNERE | 6 |
| (TRAV17*01 F) | | |
| CDR2 | | |
| Alpha Chain | ATAQTSYDKVI | 7 |
| (TRAV17*01 F) | | |
| CDR3 | | |
| DNA sequence: | ATGGCATGCCCTGGCTTCCTGTGGGCAC | 8 |
| Alpha Chain | TTGTGATCTCCACCTGTCTTGAATTTAG | |
| (TRAV38- | CATGGCTCAGACAGTCACTCAGTCTCAA | |
| 2/DV8*01 F) | CCAGAGATGTCTGTGCAGGAGGCAGAGA | |
| | CCGTGACCCTGAGCTGCACATATGACAC | |
| | CAGTGAGAGTGATTATTATTTATTCTGG | |
| | TACAAGCAGCCTCCCAGCAGGCAGATGA | |
| | TTCTCGTTATTCGCCAAGAAGCTTATAA | |
| | GCAACAGAATGCAACAGAGAATCGTTTC | |
| | TCTGTGAACTTCCAGAAAGCAGCCAAAT | |
| | CCTTCAGTCTCAAGATCTCAGACTCACA | |
| | GCTGGGGGATGCCGCGATGTATTTCTGT | |
| | GCTTATAGGAGTGGAGGAACCTACAAAT | |
| | ACATCTTTGGAACAGGCACCAGGCTGAA | |
| | GGTTTTAGCAAATATCCAGAACCCTGAC | |
| | CCTGCCGTGTACCAGCTGAGAGACTCTA | |
| | AATCCAGTGACAAGTCTGTCTGCCTATT | |
| | CACCGATTTTGATTCTCAAACAAATGTG | |
| | TCACAAAGTAAGGATTCTGATGTGTATA | |
| | TCACAGACAAAACTGTGCTAGACATGAG | |
| | GTCTATGGACTTCAAGAGCAACAGTGCT | |
| | GTGGCCTGGAGCAACAAATCTGACTTTG | |
| | CATGTGCAAACGCCTTCAACAACAGCAT | |
| | TATTCCAGAAGACACCTTCTTCCCCAGC | |
| | CCAGAAAGTTCCTGTGATGTCAAGCTGG | |
| | TCGAGAAAAGCTTTGAAACAGATACGAA | |
| | CCTAAACTTTCAAAACCTGTCAGTGATT | |
| | GGGTTCCGAATCCTCCTCCTGAAAGTGG | |
| | CCGGGTTTAATCTGCTCATGACGCTGCG | |
| | GCTGTGGTCCAGCTAA | |
| Protein sequence | MACPGFLWALVISTCLEFSMAQTVTQSQ | 9 |
| (272aa): Alpha | PEMSVQEAETVTLSCTYDTSESDYYLFW | |
| Chain (TRAV38- | YKQPPSRQMILVIRQEAYKQQNATENRF | |
| 2/DV8*01 F) | SVNFQKAAKSFSLKISDSQLGDAAMYFC | |
| | AYRSGGTYKYIFGTGTRLKVLANIQNPD | |
| | PAVYQLRDSKSSDKSVCLFTDFDSQTNV | |
| | SQSKDSDVYITDKTVLDMRSMDFKSNSA | |
| | VAWSNKSDFACANAFNNSIIPEDTFFPS | |
| | PESSCDVKLVEKSFETDTNLNFQNLSVI | |
| | GFRILLLKVAGFNLLMTLRLWSS | |
| Variable region: | AQTVTQSQPEMSVQEAETVTLSCTYDTS | 10 |
| Alpha Chain | ESDYYLFWYKQPPSRQMILVIRQEAYKQ | |
| (TRAV38- | QNATENRFSVNFQKAAKSFSLKISDSQL | |
| 2/DV8*01 F) | GDAAMYFCAYRSGGTYKYIFGTGTRLKV | |
| | LAN | |
| Leader peptide: | MACPGFLWALVISTCLEFSM | 11 |
| Alpha Chain | | |
| (TRAV38- | | |
| 2/DV8*01 F) | | |
| Alpha Chain | TSESDYY | 12 |
| (TRAV38- | | |
| 2/DV8*01 F) | | |
| CDR1 | | |
| Alpha Chain | QEAYKQQN | 13 |
| (TRAV38- | | |
| 2/DV8*01 F) | | |
| CDR2 | | |
| Alpha Chain | AYRSGGTYKYI | 14 |
| (TRAV38- | | |
| 2/DV8*01 F) | | |
| CDR3 | | |
| DNA sequence: | ATGGGCTTCAGGCTCCTCTGCTGTGTGG | 15 |
| Beta Chain | CCTTTTGTCTCCTGGGAGCAGGCCCAGT | |
| (TRBV9*02 F) | GGATTCTGGAGTCACACAAACCCCAAAG | |
| | CACCTGATCACAGCAACTGGACAGCGAG | |
| | TGACGCTGAGATGCTCCCCTAGGTCTGG | |
| | AGACCTCTCTGTGTACTGGTACCAACAG | |
| | AGCCTGGACCAGGGCCTCCAGTTCCTCA | |
| | TTCACTATTATAATGGAGAAGAGAGAGC | |
| | AAAAGGAAACATTCTTGAACGATTCTCC | |
| | GCACAACAGTTCCCTGACTTGCACTCTG | |
| | AACTAAACCTGAGCTCTCTGGAGCTGGG | |
| | GGACTCAGCTTTGTATTTCTGTGCCAGC | |
| | AGCGTTGAGGGGCCCACCTACAATGAGC | |
| | AGTTCTTCGGGCCAGGGACACGGCTCAC | |
| | CGTGCTAGAGGACCTGAAAAACGTGTTC | |
| | CCACCCGAGGTCGCTGTGTTTGAGCCAT | |
| | CAGAAGCAGAGATCTCCCACACCCAAAA | |
| | GGCCACACTGGTGTGCCTGGCCACAGGC | |
| | TTCTTCCCTGACCACGTGGAGCTGAGCT | |
| | GGTGGGTGAATGGGAAGGAGGTGCACAG | |
| | TGGGGTCAGCACGGACCCGCAGCCCCTC | |
| | AAGGAGCAGCCCGCCCTCAATGACTCCA | |
| | GATACTGCCTGAGCAGCCGCCTGAGGGT | |
| | CTCGGCCACCTTCTGGCAGAACCCCCGC | |
| | AACCACTTCCGCTGTCAAGTCCAGTTCT | |
| | ACGGGCTCTCGGAGAATGACGAGTGGAC | |
| | CCAGGATAGGGCCAAACCCGTCACCCAG | |
| | ATCGTCAGCGCCGAGGCCTGGGGTAGAG | |
| | CAGACTGTGGCTTTACCTCGGTGTCCTA | |
| | CCAGCAAGGGGTCCTGTCTGCCACCATC | |
| | CTCTATGAGATCCTGCTAGGGAAGGCCA | |
| | CCCTGTATGCTGTGCTGGTCAGCGCCCT | |
| | TGTGTTGATGGCCATGGTCAAGAGAAAG | |
| | GATTTCTAA | |
| Protein sequence | MGFRLLCCVAFCLLGAGPVDSGVTQTPK | 16 |
| (272aa): Beta | HLITATGQRVTLRCSPRSGDLSVYWYQQ | |
| Chain (TRBV9*02 | SLDQGLQFLIHYYNGEERAKGNILERFS | |
| F) | AQQFPDLHSELNLSSLELGDSALYFCAS | |
| | SVEGPTYNEQFFGPGTRLTVLEDLKNVF | |
| | PPEVAVFEPSEAEISHTQKATLVCLATG | |
| | FFPDHVELSWWVNGKEVHSGVSTDPQPL | |
| | KEQPALNDSRYCLSSRLRVSATFWQNPR | |
| | NHFRCQVQFYGLSENDEWTQDRAKPVTQ | |
| | IVSAEAWGRADCGFTSVSYQQGVLSATI | |
| | LYEILLGKATLYAVLVSALVLMAMVKRK | |
| | DF | |
| Variable region: | DSGVTQTPKHLITATGQRVTLRCSPRSG | 17 |
| Beta Chain | DLSVYWYQQSLDQGLQFLIHYYNGEERA | |
| (TRBV9*02 F) | KGNILERFSAQQFPDLHSELNLSSLELG | |
| | DSALYFCASSVEGPTYNEQFFGPGTRLT | |
| | VL | |
| Leader peptide: | MGFRLLCCVAFCLLGAGPV | 18 |
| Beta Chain | | |
| (TRBV9*02 F) | | |
| Beta Chain | SGDLS | 19 |
| (TRBV9*02 F) | | |
| CDR1 | | |
| Beta Chain | YYNGEE | 20 |
| (TRBV9*02 F) | | |
| CDR2 | | |
| Beta Chain | ASSVEGPTYNEQF | 21 |
| (TRBV9*02 F) | | |
| CDR3 | | |
| DNA sequence: | ATGGCCTCTGCACCCATCTCGATGCTTG | 86 |
| Alpha Chain | CGATGCTCTTCACATTGAGTGGGCTGAG | |
| (TRAV3*01 F); | AGCTCAGTCAGTGGCTCAGCCGGAAGAT | |
| NSP13-134 TCR | CAGGTCAACGTTGCTGAAGGGAATCCTC | |
| | TGACTGTGAAATGCACCTATTCAGTCTC | |
| | TGGAAACCCTTATCTTTTTTGGTATGTT | |
| | CAATACCCCAACCGAGGCCTCCAGTTCC | |
| | TTCTGAAATACATCACAGGGGATAACCT | |
| | GGTTAAAGGCAGCTATGGCTTTGAAGCT | |
| | GAATTTAACAAGAGCCAAACCTCCTTCC | |
| | ACCTGAAGAAACCATCTGCCCTTGTGAG | |
| | CGACTCCGCTTTGTACTTCTGTGCTGTG | |
| | AGAGACAGAGGCTTTGGGAATGTGCTGC | |
| | ATTGCGGGTCCGGCACTCAAGTGATTGT | |
| | TTTACCACATATCCAGAACCCTGACCCT | |
| | GCCGTGTACCAGCTGAGAGACTCTAAAT | |
| | CCAGTGACAAGTCTGTCTGCCTATTCAC | |
| | CGATTTTGATTCTCAAACAAATGTGTCA | |
| | CAAAGTAAGGATTCTGATGTGTATATCA | |
| | CAGACAAAACTGTGCTAGACATGAGGTC | |
| | TATGGACTTCAAGAGCAACAGTGCTGTG | |
| | GCCTGGAGCAACAAATCTGACTTTGCAT | |
| | GTGCAAACGCCTTCAACAACAGCATTAT | |
| | TCCAGAAGACACCTTCTTCCCCAGCCCA | |
| | GAAAGTTCCTGTGATGTCAAGCTGGTCG | |
| | AGAAAAGCTTTGAAACAGATACGAACCT | |
| | AAACTTTCAAAACCTGTCAGTGATTGGG | |
| | TTCCGAATCCTCCTCCTGAAAGTGGCCG | |
| | GGTTTAATCTGCTCATGACGCTGCGGCT | |
| | GTGGTCCAGCTAA | |
| Protein sequence | MASAPISMLAMLFTLSGLRAQSVAQPED | 87 |
| (274aa): Alpha | QVNVAEGNPLTVKCTYSVSGNPYLFWYV | |
| Chain | QYPNRGLQFLLKYITGDNLVKGSYGFEA | |
| (TRAV3*01 F); | EFNKSQTSFHLKKPSALVSDSALYFCAV | |
| NSP13-134 TCR | RDRGFGNVLHCGSGTQVIVLPHIQNPDP | |
| | AVYQLRDSKSSDKSVCLFTDFDSQTNVS | |
| | QSKDSDVYITDKTVLDMRSMDFKSNSAV | |
| | AWSNKSDFACANAFNNSIIPEDTFFPSP | |
| | ESSCDVKLVEKSFETDTNLNFQNLSVIG | |
| | FRILLLKVAGFNLLMTLRLWSS | |
| Variable region: | AQSVAQPEDQVNVAEGNPLTVKCTYSVS | 88 |
| Alpha Chain | GNPYLFWYVQYPNRGLQFLLKYITGDNL | |
| (TRAV3*01 F); | VKGSYGFEAEFNKSQTSFHLKKPSALVS | |
| NSP13-134 TCR | DSALYFCAVRDRGFGNVLHCGSGTQVIV | |
| | LPHI | |
| Leader peptide: | MASAPISMLAMLFTLSGLR | 89 |
| Alpha Chain | | |
| (TRAV3*01 F); | | |
| NSP13-134 TCR | | |
| Alpha Chain | VSGNPY | 90 |
| (TRAV3*01 F) | | |
| CDR1 | | |
| Alpha Chain | YITGDNLV | 91 |
| (TRAV3*01 F) | | |
| CDR2 | | |
| Alpha Chain | AVRDRGFGNVLH | 92 |
| (TRAV3*01 F) | | |
| CDR3 | | |
| DNA sequence: | ATGCTGAGTCTTCTGCTCCTTCTCCTGG | 93 |
| Beta Chain | GACTAGGCTCTGTGTTCAGTGCTGTCAT | |
| (TRBV29-1*01 | CTCTCAAAAGCCAAGCAGGGATATCTGT | |
| F); NSP13-134 | CAACGTGGAACCTCCCTGACGATCCAGT | |
| TCR | GTCAAGTCGATAGCCAAGTCACCATGAT | |
| | GTTCTGGTACCGTCAGCAACCTGGACAG | |
| | AGCCTGACACTGATCGCAACTGCAAATC | |
| | AGGGCTCTGAGGCCACATATGAGAGTGG | |
| | ATTTGTCATTGACAAGTTTCCCATCAGC | |
| | CGCCCAAACCTAACATTCTCAACTCTGA | |
| | CTGTGAGCAACATGAGCCCTGAAGACAG | |
| | CAGCATATATCTCTGCAGCGCCCGGTTG | |
| | AAGGACTCCTACGAGCAGTACTTCGGGC | |
| | CGGGCACCAGGCTCACGGTCACAGAGGA | |
| | CCTGAAAAACGTGTTCCCACCCGAGGTC | |
| | GCTGTGTTTGAGCCATCAGAAGCAGAGA | |
| | TCTCCCACACCCAAAAGGCCACACTGGT | |
| | ATGCCTGGCCACAGGCTTCTACCCCGAC | |
| | CACGTGGAGCTGAGCTGGTGGGTGAATG | |
| | GGAAGGAGGTGCACAGTGGGGTCAGCAC | |
| | AGACCCGCAGCCCCTCAAGGAGCAGCCC | |
| | GCCCTCAATGACTCCAGATACTGCCTGA | |
| | GCAGCCGCCTGAGGGTCTCGGCCACCTT | |
| | CTGGCAGAACCCCCGCAACCACTTCCGC | |
| | TGTCAAGTCCAGTTCTACGGGCTCTCGG | |
| | AGAATGACGAGTGGACCCAGGATAGGGC | |
| | CAAACCCGTCACCCAGATCGTCAGCGCC | |
| | GAGGCCTGGGGTAGAGCAGACTGTGGCT | |
| | TTACCTCGGTGTCCTACCAGCAAGGGGT | |
| | CCTGTCTGCCACCATCCTCTATGAGATC | |
| | CTGCTAGGGAAGGCCACCCTGTATGCTG | |
| | TGCTGGTCAGCGCCCTTGTGTTGATGGC | |
| | CATGGTCAAGAGAAAGGATTTCTAA | |
| Protein sequence | MLSLLLLLLGLGSVFSAVISQKPSRDIC | 94 |
| (306aa): Beta | QRGTSLTIQCQVDSQVTMMFWYRQQPGQ | |
| Chain (TRBV29- | SLTLIATANQGSEATYESGFVIDKFPIS | |
| 1*01 F); NSP13- | RPNLTFSTLTVSNMSPEDSSIYLCSARL | |
| 134 TCR | KDSYEQYFGPGTRLTVTEDLKNVFPPEV | |
| | AVFEPSEAEISHTQKATLVCLATGFYPD | |
| | HVELSWWVNGKEVHSGVSTDPQPLKEQP | |
| | ALNDSRYCLSSRLRVSATFWQNPRNHFR | |
| | CQVQFYGLSENDEWTQDRAKPVTQIVSA | |
| | EAWGRADCGFTSVSYQQGVLSATILYEI | |
| | LLGKATLYAVLVSALVLMAMVKRKDF | |
| Variable region: | SAVISQKPSRDICQRGTSLTIQCQVDSQ | 95 |
| Beta Chain | VTMMFWYRQQPGQSLTLIATANQGSEAT | |
| (TRBV29-1*01 | YESGFVIDKFPISRPNLTESTLTVSNMS | |
| F); NSP13-134 | PEDSSIYLCSARLKDSYEQYFGPGTRLT | |
| TCR | VTE | |
| Leader peptide: | MLSLLLLLLGLGSVF | 96 |
| Beta Chain | | |
| (TRBV29-1*01 | | |
| F); NSP13-134 | | |
| TCR | | |
| Beta Chain | SQVTM | 97 |
| (TRBV29-1*01 F) | | |
| CDR1 | | |
| Beta Chain | ANQGSEA | 98 |
| (TRBV29-1*01 F) | | |
| CDR2 | | |
| Beta Chain | SARLKDSYEQY | 99 |
| (TRBV29-1*01 F) | | |
| CDR3 | | |
| DNA sequence: | ATGACACGAGTTAGCTTGCTGTGGGCAG | 100 |
| Alpha Chain | TCGTGGTCTCCACCTGTCTTGAATCCGG | |
| (TRAV38-1*01 | CATGGCCCAGACAGTCACTCAGTCTCAA | |
| F); NSP13-242 | CCAGAGATGTCTGTGCAGGAGGCAGAGA | |
| TCR | CTGTGACCCTGAGTTGCACATATGACAC | |
| | CAGTGAGAATAATTATTATTTGTTCTGG | |
| | TACAAGCAGCCTCCCAGCAGGCAGATGA | |
| | TTCTCGTTATTCGCCAAGAAGCTTATAA | |
| | GCAACAGAATGCAACGGAGAATCGTTTC | |
| | TCTGTGAACTTCCAGAAAGCAGCCAAAT | |
| | CCTTCAGTCTCAAGATCTCAGACTCACA | |
| | GCTGGGGGACACTGCGATGTATTTCTGT | |
| | GCTTTCATGAACTCCAACAATGCCAGAC | |
| | TCATGTTTGGAGATGGAACTCAGCTGGT | |
| | GGTGAAGCCCAATATCCAGAACCCTGAC | |
| | CCTGCCGTGTACCAGCTGAGAGACTCTA | |
| | AATCCAGTGACAAGTCTGTCTGCCTATT | |
| | CACCGATTTTGATTCTCAAACAAATGTG | |
| | TCACAAAGTAAGGATTCTGATGTGTATA | |
| | TCACAGACAAAACTGTGCTAGACATGAG | |
| | GTCTATGGACTTCAAGAGCAACAGTGCT | |
| | GTGGCCTGGAGCAACAAATCTGACTTTG | |
| | CATGTGCAAACGCCTTCAACAACAGCAT | |
| | TATTCCAGAAGACACCTTCTTCCCCAGC | |
| | CCAGAAAGTTCCTGTGATGTCAAGCTGG | |
| | TCGAGAAAAGCTTTGAAACAGATACGAA | |
| | CCTAAACTTTCAAAACCTGTCAGTGATT | |
| | GGGTTCCGAATCCTCCTCCTGAAAGTGG | |
| | CCGGGTTTAATCTGCTCATGACGCTGCG | |
| | GCTGTGGTCCAGCTAA | |
| Protein sequence | MTRVSLLWAVVVSTCLESGMAQTVTQSQ | 101 |
| (275aa): Alpha | PEMSVQEAETVTLSCTYDTSENNYYLFW | |
| Chain (TRAV38- | YKQPPSRQMILVIRQEAYKQQNATENRF | |
| 1*01 F); NSP13- | SVNFQKAAKSFSLKISDSQLGDTAMYFC | |
| 242 TCR | AFMNSNNARLMFGDGTQLVVKPNIQNPD | |
| | PAVYQLRDSKSSDKSVCLFTDFDSQTNV | |
| | SQSKDSDVYITDKTVLDMRSMDFKSNSA | |
| | VAWSNKSDFACANAFNNSIIPEDTFFPS | |
| | PESSCDVKLVEKSFETDTNLNFQNLSVI | |
| | GFRILLLKVAGFNLLMTLRLWSS | |
| Variable region: | AQTVTQSQPEMSVQEAETVTLSCTYDTS | 102 |
| Alpha Chain | ENNYYLFWYKQPPSRQMILVIRQEAYKQ | |
| (TRAV38-1*01 | QNATENRFSVNFQKAAKSFSLKISDSQL | |
| F); NSP13-242 | GDTAMYFCAFMNSNNARLMFGDGTQLVV | |
| TCR | KPN | |
| Leader peptide: | MTRVSLLWAVVVSTCLESGM | 103 |
| Alpha Chain | | |
| (TRAV38-1*01 | | |
| F); NSP13-242 | | |
| TCR | | |
| Alpha Chain | TSENNYY | 104 |
| (TRAV38-1*01 F) | | |
| CDR1 | | |
| Alpha Chain | QEAYKQQN | 105 |
| (TRAV38-1*01 F) | | |
| CDR2 | | |
| Alpha Chain | AFMNSNNARLM | 106 |
| (TRAV38-1*01 F) | | |
| CDR3 | | |
| DNA sequence: | ATGGGCACCAGGCTCTTCTTCTATGTGG | 107 |
| Beta Chain | CCCTTTGTCTGCTGTGGGCAGGACACAG | |
| (TRBV10-2*02 | GGATGCTGGAATCACCCAGAGCCCAAGA | |
| F); NSP13-242 | TACAAGATCACAGAGACAGGAAGGCAGG | |
| TCR | TGACCTTGATGTGTCACCAGACTTGGAG | |
| | CCACAGCTATATGTTCTGGTATCGACAA | |
| | GACCTGGGACATGGGCTGAGGCTGATCT | |
| | ATTACTCAGCAGCTGCTGATATTACAGA | |
| | TAAAGGAGAAGTCCCCGATGGCTACGTT | |
| | GTCTCCAGATCCAAGACAGAGAATTTCC | |
| | CCCTCACTCTGGAGTCAGCTACCCGCTC | |
| | CCAGACATCTGTGTATTTCTGCGCCAGC | |
| | AGCCCGGGGTCCGTCCCAGGCGGCTACA | |
| | CCTTCGGTTCGGGGACCAGGTTAACCGT | |
| | TGTAGAGGACCTGAACAAGGTGTTCCCA | |
| | CCCGAGGTCGCTGTGTTTGAGCCATCAG | |
| | AAGCAGAGATCTCCCACACCCAAAAGGC | |
| | CACACTGGTGTGCCTGGCCACAGGCTTC | |
| | TTCCCCGACCACGTGGAGCTGAGCTGGT | |
| | GGGTGAATGGGAAGGAGGTGCACAGTGG | |
| | GGTCAGCACAGACCCGCAGCCCCTCAAG | |
| | GAGCAGCCCGCCCTCAATGACTCCAGAT | |
| | ACTGCCTGAGCAGCCGCCTGAGGGTCTC | |
| | GGCCACCTTCTGGCAGAACCCCCGCAAC | |
| | CACTTCCGCTGTCAAGTCCAGTTCTACG | |
| | GGCTCTCGGAGAATGACGAGTGGACCCA | |
| | GGATAGGGCCAAACCCGTCACCCAGATC | |
| | GTCAGCGCCGAGGCCTGGGGTAGAGCAG | |
| | ACTGTGGCTTTACCTCGGTGTCCTACCA | |
| | GCAAGGGGTCCTGTCTGCCACCATCCTC | |
| | TATGAGATCCTGCTAGGGAAGGCCACCC | |
| | TGTATGCTGTGCTGGTCAGCGCCCTTGT | |
| | GTTGATGGCCATGGTCAAGAGAAAGGAT | |
| | TTCTAA | |
| Protein sequence | MGTRLFFYVALCLLWAGHRDAGITQSPR | 108 |
| (306aa): Beta | YKITETGRQVTLMCHQTWSHSYMFWYRQ | |
| Chain (TRBV10- | DLGHGLRLIYYSAAADITDKGEVPDGYV | |
| 2*02 F); NSP13- | VSRSKTENFPLTLESATRSQTSVYFCAS | |
| 242 TCR | SPGSVPGGYTFGSGTRLTVVEDLNKVFP | |
| | PEVAVFEPSEAEISHTQKATLVCLATGF | |
| | FPDHVELSWWVNGKEVHSGVSTDPQPLK | |
| | EQPALNDSRYCLSSRLRVSATFWQNPRN | |
| | HFRCQVQFYGLSENDEWTQDRAKPVTQI | |
| | VSAEAWGRADCGFTSVSYQQGVLSATIL | |
| | YEILLGKATLYAVLVSALVLMAMVKRKD | |
| | F | |
| Variable region: | DAGITQSPRYKITETGRQVTLMCHQTWS | 109 |
| Beta Chain | HSYMFWYRQDLGHGLRLIYYSAAADITD | |
| (TRBV10-2*02 | KGEVPDGYVVSRSKTENFPLTLESATRS | |
| F); NSP13-242 | QTSVYFCASSPGSVPGGYTFGSGTRLTV | |
| TCR | V | |
| Leader peptide: | MGTRLFFYVALCLLWAGHR | 110 |
| Beta Chain | | |
| (TRBV10-2*02 | | |
| F); NSP13-242 | | |
| TCR | | |
| Beta Chain | WSHSY | 111 |
| (TRBV10-2*02 F) | | |
| CDR1 | | |
| Beta Chain | SAAADI | 112 |
| (TRBV10-2*02 F) | | |
| CDR2 | | |
| Beta Chain | ASSPGSVPGGYT | 113 |
| (TRBV10-2*02 F) | | |
| CDR3 | | |
| DNA sequence: | ATGATGATATCCTTGAGAGTTTTACTGG | 114 |
| Alpha Chain | TGATCCTGTGGCTTCAGTTAAGCTGGGT | |
| (TRAV12-1*01 | TTGGAGCCAACGGAAGGAGGTGGAGCAG | |
| F); NSP13-400 | GATCCTGGACCCTTCAATGTTCCAGAGG | |
| TCR | GAGCCACTGTCGCTTTCAACTGTACTTA | |
| | CAGCAACAGTGCTTCTCAGTCTTTCTTC | |
| | TGGTACAGACAGGATTGCAGGAAAGAAC | |
| | CTAAGTTGCTGATGTCCGTATACTCCAG | |
| | TGGTAATGAAGATGGAAGGTTTACAGCA | |
| | CAGCTCAATAGAGCCAGCCAGTATATTT | |
| | CCCTGCTCATCAGAGACTCCAAGCTCAG | |
| | TGATTCAGCCACCTACCTCTGTGTGGTG | |
| | AAAGACAGCTGGGGGAAATTGCAGTTTG | |
| | GAGCAGGGACCCAGGTTGTGGTCACCCC | |
| | AGATATCCAGAACCCTGACCCTGCCGTG | |
| | TACCAGCTGAGAGACTCTAAATCCAGTG | |
| | ACAAGTCTGTCTGCCTATTCACCGATTT | |
| | TGATTCTCAAACAAATGTGTCACAAAGT | |
| | AAGGATTCTGATGTGTATATCACAGACA | |
| | AAACTGTGCTAGACATGAGGTCTATGGA | |
| | CTTCAAGAGCAACAGTGCTGTGGCCTGG | |
| | AGCAACAAATCTGACTTTGCATGTGCAA | |
| | ACGCCTTCAACAACAGCATTATTCCAGA | |
| | AGACACCTTCTTCCCCAGCCCAGAAAGT | |
| | TCCTGTGATGTCAAGCTGGTCGAGAAAA | |
| | GCTTTGAAACAGATACGAACCTAAACTT | |
| | TCAAAACCTGTCAGTGATTGGGTTCCGA | |
| | ATCCTCCTCCTGAAAGTGGCCGGGTTTA | |
| | ATCTGCTCATGACGCTGCGGCTGTGGTC | |
| | CAGCTAA | |
| Protein sequence | MMISLRVLLVILWLQLSWVWSQRKEVEQ | 115 |
| (275aa): Alpha | DPGPFNVPEGATVAFNCTYSNSASQSFF | |
| Chain (TRAV12- | WYRQDCRKEPKLLMSVYSSGNEDGRFTA | |
| 1*01 F); NSP13- | QLNRASQYISLLIRDSKLSDSATYLCVV | |
| 400 TCR | KDSWGKLQFGAGTQVVVTPDIQNPDPAV | |
| | YQLRDSKSSDKSVCLFTDFDSQTNVSQS | |
| | KDSDVYITDKTVLDMRSMDFKSNSAVAW | |
| | SNKSDFACANAFNNSIIPEDTFFPSPES | |
| | SCDVKLVEKSFETDTNLNFQNLSVIGFR | |
| | ILLLKVAGFNLLMTLRLWSS | |
| Variable region: | RKEVEQDPGPFNVPEGATVAFNCTYSNS | 116 |
| Alpha Chain | ASQSFFWYRQDCRKEPKLLMSVYSSGNE | |
| (TRAV12-1*01 | DGRFTAQLNRASQYISLLIRDSKLSDSA | |
| F); NSP13-400 | TYLCVVKDSWGKLQFGAGTQVVVTPD | |
| TCR | | |
| Leader peptide: | MMISLRVLLVILWLQLSWVWSQ | 117 |
| Alpha Chain | | |
| (TRAV12-1*01 | | |
| F); NSP13-400 | | |
| TCR | | |
| Alpha Chain | NSASQS | 118 |
| (TRAV12-1*01 F) | | |
| CDR1 | | |
| Alpha Chain | VYSSGN | 119 |
| (TRAV12-1*01 F) | | |
| CDR2 | | |
| Alpha Chain | VVKDSWGKLQ | 120 |
| (TRAV12-1*01 F) | | |
| CDR3 | | |
| DNA sequence: | ATGGCCACCAGGCTCCTCTGCTGTGTGG | 121 |
| Beta Chain | TTCTTTGTCTCCTGGGAGAAGAGCTTAT | |
| (TRBV12-5*01F); | AGATGCTAGAGTCACCCAGACACCAAGG | |
| NSP13-400 TCR | CACAAGGTGACAGAGATGGGACAAGAAG | |
| | TAACAATGAGATGTCAGCCAATTTTAGG | |
| | CCACAATACTGTTTTCTGGTACAGACAG | |
| | ACCATGATGCAAGGACTGGAGTTGCTGG | |
| | CTTACTTCCGCAACCGGGCTCCTCTAGA | |
| | TGATTCGGGGATGCCGAAGGATCGATTC | |
| | TCAGCAGAGATGCCTGATGCAACTTTAG | |
| | CCACTCTGAAGATCCAGCCCTCAGAACC | |
| | CAGGGACTCAGCTGTGTATTTTTGTGCT | |
| | AGTGGTCCTTCAGGGGGCTGGAACACCA | |
| | TATATTTTGGAGAGGGAAGTTGGCTCAC | |
| | TGTTGTAGAGGACCTGAACAAGGTGTTC | |
| | CCACCCGAGGTCGCTGTGTTTGAGCCAT | |
| | CAGAAGCAGAGATCTCCCACACCCAAAA | |
| | GGCCACACTGGTGTGCCTGGCCACAGGC | |
| | TTCTTCCCTGACCACGTGGAGCTGAGCT | |
| | GGTGGGTGAATGGGAAGGAGGTGCACAG | |
| | TGGGGTCAGCACGGACCCGCAGCCCCTC | |
| | AAGGAGCAGCCCGCCCTCAATGACTCCA | |
| | GATACTGCCTGAGCAGCCGCCTGAGGGT | |
| | CTCGGCCACCTTCTGGCAGAACCCCCGC | |
| | AACCACTTCCGCTGTCAAGTCCAGTTCT | |
| | ACGGGCTCTCGGAGAATGACGAGTGGAC | |
| | CCAGGATAGGGCCAAACCCGTCACCCAG | |
| | ATCGTCAGCGCCGAGGCCTGGGGTAGAG | |
| | CAGACTGTGGCTTTACCTCGGTGTCCTA | |
| | CCAGCAAGGGGTCCTGTCTGCCACCATC | |
| | CTCTATGAGATCCTGCTAGGGAAGGCCA | |
| | CCCTGTATGCTGTGCTGGTCAGCGCCCT | |
| | TGTGTTGATGGCCATGGTCAAGAGAAAG | |
| | GATTTCTAA | |
| Protein sequence | MATRLLCCVVLCLLGEELIDARVTQTPR | 122 |
| (306aa): Beta | HKVTEMGQEVTMRCQPILGHNTVFWYRQ | |
| Chain (TRBV10- | TMMQGLELLAYFRNRAPLDDSGMPKDRF | |
| 2*02 F); NSP13- | SAEMPDATLATLKIQPSEPRDSAVYFCA | |
| 400 TCR | SGPSGGWNTIYFGEGSWLTVVEDLNKVF | |
| | PPEVAVFEPSEAEISHTQKATLVCLATG | |
| | FFPDHVELSWWVNGKEVHSGVSTDPQPL | |
| | KEQPALNDSRYCLSSRLRVSATFWQNPR | |
| | NHFRCQVQFYGLSENDEWTQDRAKPVTQ | |
| | IVSAEAWGRADCGFTSVSYQQGVLSATI | |
| | LYEILLGKATLYAVLVSALVLMAMVKR | |
| | KDF | |
| Variable region: | DARVTQTPRHKVTEMGQEVTMRCQPILG | 123 |
| Beta Chain | HNTVFWYRQTMMQGLELLAYFRNRAPLD | |
| (TRBV10-2*02 | DSGMPKDRFSAEMPDATLATLKIQPSEP | |
| F); NSP13-400 | RDSAVYFCASGPSGGWNTIYFGEGSWLT | |
| TCR | VV | |
| Leader peptide: | MATRLLCCVVLCLLGEELI | 124 |
| Beta Chain | | |
| (TRBV10-2*02 | | |
| F); NSP13-400 | | |
| TCR | | |
| Beta Chain | LGHNT | 125 |
| (TRBV12-5*01F) | | |
| CDR1 | | |
| Beta Chain | FRNRAP | 126 |
| (TRBV12-5*01F) | | |
| CDR2 | | |
| Beta Chain | ASGPSGGWNTIY | 127 |
| (TRBV12-5*01F) | | |
| CDR3 | | |
| DNA sequence: | ATGCTCCTGCTGCTCGTCCCAGTGCTCG | 128 |
| Alpha Chain | AGGTGATTTTTACCCTGGGAGGAACCAG | |
| (TRAV8-4*01 F); | AGCCCAGTCGGTGACCCAGCTTGGCAGC | |
| NSP13-448 TCR | CACGTCTCTGTCTCTGAAGGAGCCCTGG | |
| | TTCTGCTGAGGTGCAACTACTCATCGTC | |
| | TGTTCCACCATATCTCTTCTGGTATGTG | |
| | CAATACCCCAACCAAGGACTCCAGCTTC | |
| | TCCTGAAGTACACATCAGCGGCCACCCT | |
| | GGTTAAAGGCATCAACGGTTTTGAGGCT | |
| | GAATTTAAGAAGAGTGAAACCTCCTTCC | |
| | ACCTGACGAAACCCTCAGCCCATATGAG | |
| | CGACGCGGCTGAGTACTTCTGTGCTGTG | |
| | AGTAATTCAGGAGGAGGTGCTGACGGAC | |
| | TCACCTTTGGCAAAGGGACTCATCTAAT | |
| | CATCCAGCCCTATATCCAGAACCCTGAC | |
| | CCTGCCGTGTACCAGCTGAGAGACTCTA | |
| | AATCCAGTGACAAGTCTGTCTGCCTATT | |
| | CACCGATTTTGATTCTCAAACAAATGTG | |
| | TCACAAAGTAAGGATTCTGATGTGTATA | |
| | TCACAGACAAAACTGTGCTAGACATGAG | |
| | GTCTATGGACTTCAAGAGCAACAGTGCT | |
| | GTGGCCTGGAGCAACAAATCTGACTTTG | |
| | CATGTGCAAACGCCTTCAACAACAGCAT | |
| | TATTCCAGAAGACACCTTCTTCCCCAGC | |
| | CCAGAAAGTTCCTGTGATGTCAAGCTGG | |
| | TCGAGAAAAGCTTTGAAACAGATACGAA | |
| | CCTAAACTTTCAAAACCTGTCAGTGATT | |
| | GGGTTCCGAATCCTCCTCCTGAAAGTGG | |
| | CCGGGTTTAATCTGCTCATGACGCTGCG | |
| | GCTGTGGTCCAGCTAA | |
| Protein sequence | MLLLLVPVLEVIFTLGGTRAQSVTQLGS | 129 |
| (275aa): Alpha | HVSVSEGALVLLRCNYSSSVPPYLFWYV | |
| Chain (TRAV8- | QYPNQGLQLLLKYTSAATLVKGINGFEA | |
| 4*01 F); NSP13- | EFKKSETSFHLTKPSAHMSDAAEYFCAV | |
| 448 TCR | SNSGGGADGLTFGKGTHLIIQPYIQNPD | |
| | PAVYQLRDSKSSDKSVCLFTDFDSQTNV | |
| | SQSKDSDVYITDKTVLDMRSMDFKSNSA | |
| | VAWSNKSDFACANAFNNSIIPEDTFFPS | |
| | PESSCDVKLVEKSFETDTNLNFQNLSVI | |
| | GFRILLLKVAGFNLLMTLRLWSS | |
| Variable region: | AQSVTQLGSHVSVSEGALVLLRCNYSSS | 130 |
| Alpha Chain | VPPYLFWYVQYPNQGLQLLLKYTSAATL | |
| (TRAV8-4*01 F); | VKGINGFEAEFKKSETSFHLTKPSAHMS | |
| NSP13-448 TCR | DAAEYFCAVSNSGGGADGLTFGKGTHLI | |
| | IQPY | |
| Leader peptide: | MLLLLVPVLEVIFTLGGTR | 131 |
| Alpha Chain | | |
| (TRAV8-4*01 F); | | |
| NSP13-448 TCR | | |
| Alpha Chain | SSVPPY | 132 |
| (TRAV8-4*01 F) | | |
| CDR1 | | |
| Alpha Chain | YTSAATLV | 133 |
| (TRAV8-4*01 F) | | |
| CDR2 | | |
| Alpha Chain | AVSNSGGGADGLT | 134 |
| (TRAV8-4*01 F) | | |
| CDR3 | | |
| DNA sequence: | ATGAGCAACCAGGTGCTCTGCTGTGTGG | 135 |
| Beta Chain | TCCTTTGTTTCCTGGGAGCAAACACCGT | |
| (TRBV19*01 F); | GGATGGTGGAATCACTCAGTCCCCAAAG | |
| NSP13-448 TCR | TACCTGTTCAGAAAGGAAGGACAGAATG | |
| | TGACCCTGAGTTGTGAACAGAATTTGAA | |
| | CCACGATGCCATGTACTGGTACCGACAG | |
| | GACCCAGGGCAAGGGCTGAGATTGATCT | |
| | ACTACTCACAGATAGTAAATGACTTTCA | |
| | GAAAGGAGATATAGCTGAAGGGTACAGC | |
| | GTCTCTCGGGAGAAGAAGGAATCCTTTC | |
| | CTCTCACTGTGACATCGGCCCAAAAGAA | |
| | CCCGACAGCTTTCTATCTCTGTGCCAGT | |
| | AGTATTATGGCAGGGGGTCACGGGGAGC | |
| | TGTTTTTTGGAGAAGGCTCTAGGCTGAC | |
| | CGTACTGGAGGACCTGAAAAACGTGTTC | |
| | CCACCCGAGGTCGCTGTGTTTGAGCCAT | |
| | CAGAAGCAGAGATCTCCCACACCCAAAA | |
| | GGCCACACTGGTGTGCCTGGCCACAGGC | |
| | TTCTTCCCCGACCACGTGGAGCTGAGCT | |
| | GGTGGGTGAATGGGAAGGAGGTGCACAG | |
| | TGGGGTCAGCACAGACCCGCAGCCCCTC | |
| | AAGGAGCAGCCCGCCCTCAATGACTCCA | |
| | GATACTGCCTGAGCAGCCGCCTGAGGGT | |
| | CTCGGCCACCTTCTGGCAGAACCCCCGC | |
| | AACCACTTCCGCTGTCAAGTCCAGTTCT | |
| | ACGGGCTCTCGGAGAATGACGAGTGGAC | |
| | CCAGGATAGGGCCAAACCCGTCACCCAG | |
| | ATCGTCAGCGCCGAGGCCTGGGGTAGAG | |
| | CAGACTGTGGCTTTACCTCGGTGTCCTA | |
| | CCAGCAAGGGGTCCTGTCTGCCACCATC | |
| | CTCTATGAGATCCTGCTAGGGAAGGCCA | |
| | CCCTGTATGCTGTGCTGGTCAGCGCCCT | |
| | TGTGTTGATGGCCATGGTCAAGAGAAAG | |
| | GATTTCTAA | |
| Protein sequence | MSNQVLCCVVLCFLGANTVDGGITQSPK | 136 |
| (306aa): Beta | YLFRKEGQNVTLSCEQNLNHDAMYWYRQ | |
| Chain | DPGQGLRLIYYSQIVNDFQKGDIAEGYS | |
| (TRBV19*01 F); | VSREKKESFPLTVTSAQKNPTAFYLCAS | |
| NSP13-448 TCR | SIMAGGHGELFFGEGSRLTVLEDLKNVF | |
| | PPEVAVFEPSEAEISHTQKATLVCLATG | |
| | FFPDHVELSWWVNGKEVHSGVSTDPQPL | |
| | KEQPALNDSRYCLSSRLRVSATFWQNPR | |
| | NHFRCQVQFYGLSENDEWTQDRAKPVTQ | |
| | IVSAEAWGRADCGFTSVSYQQGVLSATI | |
| | LYEILLGKATLYAVLVSALVLMAMVKRK | |
| | DF | |
| Variable region: | DGGITQSPKYLFRKEGQNVTLSCEQNLN | 137 |
| Beta Chain | HDAMYWYRQDPGQGLRLIYYSQIVNDFQ | |
| (TRBV19*01 F); | KGDIAEGYSVSREKKESFPLTVTSAQKN | |
| NSP13-448 TCR | PTAFYLCASSIMAGGHGELFFGEGSRLT | |
| | VL | |
| Leader peptide: | MSNQVLCCVVLCFLGANTV | 138 |
| Beta Chain | | |
| (TRBV19*01 F); | | |
| NSP13-400 TCR | | |
| Beta Chain | LNHDA | 139 |
| (TRBV19*01 F) | | |
| CDR1 | | |
| Beta Chain | SQIVND | 140 |
| (TRBV19*01 F) | | |
| CDR2 | | |
| Beta Chain | ASSIMAGGHGELF | 141 |
| (TRBV19*01 F) | | |
| CDR3 | | |

### X. Proteinaceous Compositions

As used herein, a “protein” “peptide” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 1000, 1200, 1400, 1600, 1800, or 2000 amino acid residues or nucleic acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.).

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107,108, 109, 110, 111, 112,113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS:1-141. In specific aspects, the peptide or polypeptide is or is based on a human sequence. In certain aspects, the peptide or polypeptide is not naturally occurring and/or is in a combination of peptides or polypeptides.

In some aspects, the protein, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS:1-141.

In some aspects, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the peptide or polypeptide of one of SEQ ID NOS:1-141 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids or nucleic acids of SEQ ID NOS:1-141.

In some aspects, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids of SEQ ID NOS:1-141 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOS:1-141.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 of any of SEQ ID NOS:1-141 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 (or any derivable range therein) contiguous amino acids or nucleotides of any of SEQ ID NOS:1-141.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein structure.

In some aspects of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the antibody that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

### XI. Nucleic Acids

In certain aspects, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one aspect, one or more particular amino acid residues are changed using, for example, a site-directed mutagenesis protocol. In another aspect, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another aspect, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred aspect, the nucleic acid molecules are oligonucleotides. In a more preferred aspect, the oligonucleotides are from highly variable regions of the heavy and light or alpha and beta chains of the antibody or TCR of interest. In an even more preferred aspect, the oligonucleotides encode all or part of one or more of the CDRs or TCRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### XII. Polypeptide Expression

In some aspects, there are nucleic acid molecule encoding polypeptides or peptides of the disclosure (e.g TCR genes). These may be generated by methods known in the art, e.g., isolated from B cells of mice that have been immunized and isolated, phage display, expressed in any suitable recombinant expression system and allowed to assemble to form antibody molecules or by recombinant methods.

### Expression

The nucleic acid molecules may be used to express large quantities of polypeptides. If the nucleic acid molecules are derived from a non-human, non-transgenic animal, the nucleic acid molecules may be used for humanization of the TCR genes.

### B. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, alpha chain, beta chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin or TCR sequence with appropriate restriction sites engineered so that any variable region sequences can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### C. Expression Systems

Numerous expression systems exist that comprise at least a part or all of the expression vectors discussed above. Prokaryote- and/or eukaryote-based systems can be employed for use with an aspect to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### D. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### 2. Isolation

The nucleic acid molecule encoding either or both of the entire heavy, light, alpha, and beta chains of an antibody or TCR, or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### XIII. Formulations and Culture of the Cells

In particular aspects, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain aspects, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific aspects, one or more of these may be explicitly excluded.

In some aspects, the medium further comprises vitamins. In some aspects, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some aspects, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some aspects, the vitamins include or consist essentially of biotin, DL alpha tocopherol acetate, DL alpha-tocopherol, vitamin A, or combinations or salts thereof. In some aspects, the medium further comprises proteins. In some aspects, the proteins comprise albumin or bovine serum albumin, a fraction of BSA, catalase, insulin, transferrin, superoxide dismutase, or combinations thereof. In some aspects, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some aspects, the medium comprises one or more of the following: a B-27® supplement, xeno-free B-27® supplement, GS21TM supplement, or combinations thereof. In some aspects, the medium comprises or futher comprises amino acids, monosaccharides, inorganic ions. In some aspects, the amino acids comprise arginine, cystine, isoleucine, leucine, lysine, methionine, glutamine, phenylalanine, threonine, tryptophan, histidine, tyrosine, or valine, or combinations thereof. In some aspects, the inorganic ions comprise sodium, potassium, calcium, magnesium, nitrogen, or phosphorus, or combinations or salts thereof. In some aspects, the medium further comprises one or more of the following: molybdenum, vanadium, iron, zinc, selenium, copper, or manganese, or combinations thereof. In certain aspects, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific aspects, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific aspects, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific aspects, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular aspects the cells are in a formulated cell suspension that is stable at room temperature for 1, 2, 3, or 4 hours or more from time of thawing.

In particular aspects, the cells of the disclosure comprise an exogenous TCR, which may be of a defined antigen specificity. In some aspects, the TCR can be selected based on absent or reduced alloreactivity to the intended recipient. In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some aspects, the choice of exogenous TCR may not necessarily be defined based on lack of alloreactivity. In some aspects, the endogenous TCR genes have been modified by genome editing so that they do not express a protein. Methods of gene editing such as methods using the CRISPR/Cas9 system are known in the art and described herein.

In some aspects, the cells of the disclosure further comprise one or more chimeric antigen receptors (CARs). Examples of tumor cell antigens to which a CAR may be directed include at least 5T4, 8H9, av$6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FRa, GD2, G250/CAIX, GD3, Glypican-3 (GPC3), Her2, IL-13Rc2, Lambda, Lewis-Y, Kappa, KDR, MAGE, MCSP, Mesothelin, Mucd, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pme117, TRP-1/-2, P. polypeptide, MC1R, Prostate-specific antigen, 0-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two nonidentical antigens, or it may be specific for more than two nonidentical antigens.

### XIV. Administration of Therapeutic Compositions

The therapy provided herein may comprise administration of a combination of therapeutic agents, such as a first anti-viral therapy and a second anti-viral therapy. The therapies may be administered in any suitable manner known in the art. For example, the first and second anti-viral treatment may be administered sequentially (at different times) or concurrently (at the same time). In some aspects, the first and second anti-viral treatments are administered in a separate composition. In some aspects, the first and second anti-viral treatments are in the same composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic compositions of the disclosure may be administered by the same route of administration or by different routes of administration. In some aspects, the peptides, polypeptides, engineered TCRs, engineered T cells, nucleic acids, anti-viral therapies, or pharmaceutical compositions are administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### XV. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain aspects the sample is obtained from a biopsy from ovarian or endometrial tissue by any of the biopsy methods previously mentioned. The sample may be obtained from any other source including but not limited to blood, serum, plasma, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple plasma or serum samples may be obtained for diagnosis by the methods described herein. In other cases, multiple samples, such as one or more samples from one tissue type (for example ovaries or related tissues) and one or more samples from another specimen (for example serum) may be obtained for diagnosis by the methods. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, blood draw, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### XVI. Detection and Vaccination Kits

A peptide or antibody of the disclosure may be included in a kit. The peptide or antibody in the kit may be detectably labeled or immobilized on a surface of a support substrate also comprised in the kit. The peptide(s) or antibody may, for example, be provided in the kit in a suitable form, such as sterile, lyophilized, or both.

The support substrate comprised in a kit of the invention may be selected based on the method to be performed. By way of nonlimiting example, a support substrate may be a multi-well plate or microplate, a membrane, a filter, a paper, an emulsion, a bead, a microbead, a microsphere, a nanobead, a nanosphere, a nanoparticle, an ethosome, a liposome, a niosome, a transferosome, a dipstick, a card, a celluloid strip, a glass slide, a microslide, a biosensor, a lateral flow apparatus, a microchip, a comb, a silica particle, a magnetic particle, or a self-assembling monolayer.

As appropriate to the method being performed, a kit may further comprise one or more apparatuses for delivery of a composition to a subject or for otherwise handling a composition of the invention. By way of nonlimiting example, a kit may include an apparatus that is a syringe, an eye dropper, a ballistic particle applicator (e.g., applicators disclosed in U.S. Pat. Nos. 5,797,898, 5,770,219 and 5,783,208, and U.S. Patent Application 2005/0065463), a scoopula, a microslide cover, a test strip holder or cover, and such like.

A detection reagent for labeling a component of the kit may optionally be comprised in a kit for performing a method of the present invention. In particular aspects, the labeling or detection reagent is selected from a group comprising reagents used commonly in the art and including, without limitation, radioactive elements, enzymes, molecules which absorb light in the UV range, and fluorophores such as fluorescein, rhodamine, auramine, Texas Red, AMCA blue and Lucifer Yellow. In other aspects, a kit is provided comprising one or more container means and a BST protein agent already labeled with a detection reagent selected from a group comprising a radioactive element, an enzyme, a molecule which absorbs light in the UV range, and a fluorophore.

When reagents and/or components comprising a kit are provided in a lyophilized form (lyophilisate) or as a dry powder, the lyophilisate or powder can be reconstituted by the addition of a suitable solvent. In particular aspects, the solvent may be a sterile, pharmaceutically acceptable buffer and/or other diluent. It is envisioned that such a solvent may also be provided as part of a kit.

When the components of a kit are provided in one and/or more liquid solutions, the liquid solution may be, by way of non-limiting example, a sterile, aqueous solution. The compositions may also be formulated into an administrative composition. In this case, the container means may itself be a syringe, pipette, topical applicator or the like, from which the formulation may be applied to an affected area of the body, injected into a subject, and/or applied to or mixed with the other components of the kit.

### XVII. Sequences

TABLE 1

| | SEQ | | |
|---|---|---|---|
| Peptide | ID | | |
| Sequence | NO: | SARS-CoV-2 Gene | HLA Type |
| VATSRTLSY | 23 | Membrane glyco- | HLA-A0101 |
| | | protein | |
| FVLAAVYRI | 22 | Membrane glyco- | HLA-A0201 |
| | | protein | |
| LSYFIASFR | 24 | Membrane glyco- | HLA-A0301 |
| | | protein | |
| LSYFIASFR | 24 | Membrane glyco- | HLA-A1101 |
| | | protein | |
| LWLLWPVTL | 25 | Membrane glyco- | HLA-A2402 |
| | | protein | |
| CVADYSVLY | 26 | Spike protein | HLA-A0101 |
| SFIEDLLFNKV | 27 | Spike protein | HLA-A0201 |
| LSSTASALGK | 28 | Spike protein | HLA-A0301 |
| GTHWFVTQR | 29 | Spike protein | HLA-A1101 |
| LFVTVYSHL | 30 | Spike protein | HLA-C0304 |
| FQPTNGVGY | 31 | Spike protein | HLA-C0304 |
| GVLTESNKK | 32 | Spike protein | HLA-A1101 |
| VYSSANNCTF | 33 | Spike protein | HLA-A2402 |
| IVDTVSALVY | 34 | NSP13 Helicase | HLA-A0101 |
| TLVPQEHYV | 35 | NSP13 Helicase | HLA-A0201 |
| KLFAAETLK | 36 | NSP13 Helicase | HLA-A0301 |
| KLFAAETLK | 36 | NSP13 Helicase | HLA-A1101 |
| VYIGDPAQL | 37 | NSP13 Helicase | HLA-A2402 |
| VVYRGTTTY | 38 | NSP13 Helicase | HLA-B1501 |

### XVIII. Examples

The following examples are included to demonstrate preferred aspects of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the disclosure.

### Example 1: SARS-Cov-2 Membrane Glyco-Protein-65 (MGP-65) Peptide, CTL and TCR

The HLA-A2 restricted SARS-Cov-2 epitope of SEQ ID NO:22 can be used to detect the immunoresponse of patients with COVID-19 infection or for vaccination of patients to prevent the disease. This peptide will allow for work that will lead to a better understanding of COVIDl 9 immunity and will directly impact the management of patients with COVID-19 infection. Furthermore, this peptide, CTL., as well as corresponding TCR can be used to generate third-patty SARS-Cov-2-specific T cell against HLA-matched targets, thus providing off-the-shelfT cell therapy for patients with COVID19 disease. The ability to then bank off-the-shelf allogeneic T cell therapy for the treatment of life-threatening viral diseases is not unprecedented and can be readily applied to COVIDl9-specific T cell therapy.

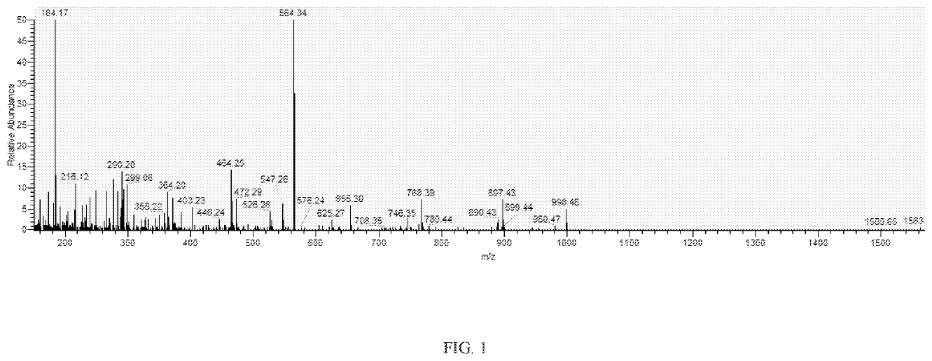

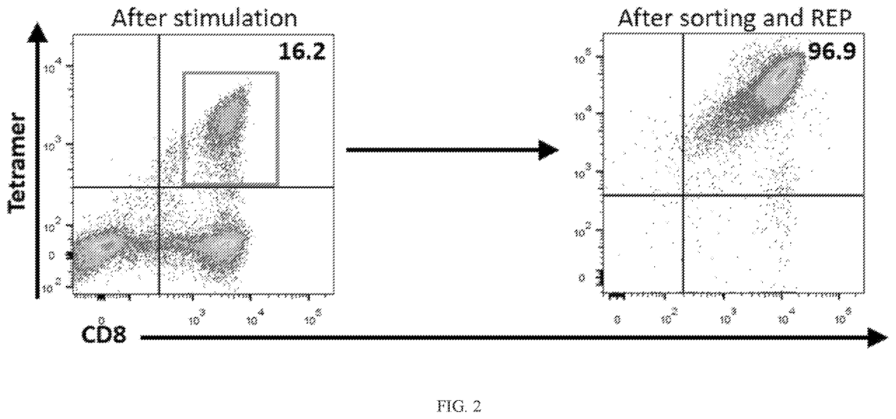

A375 cell line (HLA-A0201+) forced expressing MGP was lysed using NP-40 buffer and the lysate was incubated with Sepharose Fast Flow beads coupled with anti-MHC class I antibody (W6/32). After washing unbound protein, the MHC binding peptide was eluted using acetic acid. The eluted peptide solution was concentrated and analyzed using tandem mass spectrum. One peptide hit (MGP-65, FVLAAVYRI (SEQ ID NO:22) of the MGP sequence was found. (FIG. 1)

MGP-65 (FVLAAVYRI—SEQ ID NO:22) peptide was pulsed to a healthy donor's HLA-A0201 mature dendritic cells (MDC) and then co-cultured with autologous cells in 48 well plates. After two rounds of stimulation, part of the T cells from the each well were collected for flow cytometry detection with MGP-65 tetramer and anti-CD8 staining. A small CD8+/Tetramer+ population were observed after stimulation. The T cells in the wells which show tetramer+/CD8+ population were pooled, and the tetramer+/CD8+ population were sorted and expanded with rapid expansion protocol (REP). After REP for two weeks, high purity CTL (tetramer+ population over 90%) were detected (FIG. 2).

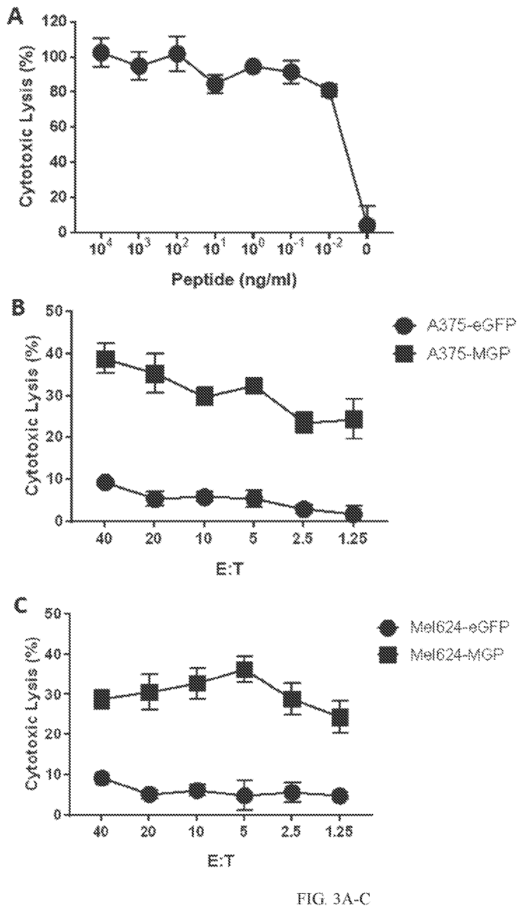

T2 cells pulsed with various concentrations of MGP-65 peptide were used as targets for cell lysis experiments. The lysis ability of MGP-65 specific CTL cell lines was detected with Cr51 release assay (CRA). The effector to target (E:T) ratio used was 20:1 (FIG. 3A). A375 (HLA-A0201+). Mel624 (HLA-A0201+) cell lines with forced expression of eGFP or SARS-Cov-2 MGP were used as the targets and co-cultured with MGP-65 specific CTL cell lines. The effector to target (E:T) ratio used was from 40:1 to 1.25:1. The lysis ability of MGP-65 CTL cell lines to different targets was detected with Cr51 release assay (CRA) (FIG. 3B-C). MGP-65 CTL cell lines show significantly higher killing lever to positive targets than negative control (FIG. 3).

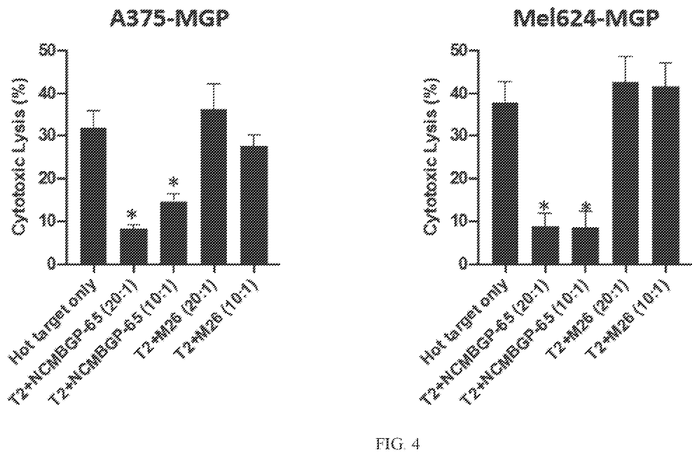

A375-MGP and Mel624-MGP were used as “hot” target cells and were labeled with Cr51. The T2 cells pulsed with MGP-65 peptide without Cr51 labeling represent the “cold” target. T2 cells pulsed with irrelevant peptide M26 were used as control “cold” target. The E:T used was 20:1. The cold target:Hot target ratio used was 10:1 or 20:1. The killing inhibition of cold target to MGP-65 CTL was detected with CRA. When using T2 pulsed with Hormad1-56 peptide as cold target, the killing of MGP-65 CTL to hot target was significantly inhibited compared with the hot target only group. T2 pulsed with M26 negative control did not inhibit the killing of MGP-65 CTL to hot target (FIG. 4).

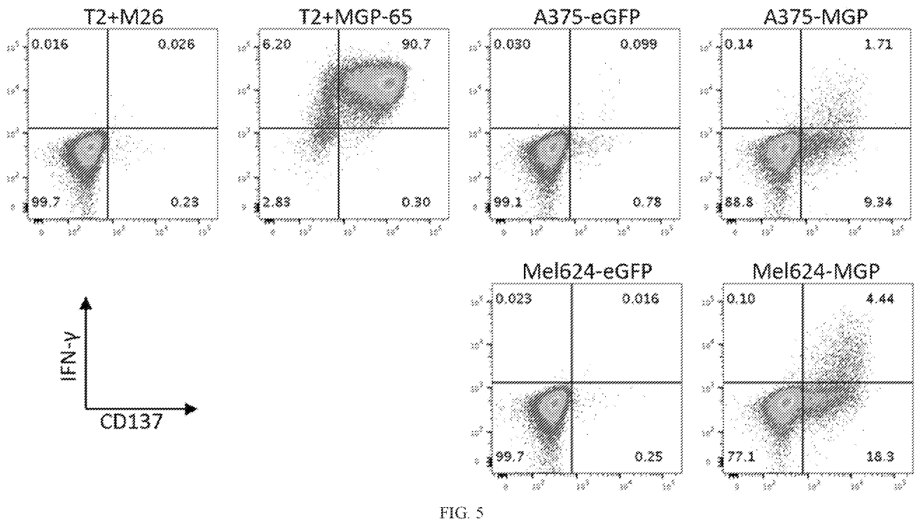

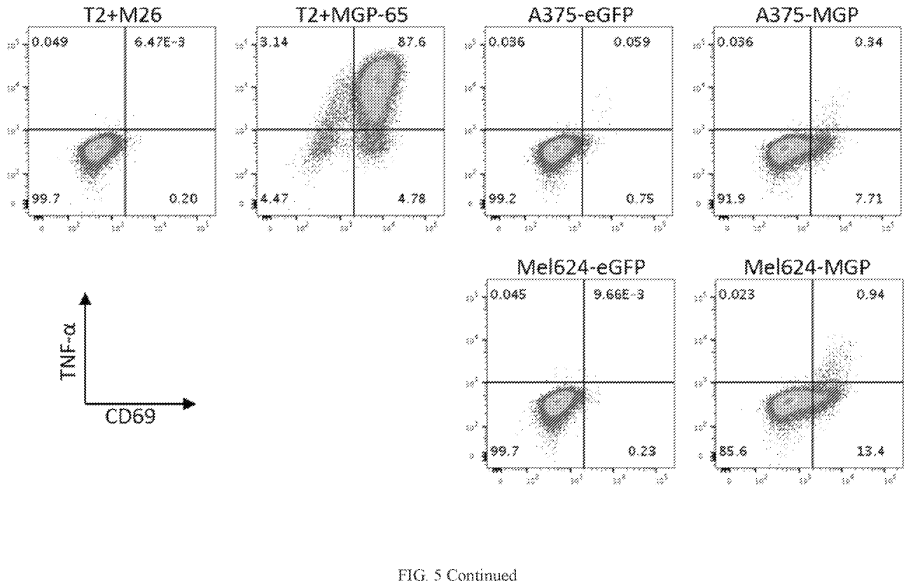

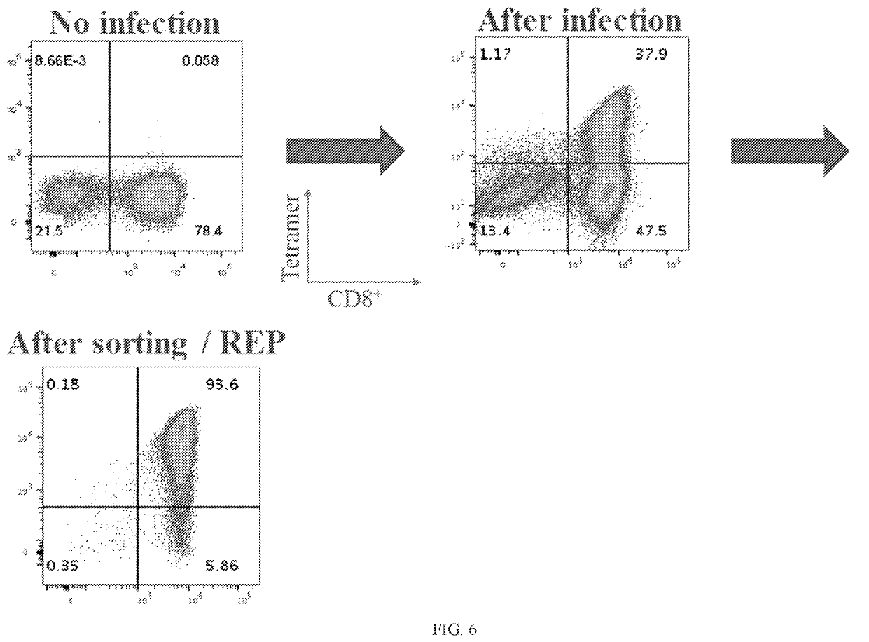

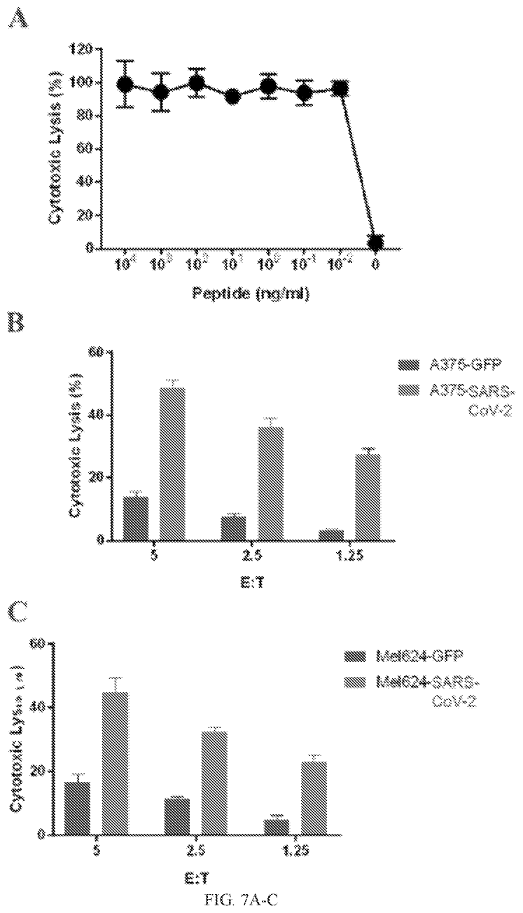

MGP-65 specific CTL cell lines were co-cultured with T2+M26, T2+MGP-65, A375-eGFP, A375-MGP, Mel624-eGFP and Mel624-MGP (E:TN1N:1). After overnight co-culturing, the TCR pathway down-stream activated marker, CD137, CD69, IFN-d and TNF-α were detected with ICS assay. the level of CD137, CD69, IFN-γ and TNF-α of MGP-65 specific CTL cell lines were significantly enhanced when they were co-cultured with positive targets compared with negative control targets FIG. 5).

The T cell receptor (TCR) including alpha chain and beta chain were cloned out using 5-RACE PCR protocol. The sequences were annotated using website tool of IMGT (IMGT/V-QUEST) (Tables 1-3).

TABLE 1

| | Productive TRA rearranged sequence | | |
|---|---|---|---|
| Result summary: | (no stop codon and in-frame junction) | | |
| V-GENE and allele | Homsap | score = | identity = |
| | TRAV17*01 F | 1330 | 100.00% (267/267 nt) |
| J-GENE and allele | Homsap | score = | identity = |
| | TRAJ50*01 F | 273 | 95.00% (57/60 nt) |
| FR-IMGT lengths, | [26.17.34.11] | [5.7.11] | CATAQTSYDKVIF |
| CDR-IMGT lengths | | | |
| and AA JUNCTION | | | |

TABLE 2

| | Productive TRA rearranged sequence | | |
|---|---|---|---|
| Result summary: | (no stop codon and in-frame junction) | | |
| V-GENE and allele | Homsap | score = | identity = |
| | TRAV38- | 1375 | 100.00% (276/276 nt) |
| | 2/DV8*01 F | | |
| J-GENE and allele | Homsap | score = | identity = |
| | TRAJ40*01 F | 253 | 94.64% (53/56 nt) |
| FR-IMGT lengths, | [26.17.34.11] | [7.8.11] | CAYRSGGTYKYIF |
| CDR-IMGT lengths | | | |
| and AA JUNCTION | | | |

TABLE 3

| | Productive TRB rearranged sequence | | |
|---|---|---|---|
| Result summary: | (no stop codon and in-frame junction) | | |
| V-GENE and allele | Homsap | score = | identity = |
| | TRBV9*02 F | 1360 | 100.00% (273/273 nt) |
| J-GENE and allele | Homsap | score = | identity = |
| | TRBJ2-1*01 F | 241 | 98.00% (49/50 nt) |
| D-GENE and allele | Homsap | D-REGION is in reading frame 2 | |
| by IMGT/ | TRBD1*01 F | | |
| JunctionAnalysis | | | |
| FR-IMGT lengths, | [26.17.37.10] | [5.6.13] | CASSVEGPTYNEQFF |
| CDR-IMGT lengths | | | |
| and AA JUNCTION | | | |

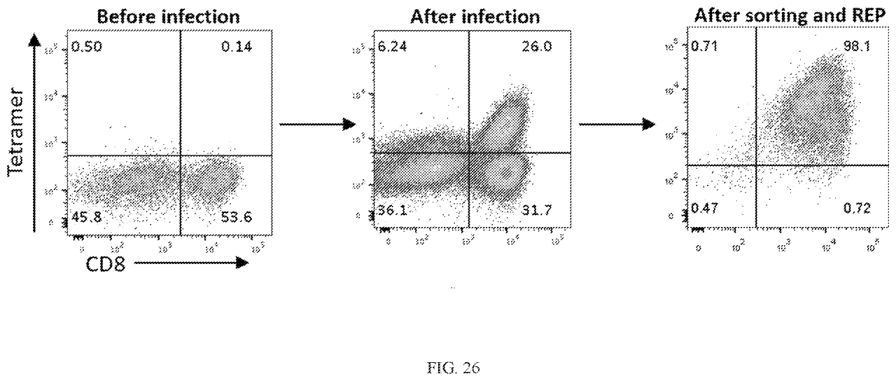

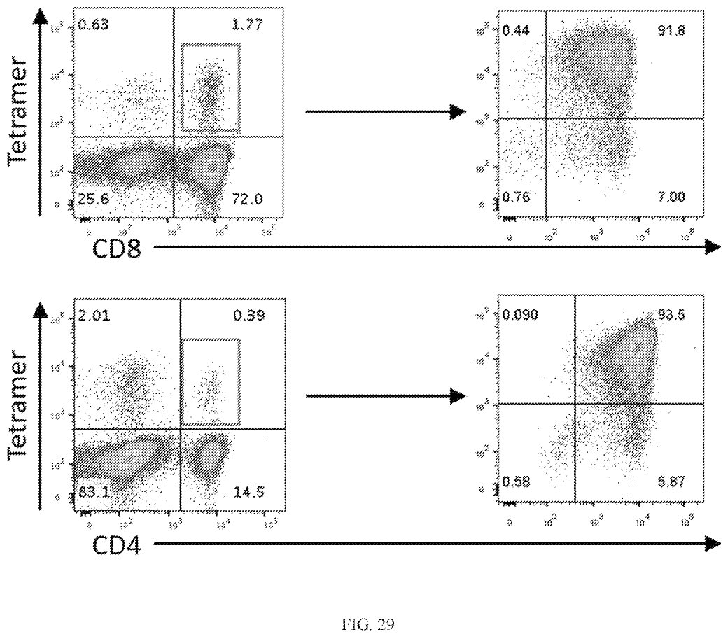

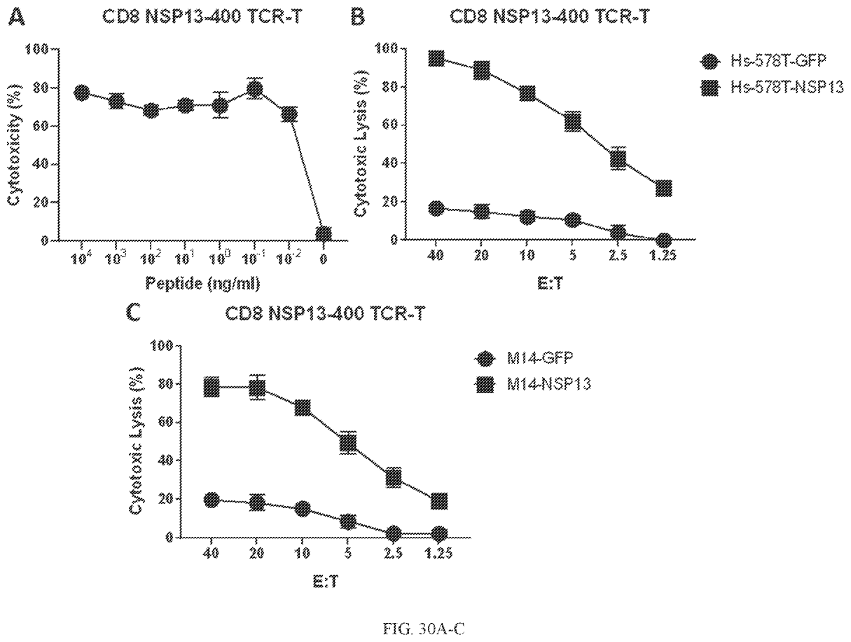

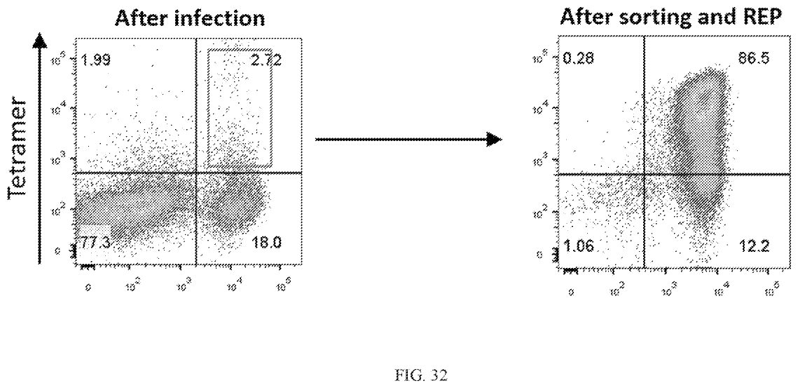

TCR alpha and beta chains were cloned and inserted into a retroviral vector. The recombinant retroviral vector was used to infect PBMC. A CD8+/Tetramer+ population was detected after infection. After tetramer guided sorting and expansion, a highly pure TCR-T population was generated (FIG. 6). Using similar methods, TCR-T cell populations were also generated to the NSP13-134 peptide (KLFAAETLK—SEQ ID NO:36) (FIG. 23), NSP13-242 peptide (TLVPQEHYV—SEQ ID NO:35) (FIG. 26), NSP13-400 peptide (VYIGDPAQL—SEQ ID NO:37) (FIG. 29), and NSP13-448 peptide (IVDTVSALVY—SEQ ID NO:34) (FIG. 32)

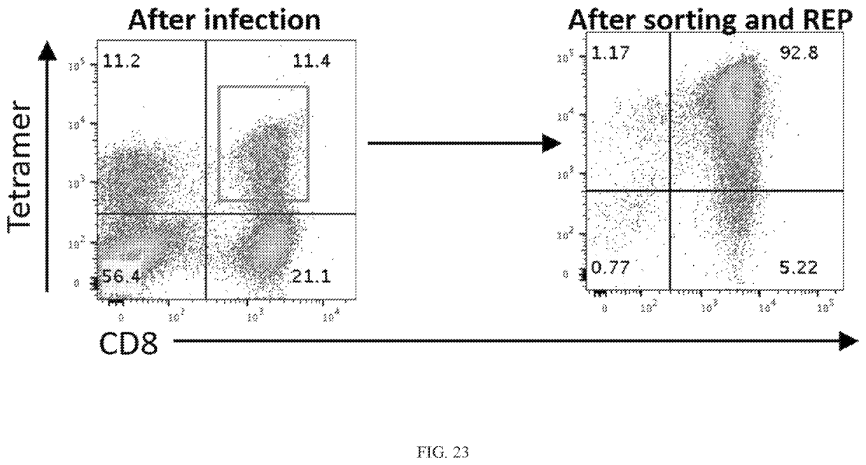

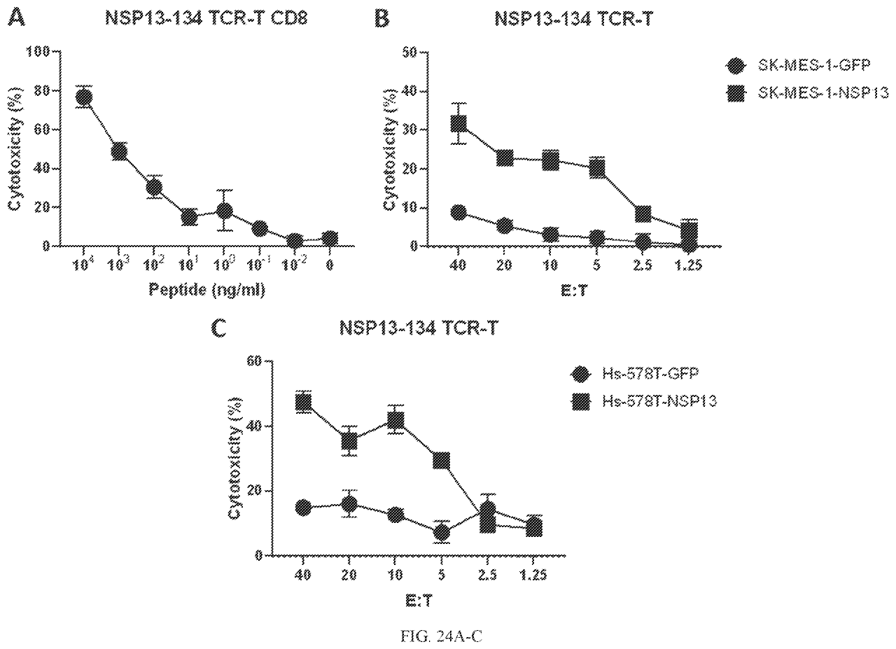

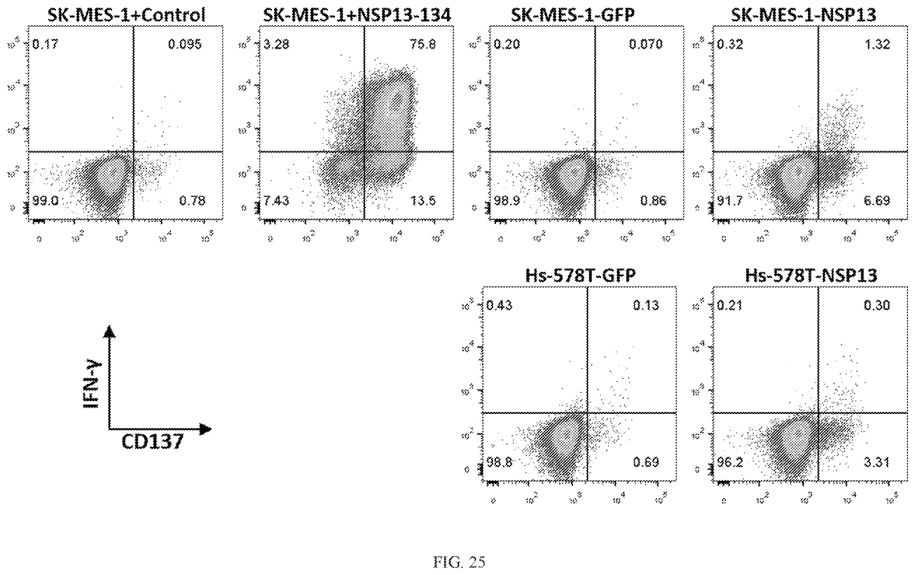

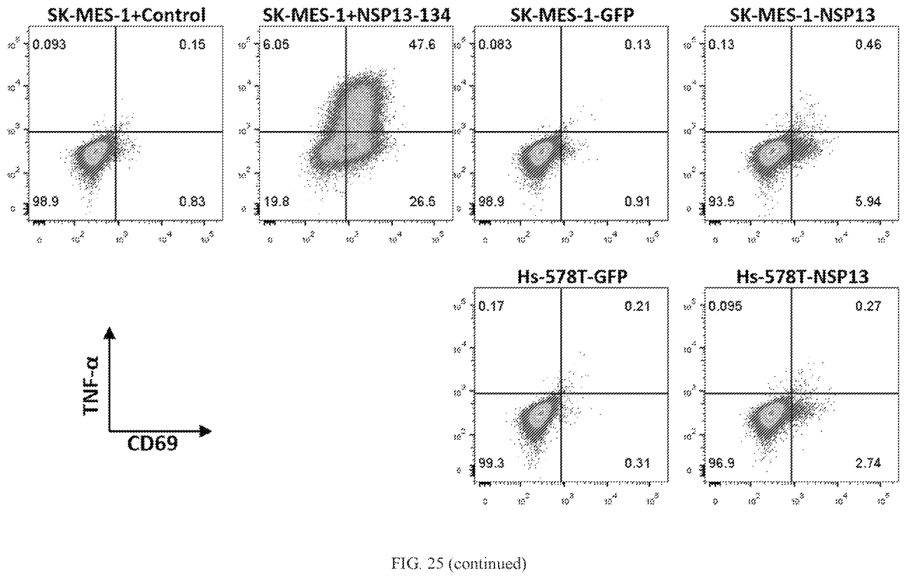

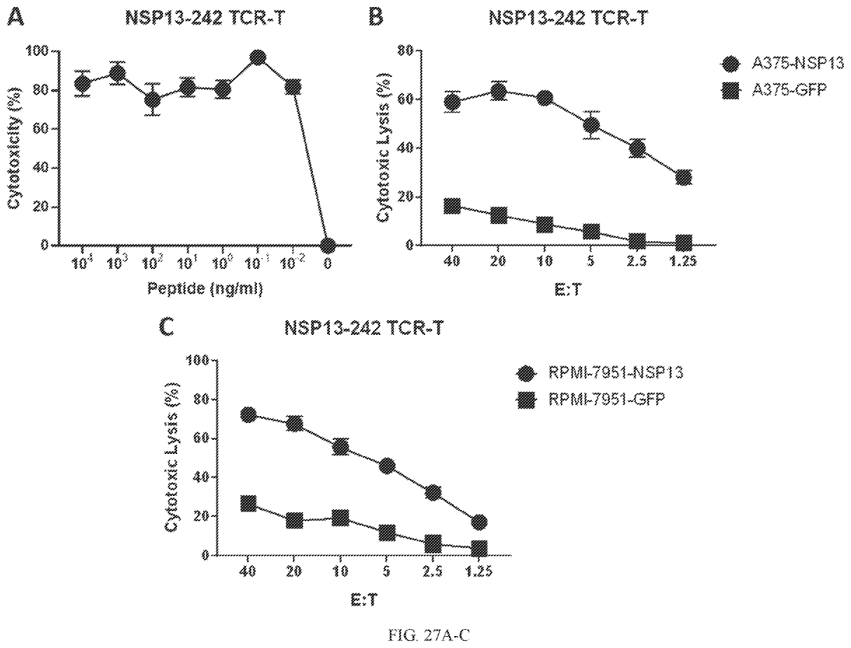

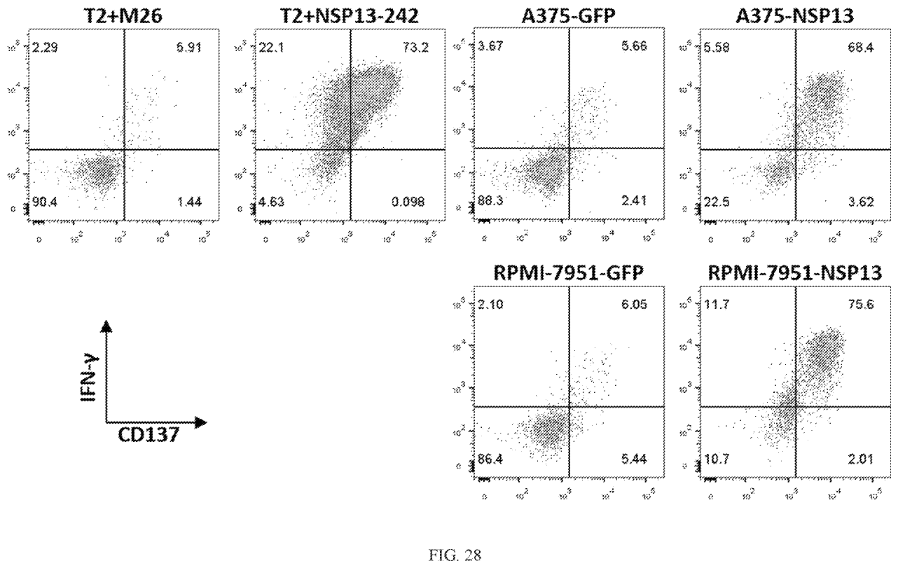

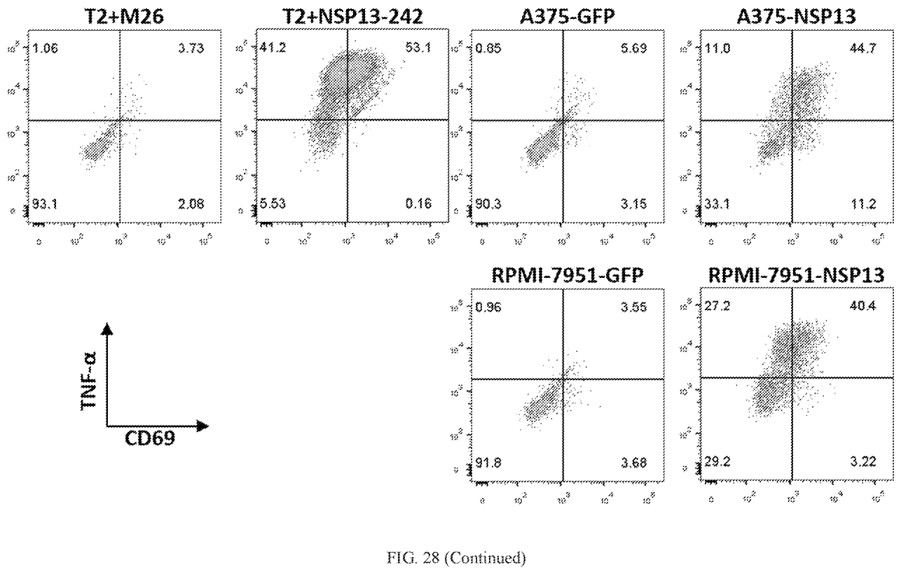

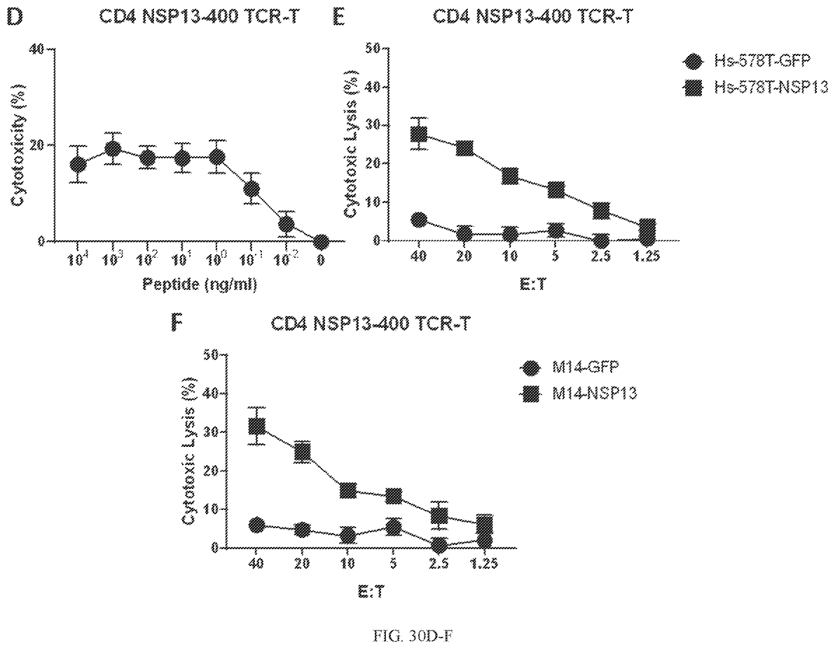

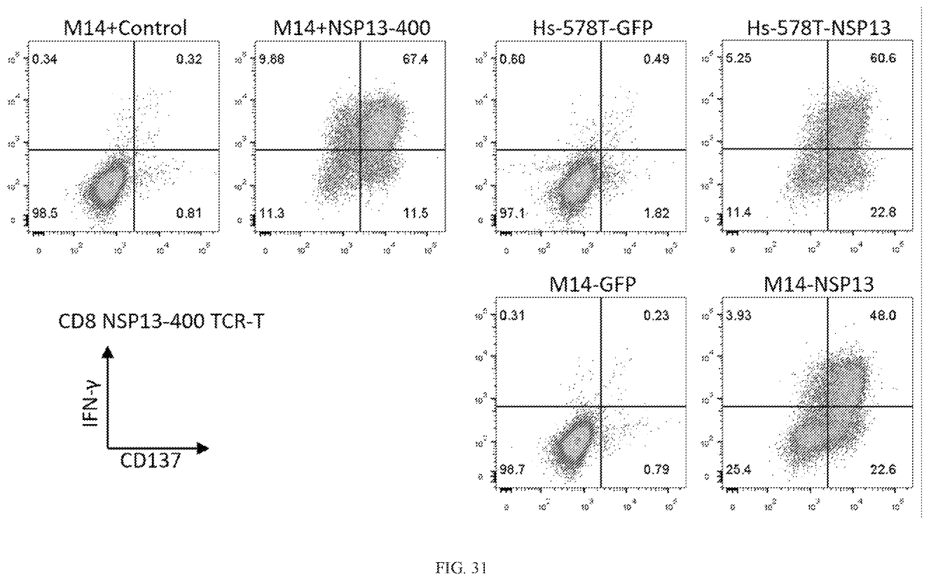

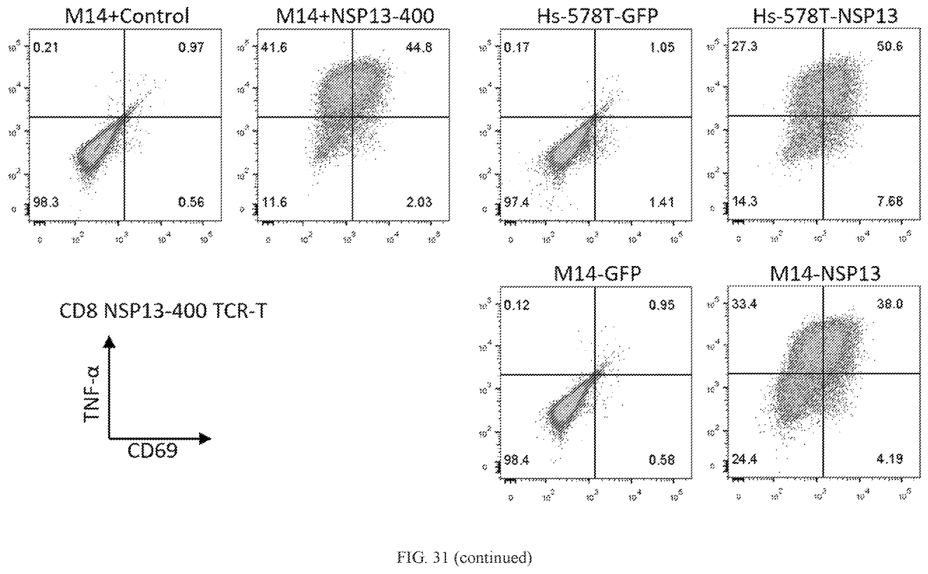

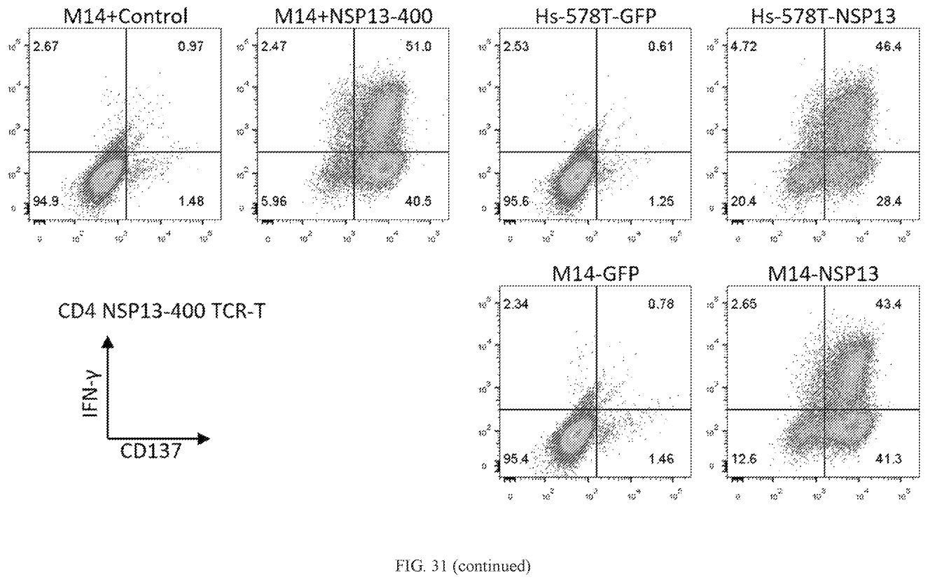

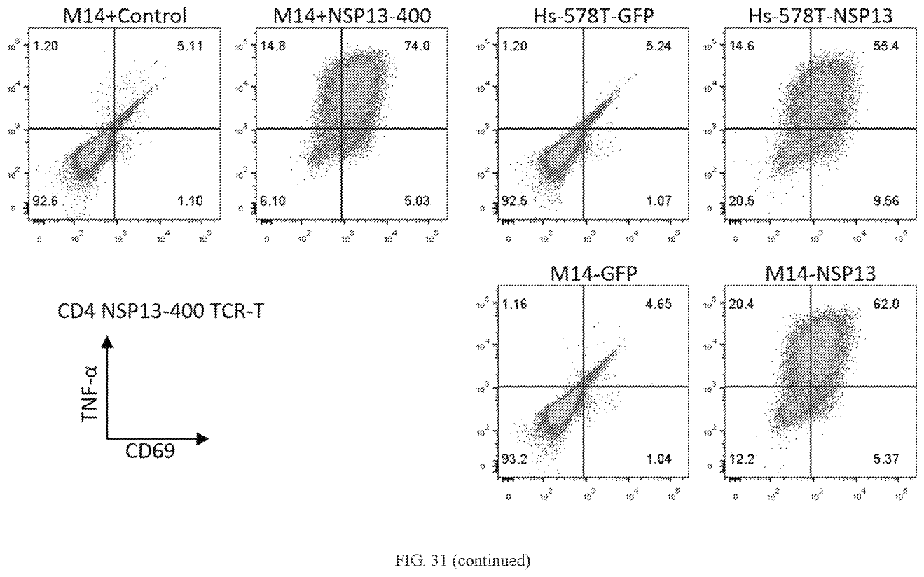

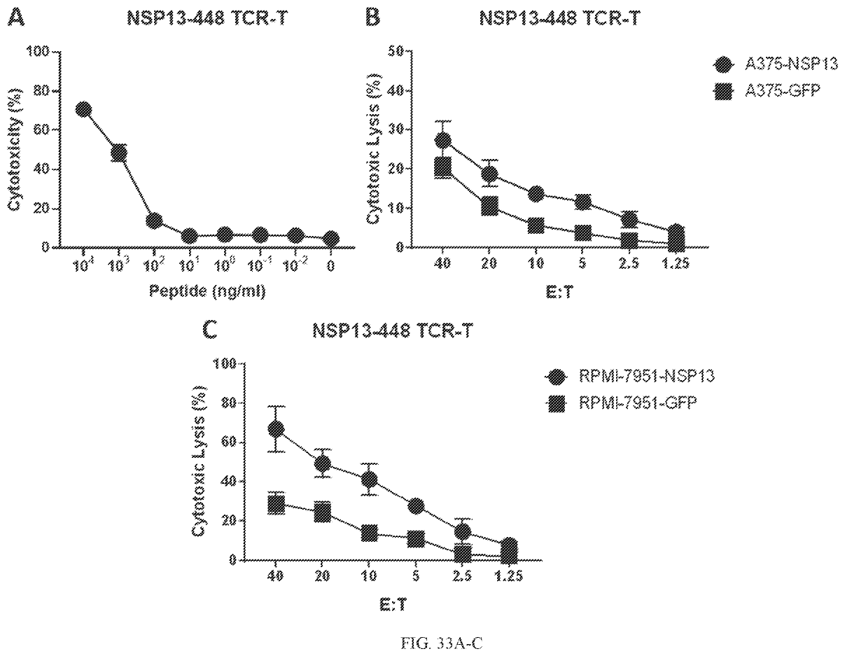

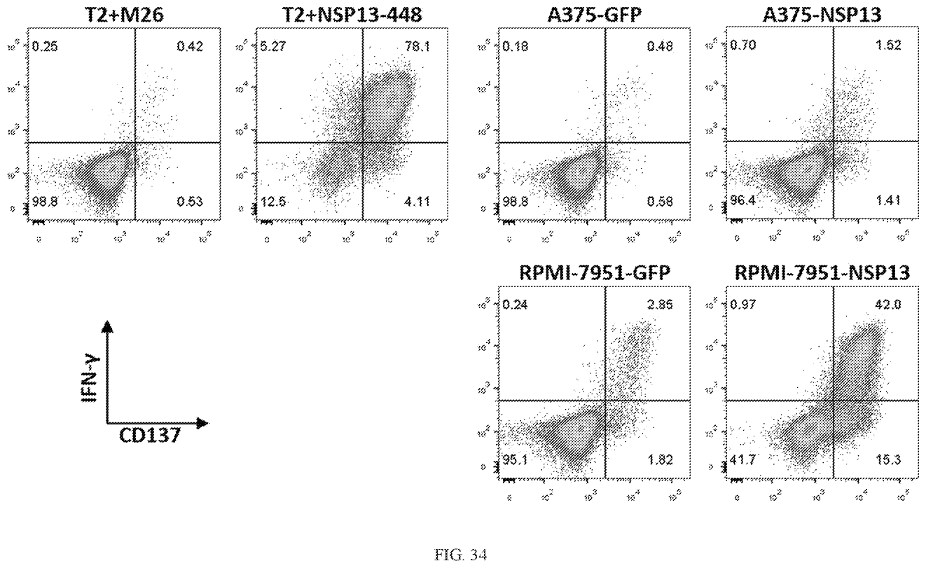

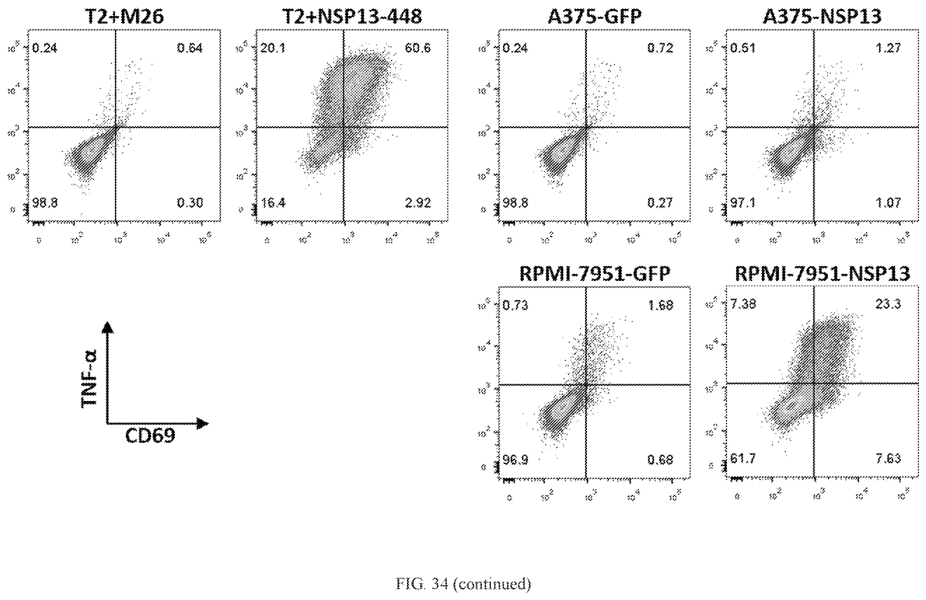

The ability of SARS-CoV-2 specific TCR-T to lyse target cells was tested by a Cr51 release assay. As shown in FIGS. 7, 24, 27, 30, and 33, SARS-CoV-2 TCR-T show significantly higher lysing of positive targets relative to the negative control. NSP13-134 specific TCR-T were co-cultured with SK-MES-1+control A0301 peptide, SK-MES-1+NSP13-134, SK-MES-1-GFP, SK-MES-1-NSP13, Hs-578T-GFP and Hs-578T-NSP13 (E:T=10:1) (FIG. 25). NSP13-242 specific TCR-T were co-cultured with T2+M26, T2+NSP13-242, A375-GFP, A375-NSP13, RPMI-7951-GFP and RPMI-7951-NSP13 (E:T=10:1) (FIG. 28). NSP13-400 specific CD8+ and CD4+ TCR-T were co-cultured with M14+control peptide, M14+NSP13-400, Hs-578T-GFP, Hs-578T-NSP13, M14-GFP and M14-NSP13 (E:T=10:1) (FIG. 31). NSP13-448 specific TCR-T were co-cultured with A375+control A0101 peptide, A375+NSP13-448, A375-GFP, A375-NSP13, RPMI-7951-GFP and RPMI-7951-NSP13 (E:T=10:1) (FIG. 34). After overnight co-culturing, the TCR pathway down-stream activated marker, CD137, CD69, IFN-7 and TNF-α were detected with ICS assay. the level of CD137, CD69, IFN-7 and TNF-α of peptide-specific TCR-T were significantly enhanced when they were co-cultured with positive targets compared with negative control targets (FIGS. 25, 28, 31, and 34).

In conclusion, the inventors have generated SARS-CoV-2 specific CTL, cloned TCR and TCR engineered T cells (TCR-T) and have functionally validated the CTL and TCR T cells. Cell therapy using TCR-T can be employed as an optional therapeutic approach for the treatment of severe SARS-CoV-2 infected patients. The identified SARS-CoV-2 peptide or coding sequence can be loaded into presenting cells and co-cultured with T cells to generate antigen specific CTL cell lines or clones. Autologous or allogeneic CTL cell lines or clones can be used for adoptive immunotherapy for HLA-matched patients with SARS-CoV-2 infection.

### Example 2: Mass Spectrometric Identification of Immunogenic SARS-CoV-2 Epitopes and Cognate TCR for T Cell-Based Therapy of COVID19 Disease

### A. Introduction

Severe acute respiratory syndrome coronavirus 2 (SARS-CoV-2), the highly transmissible respiratory virus responsible for the COVID-19 pandemic outbreak, continues to render significant, lasting impact on global public health and has created an urgent need to develop accurate immunodiagnostics, and effective treatment strategies (Hui et al., 2020; Wu et al., 2020b). Rapid dissemination of the SARS-CoV-2 genomic sequence first revealed by Dr. Zhang Yongzhen led to large scale efforts around the world to develop a protective vaccine that could elicit humoral (antibody) and cellular (T cell) responses (Wu et al., 2020a). It follows that the identification of immunogenic epitopes of SARS-CoV-2 recognized by the human immune system would be critical for rational vaccine development.

Using in silico prediction algorithms, several investigators have amassed extensive panels of Class I and Class II restricted epitopes to probe SARS-CoV-2-specific T cell responses, in some cases, combining these with overlapping ‘megapools’ spanning regions conserved regions of the genome (Campbell et al., 2020; Grifoni et al., 2020a). These peptides have been used to track responses in infected and convalescent individuals (Kar et al., 2020; Peng et al., 2020), design multi-epitope vaccines and used directly or indirectly to measure the breadth and severity of COVID19 disease (Braun et al., 2020; Grifoni et al., 2020b; Kar et al., 2020; Le Bert et al., 2020; Nolan et al., 2020; Snyder et al., 2020; Weiskopf et al., 2020). While these studies have uncovered insights into the T cell immunobiology of COVID19, the accuracy of T cell responses using in silico predicted responses and overlapping long peptide (OLP) pools is diminished by a failure to consider whether such epitopes are immunogenic. An immunogenic epitope in this sense is defined as a peptide that is known to be presented by self-MHC, and is capable of eliciting T cells of sufficient affinity that such T cells can recognize target cells endogenously expressing antigen and presenting the antigen-derived peptide in the context of an MHC complex with sufficient surface density as to sensitize the target cell to peptide-specific T cell-mediated recognition. In essence, an immunogenic epitope of SARS-CoV-2 requires both direct sequencing of peptides presented by MHC as well as empiric validation of T cell immunogenicity.

To the inventors' knowledge, this study is the first use of tandem MS to identify T cell epitopes of SARS-CoV-2 following peptide elution from the MHC complexes of SARS-CoV-2-expressing cells, and the first study to empirically validate immunogenicity by in vitro generation of SARS-CoV-2-specific CTL. Enabling technology developed by the inventors' group for the isolation of rare tumor-reactive T cells from very low precursor frequency populations in the peripheral blood was applied (Chapuis et al., 2017). The inventors present data on the identification of five immunogenic epitopes of a highly conserved region of membrane glycoprotein and the non-structural protein region of the SARS-CoV-2 genome and demonstrate that such MGP65- and NSP13-specific CTL recognize and kill SARS-CoV-2 antigen expressing target cells; the inventors have further sequenced the TCR alpha and beta chains and demonstrate that specificity can be transferred by engineering expression of this TCR in polyclonal lymphocytes.

### B. Results

1. SARS-CoV-2 Peptides Defined ‘in Silico’, Fail to Elicit T Cells that Recognize SARS-CoV-2 Antigen Expressing Targets.

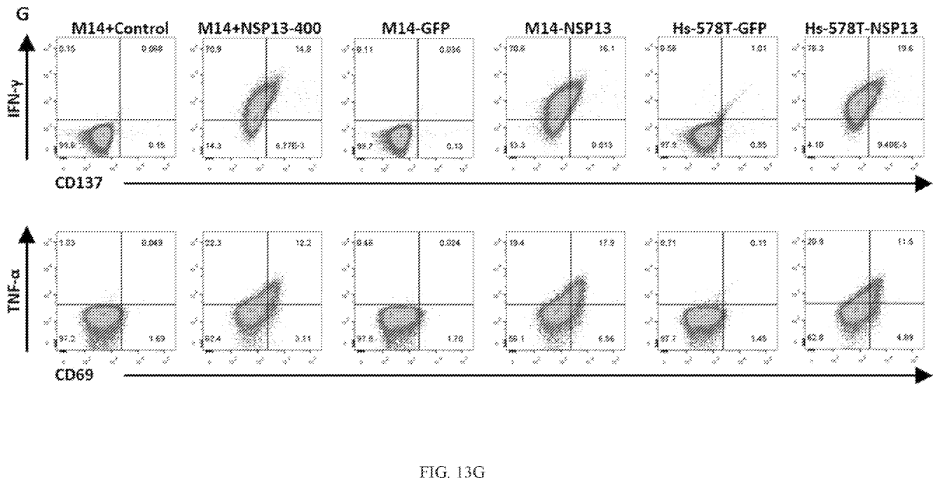

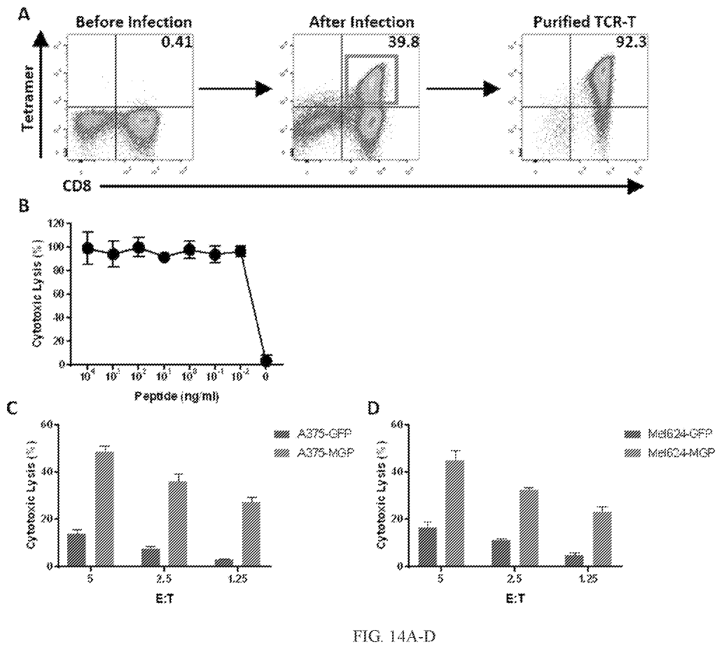

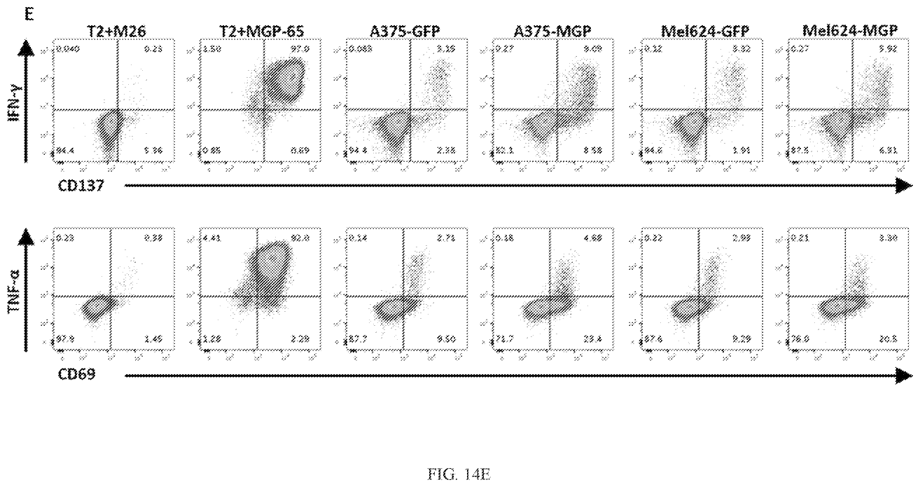

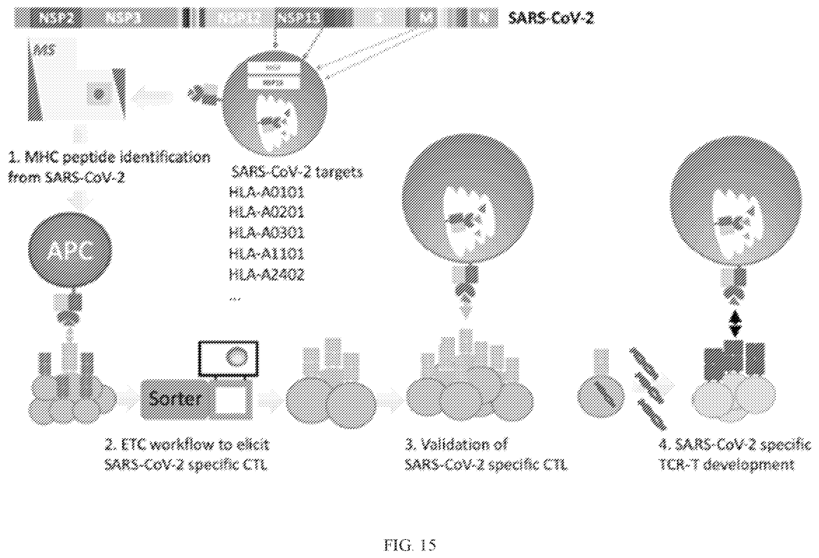

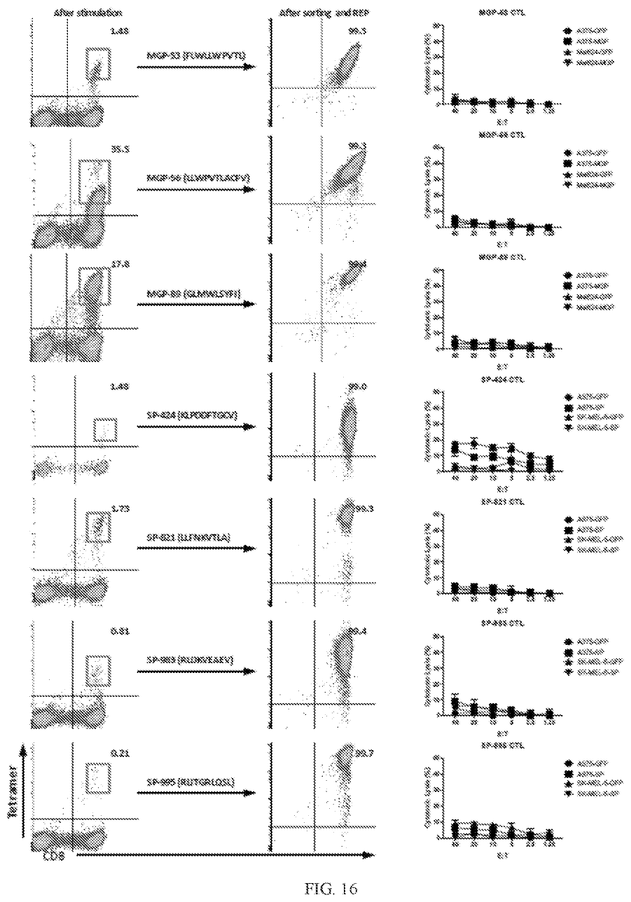

As an initial screen of known predicted epitopes for immunogenicity, the inventors selected Class I-restricted peptides to the SARS-CoV-2 Spike protein and membrane glycoprotein (MGP) based on a literature search of studies where such ‘in silico’ predicted peptides were described as ‘immunodominant’. These peptides have previously been reported to be ‘immunodominant’ on the basis of their ability to generate high levels of peptide-specific responses from the PBMC of COVID19+ patients and, surprisingly, in some healthy donors as well (apparently as a result of cross-reactive responses from T cells elicited in the past to non-pathogenic SARS viruses) (Agerer et al., 2021; Ahmed et al., 2020; Gao et al., 2020; Grifoni et al., 2020a; Kar et al., 2020; Safavi et al., 2020; Shomuradova et al., 2020; Sohail et al., 2021; V. Gauttier, 2020; William Chour, 2020). The inventors synthesized 4 of these spike protein peptides and 3 of the membrane glycoprotein (MGP) peptides. Using the endogenous T cell (ETC) generation workflow (see Methods section), the inventors generated individual T cell cultures against all 4 Spike peptide and all 3 MGP peptides (FIG. 16). However, when these highly enriched (>80% tetramer+) T cell cultures were tested against HLA-matched target cells engineered to express the relevant SARS-CoV-2 Spike protein or membrane glycoprotein, no evidence of target cell killing was observed (FIG. 16). The inventors postulated that these in silico predicted peptides were not endogenously presented; and that a more accurate means of identifying endogenously presented, immunogenic epitopes would be desirable and could be achieved by directly eluting and sequencing peptides from the MHC of SARS-CoV-2-expressing cells.

### 2. Profiling of MHC Class-I Restricted Epitope of SARS-CoV-2

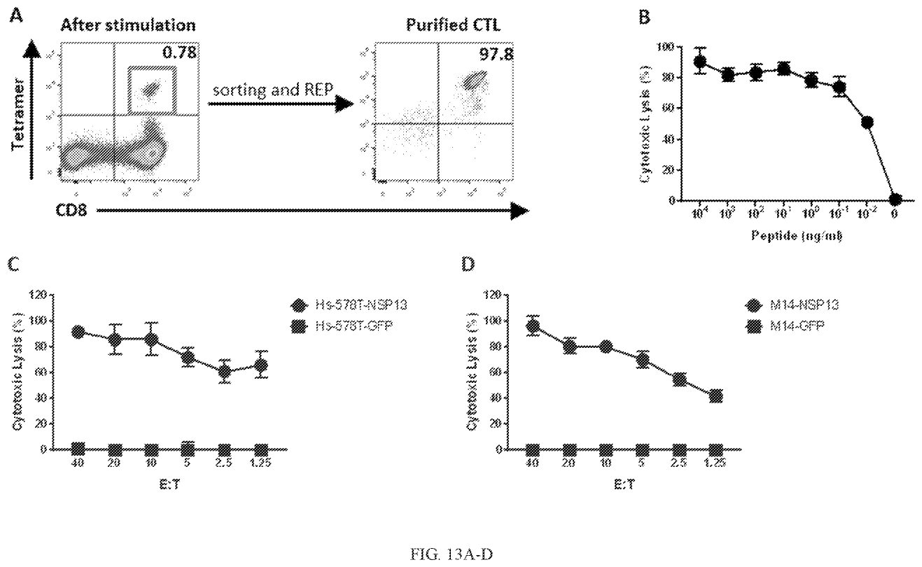

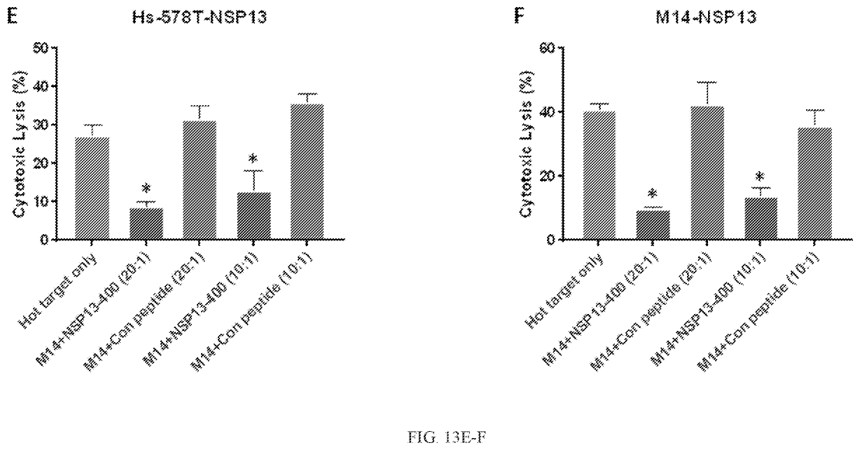

The antigen discovery platform for SARS-CoV-2 is comprised of four steps: (1) peptide elution and identification with mass spectrometry (MS) for SARS-CoV-2 targets; (2) endogenous T cell (ETC) generation workflow to elicit peptide-specific CTL; (3) empiric validation of antigen specific CTL against SARS-CoV2 targets; and (4) SARS-CoV-2 specific T cell receptor (TCR) engineered T cell (TCR-T) development (FIG. 15). In order to elute and sequence the MHC bound peptide derived from SARS-CoV-2, the SARS-CoV-2 genes were overexpressed in targets cells with different HLA allele expression. Lentiviral expression vectors spanning highly conserved regions of SARS-CoV-2 regions: membrane glycol-protein (MGP) or Non-structure protein helicase (NSP13)(Keller et al., 2020; Le Bert et al., 2020), were constructed and used to infect target cell line A375 (HLA-A0101/0201), Me1624 (HLA-A0201), RPMI-7951 (HLA-A0101/0201), Hs-578T (HLA-A0301/A2402) and M14 (HLA-A1101/2402). Following puromycin selection and expansion of MGP or NSP13 stably-expressing cell lines, purity of over 90% was achieved (data not showed). The MGP or NSP13-expressing cell lines were expanded to 300-500 million, harvested, lysed with NP40 detergent lysis buffer and subjected to total HLA class I immunoprecipitation (anti-HLA-A, B, C) and acid elution, followed by tandem mass spectrometry (MS) to analyze the HLA-bound peptides.

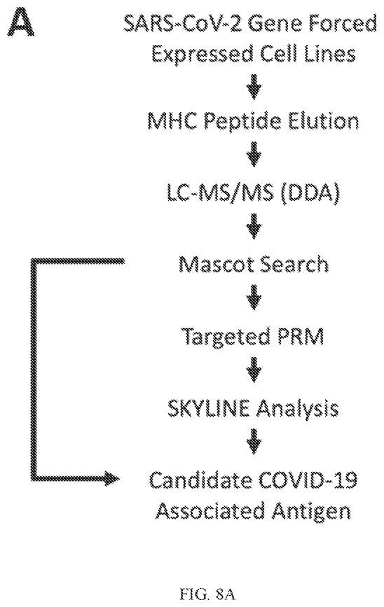

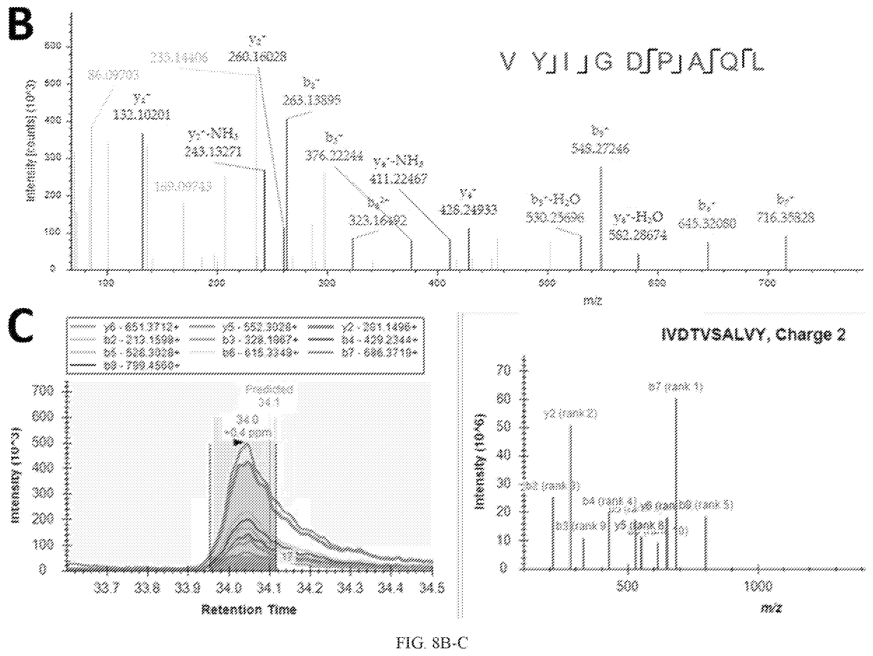

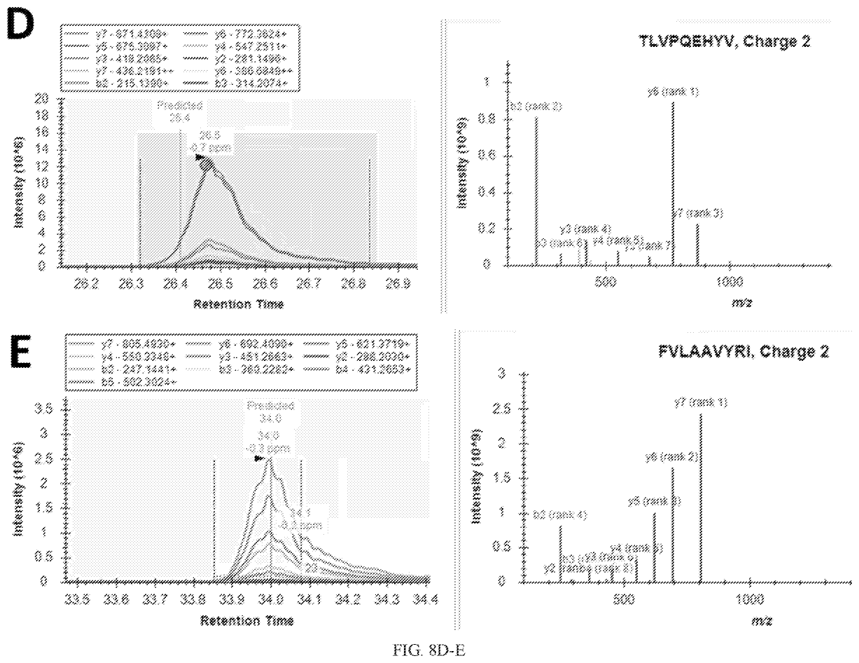

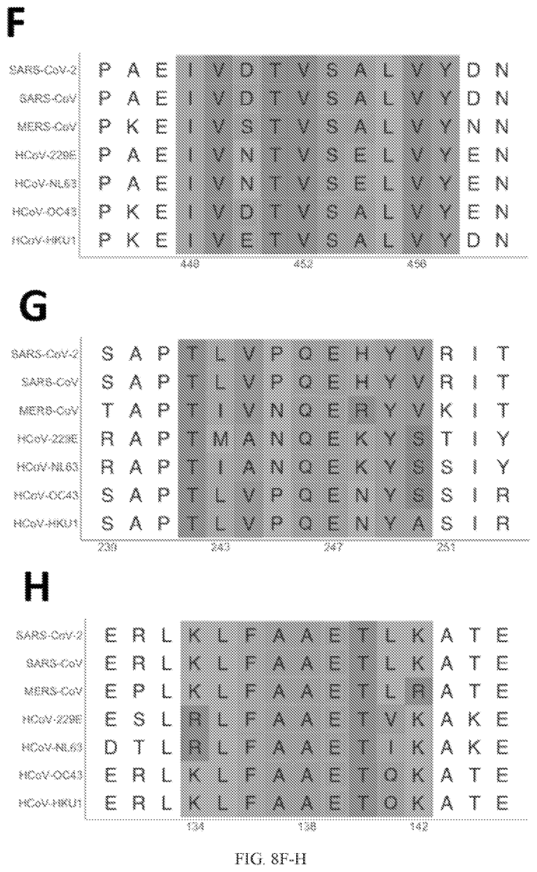

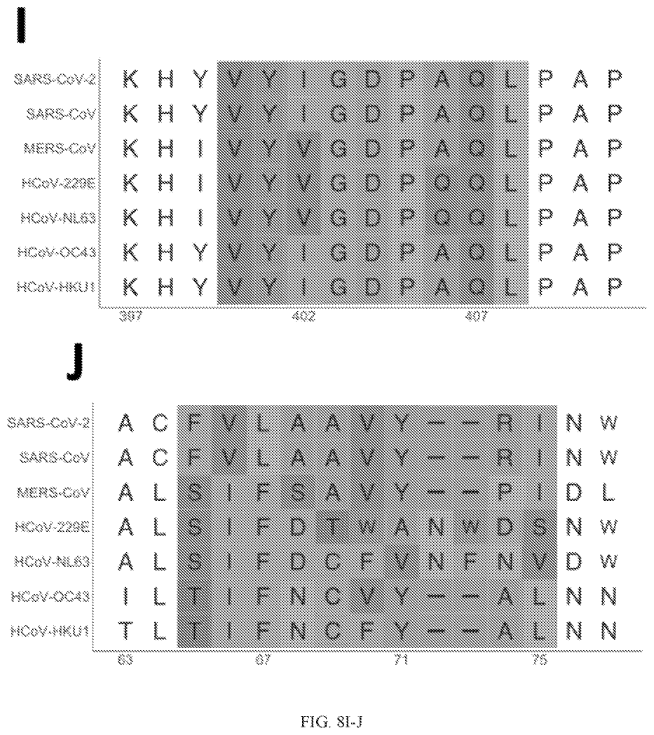

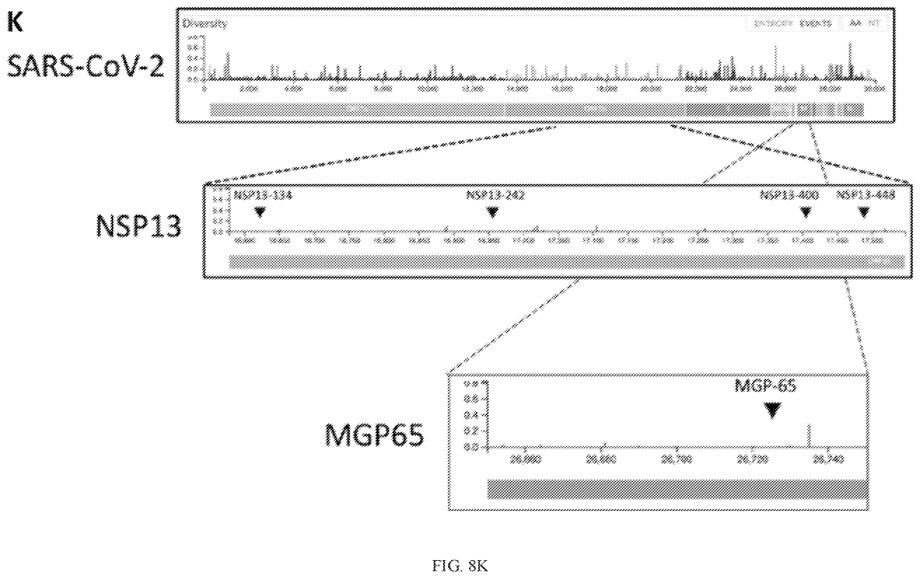

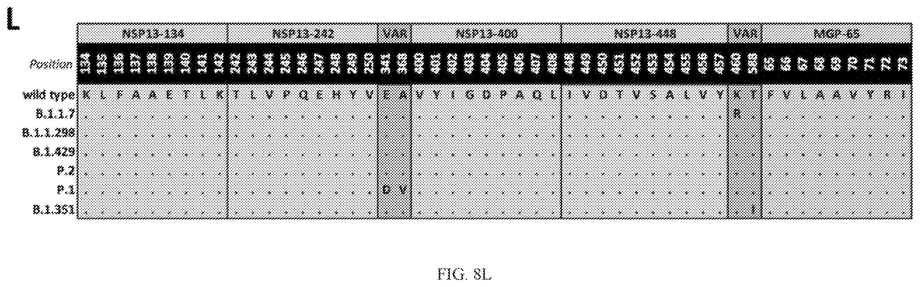

The inventors initially analyzed the eluted HLA bound peptides derived from the SARS-CoV-2 targets established above by using data dependent analysis liquid chromatography tandem mass spectrometry (DDA MS/MS). The eluted spectra were searched using the Mascot search engine node (version 2.6) within Proteome Discoverer (version 2.3) processing workflow with the Swiss-Prot human proteome database (version 2020_05) followed by virus proteome database (version 2020_05). To reduce false positive hits from human proteome, the “Spectrum Confidence Filter” node within Proteome Discoverer processing workflow filtered out all spectra with highly confident peptide-spectrum matches annotated from the human proteome. The remaining spectra were further searched against the virus proteome (FIG. 8A). In total, 12,770 MS/MS were acquired, 9731 Peptide-Spectrum-Match (PSM) were annotated yielding 357 peptides with Mascot Ions Score ≥25. Among the 357 peptides was annotated a peptide derived from NSP13 (NSP13-400, VYIGDPAQL—SEQ ID NO:37) with Mascot Ions Score=27 (FIG. 8B). This peptide was eluted from M14-NSP13 cells (HLA-A1101/A2402). From the HLA binding assay (IEDB tool), the NSP13-400 peptide scored high predicted binding affinity to HLA-A2402 allele (Table 2), suggesting that NSP13-400 peptide is likely to be presented by HLA-A2402.

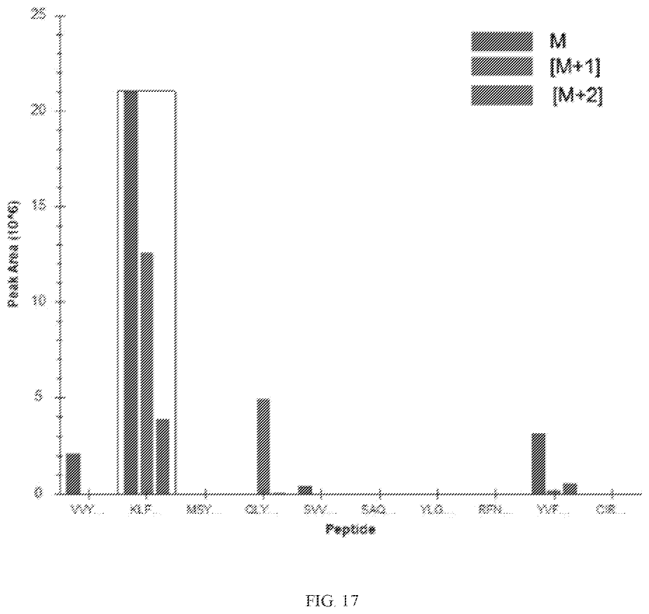

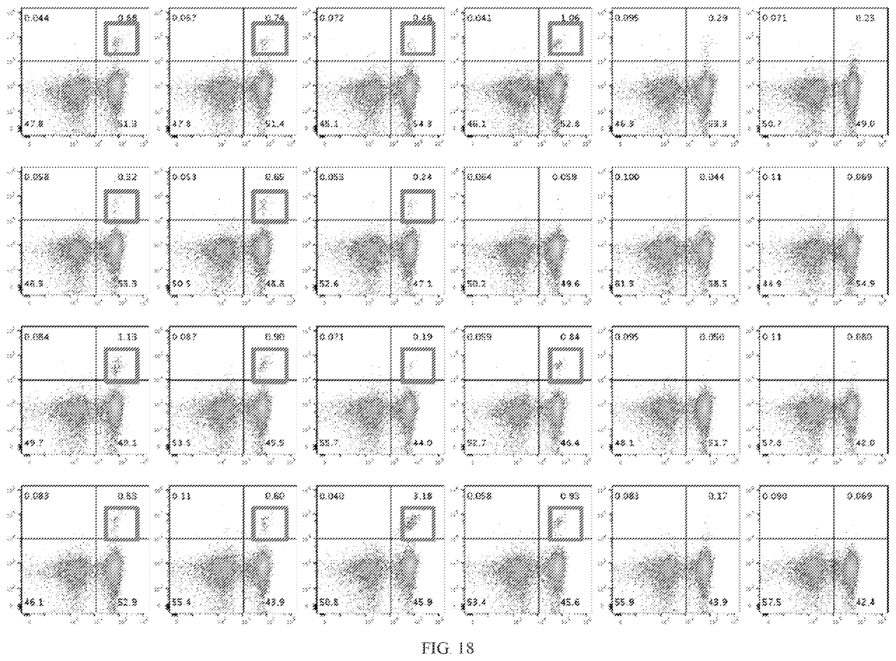

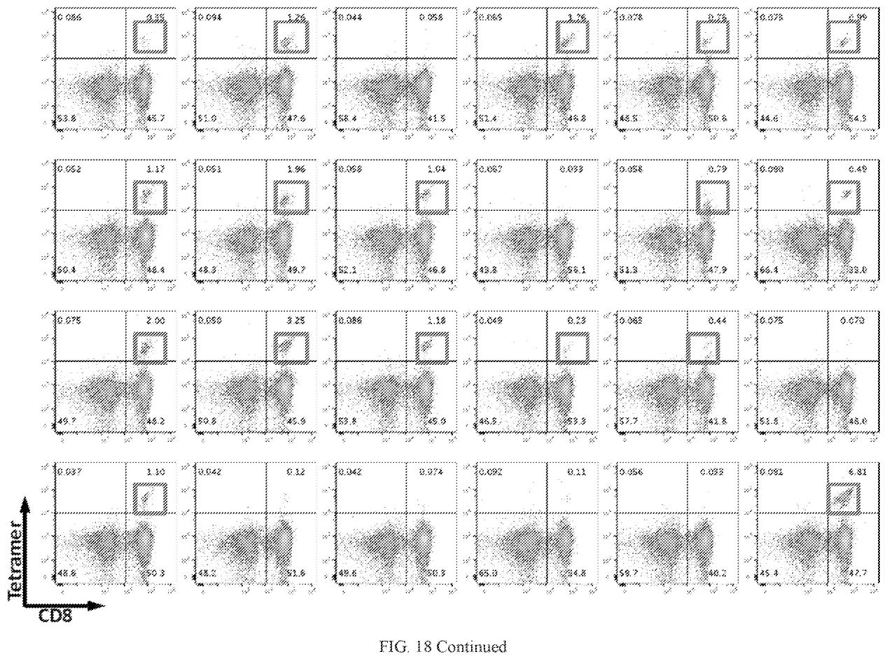

To enable more comprehensive profiling of potential HLA Class-I restricted peptides from SARS-CoV-2, the inventors further analyzed eluted peptides by parallel reaction monitoring mass spectrometry (PRM-MS) to focus predicted high probability HLA binding peptides derived from SARS-CoV-2 but not successfully detected by DDA approach. Prior to the PRM-MS, ten predicted high potential HLA-A0101, HLA-0201 or HLA-A0301 binding peptides from MGP or NSP13 were selected, respectively (Table S1). For the eluted peptide from A375-NSP13, precursor ion inclusion lists of 10 potential peptides were generated using Skyline, and the inventors targeted and monitored these 10 peptides using nanoflow LC-PRM-MS with high mass accuracy and resolution. Pierce™ Peptide Retention Time Calibration Mixture peptides were used to monitor retention time drifts and adjust the scheduled PRM method. The inventors first generated spectral library using synthetic peptides, and the inventors used both synthetic peptides and Pierce™ Peptide Retention Time Calibration Mixture peptides to define iRT, a normalized dimensionless peptide-specific value, to accurately predict retention time of each targeted peptide. The inventors detected IVDTVSALVY (SEQ ID NO:34) (NSP13-448) with average product ion ppm error at −0.7 ppm (FIG. 8C), TLVPQEHYV (SEQ ID NO:35) (NSP13-242) with average product ion ppm error at −0.7 ppm (FIG. 8D). Same rules applied. From A375-MGP, the inventors detected FVLAAVYRI (SEQ ID NO:22) (MGP-65) with average product ion ppm error at −0.3 ppm (FIG. 8E). These XIC MS1 analyses reported sufficient chromatographic separations and high-resolution MS1 detection with a narrow m/z window, suggesting that these targeted peptides exist in eluted peptide samples. In addition, one potential peptide KLFAAETLK (SEQ ID NO:36) (NSP13-134) was selected from PRM-MS screen (FIG. 17) for further immunogenic functional assay (MS/MS not shown).

To evaluate if these five candidates SARS-CoV-2 HLA Class-I restricted peptides identified with DDA or PRM-MS are homologous to other coronaviruses including SARS-CoV, Middle East respiratory syndrome coronavirus (MERS-CoV), as well as other four coronavirus 229E, NL63, OC43 and HKU1, multiple sequence alignment (MSA) analysis was performed. NSP13-242 (FIG. 8G), NSP13-134 (FIG. 8H), and MGP-65 (FIG. 8J) show a high degree of homology to the sequence of SARS-CoV. NSP13-400 (FIG. 8I) shows a high degree of homology to the sequence of SARS-CoV, HCoV-OC43 and HCoV-HKU1 (underlined in red). NSP13-448 (FIG. 8F) shows a high degree of homology to the sequence of SARS-CoV, HCoV-OC43 (underlined in red). In order to evaluate if these five candidates are homologous to non-coronaviruses species, the peptides were analyzed by using BLAST searches to identify the all potential source proteins. The Top 250 hits for each target sequence reported up to 88.99% identity or 100% identity but coverage up to 88.99% or 100% identity to related-coronavirus, indicated these five candidate peptide are only homologous with coronavirus but no other species (data not shown).

### 3. Newly-Defined Epitopes are Found in Highly Conserved Regions of SARS-CoV-2 and SARS-CoV2 Variants

Olvera et. al recently described the development of a COVID19 vaccine using the overlapping of SARS-CoV-2 consensus sequence (Olvera et al., 2020). This paper utilized an entropy-based calculation on more than 1700 viral genome entries in NCBI and encompassed all described SARS-CoV-2 open reading frames (ORF), including recently described frame-shifted and length variant ORF. The Nextstrain project (found on-line at nextstrain.org), an open-source project that provides a continually-updated view of publicly available data alongside powerful analytic and visualization tools to aid epidemiological understanding and improve outbreak response, provides a means to analyze genetic diversity across the SARS-CoV-2 genome. Using both these sources, the inventors verified that these five peptides were located in a highly conserved region of SARS-CoV-2 genome (FIG. 8K). Recently, genetic variants of SARS-CoV-2 have emerged on a global scale, e.g., mutation 23403A>G-(D614G) located on the spike protein, believed to render SARS-CoV-2 more infectious (Garcia-Beltran et al., 2021; Tzou et al., 2020). To investigate if recently reported variants are located withing these five candidates SARS-CoV-2 HLA Class-I restricted epitopes, the inventors focused on variants of concern first described in the United Kingdom (B.1.1.7), Denmark (B.1.1.298), United States (B.1.429), Brazil and Japan (P.2 and P.1), and South Africa (B.1.351). For NSP-13 region, there are two mutations, E341Y and A368V, present in P.1 variants, one mutation K460R present in B.1.1.7 variant, one mutation T588I present in B.1.351 variant, none of which overlap with these four NSP-13 HLA Class-I restricted peptides. No variant strain has been reported from the membrane glycol-protein (MGP) region (FIG. 8L).

4. MGP-65 Peptide Specific Cytotoxic T Cells Generated from the Peripheral Blood Recognize SARS-CoV-2-MGP-Expressing Target Cells

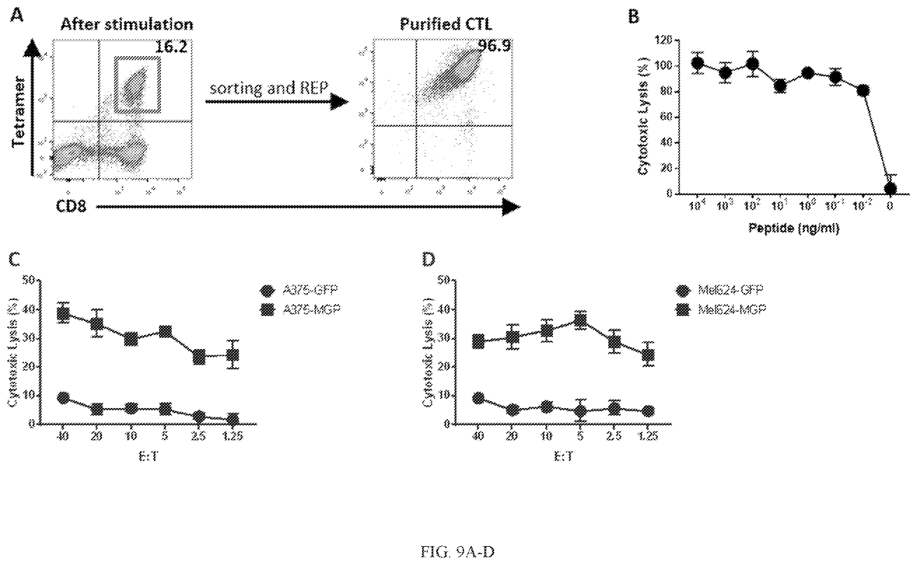

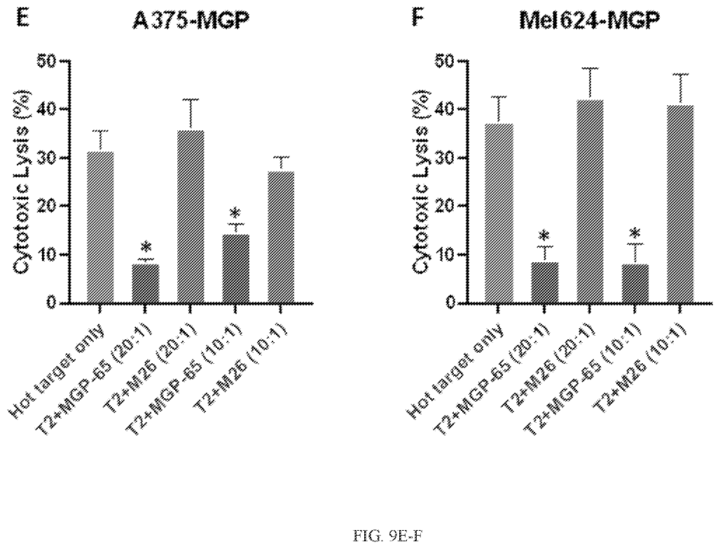

Following leukapheresis, HLA-A0201 healthy donor PBMC were stimulated with MGP-65 peptide (FVLAAVYRI—SEQ ID NO:22)-pulsed autologous DCs. After two rounds of stimulation, MGP-65-A2 tetramer-positive staining populations were detected (FIG. 9A). About 33 wells from one 48 well plate showed clear MGP-65 peptide tetramer positive CD8+ T cell population (FIG. 8), indicating that MGP-65 peptide specific T cells are easily expanded with cognate peptide stimulation, even in the PBMC of healthy donors without a history of SARS-CoV-2 infection. Expanded MGP-65 CTLs were tested functionally using standard ⁵¹Cr release assays (CRA). T2 cells (HLA-A0201), pulsed with titrated amounts of MGP-65 peptide, elicited CTL recognition and killing at peptide concentrations as low as 10 pM (FIG. 9B), indicating very high recognition affinity of MGP-65 CTLs for cognate peptide.

To verify that MGP-65 specific CTLs recognized the endogenously presented cognate peptide, HLA-A0201+target cells were engineered to express the SARS-CoV-2 MGP gene (A375-MGP, Mel624-MGP). MGP-65 specific CTL were able to lyse A375-MGP and Mel624-MGP cell lines, but not A375-GFP and Mel624-GFP control cell lines (FIGS. 9C and 9D). To further confirm that target recognition of MGP-65 specific CTL was through engagement of endogenously presented cognate peptide, cold target inhibition assay was performed. ⁵¹Cr pulsed MGP-expressing cells were radiolabeled with ⁵¹Cr while MGP-65 or M26 irrelevant peptide pulsed T2 cells were left unlabeled and used as cold targets or control cold targets, respectively. When adding cold targets at both 20:1 and 10:1 cold to hot target (C:H) ratio, the cytotoxicity of MGP-65 specific CTL for radiolabeled MGP-65 peptide targets was significantly inhibited (FIGS. 9E and 9F). However, there was no inhibition if control cold targets were added, indicating MGP-65 CTLs were able to then lyse MGP-65 targets via recognition of endogenously presented cognate peptide. This data provided further evidence that MGP-65 peptide is the natural endogenously presented MHC peptide.

To further evaluate function of the MGP-65 specific CTLs, intra-cellular staining (ICS) assay was performed to detect IFN-7 and TNF-α production. Co-culture of MGP-65 specific CTLs with MGP-65 peptide pulsed or MGP engineered target cells demonstrated specific recognition by IFN-7 and TNF-α produced compared with control targets pulsed with irrelevant peptide or engineered to express control GFP (FIG. 9G). A commensurate increase in T cell activation markers, CD137 and CD69, were also specifically and significantly elevated compared with the control group (FIG. 9G), when encountering relevant SARS-CoV-2 targets.

5. NSP13-242-Peptide Specific Cytotoxic T Cells Generated from the Peripheral Blood Recognize SARS-CoV-2-NSP13-Expressing Target Cells

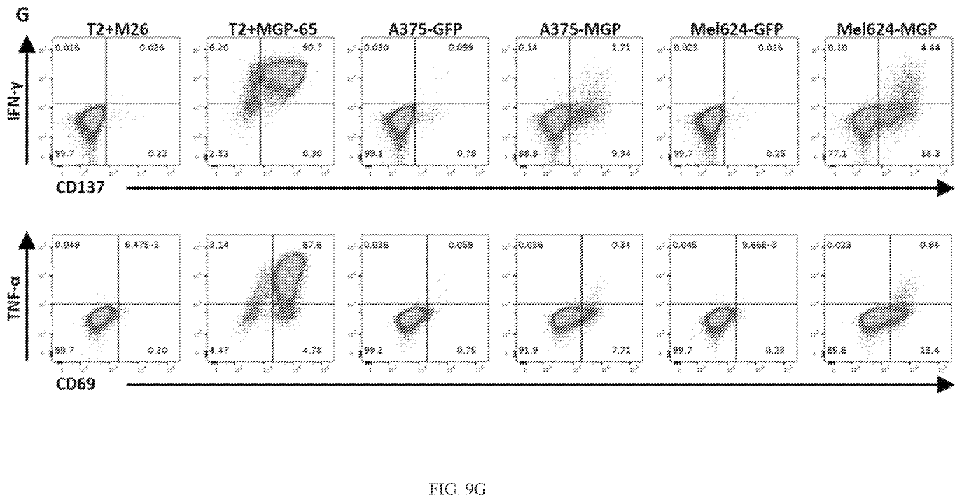

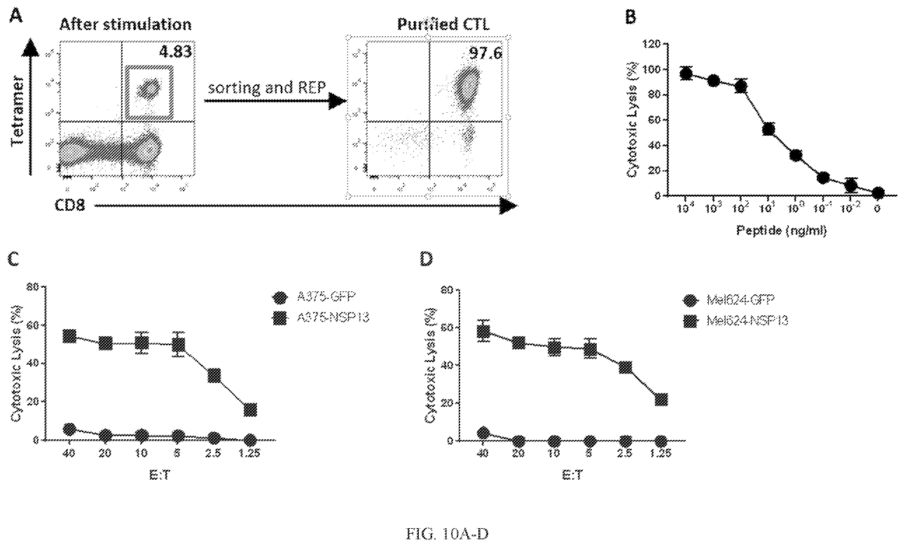

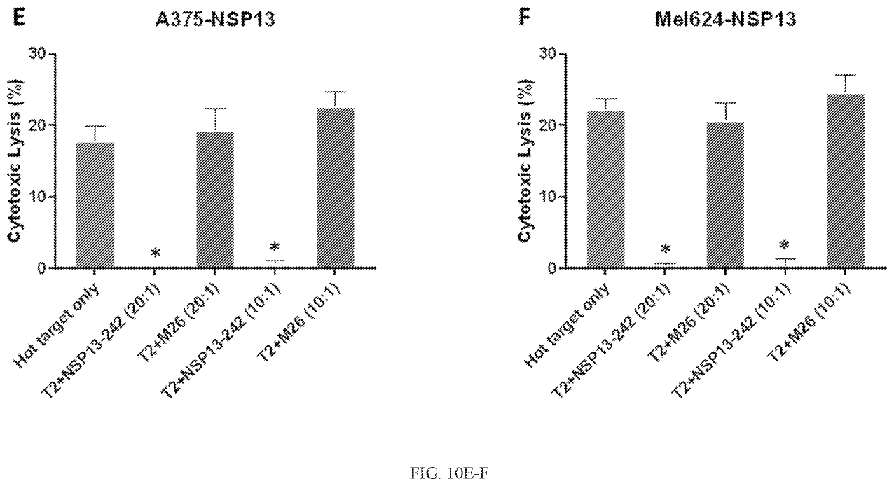

In contrast to structural proteins such as MGP and Spike protein, non-structural proteins of SARS-CoV-2 have a lower likelihood of inducing humoral responses and neutralizing antibodies as they are not expressed on the virion surface. However, non-structural proteins of SARS-CoV-2 infected cells can be presented as MHC bound peptides and induce cellular immune responses which can be long-lasting. Here, using the same workflow, the HLA-A0201 restricted peptide, NSP13-242 (TLVPQEHYV—SEQ ID NO:35) derived from NSP13 helicase was identified by MS/MS. Similar to MGP-65, NSP13-242 specific T cells were readily generated using the ETC workflow (FIG. 10A). Surprisingly, after stimulation with NSP13-242 peptide, all 48 wells from one 48 well plates showed clear NSP13-242 peptide tetramer positive CD8+ T cell population (FIG. 19), suggesting that NSP13-242 peptide may be highly immunogenic.

Cytotoxicity assay also demonstrated that NSP13-242 specific CTLs were able to recognize cognate peptide as low as 100 pM (FIG. 10B), indicating expression of high affinity TCR. More importantly, NSP13-242 specific CTLs were able to lyse NSP13 expressing targets A375-NSP13 and Mel624-NSP13, even at very low E:T ratio (2.5:1), but not control targets (FIGS. 10C and 10D), indicating that NSP13-242 specific CTLs can recognize the endogenous presented peptide of NSP13 protein. Similar to MGP-65 CTL, cold target inhibition assay also showed that when cold targets are added, the lytic capacity of NSP13-242 specific CTLs to hot targets, A375-NSP13 and Mel624-NSP13, was inhibited significantly (FIGS. 10E and 10F), further confirming that NSP13-242 specific CTLs lyse the targets via recognition of endogenously presented cognate peptide.

ICS assay demonstrated that NSP13-242 specific CTLs produce higher level of inflammatory cytokine IFN-γ and TNF-α and express higher levels of antigen-driven activation markers CD137 and CD69 when co-cultured with NSP13-242 peptide pulsed targets or NSP13 expressing targets, compared with the control targets (FIG. 10G). Thus, similar to MGP-65 specific CTLs, NSP13-242 specific CTLs will also initiate specific cellular immune response when encountering SARS-CoV-2.

6. NSP13-448 Peptide Specific Cytotoxic T Cells Generated from the Peripheral Blood Recognize SARS-CoV-2-NSP13-Expressing Target Cells

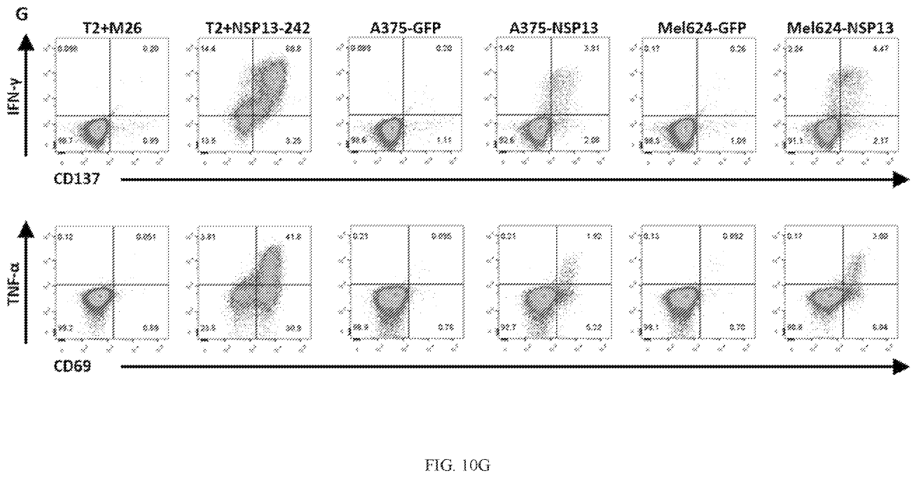

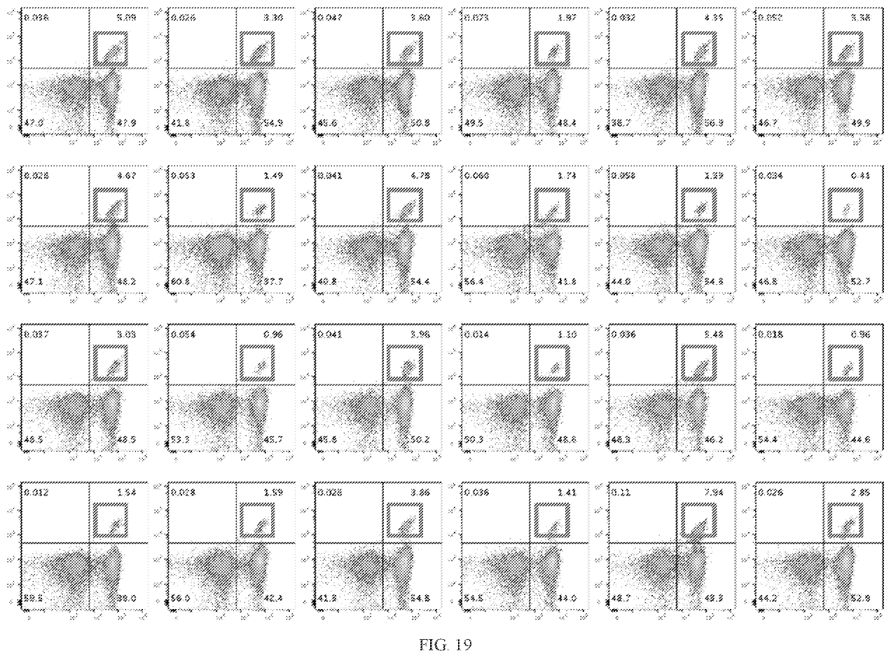

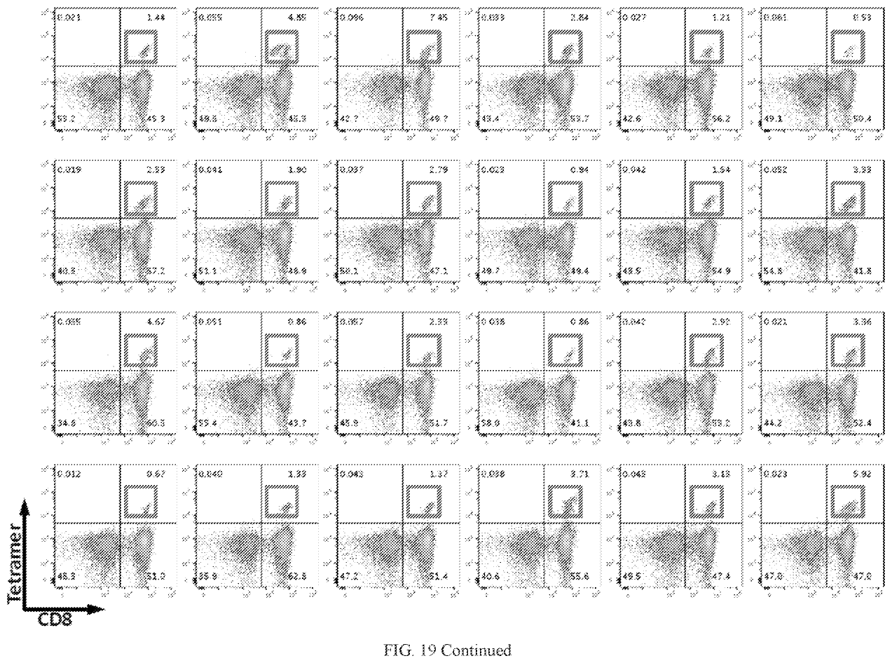

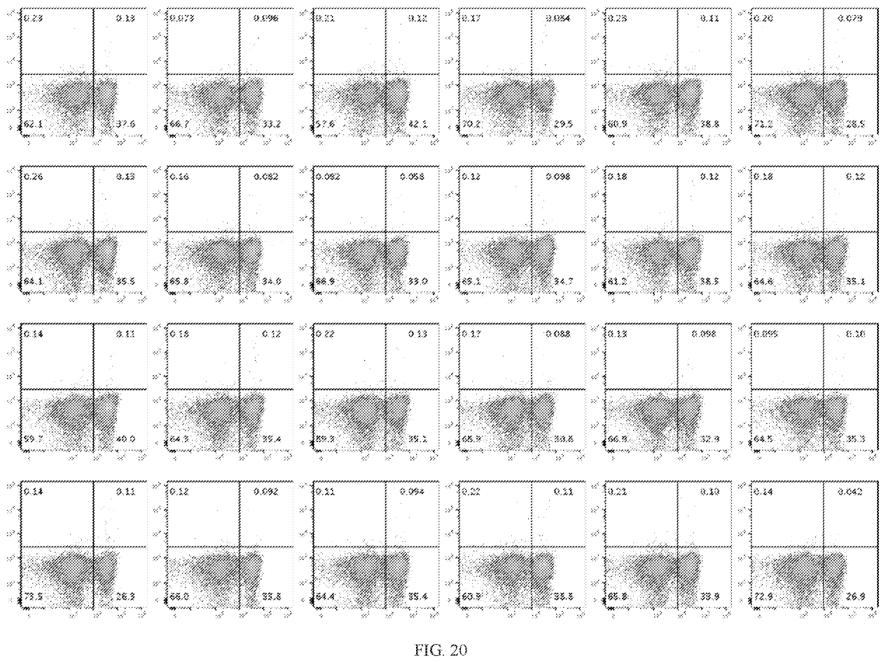

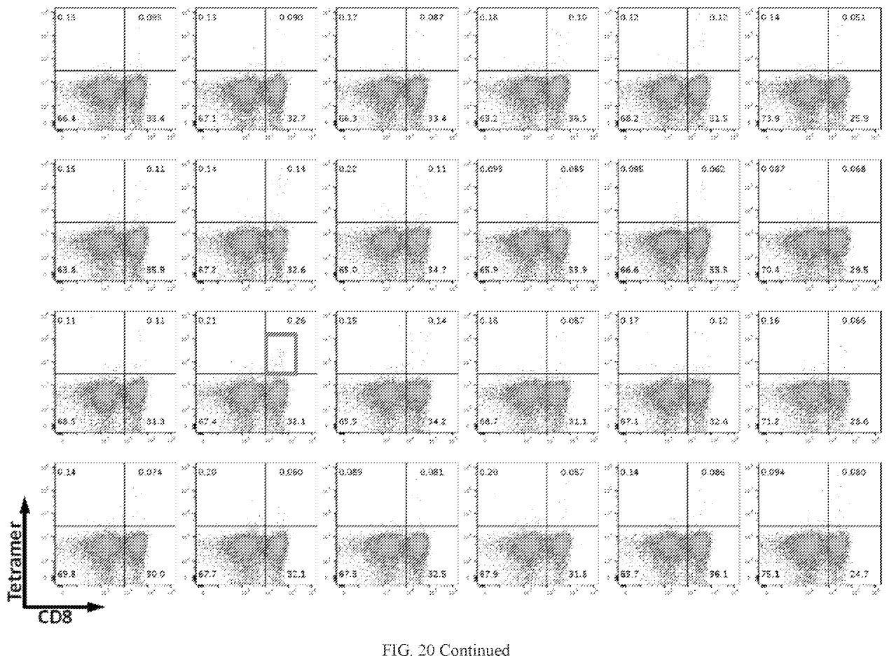

HLA-A0201 allele is expressed in about 45% of the Caucasian and Asian population (Kessler et al., 2003). Specific T cell targeting of other highly prevalent HLA-A alleles of SARS-CoV-2 would be desirable given the global reach of COVID19. Using the same workflow for MGP65 and NSP13-242 peptide, the inventors identified an HLA-A0101 restricted peptide, NSP13-448 (IVDTVSALVY—SEQ ID NO:34) derived NSP13 protein by MS/MS; this allele covers about 26% of Caucasian and 7% of Asian population (Kessler et al., 2003). Similar to NSP13-242, NSP13-448 specific T cells were readily generated using the ETC workflow (FIG. 11A). However, after stimulation with NSP13-448 peptide, only one well from one 48 well plates showed clear NSP13-448 peptide tetramer-positive CD8+ T cell population (FIG. 20), suggesting that NSP13-448 peptide may not be highly immunogenic.

Cytotoxicity assays also demonstrated that NSP13-448 specific CTLs were able to recognize cognate peptide as low as 100 nM (FIG. 11B), indicating moderate to low TCR affinity. Interestingly, NSP13-448 specific CTLs were still able to lyse NSP13 expressing targets A375-NSP13 and RPMI-7951-NSP13, even at very low E:T ratio (2.5:1), but not control targets (FIGS. 11C and 11D), indicating that NSP13-448 specific CTLs can recognize the endogenous presented peptide of NSP13 protein. Cold target inhibition assay demonstrated that when cold targets are added, the lytic capacity of NSP13-448 specific CTLs to hot targets, A375-NSP13 and RPMI-7951-NSP13, was inhibited significantly (FIGS. 11E and 11F), further confirming that NSP13-448 specific CTLs lyse these targets via recognition of endogenously presented cognate peptide.

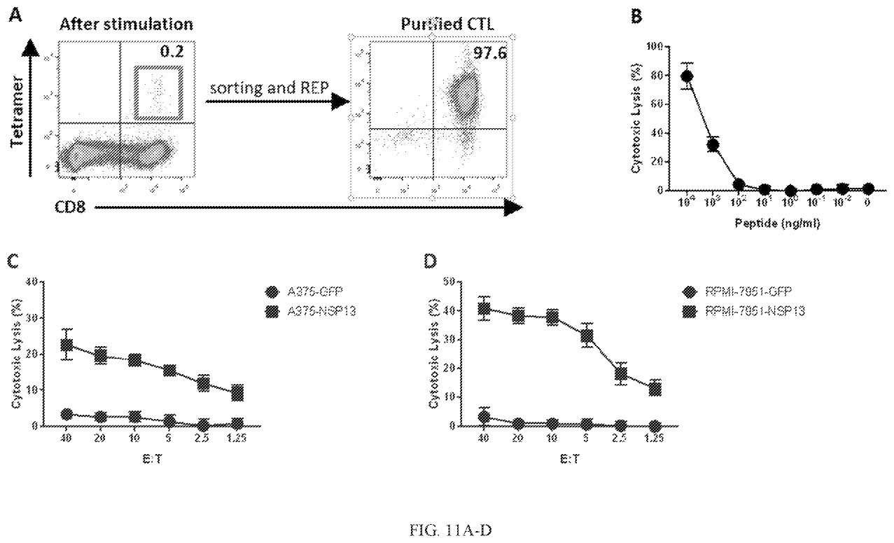

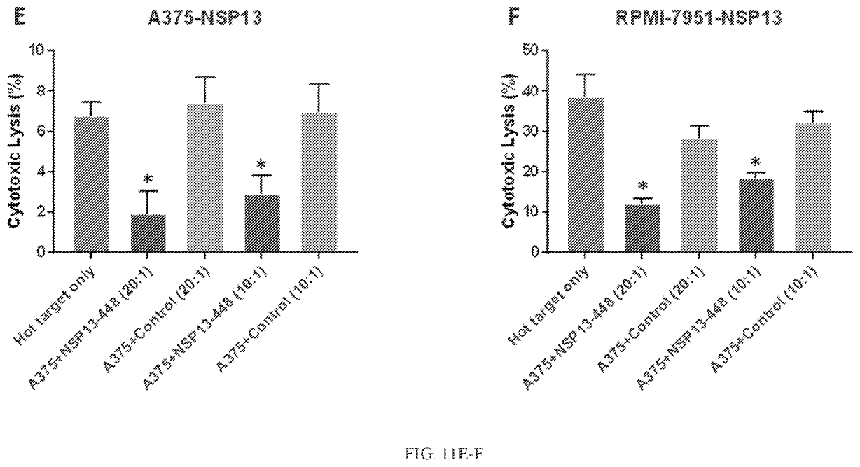

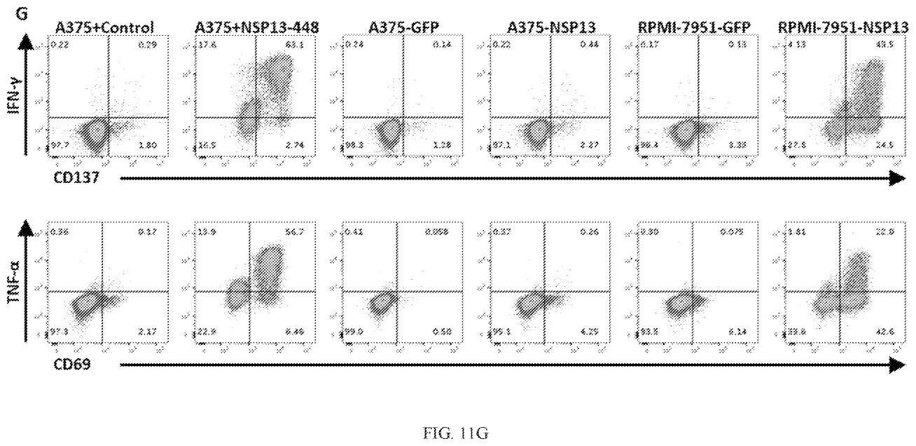

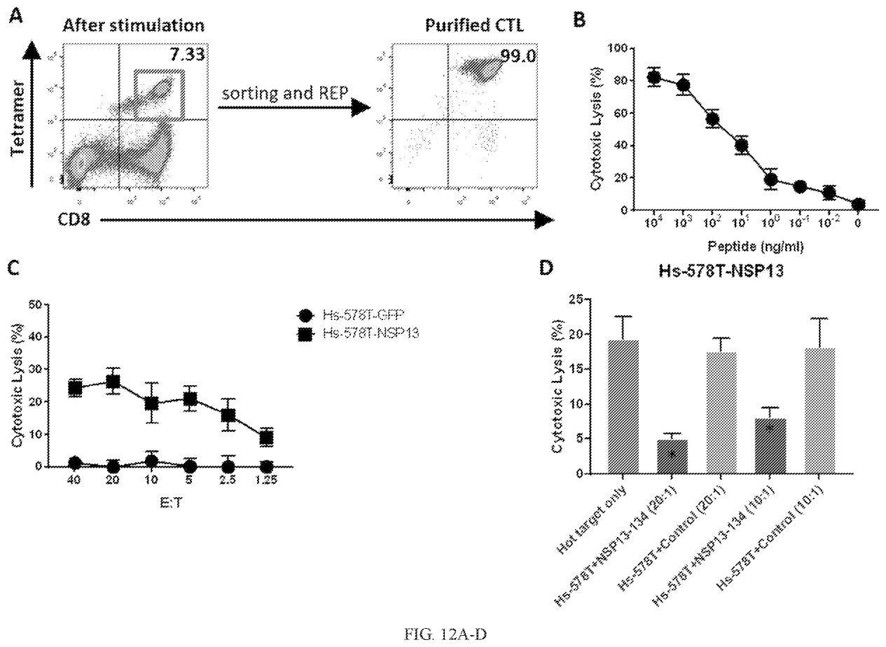

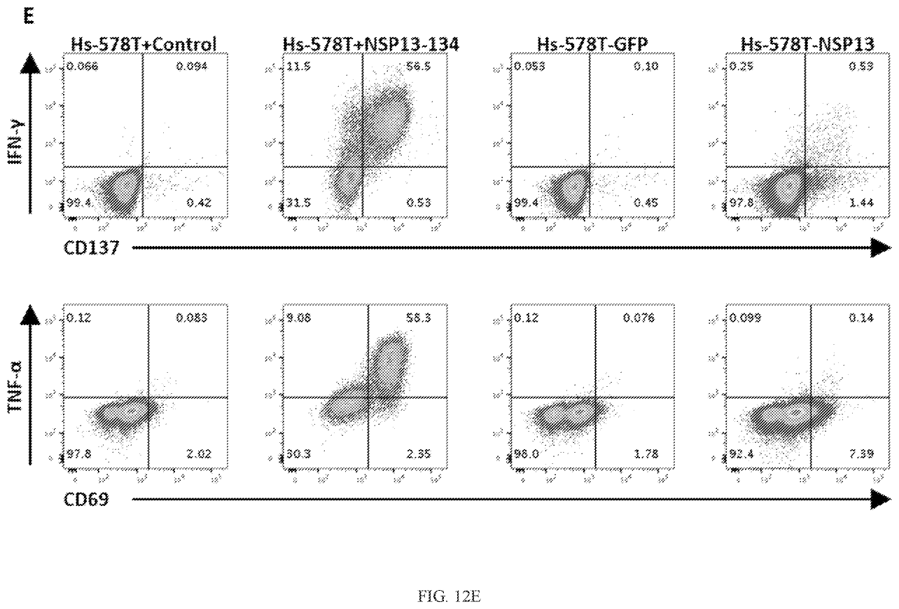

ICS assays demonstrated that NSP13-448 specific CTLs produce higher levels of inflammatory cytokines, IFN-γ and TNF-α, and express higher levels of antigen-driven activation markers CD137 and CD69 when co-cultured with NSP13-448 peptide pulsed targets or NSP13 expressing targets, compared with control targets (FIG. 12G). 7. NSP13-134 peptide specific cytotoxic T cells generated from the peripheral blood recognize SARS-CoV-2-NSP13-expressing target cells

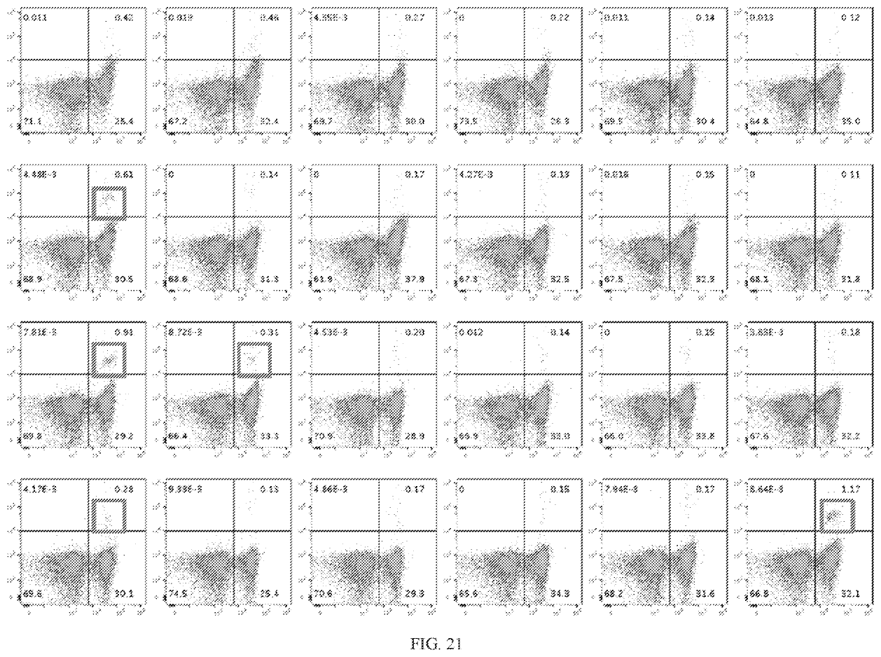

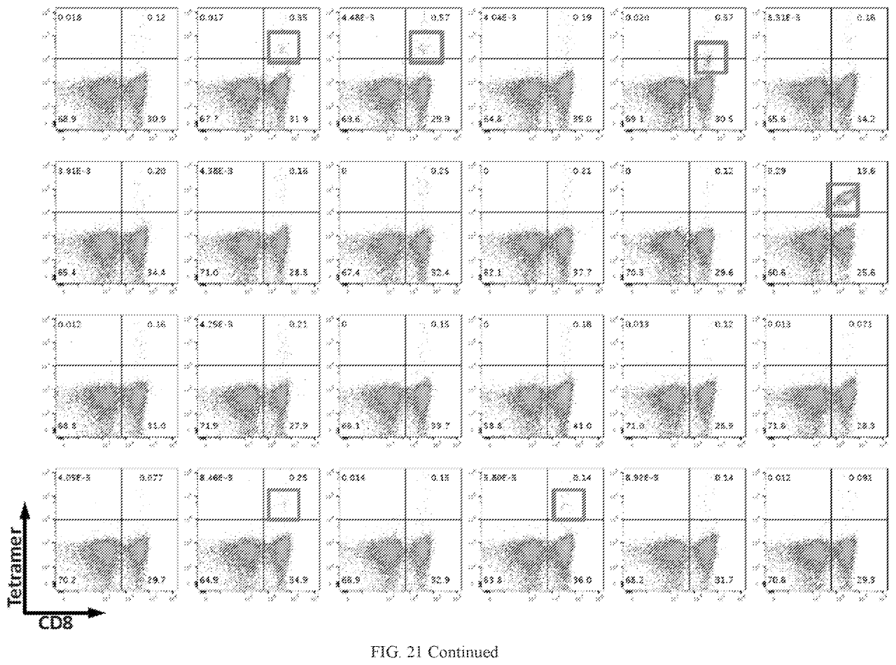

In addition to HLA-A0101 and HLA-A0201 allele, HLA-A0301 allele covers about 22% of Caucasian and 13% of African population (Kessler et al., 2003). Using the same workflow, the inventors identified an HLA-A0301 restricted peptide, NSP13-134 (KLFAAETLK—SEQ ID NO:36) derived NSP13 protein. Following in vitro stimulation using the ETC workflow, 11 wells of 48 wells showed clear NSP13-134 peptide tetramer positive CD8+ T cell population (FIG. 21), indicated that NSP13-134 peptide is sufficiently immunogenic to induce T cell responses in healthy donor without prior SARS-CoV-2 infection. After sorting and expansion, high purity of CD8+ and Tetramer+NSP13-134 CTLs were generated (FIG. 12A). Peptide titration assay showed NSP13-134 specific CTLs were able to recognize cognate peptide as low as 100 pM (FIG. 12B), indicating expression of relative high affinity TCR. Similar to MGP-65 and NSP13-242 specific CTLs, NSP13-134 specific CTLs were able to lyse NSP13 expressing targets Hs-578T-NSP13, even at low E:T ratio (2.5:1), but not control targets (FIG. 12C). Similar to MGP-65 and NSP13-242 CTL, cold target inhibition assay confirmed specific recognition of endogenously presented cognate peptide (FIG. 12D). ICS assay also confirmed specific recognition of NS13 expressing targets (FIG. 12E).

8. Expansion of NSP13-400 Peptide Specific Cytotoxic T Cells from the Peripheral Blood of Healthy Donor and Functional Assay

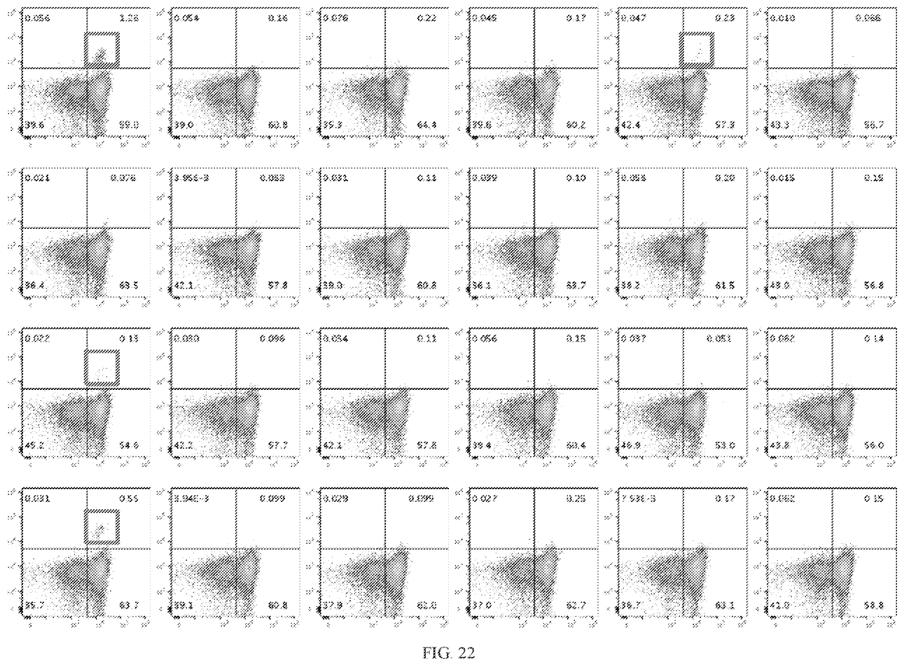

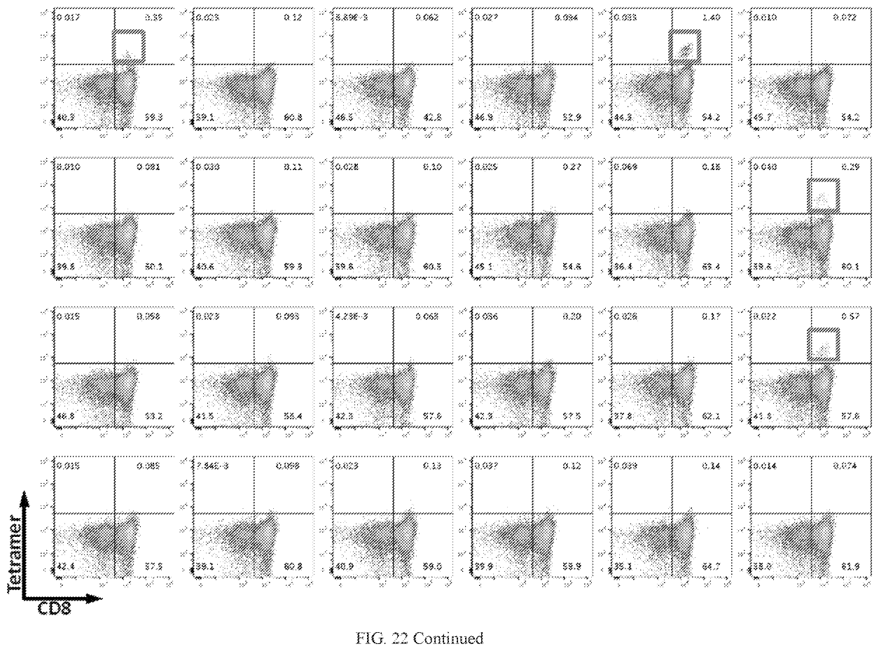

In addition to HLA-A0101, HLA-A0201 and HLA-A0301, HLA-A2402 allele covers an additional of 40% Asians and 20% Caucasians (Kessler et al., 2003). Using the same workflow, the inventors identified the HLA-A2402 restricted peptide, NSP13-400 (VYIGDPAQL—SEQ ID NO:37) of NSP13. Following in vitro stimulation using 9 of 48 wells showed clear NSP13-400 peptide tetramer positive CD8+ T cell population (FIG. 22), After sorting and expansion, high purity of CD8+ and Tetramer+NSP13-400 CTLs were generated (FIG. 13A). Similar to MGP-65 CTLs, NSP13-400 specific CTLs showed very high recognition affinity for the cognate peptide in peptide titration assay, as low as 10 pM concentration (FIG. 13B) and in accordance with peptide titration assay, very high, specific lysis of NSP13 expressing targets Hs-578T-NSP13 and M14-NSP13, even at E:T ratios as low as 1.25:1 (FIGS. 13C and 13D). Similarly, cold target inhibition assay showed specific level of NSP13-400 CTLs to hot target Hs-578T-NSP13 and M14-NSP13 which were significantly inhibited with addition of cold targets (FIGS. 13E and 13F), further confirmed that NSP13-400 specific CTLs lyse the SARS-CoV-2 targets via recognition of endogenously presented cognate peptide.

ICS assay also confirmed specific recognition of NS13 expressing targets (FIG. 13G). IFN-γ and TNF-α level were strikingly elevated in comparison with other SARS-CoV-2 CTL target assays suggesting high density endogenous presentation.

In summary, using the inventors' MHC IP elution and MS identification workflow, the inventors discovered five HLA Class-I restricted peptide derived from structure protein MGP and non-structure protein NSP13 of SARS-CoV-2 presented by several HLA alleles (HLA-A0101, HLA-A0201, HLA-A0301 and HLA-A2402) which cover approximately 80% of Caucasian and Asian populations. All five peptide were highly immunogenic and capable of readily eliciting T cell responses among healthy COVID19-negative donors. All five SARS-CoV-2 specific CTLs recognize endogenously presented cognate peptide and specifically lyse SARS-CoV-2+targets.

### 9. MGP-65 Specific T Cell Receptor Engineered T Cell (TCR-T) Recognize SARS-CoV-2 MGP-65 Expressing Target Cells

As proof of principle that these discoveries can lead to development of “off-the-shelf” SARS-CoV-2 specific T cell therapy of COVID19 patients, the inventors sequenced and cloned the TCR alpha and beta chains from MGP-65 specific T cells and determined if it was possible to transfer specificity and function to peripheral blood lymphocytes (PBLs). The sequence annotation revealed an alpha chain (TCR-a) belonging to TRAV17*01F/TRAJ50*01F subtype and beta chain (TCR-β) belonging to TRBV9*02F/TRBJ2-1*01F/TRBD1*01F (Table 3). The retroviral vector pMSGV1 containing whole length of TCR alpha chain and beta chain linked with cleavage peptide Furin and P2A was constructed and used to infect to OKT3 activated allogeneic PBL of another HLA-A0201 healthy donor. After 5 days of infection, about 37% CD8+Tetramer+ T cell population was observed (FIG. 14A), indicating successful exogenous TCR pairing in allogeneic PBL. After sorting and expansion, high purity MGP-65 specific TCR-T cells were generated (FIG. 14A).

To evaluate the function and specificity of MGP-65 specific TCR-T, a cytotoxicity ⁵¹Cr release assay (CRA) and intra-cellular staining (ICS) assay were performed and compared to the parental MGP-65-specific CTL line. MGP-65 TCR-T were able to recognize titrated peptide pulsing targets at peptide concentrations as low as 10 pM (FIG. 14B), indicating that the TCR-T also displayed high affinity recognition of cognate peptide. Furthermore, MGP-65 TCR-T specifically lysed MGP expressing targets A375-MGP and M624-MGP, (FIGS. 14C and 14D), commensurate with, ICS assays demonstrating that compared to the parental CTL, MGP-65 specific TCR-T produced higher levels of IFN-7 and TNF-α, as well as activation markers, CD137 and CD69 when co-cultured with MGP expressing targets or MGP-65 peptide pulsing targets (FIG. 14E).

### C. Discussion

To date, nearly 1,500 predicted Class I epitopes for SARS-CoV2 have been identified by in silico prediction methods, and in some cases, ‘validated’ by eliciting T cell responses using PBMC of patients with COVID19 (Campbell et al., 2020; Grifoni et al., 2020a). These peptides have been used extensively to evaluate the T cell response of patients, and occasionally healthy donors, to COVID19, and COVID19 vaccines, and increasingly, to develop T cell-based therapies. What has not been demonstrated however, is whether any of these 1,500 predicted peptides are in fact processed and presented by SARS-CoV2+ cells and represent naturally-occurring epitopes recognized by T cells. A preliminary screen of predicted SARS-CoV2 epitopes considered “immunodominant” among widely cited reports appears to support this premise: the inventors found that 7 of these 8 predicted peptides were unable to elicit a T cell response that would lead to recognition of SARS-CoV2+ targets suggesting that responses to these peptides may be artifactual, or at best cross-reactive (FIG. 15) (Agerer et al., 2021; Ahmed et al., 2020; Gao et al., 2020; Grifoni et al., 2020a; Kar et al., 2020; Safavi et al., 2020; Shomuradova et al., 2020; Sohail et al., 2021; V. Gauttier, 2020; William Chour, 2020). To date, there has been no empiric validation of SARS-CoV2 epitopes for immunogenicity.

The inventors postulate that immunogenic epitopes for SARS-CoV-2 are best defined empirically by directly analyzing peptides eluted from MHC and then validating immunogenicity by determining if such peptides can elicit T cells recognizing SARS-CoV-2 antigen-expressing targets. Mass spectrometry (MS) is an ideal analytical approach to precisely identify the naturally expressed antigenic epitopes and enables investigators to address the complexity associated with differential expression and processing of antigenic proteins by infected cells. Based on immunoaffinity capture of the MHC-antigenic peptide complex from cells engineered to express SARS-CoV2 genes, this approach allowed for direct profiling and identification of the SARS-CoV2 immunopeptidome. By eliciting T cell responses against these candidate epitopes, the inventors confirm empiric recognition of SARS-CoV2+ cells and endogenous presentation of these peptides.

In this study, the inventors identify and validate 5 Class I-restricted SARS-CoV2 epitopes expressed by structural (MGP) and non-structural genes (NSP13), presented by Class I alleles (HLA-A*0101, A*0201, A*0301, HLA-A*2402) prevalent among >75% of the general population. Using recombinant vectors encoding these alleles, the inventors engineered expression of highly conserved regions of SARS-CoV2 membrane glycoprotein (MGP) and non-structural protein-13 (NSP13) genes, recovered MHC, eluted peptides and applied data dependent analysis liquid chromatography-tandem mass spectrometry (DDA MS/MS) yielding over 12,000 spectra, which were then deconvoluted and filtered to a handful of candidate peptide epitopes. The immunogenicity of 5 peptides was validated on the basis of their ability to elicit peptide-specific T cells capable of recognizing and killing SARS-CoV2-expressing target cells, and in one example, redirecting specificity of peripheral blood lymphocytes with an engineered TCR to SARS-CoV2 MGP.

The importance of eliciting a meaningful anti-viral T cell response has been well-documented; SARS- and MERS-responsive T cells were found to have a protective role (Poland et al., 2020). Emergence of SARS-CoV-2-specific T cell responses were recently shown to be associated with a sustained viral clearance and highlight the importance of developing vaccines that promote cellular immunity against SARS-CoV-2 (Gallais et al., 2021; Long et al., 2020; Sekine et al., 2020).

Recently, the emergence of mutant escape variants of SARS-CoV2 has led to global concerns over possible breaches in viral protection following immunization with current vaccines which elicits a predominantly serologic response (Abdool Karim and de Oliveira, 2021; Agerer et al., 2021; Darby and Hiscox, 2021; Kuzmina et al., 2021; Plante et al., 2021). By targeting a non-surface, non-structural protein, in this case nsp13, which encodes viral helicase, escape variants are less likely to develop; in fact, none of the known variants harbor mutations among the epitope sequences identified here. Furthermore, the strategy presented allows for identification of epitopes spanning almost any SARS-CoV2 gene; selection of virus-essential gene targets provides a rational T cell-based approach to mitigate selection of antigen-loss variant and the potential for long-term viral immunoprotection.

Equally important in defining the landscape of COVID19 infection and control, its natural history, vaccine efficacy and therapeutic intervention, is an accurate measure of the SARS-CoV2-specific immune response. While the pools of predicted peptides currently in use to evaluate Class II ad Class I-restricted responses have been used extensively and appear to provide a measure of overall immune response, SARS-CoV2-specific immunity is poorly defined when the majority of peptides may not be immunogenic; the use of a highly defined subset of peptides may provide a more accurate representation of T cell immune response to COVID19 infection Although the inventors' current panel of 5 peptides is not extensive, it does represent highly conserved regions of the SARS-CoV2 genome, presented by several highly prevalent allelotypes, and may readily be applied to Class II as well as Class I-restricted epitopes. A more extensive panel of 18 epitopes have been prepared and will be evaluated for clinical correlative studies.

Finally, as further proof of immunogenicity for epitopes defined in this manner (by tandem mass spectrometry followed by empiric in vitro validation), the inventors reconstituted functional SARS-CoV2-specific TCR using a vector encoding the alpha and beta chains of MGP65-specific T cells. This strategy additionally provides an off-the-shelf reagent for adoptive TCR-T-based therapies and one can envision a collection of TCR-T vectors recognizing a matrix of MS/MS-defined SARS-CoV2 epitopes spanning highly conserved regions of the viral genome and representing a broad panel of high prevalence HLA alleles for cell-based therapy of COVID19-infected patients.

### D. Tables

TABLE 2
Summary of identified SARS-CoV-2 epitopes

| | | | | | Bind- | |
|---|---|---|---|---|---|---|
| | | | | | ing | |
| | SEQ | | | | Predic- | Con- |
| Eluted | ID | Protein | MS | HLA | tion | sensus |
| Peptide | NO | ID | method | allele | (nM) | region |
| VYIGDPAQL | 37 | NSP13-400 | DDA | A2402 | 325.8 | Yes |
| KLFAAETLK | 36 | NSP13-134 | PRM | A0301 | 9.9 | Yes |
| TLVPQEHYV | 35 | NSP13-242 | PRM | A0201 | 72.2 | Yes |
| IVDTVSALVY | 34 | NSP13-448 | PRM | A0101 | 356.9 | Yes |
| FVLAAVYRI | 22 | MGP-65 | PRM | A0201 | 38.1 | Yes |

TABLE 3
Alpha and Beta chain summaries
Alpha Chain

| Result summary: | Productive TRA rearranged sequence | | |
|---|---|---|---|
| | (no stop codon and in-frame junction) | | |
| V-GENE and allele | Homsap | score = 1330 | identity |
| | TRAV17*01 F | | = 100.00% |
| | | | (267/267 nt) |
| J-GENE and allele | HomsapTRAJ50*01 | score = 273 | identity = 95.00% |
| | F | | (57/60 nt) |
| FR-IMGT lengths, | [26.17.34.11] | [5.7.11] | CATAQTSYDKVIF |
| CDR-IMGT lengths and | | | (SEQ ID NO: 82) |
| AA JUNCTION | | | |

Beta chain

| Result summary: | Productive TRB rearranged sequence | | |
|---|---|---|---|
| | (no stop codonand in-frame junction) | | |
| V-GENE and allele | Homsap TRBV9*02 | score = 1360 | identity |
| | F | | =100.00% (273/273 nt) |
| J-GENE and allele | Homsap TRBJ2- | score = 241 | identity = 98.00% |
| | 1*01 F | | (49/50 nt) |
| D-GENE and allele by | Homsap TRBD1*01 | | |
| IMGT/JunctionAnalysis | F | | |
| FR-IMGT lengths, | [26.17.37.10] | [5.6.13] | CASSVEGPTYNEQFF |
| CDR-IMGT lengths and | | | (SEQ ID NO: 83) |
| AA JUNCTION | | | |

TABLE S1
Predicted high HLA binding peptide of MGP
or NSP13 for PRM-MS analysis

| MGP | | NSP13 | | NSP13 | | NSP13 | |
|---|---|---|---|---|---|---|---|
| predicted | | predicted | | predicted | | predicted | |
| HLA-A0201 | | HLA-A0101 | | HLA-A0201 | | HLA-A0301 | |
| peptide | | peptide | | peptide | | peptide | |
| | Binding | | Binding | | Binding | | Binding |
| | affinity | | affinity | | affinity | | affinity |
| Peptide | (nM) | Peptide | (nM) | Peptide | (nM) | Peptide | (nM) |
| GLMWLSYFI | 4.5 | VTDVTQLYL | 128.9 | KLVLSVNPY | 12.3 | VVYRGTTT | 8.2 |
| (SEQ ID | | (SEQ ID | | V (SEQ ID | | YK (SEQ ID | |
| NO: 43) | | NO: 44) | | NO: 45) | | NO: 46) | |
| FLYIIKLIFL | 5 | TVDSSQGSE | 167.7 | KLSYGIATV | 20.3 | KLFAAETLK | 9.9 |
| (SEQ ID | | Y (SEQ ID | | (SEQ ID | | (SEQ ID | |
| NO: 47) | | NO: 48) | | NO: 49) | | NO: 36) | |
| LIFLWLLWP | 5.6 | FAIGLALYY | 285.4 | KLNVGDYF | 22.1 | MSYYCKSH | 24.6 |
| V (SEQ ID | | (SEQ ID | | V (SEQ ID | | K (SEQ ID | |
| NO: 50) | | NO: 51) | | NO: 52) | | NO: 53) | |
| VATSRTLSY | 9.8 | DSSQGSEYD | 301.7 | SMATNYDLS | 55.6 | QLYLGGMS | 37 |
| (SEQ ID | | Y (SEQ ID | | V (SEQ ID | | YY (SEQ ID | |
| NO: 23) | | NO: 54) | | NO: 55) | | NO: 56) | |
| TLACFVLAA | 12.9 | ATEETFKLS | 348 | TLEQYVFCT | 61.5 | SVVNARLR | 48.3 |
| V (SEQ ID | | Y (SEQ ID | | V (SEQ ID | | AK (SEQ ID | |
| NO: 57) | | NO: 58) | | NO: 59) | | NO: 60) | |
| KLLEQWNL | 15.8 | IVDTVSALV | 356.9 | TLVPQEHYV | 72.8 | SAQCFKMF | 73 |
| V (SEQ ID | | Y (SEQ ID | | (SEQ ID | | YK (SEQ ID | |
| NO: 61) | | NO: 34) | | NO: 35) | | NO: 62) | |
| FLWLLWPVT | 20.9 | KSAQCFKMF | 555.3 | TLVPQEHYV | 78 | YLGGMSYY | 81.9 |
| L (SEQ ID | | Y (SEQ ID | | RI (SEQ ID | | CK (SEQ ID | |
| NO: 63) | | NO: 64) | | NO: 65) | | NO: 66) | |
| LLWPVTLAC | 34.8 | HFAIGLALY | 577.5 | YVFCTVNAL | 93 | RFNVAITRA | 83.8 |
| FV (SEQ ID | | Y (SEQ ID | | (SEQ ID | | K (SEQ ID | |
| NO: 67) | | NO: 68) | | NO: 69) | | NO: 70) | |
| FVLAAVYRI | 38.1 | TCDWTNAG | 588.2 | GLALYYPSA | 104.1 | YVFTGYRVT | 98.6 |
| (SEQ ID | | DY (SEQ ID | | (SEQ ID | | K (SEQ ID | |
| NO: 22) | | NO: 71) | | NO: 72) | | NO: 73) | |
| LMWLSYFIA | 43.1 | CTERLKLFA | 630.1 | FKVNSTLEQ | 108.7 | CIRRPFLCC | 102.8 |
| (SEQ ID | | (SEQ ID | | YV (SEQ ID | | K (SEQ ID | |
| NO: 74) | | NO: 75) | | NO: 76) | | NO: 77) | |

### E. STAR Methods

Blood donors and Cell lines: Healthy donor peripheral blood mononuclear cells (PBMC) samples expressing the HLA-A0101, HLA-A0201, HLA-A0301 or HLA-A2402 allele were purchased from HemaCare (CA, USA) as a source of responding T cells and autologous antigen presenting cells. TAP-deficient T-B cell hybrid cell line T2, melanoma cell line A375 (HLA-A0101/0201), RPMI-7951 (HLA-A0101/0201) and Phoenix-GP were purchased from ATCC (VA, USA). Melanoma cell lines Hs-578T (HLA-A0301/2402) and M14 (HLA-A 1101/2402) were purchased from NCL. Melanoma cell line Me1624 (HLA-A0201) was the gift from Dr. Steven Rosenberg (NCI). Lymphoblastoid cell lines (LCL) are EBV-transformed lymphoblastoid cell lines established in our laboratory. Cancer cell lines were maintained in RPMI-1640 media with Hepes (25 mM), L-glutamine (4 mM), penicillin (50 U/ml), streptomycin (50 mg/ml), sodium pyruvate (10 mM), nonessential amino acids (1 mM), and 10% fetal bovine serum (FBS) (Sigma, MO, USA). Phoenix-GP were cultured in DMEM media with Hepes (25 mM), L-glutamine (4 mM) and 10% FBS.

Lentivirus transduction: The cDNA of membrane glyco-protein (MGP) and Non-structure protein 13 (NSP13) of ORF1b from SARS-CoV-2 were purchased from Genscript (NJ, USA) and cloned into lentiviral vector pLVX (TAKARA, CA, USA) with fusion of GFP. In this vector, the expressing gene was driven by human EFl promoter. MGP-pLVX or NSP13-pLVX lentiviral vector were transfected into package cell line 293T, together with package vectors contain VSVG envelop vector to make lentivirus. A375, Mel624, RPMI-7951, Hs-578T and M14 cell lines were infected with MGP-pLVX or NSP13-pLVX lentiviral vectors and the stable cell lines were screened with puromycin selection. MGP or NSP13 gene expressing efficiency was detected by analyzing the percentage of GFP using flow cytometry (NovoCyte Flow Cytometer Systems, Agilent, CA, USA).

HLA Class-I binding peptide identification: HLA Class-I binding peptide isolation and identification via immune-precipitation (IP) and tandem mass spectrometry (MS) methods are referenced from prior study (Bradley et al., 2020). Briefly, about 300 to 500 million cells engineered to express the SARS-CoV-2 MGP or NSP13 gene were homogenized in cold NP40 lysis buffer supplemented with protease inhibitor cocktail (Roche, CA, USA). Lysates were cleared by subsequent centrifugation and filtering steps. HLA class I molecules from the cleared lysate were incubated with anti-HLA-A, B, C monoclonal antibody (W6/32) coupled Sepharose-4B resin (GE Healthcare, IL, USA) at room temperature for 2 hours. The un-bound protein was washed by PBS. The HLA molecules with their bound peptides were then eluted from the affinity column with 0.1N acetic acid. The detached peptides were separated from HLA molecules using 3 kDa cut-off centrifugal ultrafilters (Millipore, MO, USA), and then concentrated using vacuum centrifugation.

Peptides were reconstituted in 0.1% formic acid in water prior to mass spectrometry acquisition. New Objective PicoFrit nanospray column (360 μm OD×75 μm ID) was packed with Dr. Maisch 3 m ReproSil-Pur C18 beads to 30 cm. The same C18 beads were used to pack 25 mm trap column using 360 μm×OD 150 m ID fused silica capillary fitted with Kasil on one end. Peptides were separated using Thermo Scientific EASY-nLC 1200. Solvent A was 0.1% formic acid in water, and solvent B was 0.1% formic acid in 80% acetonitrile. For each injection, 10-15 μL was loaded and eluted using 25-60 minute gradient from 5 to 40% solvent B at 250-300 nL/min. Thermo Scientific Q-Exactive HF or Orbitrap Exploris 480 tandem mass spectrometer was used to acquire mass spectra using data-dependent acquisition (DDA) or Parallel reaction monitoring (PRM).

DDA acquisition on Q-Exactive HF: Precursor spectra (400-1600 m/z) were collected at 60,000 resolution with Automatic Gain Control (AGC) target set at 3e6 and maximum inject time of 100 ms. Fragment spectra were collected at 15,000 resolution with AGC target set at 1e5 and maximum inject time of 25 ms. The isolation width was set to 1.6 m/z. Normalized collision energy was set at 27. Top-20 most intense precursor ions for fragmentation was selected. Charge exclusion was enabled to include only precursor charges between +2 and +4 with AGC threshold of 5e3. Dynamic exclusion was set to 10 seconds to exclude all isotopes clusters.

PRM acquisition Orbitrap Exploris 480: Precursor spectra (400-1600 m/z) were collected at 30,000 resolution with standard AGC target set and automatic maximum inject time. RF lens was set at 50%, Cycle time, at 3 seconds. Fragment spectra scan range was set at 500-1600 and collected at 15,000 resolution with AGC target set at standard and automatic injection time. The isolation width was set to 2 m/z with unscheduled time mode. Normalized collision energy was set at 30%. An inclusion list containing m/z values of protonated precursor peptide ions of interest was generated in Skyline-daily (version 20.2.1.135).

Peptide selection and validation: To analyze the acquired MS/MS spectra, the spectra were searched against the Swiss-Prot protein database (version 2020_05) by using Mascot search engine node (version 2.6) within Proteome Discoverer 2.3. The searches were performed with a precursor peptide mass tolerance of 15 ppm and fragment ion mass tolerance of 15 ppm using monoisotopic parent and fragment ion masses allowing for two missed cleavages without enzyme specification. Taxonomy was restricted to Homo sapiens (20,386 sequences) and Viruses (17,008 sequences). The False Discovery Rate (FDR) was determined using “Target Decoy PSM Validator” node within Proteome Discoverer (version 2.3) and protein/peptide with an FDR of 1% being retained for further analysis. The predicted binding to these putative peptides was determined and filtered by IEDB T Cell Epitope Prediction Tools (http://tools.iedb.org/main/tcell/).

Generation of SARS-CoV-2 specific T cells: Generation of antigen-specific T cell stimulation was performed according to our endogenous T cell (ETC) generation workflow (Chapuis et al., 2016). Briefly, adherent PBMCs were treated with GM-CSF (800 U/mL) and IL-4 (500 U/mL) for 6 days to generate immature DC (iDC), and the iDC were then matured with a cytokine cocktail containing TNF-α (10 ng/mL), IL-10 (2 ng/mL), IL-6 (1000 U/mL), PGE-2 (1000 ng/mL) for an additional 2 days. Candidate SARS-CoV-2 peptide was pulsed on HLA-matched mature DC for MGP-65 (FVLAAVYRI—SEQ ID NO:22, HLA-A0201), NSP13-448 (IVDTVSALVY—SEQ ID NO:34, HLA-A0101), NSP13-242 (TLVPQEHYV—SEQ ID NO:35, HLA-A0201), NSP13-134 (KLFAAETLK—SEQ ID NO:36, HLA-A0301), NSP13-400 (VYIGDPAQL—SEQ ID NO:37, HLA-A2402) (all purchased from Genscript, NJ, USA) in PBS/HSA. The peptide pulsed DCs were then co-cultured with autologous PBMC in RPMI-1640 with Hepes (25 mM), L-glutamine (4 mM), penicillin (50 U/ml), streptomycin (50 mg/ml), sodium pyruvate (10 mM), and 10% human AB serum. After 7 days in culture, T cell cultures were restimulated with peptide-pulsed DC as before. IL-2 (10 U/mL) and IL-7 (5 ng/mL) were added on the second day.

Sorting and expansion: After two stimulation cycles, an aliquot of each well was stained with custom PE-conjugated MHC tetramer folded with HLA matched SARS-CoV2 peptide, and with APC-Cy7 conjugated anti-CD8 antibody (Biolegend, CA, USA). Cells were washed and analyzed by flow cytometry (NovoCyte Flow Cytometer Systems, Agilent, CA, USA). The tetramer positive staining wells were pooled and CD8/Tetramer double positive population were sorted using flow cytometric sorting (ARIA II sorter, BD, CA, USA) and then expanded using a rapid expansion protocol (REP) in a sterile 25 mL flask containing RPMI-1640 with Hepes (25 mM), L-glutamine (4 mM), penicillin (50 U/ml), streptomycin (50 mg/ml), sodium pyruvate (10 mM), 10% fetal bovine serum (FBS), irradiated PBMC and LCL feeder cells, as previously described (Chapuis et al., 2012). After expansion, the purity of antigen specific T cells were determined with anti-CD8 antibody and MGP-65, NSP13-448, NSP13-242, NSP13-134, or NSP13-400 tetramer staining again.

Function analysis of SARS-CoV-2 specific T cells: The cytotoxicity of purified SARS-CoV-2 specific T cells following expansion was confirmed using standard chromium (⁵¹Cr) release assay (CRA). Peptide dose titration experiments were performed to test cognate peptide recognition of SARS-CoV-2 CTL. Titrating concentrations of MGP-65 or NSP13-242 peptide-pulsed T2 cells (HLA-A2+) were used for evaluating MGP-65 or NSP13-242 CTL; NSP13-448 peptide pulsed A375 cells (HLA-A1+), for NSP13-448 CTL; NSP13-134 peptide pulsed Hs-578T cells (HLA-A3+) for NSP13-134 CTL, and NSP13-400 pulsed M14 cells (HLA-A24+) for NSP13-400 CTL. Target cells were labeled with 100 Ci ⁵¹Cr (Perkin Elmer, CA, USA) in 1 ml of tumor cell culture media for 1 hour, then washed and plated at 2,000 target cells per well in triplicate. MGP-65, NSP13-448, NSP13-242, NSP13-134 or NSP13-400 specific T cells were added at effector-to-target (E:T) of 20:1 cell ratio for 4 hours. Supernatant was collected from the wells and ⁵¹Cr measured with a gamma radiation counter. The percentage of specific target cell lysis was calculated, correcting for background ⁵¹Cr release and relative to a maximum ⁵¹Cr release as measured by NP40 lysed target cells (Pollack et al., 2014).

Tumor cell lines engineered to express MGP or NSP13 genes were used as targets to evaluate SARS-CoV-2 specific T cell recognition of endogenously presented epitopes. A375-MGP, Mel624-MGP (HLA-A0201+, MGP+), A375-GFP, Mel624-GFP (HLA-A0201+, GFP+), were used to evaluate MGP-65 specific T cell activity; A375-NSP13, RPMI-7951-NSP13 (HLA-A0101+, MGP+), A375-GFP, RPMI-7951-GFP (HLA-A0101+, GFP+), for NSP13-448 specific T cell activity; A375-NSP13, Mel624-NSP13 (HLA-A0201+, NSP13+), A375-GFP, Mel624-GFP, for NSP13-242 specific T cell activity; Hs-578T-NSP13 (HLA-A0301+, HLA-A2402+, NSP13+), Hs-578T-GFP (HLA-A0301+, HLA-A2402+, GFP+), for NSP13-134 specific T cell activity and M14-NSP13 (HLA-A2402+, NSP13+), Hs-578T-NSP13, M14-GFP (HLA-A2402+, GFP+), Hs-578T-GFP, for NSP13-400 specific T cell activity. ⁵¹Cr labeled target cells were co-cultured with SARS-CoV-2 specific T cells at varying effector-to-target (E:T) cell ratios. After the incubation period, antigen-specific target cell lysis was determined as above.

Cold target inhibition assay: To confirm, epitope and antigen-specificity against relevant tumor targets, cold target inhibition assays were performed as described previously (Park et al., 2017). For MGP-65 specific T cell test, A375-MGP and Mel624-MGP cells labeled with ⁵¹Cr were used as ‘hot’ targets. Non-radiolabeled T2 cells pulsed with MGP-65 peptide (10 g/ml) were used as cold targets. Non-radiolabeled T2 cells pulsed with M26 control peptide (ELAGIGILTV—SEQ ID NO:39, HLA-A0201) were used as control cold targets. Before co-culturing the MGP-65 specific T cells with hot target, cold targets (at 10- or 20-fold greater numbers than radiolabeled hot targets) were added and incubated with a given antigen-specific T cells for one hour. ⁵¹Cr-labeled hot targets were added at E:T ratio (20:1) and incubated for further 4 hours. After the incubation period, target cell lysis by MGP-65 specific T cells was determined above. Similarly, for NSP13-448 specific T cells, A375-NSP13 and RPMI-7951-NSP13 cells labeled with ⁵¹Cr were used as hot targets. Non-radiolabeled A375 cells pulsed with NSP13-448 peptide or VGLL1 HLA-A0101 peptide (LSELETPGKY—SEQ ID NO:40) (Bradley et al., 2020) were used as cold targets and control cold targets, respectively. For NSP13-242 specific T cells, A375-NSP13 and Mel624-NSP13 cells labeled with ⁵¹Cr were used as hot targets. Non-radiolabeled T2 cells pulsed with NSP13-242 peptide or M26 peptide were used as cold targets and control cold targets, respectively. For NSP13-134 specific T cells, Hs-578T-NSP13 cells labeled with ⁵¹Cr were used as hot targets. Non-radiolabeled Hs-578T cells pulsed with NSP13-134 peptide or A3 control peptide (KVFPCALINK—SEQ ID NO:41, HLA-A0301) were used as cold targets or control cold targets. For NSP13-400 specific T cell test, Hs-578T-NSP13 and M14-NSP13 cells labeled with ⁵¹Cr were used as hot targets. Non-radiolabeled M14 cells pulsed with NSP13-400 peptide or MAGEA4 HLA-A2402 peptide (NYKRCFPVI—SEQ ID NO:42) were used as cold targets or control cold targets.

Intracellular staining (ICS) assay: One million SARS-CoV-2 specific T cells (MGP-65, NSP13-448, NSP13-242, NSP13-134, NSP13-400) were co-cultured with 1×10⁵ relevant target cells (10:1 E:T ratio) overnight in the presence of Brefeldin A (BFA) (Biolegend, CA, USA), and the next day, stained with APC-Cy7 conjugated anti-CD8 antibody (Biolegend, CA, USA). After washing, the cells were fixed and permeabilized with Intracellular Fixation & Permeabilization Buffer Set (eBioscience™, NY, USA), and then stained with APC conjugated anti-CD137, FITC conjugated anti-CD69, PE conjugated anti-IFN-7, Pacific blue conjugated anti-TNF-α antibody (all purchased from Biolegend, CA, USA). After washing, the expressing level of CD137, CD69, IFN-7 and TNF-α were determined using flow cytometry assay (LSRFortessa X-20 Analyzer, BD, CA, USA).

T cell receptor (TCR) gene cloning and sequencing of MGP-65 specific T cells: The alpha and beta chains of the TCR were cloned from functional MGP-65 specific T cells using rapid amplification of cDNA 5′ ends (5′ RACE-PCR) protocol (TAKARA, CA, USA), as described previously (Scotto-Lavino et al., 2006), with minor modification. Briefly, total RNA was extracted from MGP-65 specific T cells using RNeasy Kit (QIAGEN, MD, USA), first-strand cDNA were generated with SMARTer RACE 5′/3′ Kit (TAKARA, CA, USA) allowing a universal sequence in the 5′-end of the total cDNA. Using this cDNA as template, the TCR alpha chain and beta chains were amplified with a 5′-end sense universal primer and 3′-end antisense specific primer annealed to the constant domain of TCR alpha chain or beta chain. For the alpha chain, the 3′-end antisense specific primer, TRAC: 5′ was used—GATTACGCCAAGCTTTGTTGCTCTTGAAGTCCATAGACCTCATGTCTAGCAC-3′ (SEQ ID NO:84). For the beta chain, the 3′-end antisense specific primer, TRBC: 5′ was used—GATTACGCCAAGCTTTTCTGATGGCTCAAACACAGCGACCTCG-3′ (SEQ ID NO:85). The bold, italicized sequence is the overlap sequence used for In-Fusion (Sigma (MO, USA)). The PCR products were purified using PureLink™ Quick Gel Extraction Kit (Life Technologies Corporation, NY, USA) and cloned into the pRACE vector using In-Fusion clone protocol (TAKARA, CA, USA). The vectors containing the TCR alpha chain or beta chain gene were then sequenced using Sanger sequencing method. The repertoire of TCR alpha and beta chains was analyzed by the IMGT/V-QUEST search tool (can be found on the world wide web at imgt.org/IMGT vquest).

MGP-65 TCR retroviral vector construction and retrovirus generation: Whole length cDNA of TCR alpha chain and beta chain derived from MGP-65 specific T cells were assembled with a Furin-SGSG-P2A self-cleaving linker peptide to allow for equal expression of both chains (Wargo et al., 2009). To improve pairing between the exogenous TCR alpha and beta chains and reduce mispairing with endogenous human alpha and beta chains, the Cys mutation was introduced in the constant domain of both TCR alpha and beta chain according to previous reports (Kuball et al., 2007). Furthermore, to enhance the expression level of exogenous TCR, codon-optimization was performed and the optimized TCR sequences were synthesized by GenScript (NJ, USA). The whole TCR fragment was cloned into retroviral vector pMSGV1 which utilizes a MSCV long terminal repeat (LTR) to drive gene expression.

Recombinant retrovirus was generated according to the protocol described previously (Wargo et al., 2009). Briefly, 10 g retroviral vector contained whole length TCR derived from MGP-65 specific CTL and 5 g envelope vector RD114 were co-transfected into package cell line Phoenix-GP with Lipofectamine 3000 reagent (Life Technologies Corporation, NY, USA) in Opti-MEM medium (Life Technologies Corporation, NY, USA). After incubation, the retrovirus supernatant was harvested, cleared with centrifuging and then used to transduce allogenic PBMC to express exogenous TCR or stored in −80° C.

MGP-65 specific TCR engineered T cells (TCR-T) generation: The TCR-T generation was performed using the spinoculation protocol described previously (Hughes et al., 2005). HLA-A0201-expressing healthy donor PBMC (1×10⁶/ml) was activated with 50 ng/ml OKT3, 300U/ml IL-2 in AIM-V media (Life Technologies Corporation, NY, USA) supplemented with 5% human AB serum for 2-3 days. 4-6 ml retrovirus supernatants were loaded onto RetroNectin-coated (20 g/ml) (TAKARA, CA, USA) non-tissue culture-treated six-well plates and centrifugation performed at 2000 g for 2 hours at 32° C. Retroviral supernatant was aspirated from the wells; 2×10\hat{ }6 activated PBMC in AIM-V media supplemented with 5% human AB serum and 300U/ml IL-2 were added to the wells followed by centrifugation at 1000 g for 10 minutes. TCR gene-transduced PBMC were cultured for further 3-5 days and TCR expression was determined by anti-CD8 and tetramer staining. CD8+, Tetramer+ cells were sorted and expanded using the expansion protocol described above to generate high purity of MGP-65 specific TCR-T. Functional analysis of MGP-65 specific TCR-T was conducted by CRA and ICS assay as described above.

Statistical analysis: Data analysis was performed using GraphPad Prism version 7.03. Normally distributed data were analyzed using parametric tests (ANOVA or unpaired t test).

All of the methods disclosed and claimed herein can be made and executed without undue experimentation in light of the present disclosure. While the compositions and methods of this invention have been described in terms of preferred embodiments or aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the publications referred to throughout the specification, to the extent that they provide exemplary procedural or other details supplementary to those set forth herein, are specifically incorporated herein by reference.
Abdool Karim, S. S., and de Oliveira, T. (2021). New SARS-CoV-2 Variants—Clinical, Public Health, and Vaccine Implications. N Engl J Med. 10.1056/NEJMc2100362.
Agerer, B., Koblischke, M., Gudipati, V., Montano-Gutierrez, L. F., Smyth, M., Popa, A., Genger, J. W., Endler, L., Florian, D. M., Muhlgrabner, V., et al. (2021). SARS-CoV-2 mutations in MHC-I-restricted epitopes evade CD8(+) T cell responses. Sci Immunol 6. 10.1126/sciimmunol.abg6461.
Ahmed, S. F., Quadeer, A. A., and McKay, M. R. (2020). Preliminary Identification of Potential Vaccine Targets for the COVID-19 Coronavirus (SARS-CoV-2) Based on SARS-CoV Immunological Studies. Viruses 12. 10.3390/v12030254.
Bradley, S. D., Talukder, A. H., Lai, I., Davis, R., Alvarez, H., Tiriac, H., Zhang, M., Chiu, Y., Melendez, B., Jackson, K. R., et al. (2020). Vestigial-like 1 is a shared targetable cancer-placenta antigen expressed by pancreatic and basal-like breast cancers. Nat Commun 11, 5332. 10.1038/s41467-020-19141-w.
Braun, J., Loyal, L., Frentsch, M., Wendisch, D., Georg, P., Kurth, F., Hippenstiel, S., Dingeldey, M., Kruse, B., Fauchere, F., et al. (2020). SARS-CoV-2-reactive T cells in healthy donors and patients with COVID-19. Nature 587, 270-274.
Campbell, K. M., Steiner, G., Wells, D. K., Ribas, A., and Kalbasi, A. (2020). Prediction of SARS-CoV-2 epitopes across 9360 HLA class I alleles. bioRxiv. 10.1101/2020.03.30.016931. Chapuis, A. G., Desmarais, C., Emerson, R., Schmitt, T. M., Shibuya, K., Lai, I., Wagener, F., Chou, J., Roberts, I. M., Coffey, D. G., et al. (2017). Tracking the Fate and Origin of Clinically Relevant Adoptively Transferred CD8(+) T Cells In Vivo. Sci Immunol 2. 10.1126/sciimmunol.aal2568.
Chapuis, A. G., Roberts, I. M., Thompson, J. A., Margolin, K. A., Bhatia, S., Lee, S. M., Sloan, H. L., Lai, I. P., Farrar, E. A., Wagener, F., et al. (2016). T-Cell Therapy Using Interleukin-21-Primed Cytotoxic T-Cell Lymphocytes Combined With Cytotoxic T-Cell Lymphocyte Antigen-4 Blockade Results in Long-Term Cell Persistence and Durable Tumor Regression. J Clin Oncol 34, 3787-3795.
Chapuis, A. G., Thompson, J. A., Margolin, K. A., Rodmyre, R., Lai, I. P., Dowdy, K., Farrar, E. A., Bhatia, S., Sabath, D. E., Cao, J., et al. (2012). Transferred melanoma-specific CD8+T cells persist, mediate tumor regression, and acquire central memory phenotype. Proc Natl Acad Sci USA 109, 4592-4597.
Darby, A. C., and Hiscox, J. A. (2021). Covid-19: variants and vaccination. BMJ 372, n771. Gallais, F., Velay, A., Nazon, C., Wendling, M. J., Partisani, M., Sibilia, J., Candon, S., and Fafi-Kremer, S. (2021). Intrafamilial Exposure to SARS-CoV-2 Associated with Cellular Immune Response without Seroconversion, France. Emerg Infect Dis 27. 10.3201/eid2701.203611.
Gao, A., Chen, Z., Segal, F. P., Carrington, M., Streeck, H., Chakraborty, A. K., and Julg, B. (2020). Predicting the Immunogenicity of T cell epitopes: From HIV to SARS-CoV-2. bioRxiv. 10.1101/2020.05.14.095885.
Garcia-Beltran, W. F., Lam, E. C., St Denis, K., Nitido, A. D., Garcia, Z. H., Hauser, B. M., Feldman, J., Pavlovic, M. N., Gregory, D. J., Poznansky, M. C., et al. (2021). Multiple SARS-CoV-2 variants escape neutralization by vaccine-induced humoral immunity. Cell. 10.1016/j.cell.2021.03.013.
Grifoni, A., Sidney, J., Zhang, Y., Scheuermann, R. H., Peters, B., and Sette, A. (2020a). A Sequence Homology and Bioinformatic Approach Can Predict Candidate Targets for Immune Responses to SARS-CoV-2. Cell Host Microbe 27, 671-680 e672.
Grifoni, A., Weiskopf, D., Ramirez, S. I., Mateus, J., Dan, J. M., Moderbacher, C. R., Rawlings, S. A., Sutherland, A., Premkumar, L., Jadi, R. S., et al. (2020b). Targets of T Cell Responses to SARS-CoV-2 Coronavirus in Humans with COVID-19 Disease and Unexposed Individuals. Cell 181, 1489-1501 e1415.
Hughes, M. S., Yu, Y. Y., Dudley, M. E., Zheng, Z., Robbins, P. F., Li, Y., Wunderlich, J., Hawley, R. G., Moayeri, M., Rosenberg, S. A., and Morgan, R. A. (2005). Transfer of a TCR gene derived from a patient with a marked antitumor response conveys highly active T-cell effector functions. Hum Gene Ther 16, 457-472.
Hui, D. S., E, I. A., Madani, T. A., Ntoumi, F., Kock, R., Dar, O., Ippolito, G., McHugh, T. D., Memish, Z. A., Drosten, C., et al. (2020). The continuing 2019-nCoV epidemic threat of novel coronaviruses to global health—The latest 2019 novel coronavirus outbreak in Wuhan, China. Int J Infect Dis 91, 264-266.
Kar, T., Narsaria, U., Basak, S., Deb, D., Castiglione, F., Mueller, D. M., and Srivastava, A. P. (2020). A candidate multi-epitope vaccine against SARS-CoV-2. Sci Rep 10, 10895. 10.1038/s41598-020-67749-1.
Keller, M. D., Harris, K. M., Jensen-Wachspress, M. A., Kankate, V. V., Lang, H., Lazarski, C. A., Durkee-Shock, J., Lee, P. H., Chaudhry, K., Webber, K., et al. (2020). SARS-CoV-2-specific T cells are rapidly expanded for therapeutic use and target conserved regions of the membrane protein. Blood 136, 2905-2917.
Kessler, J. H., Mommaas, B., Mutis, T., Huijbers, I., Vissers, D., Benckhuijsen, W. E., Schreuder, G. M., Offringa, R., Goulmy, E., Melief, C. J., et al. (2003). Competition-based cellular peptide binding assays for 13 prevalent HLA class I alleles using fluorescein-labeled synthetic peptides. Hum Immunol 64, 245-255.
Kuball, J., Dossett, M. L., Wolfl, M., Ho, W. Y., Voss, R. H., Fowler, C., and Greenberg, P. D. (2007). Facilitating matched pairing and expression of TCR chains introduced into human T cells. Blood 109, 2331-2338.
Kuzmina, A., Khalaila, Y., Voloshin, O., Keren-Naus, A., Boehm-Cohen, L., Raviv, Y., Shemer-Avni, Y., Rosenberg, E., and Taube, R. (2021). SARS-CoV-2 spike variants exhibit differential infectivity and neutralization resistance to convalescent or post-vaccination sera. Cell Host Microbe. 10.1016/j.chom.2021.03.008.
Le Bert, N., Tan, A. T., Kunasegaran, K., Tham, C. Y. L., Hafezi, M., Chia, A., Chng, M. H. Y., Lin, M., Tan, N., Linster, M., et al. (2020). SARS-CoV-2-specific T cell immunity in cases of COVID-19 and SARS, and uninfected controls. Nature 584, 457-462.
Long, Q. X., Tang, X. J., Shi, Q. L., Li, Q., Deng, H. J., Yuan, J., Hu, J. L., Xu, W., Zhang, Y., Lv, F. J., et al. (2020). Clinical and immunological assessment of asymptomatic SARS-CoV-2 infections. Nat Med 26, 1200-1204.
Nolan, S., Vignali, M., Klinger, M., Dines, J. N., Kaplan, I. M., Svejnoha, E., Craft, T., Boland, K., Pesesky, M., Gittelman, R. M., et al. (2020). A large-scale database of T-cell receptor beta (TCRbeta) sequences and binding associations from natural and synthetic exposure to SARS-CoV-2. Res Sq. 10.21203/rs.3.rs-51964/v1.
Olvera, A., Noguera-Julian, M., Kilpelainen, A., Romero-Martin, L., Prado, J. G., and Brander, C. (2020). SARS-CoV-2 Consensus-Sequence and Matching Overlapping Peptides Design for COVID19 Immune Studies and Vaccine Development. Vaccines (Basel) 8. 10.3390/vaccines8030444.
Park, J., Talukder, A. H., Lim, S. A., Kim, K., Pan, K., Melendez, B., Bradley, S. D., Jackson, K. R., Khalili, J. S., Wang, J., et al. (2017). SLC45A2: A Melanoma Antigen with High Tumor Selectivity and Reduced Potential for Autoimmune Toxicity. Cancer immunology research 5, 618-629.
Peng, Y., Mentzer, A. J., Liu, G., Yao, X., Yin, Z., Dong, D., Dejnirattisai, W., Rostron, T., Supasa, P., Liu, C., et al. (2020). Broad and strong memory CD4(+) and CD8(+) T cells induced by SARS-CoV-2 in UK convalescent individuals following COVID-19. Nat Immunol 21, 1336-1345.
Plante, J. A., Mitchell, B. M., Plante, K. S., Debbink, K., Weaver, S. C., and Menachery, V. D. (2021). The variant gambit: COVID-19's next move. Cell Host Microbe. 10.1016/j.chom.2021.02.020.
Poland, G. A., Ovsyannikova, I. G., and Kennedy, R. B. (2020). SARS-CoV-2 immunity: review and applications to phase 3 vaccine candidates. Lancet 396, 1595-1606.
Pollack, S. M., Jones, R. L., Farrar, E. A., Lai, I. P., Lee, S. M., Cao, J., Pillarisetty, V. G., Hoch, B. L., Gullett, A., Bleakley, M., et al. (2014). Tetramer guided, cell sorter assisted production of clinical grade autologous NY-ESO-1 specific CD8(+) T cells. J Immunother Cancer 2, 36. 10.1186/s40425-014-0036-y.
Safavi, A., Kefayat, A., Mahdevar, E., Abiri, A., and Ghahremani, F. (2020). Exploring the out of sight antigens of SARS-CoV-2 to design a candidate multi-epitope vaccine by utilizing immunoinformatics approaches. Vaccine 38, 7612-7628.
Scotto-Lavino, E., Du, G., and Frohman, M. A. (2006). 5′ end cDNA amplification using classic RACE. Nat Protoc 1, 2555-2562.
Sekine, T., Perez-Potti, A., Rivera-Ballesteros, O., Stralin, K., Gorin, J. B., Olsson, A., Llewellyn-Lacey, S., Kamal, H., Bogdanovic, G., Muschiol, S., et al. (2020). Robust T Cell Immunity in Convalescent Individuals with Asymptomatic or Mild COVID-19. Cell 183, 158-168 e114.
Shomuradova, A. S., Vagida, M. S., Sheetikov, S. A., Zornikova, K. V., Kiryukhin, D., Titov, A., Peshkova, I. O., Khmelevskaya, A., Dianov, D. V., Malasheva, M., et al. (2020). SARS-CoV-2 Epitopes Are Recognized by a Public and Diverse Repertoire of Human T Cell Receptors. Immunity 53, 1245-1257 e1245.
Snyder, T. M., Gittelman, R. M., Klinger, M., May, D. H., Osborne, E. J., Taniguchi, R., Zahid, H. J., Kaplan, I. M., Dines, J. N., Noakes, M. N., et al. (2020). Magnitude and Dynamics of the T-Cell Response to SARS-CoV-2 Infection at Both Individual and Population Levels. medRxiv. 10.1101/2020.07.31.20165647.
Sohail, M. S., Ahmed, S. F., Quadeer, A. A., and McKay, M. R. (2021). In silico T cell epitope identification for SARS-CoV-2: Progress and perspectives. Adv Drug Deliv Rev 171, 29-47. Tzou, P. L., Tao, K., Nouhin, J., Rhee, S. Y., Hu, B. D., Pai, S., Parkin, N., and Shafer, R. W. (2020). Coronavirus Antiviral Research Database (CoV-RDB): An Online Database Designed to Facilitate Comparisons between Candidate Anti-Coronavirus Compounds. Viruses 12. 10.3390/v12091006.
V. Gauttier, A. M., I. Girault, C. Mary, L. Belarif, A. Desselle, E. Wilhelm, T. Bourquard, S. Pengam, G. Teppaz, V. Thepenier, K. Biteau, E. De Barbeyrac, D. Kiepferld, B. Vasseur, FX. Le Flem, D. Debieuvre, D. Costantini, N. Poirier (2020). Tissue-resident memory CD8 T-cell responses elicited by a single injection of a multi-target COVID-19 vaccine. bioRxiv. https://doi.org/10.1101/2020.08.14.240093.
Wargo, J. A., Robbins, P. F., Li, Y., Zhao, Y., El-Gamil, M., Caragacianu, D., Zheng, Z., Hong, J. A., Downey, S., Schrump, D. S., et al. (2009). Recognition of NY-ESO-1+ tumor cells by engineered lymphocytes is enhanced by improved vector design and epigenetic modulation of tumor antigen expression. Cancer Immunol Immunother 58, 383-394.
Weiskopf, D., Schmitz, K. S., Raadsen, M. P., Grifoni, A., Okba, N. M. A., Endeman, H., van den Akker, J. P. C., Molenkamp, R., Koopmans, M. P. G., van Gorp, E. C. M., et al. (2020). Phenotype and kinetics of SARS-CoV-2-specific T cells in COVID-19 patients with acute respiratory distress syndrome. Sci Immunol 5. 10.1126/sciimmunol.abd2071.
William Chour, A. M. X., Alphonsus H. C. Ng, Jongchan Choi, Jingyi Xie, Dan Yuan, Diana C. DeLucia, Rick A. Edmark, Lesley C. Jones, Thomas M. Schmitt, Mary E. Chaffee, Venkata R. Duvvuri, Kim M. Murray, Songming Peng, Julie Wallick, Heather A. Algren, William R. Berrington, D. Shane O'Mahony, John K. Lee, Philip D. Greenberg, Jason D. Goldman, James R. Heath (2020). Shared Antigen-specific CD8+ T cell Responses Against the SARS-COV-2 Spike Protein in HLA-A*02:01 COVID-19 Participants. medRxiv. https://doi.org/10.1101/2020.05.04.20085779.
Wu, F., Zhao, S., Yu, B., Chen, Y. M., Wang, W., Song, Z. G., Hu, Y., Tao, Z. W., Tian, J. H., Pei, Y. Y., et al. (2020a). A new coronavirus associated with human respiratory disease in China. Nature 579, 265-269.
Wu, J. T., Leung, K., and Leung, G. M. (2020b). Nowcasting and forecasting the potential domestic and international spread of the 2019-nCoV outbreak originating in Wuhan, China: a modelling study. Lancet 395, 689-697.

What is claimed is:

1. A polypeptide comprising an antigen binding variable region comprising a CDR3 comprising the amino acid sequence of SEQ ID NO:7 or an amino acid sequence with at least 80% sequence identity to SEQ ID NO:7.

2. The polypeptide of claim 1, wherein the variable region comprises a CDR1, CDR2, and/or CDR3.

3. The polypeptide of claim 2, wherein the variable region comprises a CDR1 and/or CDR2 with the amino acid sequence of SEQ ID NO:5 and 6, respectively, or with an amino acid sequence that is at least 80% sequence identity to SEQ ID NO:5 and SEQ ID NO:6, respectively.

4. The polypeptide of any one of claims 1-3, wherein the variable region comprises an amino acid sequence or SEQ ID NO:3 or an amino acid sequence with at least 70% sequence identity to SEQ ID NO:3.

5. The polypeptide of any one of claims 1-4, wherein the polypeptide comprises a T cell receptor alpha (TCR-a) variable region.

6. The polypeptide of claim 5, wherein the polypeptide comprises a TCR-a variable and constant region.

7. The polypeptide of any one of claims 1-6, wherein the polypeptide further comprises a signal peptide.

8. The polypeptide of claim 7, wherein the signal peptide comprises the amino acid sequence of SEQ ID NO:4 or an amino acid sequence with at least 80% identity to SEQ ID NO:4.

9. A polypeptide comprising an antigen binding variable region comprising a CDR3 comprising the amino acid sequence of SEQ ID NO:14 or an amino acid sequence with at least 80% sequence identity to SEQ ID NO:14.

10. The polypeptide of claim 9, wherein the variable region comprises a CDR1, CDR2, and/or CDR3.

11. The polypeptide of claim 10, wherein the variable region comprises a CDR1 and/or CDR2 with the amino acid sequence of SEQ ID NO:12 and SEQ ID NO:13, respectively, or with at least 80% sequence identity to SEQ ID NO:12 and SEQ ID NO:13, respectively.

12. The polypeptide of any one of claims 9-11, wherein the variable region comprises the amino acid sequence of SEQ ID NO:10 or an amino acid sequence with at least 70% sequence identity to SEQ ID NO:10.

13. The polypeptide of any one of claims 9-12, wherein the polypeptide comprises a T cell receptor alpha (TCR-a) variable region.

14. The polypeptide of claim 13, wherein the polypeptide comprises a TCR-a variable and constant region.

15. The polypeptide of any one of claims 9-14, wherein the polypeptide further comprises a signal peptide.

16. The polypeptide of claim 15, wherein the signal peptide comprises the amino acid sequence of SEQ ID NO:11 or an amino acid sequence with at least 80% identity to SEQ ID NO:11.

17. A polypeptide comprising an antigen binding variable region comprising a CDR3 comprising the amino acid sequence of SEQ ID NO:21 or an amino acid sequence with at least 80% sequence identity to SEQ ID NO:21.

18. The polypeptide of claim 17, wherein the variable region comprises a CDR1, CDR2, and/or CDR3.

19. The polypeptide of claim 18, wherein the variable region comprises a CDR1 and/or CDR2 with the amino acid sequence of SEQ ID NO:19 and 20, respectively, or with an amino acid sequence that is at least 80% sequence identity to SEQ ID NO:19 and SEQ ID NO:20, respectively.

20. The polypeptide of any one of claims 17-19, wherein the variable region comprises an amino acid sequence or SEQ ID NO:17 or an amino acid sequence with at least 70% sequence identity to SEQ ID NO:17.

21. The polypeptide of any one of claims 17-20, wherein the polypeptide comprises a T cell receptor beta (TCR-b) variable region.

22. The polypeptide of claim 21, wherein the polypeptide comprises a TCR-b variable and constant region.

23. The polypeptide of any one of claims 17-22, wherein the polypeptide further comprises a signal peptide.

24. The polypeptide of claim 23, wherein the signal peptide comprises the amino acid sequence of SEQ ID NO:18 or an amino acid sequence with at least 80% identity to SEQ ID NO:18.

25. A polypeptide comprising an antigen binding variable region comprising a CDR3 comprising the amino acid sequence of SEQ ID NO:92 or an amino acid sequence with at least 80% sequence identity to SEQ ID NO:92.

26. The polypeptide of claim 25, wherein the variable region comprises a CDR1, CDR2, and/or CDR3.

27. The polypeptide of claim 26, wherein the variable region comprises a CDR1 and/or CDR2 with the amino acid sequence of SEQ ID NO:90 and 91, respectively, or with an amino acid sequence that is at least 80% sequence identity to SEQ ID NO:90 and 91, respectively.

28. The polypeptide of any one of claims 25-27, wherein the variable region comprises an amino acid sequence or SEQ ID NO:88 or an amino acid sequence with at least 70% sequence identity to SEQ ID NO:88.

29. The polypeptide of any one of claims 25-28, wherein the polypeptide comprises a T cell receptor alpha (TCR-a) variable region.

30. The polypeptide of claim 29, wherein the polypeptide comprises a TCR-a variable and constant region.

31. The polypeptide of any one of claims 25-30, wherein the polypeptide further comprises a signal peptide.

32. The polypeptide of claim 31, wherein the signal peptide comprises the amino acid sequence of SEQ ID NO:89 or an amino acid sequence with at least 80% identity to SEQ ID NO:89.

33. A polypeptide comprising an antigen binding variable region comprising a CDR3 comprising the amino acid sequence of SEQ ID NO:99 or an amino acid sequence with at least 80% sequence identity to SEQ ID NO:99.

34. The polypeptide of claim 33, wherein the variable region comprises a CDR1, CDR2, and/or CDR3.

35. The polypeptide of claim 34, wherein the variable region comprises a CDR1 and/or CDR2 with the amino acid sequence of SEQ ID NO:97 and SEQ ID NO:98, respectively, or with at least 80% sequence identity to SEQ ID NO:97 and SEQ ID NO:98, respectively.

36. The polypeptide of any one of claims 33-35, wherein the variable region comprises the amino acid sequence of SEQ ID NO:95 or an amino acid sequence with at least 70% sequence identity to SEQ ID NO:95.

37. The polypeptide of any one of claims 33-36, wherein the polypeptide comprises a T cell receptor alpha (TCR-b) variable region.

38. The polypeptide of claim 37, wherein the polypeptide comprises a TCR-b variable and constant region.

39. The polypeptide of any one of claims 33-38, wherein the polypeptide further comprises a signal peptide.

40. The polypeptide of claim 39, wherein the signal peptide comprises the amino acid sequence of SEQ ID NO:96 or an amino acid sequence with at least 80% identity to SEQ ID NO:96.

41. A polypeptide comprising an antigen binding variable region comprising a CDR3 comprising the amino acid sequence of SEQ ID NO:106 or an amino acid sequence with at least 80% sequence identity to SEQ ID NO:106.

42. The polypeptide of claim 41, wherein the variable region comprises a CDR1, CDR2, and/or CDR3.

43. The polypeptide of claim 42, wherein the variable region comprises a CDR1 and/or CDR2 with the amino acid sequence of SEQ ID NO:104 and 105, respectively, or with an amino acid sequence that is at least 80% sequence identity to SEQ ID NO:104 and 105, respectively.

44. The polypeptide of any one of claims 41-43, wherein the variable region comprises an amino acid sequence or SEQ ID NO:102 or an amino acid sequence with at least 70% sequence identity to SEQ ID NO:102.

45. The polypeptide of any one of claims 41-44, wherein the polypeptide comprises a T cell receptor alpha (TCR-a) variable region.

46. The polypeptide of claim 45, wherein the polypeptide comprises a TCR-a variable and constant region.

47. The polypeptide of any one of claims 41-46, wherein the polypeptide further comprises a signal peptide.

48. The polypeptide of claim 47, wherein the signal peptide comprises the amino acid sequence of SEQ ID NO:103 or an amino acid sequence with at least 80% identity to SEQ ID NO:103.

49. A polypeptide comprising an antigen binding variable region comprising a CDR3 comprising the amino acid sequence of SEQ ID NO:113 or an amino acid sequence with at least 80% sequence identity to SEQ ID NO:113.

50. The polypeptide of claim 49, wherein the variable region comprises a CDR1, CDR2, and/or CDR3.

51. The polypeptide of claim 50, wherein the variable region comprises a CDR1 and/or CDR2 with the amino acid sequence of SEQ ID NO:111 and SEQ ID NO:112, respectively, or with at least 80% sequence identity to SEQ ID NO:111 and SEQ ID NO:112, respectively.

52. The polypeptide of any one of claims 49-51, wherein the variable region comprises the amino acid sequence of SEQ ID NO:109 or an amino acid sequence with at least 70% sequence identity to SEQ ID NO:109.

53. The polypeptide of any one of claims 49-52, wherein the polypeptide comprises a T cell receptor alpha (TCR-b) variable region.

54. The polypeptide of claim 53, wherein the polypeptide comprises a TCR-b variable and constant region.

55. The polypeptide of any one of claims 49-54, wherein the polypeptide further comprises a signal peptide.

56. The polypeptide of claim 55, wherein the signal peptide comprises the amino acid sequence of SEQ ID NO:110 or an amino acid sequence with at least 80% identity to SEQ ID NO:110.

57. A polypeptide comprising an antigen binding variable region comprising a CDR3 comprising the amino acid sequence of SEQ ID NO:120 or an amino acid sequence with at least 80% sequence identity to SEQ ID NO:120.

58. The polypeptide of claim 57, wherein the variable region comprises a CDR1, CDR2, and/or CDR3.

59. The polypeptide of claim 58, wherein the variable region comprises a CDR1 and/or CDR2 with the amino acid sequence of SEQ ID NO:118 and 119, respectively, or with an amino acid sequence that is at least 80% sequence identity to SEQ ID NO:118 and 119, respectively.

60. The polypeptide of any one of claims 57-59, wherein the variable region comprises an amino acid sequence or SEQ ID NO:116 or an amino acid sequence with at least 70% sequence identity to SEQ ID NO:116.

61. The polypeptide of any one of claims 57-60, wherein the polypeptide comprises a T cell receptor alpha (TCR-a) variable region.

62. The polypeptide of claim 61, wherein the polypeptide comprises a TCR-a variable and constant region.

63. The polypeptide of any one of claims 57-62, wherein the polypeptide further comprises a signal peptide.

64. The polypeptide of claim 63, wherein the signal peptide comprises the amino acid sequence of SEQ ID NO:117 or an amino acid sequence with at least 80% identity to SEQ ID NO:117.

65. A polypeptide comprising an antigen binding variable region comprising a CDR3 comprising the amino acid sequence of SEQ ID NO:127 or an amino acid sequence with at least 80% sequence identity to SEQ ID NO:127.

66. The polypeptide of claim 65, wherein the variable region comprises a CDR1, CDR2, and/or CDR3.

67. The polypeptide of claim 66, wherein the variable region comprises a CDR1 and/or CDR2 with the amino acid sequence of SEQ ID NO:125 and SEQ ID NO:126, respectively, or with at least 80% sequence identity to SEQ ID NO:125 and SEQ ID NO:126, respectively.

68. The polypeptide of any one of claims 65-67, wherein the variable region comprises the amino acid sequence of SEQ ID NO:123 or an amino acid sequence with at least 70% sequence identity to SEQ ID NO:123.

69. The polypeptide of any one of claims 65-68, wherein the polypeptide comprises a T cell receptor alpha (TCR-b) variable region.

70. The polypeptide of claim 69, wherein the polypeptide comprises a TCR-b variable and constant region.

71. The polypeptide of any one of claims 65-70, wherein the polypeptide further comprises a signal peptide.

72. The polypeptide of claim 71, wherein the signal peptide comprises the amino acid sequence of SEQ ID NO:124 or an amino acid sequence with at least 80% identity to SEQ ID NO:124.

73. A polypeptide comprising an antigen binding variable region comprising a CDR3 comprising the amino acid sequence of SEQ ID NO:134 or an amino acid sequence with at least 80% sequence identity to SEQ ID NO:134.

74. The polypeptide of claim 73, wherein the variable region comprises a CDR1, CDR2, and/or CDR3.

75. The polypeptide of claim 74, wherein the variable region comprises a CDR1 and/or CDR2 with the amino acid sequence of SEQ ID NO:132 and 133, respectively, or with an amino acid sequence that is at least 80% sequence identity to SEQ ID NO:132 and 133, respectively.

76. The polypeptide of any one of claims 73-75, wherein the variable region comprises an amino acid sequence or SEQ ID NO:130 or an amino acid sequence with at least 70% sequence identity to SEQ ID NO:130.

77. The polypeptide of any one of claims 73-76, wherein the polypeptide comprises a T cell receptor alpha (TCR-a) variable region.

78. The polypeptide of claim 77, wherein the polypeptide comprises a TCR-a variable and constant region.

79. The polypeptide of any one of claims 73-78, wherein the polypeptide further comprises a signal peptide.

80. The polypeptide of claim 79, wherein the signal peptide comprises the amino acid sequence of SEQ ID NO:131 or an amino acid sequence with at least 80% identity to SEQ ID NO:131.

81. A polypeptide comprising an antigen binding variable region comprising a CDR3 comprising the amino acid sequence of SEQ ID NO:141 or an amino acid sequence with at least 80% sequence identity to SEQ ID NO:141.

82. The polypeptide of claim 81, wherein the variable region comprises a CDR1, CDR2, and/or CDR3.

83. The polypeptide of claim 82, wherein the variable region comprises a CDR1 and/or CDR2 with the amino acid sequence of SEQ ID NO:139 and SEQ ID NO:140, respectively, or with at least 80% sequence identity to SEQ ID NO:139 and SEQ ID NO:140, respectively.

84. The polypeptide of any one of claims 81-83, wherein the variable region comprises the amino acid sequence of SEQ ID NO:137 or an amino acid sequence with at least 70% sequence identity to SEQ ID NO:137.

85. The polypeptide of any one of claims 81-84, wherein the polypeptide comprises a T cell receptor alpha (TCR-b) variable region.

86. The polypeptide of claim 85, wherein the polypeptide comprises a TCR-b variable and constant region.

87. The polypeptide of any one of claims 81-86, wherein the polypeptide further comprises a signal peptide.

88. The polypeptide of claim 87, wherein the signal peptide comprises the amino acid sequence of SEQ ID NO:138 or an amino acid sequence with at least 80% identity to SEQ ID NO:138.

89. An engineered T-cell Receptor (TCR) comprising a TCR-a polypeptide and a TCR-b polypeptide, wherein the TCR-a polypeptide comprises:
(i) a CDR3 with the amino acid sequence of SEQ ID NO:7 or an amino acid sequence with at least 80% sequence identity to SEQ ID NO:7; or
(ii) a CDR3 with the amino acid sequence of SEQ ID NO:14 or an amino acid sequence with at least 80% sequence identity to SEQ ID NO:14;
and the TCR-b polypeptide comprises a CDR3 with the amino acid sequence of SEQ ID NO:21 or an amino acid sequence with at least 80% sequence identity to SEQ ID NO:21.

90. The TCR of claim 89, wherein the TCR comprises a TCR-b polypeptide comprising a variable region comprising CDR1, CDR2, and CDR3 and a TCR-a polypeptide comprising a variable region comprising CDR1, CDR2, and CDR3.

91. The TCR of claim 90, wherein the TCR-a polypeptide comprises:
(i) a CDR1 having the amino acid sequence of SEQ ID NO:5 or having an amino acid sequence with at least 80% sequence identity to SEQ ID NO:5; or
(ii) a CDR1 having the amino acid sequence of SEQ ID NO:12 or having an amino acid sequence with at least 80% sequence identity to SEQ ID NO:12;
and/or the TCR-a polypeptide comprises a CDR1 having the amino acid sequence of SEQ ID NO:19 or having an amino acid sequence with at least 80% sequence identity to SEQ ID NO:19.

92. The TCR of claim 90 or 91, wherein the TCR-a polypeptide comprises:
(i) a CDR2 having the amino acid sequence of SEQ ID NO:6 or having an amino acid sequence with at least 80% sequence identity to SEQ ID NO:6; or
(ii) a CDR2 having the amino acid sequence of SEQ ID NO:13 or having an amino acid sequence with at least 80% sequence identity to SEQ ID NO:13;
and/or the TCR-b polypeptide comprises a CDR2 having the amino acid sequence of SEQ ID NO:20 or having an amino acid sequence with at least 80% sequence identity to SEQ ID NO:20.

93. The TCR of any one of claims 90-92, wherein the CDR1, CDR2, and CDR3 of the TCR-a polypeptide comprise:
(i) the amino acid sequence of SEQ ID NO: 5, 6, and 7, respectively; or
(ii) the amino acid sequence of SEQ ID NO: 12, 13, and 14, respectively; and
and wherein the CDR1, CDR3, and CDR3 of the TCR-b polypeptide comprise the amino acid sequence of SEQ ID NO:19, 20, and 21, respectively.

94. The TCR of any one of claims 90-93, wherein the TCR-a polypeptide comprises:
(i) the amino acid sequence of SEQ ID NO:3 or an amino acid sequence with at least 70% sequence identity to SEQ ID NO:3; or
(ii) the amino acid sequence of SEQ ID NO:10 or an amino acid sequence with at least 70% sequence identity to SEQ ID NO:10;
and the TCR-b polypeptide comprises the amino acid sequence of SEQ ID NO:17 or an amino acid sequence with at least 70% sequence identity to SEQ ID NO:17.

95. An engineered T-cell Receptor (TCR) comprising a TCR-a polypeptide and a TCR-b polypeptide, wherein the TCR-a polypeptide comprises a CDR3 with the amino acid sequence of SEQ ID NO:92 or an amino acid sequence with at least 80% sequence identity to SEQ ID NO:92; and the TCR-b polypeptide comprises a CDR3 with the amino acid sequence of SEQ ID NO:99 or an amino acid sequence with at least 80% sequence identity to SEQ ID NO:99.

96. The TCR of claim 95, wherein the TCR comprises a TCR-b polypeptide comprising a variable region comprising CDR1, CDR2, and CDR3 and a TCR-a polypeptide comprising a variable region comprising CDR1, CDR2, and CDR3.

97. The TCR of claim 96, wherein the TCR-a polypeptide comprises a CDR1 having the amino acid sequence of SEQ ID NO:90 or having an amino acid sequence with at least 80% sequence identity to SEQ ID NO:90; and/or the TCR-a polypeptide comprises a CDR1 having the amino acid sequence of SEQ ID NO:97 or having an amino acid sequence with at least 80% sequence identity to SEQ ID NO:97.

98. The TCR of claim 96 or 97, wherein the TCR-a polypeptide comprises a CDR2 having the amino acid sequence of SEQ ID NO:91 or having an amino acid sequence with at least 80% sequence identity to SEQ ID NO:91; and/or the TCR-b polypeptide comprises a CDR2 having the amino acid sequence of SEQ ID NO:98 or having an amino acid sequence with at least 80% sequence identity to SEQ ID NO:98.

99. The TCR of any one of claims 96-98, wherein the CDR1, CDR2, and CDR3 of the TCR-a polypeptide comprises the amino acid sequence of SEQ ID NOS:90, 91, and 92, respectively; and wherein the CDR1, CDR3, and CDR3 of the TCR-b polypeptide comprise the amino acid sequence of SEQ ID NO:97, 98, and 99, respectively.

100. The TCR of any one of claims 96-99, wherein the TCR-a polypeptide comprises the amino acid sequence of SEQ ID NO:88 or an amino acid sequence with at least 70% sequence identity to SEQ ID NO:88; and the TCR-b polypeptide comprises the amino acid sequence of SEQ ID NO:95 or an amino acid sequence with at least 70% sequence identity to SEQ ID NO:95.

101. An engineered T-cell Receptor (TCR) comprising a TCR-a polypeptide and a TCR-b polypeptide, wherein the TCR-a polypeptide comprises a CDR3 with the amino acid sequence of SEQ ID NO:106 or an amino acid sequence with at least 80% sequence identity to SEQ ID NO:106; and the TCR-b polypeptide comprises a CDR3 with the amino acid sequence of SEQ ID NO:113 or an amino acid sequence with at least 80% sequence identity to SEQ ID NO:113.

102. The TCR of claim 101, wherein the TCR comprises a TCR-b polypeptide comprising a variable region comprising CDR1, CDR2, and CDR3 and a TCR-a polypeptide comprising a variable region comprising CDR1, CDR2, and CDR3.

103. The TCR of claim 102, wherein the TCR-a polypeptide comprises a CDR1 having the amino acid sequence of SEQ ID NO:104 or having an amino acid sequence with at least 80% sequence identity to SEQ ID NO:104; and/or the TCR-a polypeptide comprises a CDR1 having the amino acid sequence of SEQ ID NO:111 or having an amino acid sequence with at least 80% sequence identity to SEQ ID NO:111.

104. The TCR of claim 102 or 103, wherein the TCR-a polypeptide comprises a CDR2 having the amino acid sequence of SEQ ID NO:105 or having an amino acid sequence with at least 80% sequence identity to SEQ ID NO:105; and/or the TCR-b polypeptide comprises a CDR2 having the amino acid sequence of SEQ ID NO:112 or having an amino acid sequence with at least 80% sequence identity to SEQ ID NO:112.

105. The TCR of any one of claims 102-104, wherein the CDR1, CDR2, and CDR3 of the TCR-a polypeptide comprises the amino acid sequence of SEQ ID NOS:104, 105, and 106, respectively; and wherein the CDR1, CDR3, and CDR3 of the TCR-b polypeptide comprise the amino acid sequence of SEQ ID NO:111, 112, and 113, respectively.

106. The TCR of any one of claims 102-105, wherein the TCR-a polypeptide comprises the amino acid sequence of SEQ ID NO:102 or an amino acid sequence with at least 70% sequence identity to SEQ ID NO:102; and the TCR-b polypeptide comprises the amino acid sequence of SEQ ID NO:109 or an amino acid sequence with at least 70% sequence identity to SEQ ID NO:109.

107. An engineered T-cell Receptor (TCR) comprising a TCR-a polypeptide and a TCR-b polypeptide, wherein the TCR-a polypeptide comprises a CDR3 with the amino acid sequence of SEQ ID NO:120 or an amino acid sequence with at least 80% sequence identity to SEQ ID NO:120; and the TCR-b polypeptide comprises a CDR3 with the amino acid sequence of SEQ ID NO:127 or an amino acid sequence with at least 80% sequence identity to SEQ ID NO:127.

108. The TCR of claim 107, wherein the TCR comprises a TCR-b polypeptide comprising a variable region comprising CDR1, CDR2, and CDR3 and a TCR-a polypeptide comprising a variable region comprising CDR1, CDR2, and CDR3.

109. The TCR of claim 108, wherein the TCR-a polypeptide comprises a CDR1 having the amino acid sequence of SEQ ID NO:118 or having an amino acid sequence with at least 80% sequence identity to SEQ ID NO:118; and/or the TCR-a polypeptide comprises a CDR1 having the amino acid sequence of SEQ ID NO:125 or having an amino acid sequence with at least 80% sequence identity to SEQ ID NO:125.

110. The TCR of claim 108 or 109, wherein the TCR-a polypeptide comprises a CDR2 having the amino acid sequence of SEQ ID NO:119 or having an amino acid sequence with at least 80% sequence identity to SEQ ID NO:119; and/or the TCR-b polypeptide comprises a CDR2 having the amino acid sequence of SEQ ID NO:126 or having an amino acid sequence with at least 80% sequence identity to SEQ ID NO:126.

111. The TCR of any one of claims 108-110, wherein the CDR1, CDR2, and CDR3 of the TCR-a polypeptide comprises the amino acid sequence of SEQ ID NOS:118, 119, and 120, respectively; and wherein the CDR1, CDR3, and CDR3 of the TCR-b polypeptide comprise the amino acid sequence of SEQ ID NO:125, 126, and 127, respectively.

112. The TCR of any one of claims 108-111, wherein the TCR-a polypeptide comprises the amino acid sequence of SEQ ID NO:116 or an amino acid sequence with at least 70% sequence identity to SEQ ID NO:116; and the TCR-b polypeptide comprises the amino acid sequence of SEQ ID NO:123 or an amino acid sequence with at least 70% sequence identity to SEQ ID NO:123.

113. An engineered T-cell Receptor (TCR) comprising a TCR-a polypeptide and a TCR-b polypeptide, wherein the TCR-a polypeptide comprises a CDR3 with the amino acid sequence of SEQ ID NO:134 or an amino acid sequence with at least 80% sequence identity to SEQ ID NO:134; and the TCR-b polypeptide comprises a CDR3 with the amino acid sequence of SEQ ID NO:141 or an amino acid sequence with at least 80% sequence identity to SEQ ID NO:141.

114. The TCR of claim 113, wherein the TCR comprises a TCR-b polypeptide comprising a variable region comprising CDR1, CDR2, and CDR3 and a TCR-a polypeptide comprising a variable region comprising CDR1, CDR2, and CDR3.

115. The TCR of claim 114, wherein the TCR-a polypeptide comprises a CDR1 having the amino acid sequence of SEQ ID NO:132 or having an amino acid sequence with at least 80% sequence identity to SEQ ID NO:132; and/or the TCR-a polypeptide comprises a CDR1 having the amino acid sequence of SEQ ID NO:139 or having an amino acid sequence with at least 80% sequence identity to SEQ ID NO:139.

116. The TCR of claim 114 or 115, wherein the TCR-a polypeptide comprises a CDR2 having the amino acid sequence of SEQ ID NO:133 or having an amino acid sequence with at least 80% sequence identity to SEQ ID NO:133; and/or the TCR-b polypeptide comprises a CDR2 having the amino acid sequence of SEQ ID NO:140 or having an amino acid sequence with at least 80% sequence identity to SEQ ID NO:140.

117. The TCR of any one of claims 114-116, wherein the CDR1, CDR2, and CDR3 of the TCR-a polypeptide comprises the amino acid sequence of SEQ ID NOS:132, 133, and 134, respectively; and wherein the CDR1, CDR3, and CDR3 of the TCR-b polypeptide comprise the amino acid sequence of SEQ ID NO:139, 140, and 141, respectively.

118. The TCR of any one of claims 114-117, wherein the TCR-a polypeptide comprises the amino acid sequence of SEQ ID NO:130 or an amino acid sequence with at least 70% sequence identity to SEQ ID NO:130; and the TCR-b polypeptide comprises the amino acid sequence of SEQ ID NO:137 or an amino acid sequence with at least 70% sequence identity to SEQ ID NO:137.

119. The TCR of any one of claims 89-118, wherein the TCR comprises a modification or is chimeric.

120. The TCR of any one of claims 89-119, wherein the TCR-b polypeptide and TCR-a polypeptide are operably linked.

121. The TCR of claim 120, wherein the TCR-b polypeptide and TCR-a polypeptide are operably linked through a peptide bond.

122. The TCR of claim 121, wherein the TCR is a single chain TCR.

123. The TCR of claim 121, wherein the TCR-b polypeptide and TCR-a polypeptide are on the same polypeptide and wherein the TCR-b is amino-proximal to the TCR-a.

124. The TCR of claim 121, wherein the TCR-b polypeptide and TCR-a polypeptide are on the same polypeptide and wherein the TCR-a is amino-proximal to the TCR-b.

125. The TCR of any one of claims 122-124, wherein the TCR comprises a linker between the TCR-a and TCR-b polypeptide.

126. The TCR of claim 125, wherein the linker comprises glycine and serine residues.

127. A peptide comprising at least 60% sequence identity to a peptide of one of SEQ ID NOS:22-81.

128. The peptide of claim 127, wherein the peptide comprises SEQ ID NO:22.

129. The peptide of claim 127, wherein the peptide comprises at least 6 contiguous amino acids of one of SEQ ID NO:22-81.

130. The peptide of claim 127, wherein the peptide comprises at least 7 contiguous amino acids of a peptide of one of SEQ ID NOS:22-81.

131. The peptide of claim 127, wherein the peptide comprises at least 8 contiguous amino acids of a peptide of one of SEQ ID NOS:22-81.

132. The peptide of claim 127, wherein the peptide comprises at least 9 contiguous amino acids of a peptide of one of SEQ ID NOS:22-81.

133. The peptide of claim 127, wherein the peptide comprises at least 10 contiguous amino acids of a peptide of one of SEQ ID NOS:22-81.

134. The peptide of any one of claims 127-133 wherein the peptide is 13 amino acids or fewer in length.

135. The peptide of any one of claims 127-134, wherein the peptide comprises at least 63% sequence identity to a peptide of one of SEQ ID NOS:22-81.

136. The peptide of any one of claims 127-134, wherein the peptide comprises at least 66% sequence identity to a peptide of one of SEQ ID NOS:22-81.

137. The peptide of any one of claims 127-134, wherein the peptide comprises at least 70% sequence identity to a peptide of one of SEQ ID NOS:22-81.

138. The peptide of any one of claims 127-134, wherein the peptide comprises at least 72% sequence identity to a peptide of one of SEQ ID NOS:22-81.

139. The peptide of any one of claims 127-134, wherein the peptide comprises at least 77% sequence identity to a peptide of one of SEQ ID NOS:22-81.

140. The peptide of any one of claims 127-134, wherein the peptide comprises at least 80% sequence identity to a peptide of one of SEQ ID NOS:22-81.

141. The peptide of any one of claims 127-134, wherein the peptide comprises at least 81% sequence identity to a peptide of one of SEQ ID NOS:22-81.

142. The peptide of any one of claims 127-134, wherein the peptide comprises at least 88% sequence identity to a peptide of one of SEQ ID NOS:22-81.

143. The peptide of any one of claims 127-134, wherein the peptide comprises at least 90% sequence identity to a peptide of one of SEQ ID NOS:22-81.

144. The peptide of any one of claims 127-134, wherein the peptide comprises 100% sequence identity to a peptide of one of SEQ ID NOS:22-81.

145. The peptide of any one of claims 134-144, wherein the peptide consists of 9 amino acids.

146. The peptide of any one of claims 134-144, wherein the peptide consists of 10 amino acids.

147. The peptide of any one of claims 134-144, wherein the peptide consists of 11 amino acids.

148. The peptide of any one of claims 134-144, wherein the peptide consists of 12 amino acids.

149. The peptide of any one of claims 134-144, wherein the peptide consists of 13 amino acids.

150. The peptide of any one of claims 127-149, wherein the peptide consists of a peptide of one of SEQ ID NOS:22-81.

151. The peptide of any one of claims 127-150, wherein the peptide is immunogenic.

152. The peptide of any one of claims 127-151, wherein the peptide is modified.

153. The peptide of claim 152, wherein the modification comprises conjugation to a molecule.

154. The peptide of claim 152 or 153, wherein the molecule comprises an antibody, a lipid, an adjuvant, or a detection moiety.

155. The peptide of any one of claims 127-154, wherein the peptide comprises or consists of one of SEQ ID NOS:22-38 or a peptide comprising at least 60% sequence identity to one of SEQ ID NOS:22-38.

156. The peptide of any of claims 127-155, wherein the peptide has 1, 2 or 3 substitutions relative to a peptide of one of SEQ ID NOS:22-81.

157. A polypeptide comprsing the peptide of any one of claims 127-156.

158. A composition comprising at least one MHC polypeptide and the peptide or polypeptide of any one of claims 1-157.

159. The composition of claim 158, wherein the MHC polypeptide is and/or peptide is conjugated to a detection tag.

160. The composition of claim 158 or 159, wherein the MHC polypeptide and peptide are operatively linked.

161. The composition of claim 160, wherein the MHC polypeptide and peptide are operatively linked through a peptide bond.

162. The composition of claim 161, wherein the MHC polypeptide and peptide are operatively linked through van der Waals forces.

163. The composition of any one of claims 158-162, wherein at least two MHC polypeptides are linked to one peptide.

164. The composition of any one of claims 158-163, wherein the average ratio of MHC polypeptides to peptides is 4:1.

165. A molecular complex comprising the peptide of any one of claims 127- or the polypeptide of claim 157 and a MHC polypeptide.

166. A peptide-specific binding molecule, wherein the molecule specifically binds to a peptide or polypeptide of any one of claim 127-157 or the molecular complex of claim 165.

167. The binding molecule of claim 166, wherein the binding molecule is an antibody, TCR mime antibody, scFV, camellid, aptamer, or DARPIN.

168. A method of producing coronavirus-specific immune effector cells comprising:
(a) obtaining a starting population of immune effector cells; and
(b) contacting the starting population of immune effector cells with a peptide or polypeptide of any one of claims 127-157 or the molecular complex of claim 165, thereby generating peptide-specific immune effector cells.

169. The method of claim 168, wherein the coronavirus is a coronavirus isolated from bats.

170. The method of claim 168 or 169, wherein the coronavirus is SARS-CoV or SARS-CoV-2.

171. The method of any one of claims 168-170, wherein contacting is further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface.

172. The method of claim 171, wherein the APCs are dendritic cells.

173. The method of any one of claims 168-172, wherein the immune effector cells are T cells, peripheral blood lymphocytes, NK cells, invariant NK cells, NKT cells.

174. The method of any one of claims 168-173, wherein the immune effector cells have been differentiated from mesenchymal stem cell (MSC) or induced pluripotent stem (iPS) cells.

175. The method of claim 173, wherein the T cells are CD8+ T cells, CD4+ T cells, or T6 T cells.

176. The method of claim 173, wherein the T cells are cytotoxic T lymphocytes (CTLs).

177. The method of any one of claims 168-176, wherein obtaining comprises isolating the starting population of immune effector cells from peripheral blood mononuclear cells (PBMCs).

178. The method of any one of claims 168-177, wherein the starting population of immune effector cells is obtained from a subject.

179. The method of claim 178, wherein the subject is a human.

180. The method of claim 179, wherein the subject has a coronavirus infection.

181. The method of claim 180, wherein the coronavirus infection comprises COVID-19 or SARS.

182. The method of any one of claims 168-181, wherein the subject has one or more symptoms of a coronavirus infection.

183. The method of any one of claims 168-181, wherein the subject does not have any symptoms of a coronavirus infection.

184. The method of any one of claims 168-181, wherein the subject has been diagnosed with a coronavirus infection.

185. The method of any one of claims 178-183, wherein the subject has not been diagnosed with a coronavirus infection.

186. The method of any one of claims 178-185, wherein the subject has been previously treated for a coronavirus infection.

187. The method of any one of claims 178-186, wherein the method further comprises introducing the peptide or a nucleic acid encoding the peptide into the dendritic cells prior to the co-culturing.

188. The method of claim 187, where the peptide or nucleic acids encoding the peptide are introduced by electroporation.

189. The method of claim 187, wherein the peptide or nucleic acids encoding the peptide are introduced by adding the peptide or nucleic acid encoding the peptide to the dendritic cell culture media.

190. The method of any one of claims 187-189, wherein the immune effector cells are co-cultured with a second population of dendritic cells into which the peptide or the nucleic acid encoding the peptide has been introduced.

191. The method of any one of claims 187-190, wherein a population of CD8 or CD4-positive and coronavirus peptide MHC tetramer-positive T cells are purified from the immune effector cells following the co-culturing.

192. The method of claim 191, wherein a clonal population of coronavirus-specific immune effector cells are generated by limiting or serial dilution followed by expansion of individual clones by a rapid expansion protocol.

193. The method of claim 192, wherein the method further comprises cloning of a T cell receptor (TCR) from the clonal population of peptide-specific immune effector cells.

194. The method of claim 193, wherein cloning of the TCR is cloning of a TCR alpha and a beta chain.

195. The method of claim 193 or claim 194, wherein the TCR is cloned using a 5′-Rapid amplification of cDNA ends (RACE) method.

196. The method of claim 195, wherein the cloned TCR is subcloned into an expression vector.

197. The method of claim 196, wherein the expression vector is a retroviral or lentiviral vector.

198. The method of claim 196 or 197, where the method further comprises transducing a host cell with the expression vector to generate an engineered cell that expresses the TCR.

199. The method of claim 198, wherein the host cell is an immune cell.

200. The method of any one of claims 168-199, wherein the immune cell is a T cell and the engineered cell is an engineered T cell.

201. The method of claim 200, wherein the T cell is a CD8+ T cell, CD4+ T cell, or T6 T cell and the engineered cell is an engineered T cell.

202. The method of any one of claims 168-201, wherein the starting population of immune effector cells is obtained from a subject with a SARS-Cov-2 infection and the host cell is allogeneic or autologous to the subject.

203. The method of any one of claims 198-202, wherein a population of CD8 or CD4-positive and peptide MHC tetramer-positive engineered T cells are purified from the transduced host cells.

204. The method of any one of claims 168-203, wherein a clonal population of peptide-specific engineered T cells are generated by limiting or serial dilution followed by expansion of individual clones by a rapid expansion protocol.

205. A method of cloning a coronavirus T cell receptor (TCR), the method comprising
(a) obtaining a starting population of immune effector cells;
(b) contacting the starting population of immune effector cells with the coronavirus peptide or polypeptide of any one of claims 216-157, thereby generating coronavirus-specific immune effector cells;
(c) purifying immune effector cells specific to the coronavirus peptide,
(d) isolating a TCR sequence from the purified immune effector cells.

206. The method of claim 205, wherein the coronavirus is a coronavirus isolated from bats.

207. The method of claim 205 or 206, wherein the coronavirus is SARS-CoV or SARS-CoV-2.

208. The method of any one of claims 206-207, wherein contacting is further defined as co-culturing the starting population of immune effector cells with antigen presenting cells (APCs), wherein the APCs present the coronavirus peptide on their surface.

209. The method of claim 208, wherein the APCs are dendritic cells.

210. The method of any one of claims 205-209, wherein the immune effector cells are T cells, peripheral blood lymphocytes, NK cells, invariant NK cells, NKT cells.

211. The method of any one of claims 205-210, wherein the immune effector cells have been differentiated from mesenchymal stem cell (MSC) or induced pluripotent stem (iPS) cells.

212. The method of claim 210 or 211, wherein the T cells are CD8⁺ T cells, CD4⁺ T cells, or γδ T cells.

213. The method of any one of claims 210-212, wherein the T cells are cytotoxic T lymphocytes (CTLs).

214. The method of any one of claims 205-213, wherein obtaining comprises isolating the starting population of immune effector cells from peripheral blood mononuclear cells (PBMCs).

215. The method of any of claims 205-214, wherein the starting population of immune effector cells is obtained from a subject.

216. The method of claim 215, wherein the subject is a human.

217. The method of claim 215 or 216, wherein the subject has a coronavirus infection.

218. The method of claim 217, wherein the subject has COVID-19 or SARS.

219. The method of any one of claims 205-218, wherein the method further comprises introducing the coronavirus peptide or a nucleic acid encoding the coronavirus peptide into the dendritic cells prior to the co-culturing.

220. The method of claim 219, where the peptide or nucleic acid encoding the peptide are introduced by electroporation.

221. The method of claim 219, wherein the peptide or nucleic acid encoding the peptide are introduced by adding the peptide or nucleic acid encoding the peptide to the media of the dendritic cells.

222. The method of claim 219, wherein the immune effector cells are co-cultured with a second population of dendritic cells into which the coronavirus peptide or a nucleic acid encoding the coronavirus peptide has been introduced.

223. The method of claim 219, wherein purifying is defined as purifying a population of CD8-positive and coronavirus peptide MHC tetramer-positive T cells from the immune effector cells following the co-culturing.

224. The method of claim 223, wherein the population of CD8-positive and coronavirus peptide MHC tetramer-positive T cells are purified by fluorescence activated cell sorting (FACS).

225. The method of claim 224, wherein purifying further comprises generation of a clonal population of coronavirus-specific immune effector cells by limiting or serial dilution of sorted cells followed by expansion of individual clones by a rapid expansion protocol.

226. The method of claim 225, wherein isolating is defined as cloning of a T cell receptor (TCR) from the clonal population of coronavirus-specific immune effector cells.

227. The method of any one of claims 205-226, wherein the method further comprises sequencing the TCR alpha and/or beta gene(s) and/or performing grouping of lymphocyte interactions by paratope hotspots (GLIPH) analysis.

228. The method of claim 226 or 227, wherein cloning of the TCR is cloning of a TCR alpha and a beta chain.

229. The method of claim 228, wherein the TCR alpha and beta chains are cloned using a 5′-Rapid amplification of cDNA ends (RACE) method.

230. The method of claim 229, wherein the cloned TCR is subcloned into an expression vector.

231. The method of claim 230, wherein the expression vector comprises a linker domain between the TCR alpha sequence and TCR beta sequence.

232. The method of claim 231, wherein the linker domain comprises a sequence encoding one or more peptide cleavage sites.

233. The method of claim 232, wherein the one or more cleavage sites are a Furin cleavage site and/or a P2A cleavage site.

234. The method of claim 233, wherein the TCR alpha sequence and TCR beta sequence are linked by an IRES sequence.

235. The method of any of claims 230-234, wherein the expression vector is a retroviral or lentiviral vector.

236. The method of claim 235, where a host cell is transduced with the expression vector to generate an engineered cell that expresses the TCR alpha and beta chains.

237. The method of claim 236, wherein the host cell is an immune cell.

238. A coronavirus-specific engineered T cell produced according to any one of the methods of claims 168-267.

239. A TCR produced by the method of any one of claims 168-237.

240. A fusion protein comprising the TCR of any one of claims 89-126 or 239, or a peptide-binding fragment thereof and a CD3 binding region.

241. The fusion protein of claim 240, wherein the CD3 binding region comprises a CD3-specific fragment antigen binding (Fab), single chain variable fragment (scFv), single domain antibody, or single chain antibody.

242. The TCR of any one of claims 89-126 or 239, or the fusion protein of claim 240 or 241, wherein the TCR or fusion protein is conjugated to a detection or therapeutic agent.

243. The TCR or fusion protein of claim 242, wherein the agent comprises a fluorescent molecule, radiative molecule, or toxin.

244. A nucleic acid encoding the polypeptide of any one of claims 1-88 or 157, the TCR of any one of claims 89-126, 239, 242, or 243, the peptide of any one of claims 127-156 or the fusion protein of any one of claims 240-243.

245. The nucleic acid of claim 244, wherein the nucleic acid is RNA.

246. The nucleic acid of claim 245, wherein the nucleic acid is DNA or a cDNA encoding the peptide or polypeptide or a complement of the peptide or polypeptide.

247. The nucleic acid of claim 244, wherein the nucleic acid has at least 70% sequence identity to SEQ ID NO:1, 8, 15, or a fragment thereof.

248. A nucleic acid expression vector comprising the nucleic acid(s) of any one of claims 244-247.

249. The vector of claim 248, wherein the vector comprises a promoter that directs the expression of the nucleic acid.

250. The vector of claim 249, wherein the promoter comprises a murine stem cell virus (MSCV) promoter.

251. The vector of any one of claims 248-250, wherein the vector comprises the TCR-a and TCR-b genes.

252. A cell comprising the polypeptide of any one of claims 1-88 or 157, the TCR of any one of claims 89-126, 239, 242, or 243, the peptide of any one of claims 127-156, the fusion protein of any one of claims 240-243, the nucleic acid(s) of any one of claims 244-247, or the vector of any one of claims 248-252.

253. The cell of claim 252, wherein the cell comprises a stem cell, a progenitor cell, an immune cell, or a natural killer (NK) cell.

254. The cell of claim 252, wherein the cell comprises a hematopoietic stem or progenitor cell, a T cell, a cell differentiated from mesenchymal stem cells (MSCs) or an induced pluripotent stem cell (iPSC).

255. The cell of any one of claims 252-254, wherein the cell is isolated or derived from peripheral blood mononuclear cell (PBMCs).

256. The cell of any one of claims 252-255, wherein the T cell comprises a cytotoxic T lymphocyte (CTL), a CD8⁺ T cell, a CD4⁺ T cell, an invariant NK T (iNKT) cell, a gamma-delta T cell, a NKT cell, or a regulatory T cell.

257. The cell of any one of claims 252-256, wherein the cell is isolated from a subject having SARS-Cov-2.

258. An in vitro isolated dendritic cell comprising the peptide or polypeptide of any one of claims 127-157, the nucleic acid(s) of any one of claims 244-247, or the vector of any one of claims 248-252.

259. The dendritic cell of claim 258, wherein the dendritic cell is a mature dendritic cell.

260. The dendritic cell of claim 258 or 259, wherein the cell is a cell with an HLA-A, HLA-B, or HLA-C type.

261. A method of making a cell comprising transferring the nucleic acid(s) of any one of claims 244-247 or the vector of any one of claims 248-252 into the cell.

262. The method of claim 261, wherein the method further comprises isolating the expressed peptide or polypeptide.

263. An in vitro method for making a therapeutic T cell vaccine comprising co-culturing T cells with the peptide or polypeptide of any one of claims 127-157.

264. The method of claim 263, wherein the T cell comprises a CD8+ T cell.

265. The method of claim 263 or 264, wherein the peptide is complexed with MHC.

266. The method of claim 265, wherein the MHC comprises HLA-A, HLA-B, or HLA-C type.

267. The method of any one of claims 263-266, wherein the peptides are loaded onto dendritic cells, lymphoblastoid cells, peripheral blood mononuclear cells (PBMCs), artificial antigen presentation cells (aAPC), or artificial antigen presentation surface.

268. A T cell made by the method of any one of claims 263-267

269. A T cell comprising a TCR that specifically binds to a peptide of one of SEQ ID NOS:22-81.

270. The T cell of claim 268 or 269, wherein the T cell comprises a CD8+ T cell or CD4+ T cell.

271. The T cell of claim 270, wherein the T cell comprises a cytotoxic T lymphocyte.

272. A pharmaceutical composition comprising the polypeptide of any one of claims 1-88 or 157, the TCR of any one of claims 89-126, 239, 242, or 243, the peptide of any one of claims 127-156, the molecular complex of claim 165, the peptide-specific binding molecule of claim 166 or 167, the peptide-specific T cell of claim 238 or 268-271 or the dendritic cell of any one of claims 258-260, and a pharmaceutical carrier.

273. The pharmaceutical composition of claim 272, wherein the pharmaceutical composition is formulated for parenteral administration, intravenous injection, intramuscular injection, inhalation, or subcutaneous injection.

274. The pharmaceutical composition of claim 272 or 273, wherein the peptide is comprised in a liposome, lipid-containing nanoparticle, or in a lipid-based carrier.

275. The pharmaceutical composition of any one of claims 272-274, wherein the pharmaceutical preparation is formulated for injection or inhalation as a nasal spray.

276. The pharmaceutical composition of any one of claims 272-275, wherein the composition is formulated as a vaccine.

277. The pharmaceutical composition of any one of claims 272-276, wherein the composition further comprises an adjuvant.

278. The pharmaceutical composition of any one of claims 272-277, wherein the composition has been determined to be serum-free, mycoplasma-free, endotoxin-free, and/or sterile.

279. A method of making an engineered cell comprising transferring the nucleic acid of any one of claims 244-247 or the vector of any one of claims 248-251 into a cell.

280. The method of claim 279, wherein the method further comprises culturing the cell in media, incubating the cell at conditions that allow for the division of the cell, screening the cell, and/or freezing the cell.

281. The method of claim 279 or 280, wherein the method further comprises isolating the expressed peptide or polypeptide.

282. A method for treating or preventing a coronavirus infection in a subject comprising administering the composition of any one of claims 158-164 or 272-278 or the cells of any one of claims 238 or 268-271 to a subject in need thereof.

283. A method of stimulating an immune response in a subject, the method comprising administering the composition of any one of claims 158-164 or 272-278 or the cells of any one of claims 238 or 268-271 to the subject.

284. The method of claim 282 or 283, wherein the subject is a human subject.

285. The method of any one of claims 282-284, wherein the cells are autologous.

286. The method of any one of claims 282-284, wherein the cells are allogenic.

287. The method of claim 282 or 284, wherein the subject has one or more symptoms of a coronavirus infection.

288. The method of claim 282 or 284, wherein the subject does not have any symptoms of a coronavirus infection.

289. The method of any one of claims 282-288, wherein the subject has been diagnosed with a coronavirus infection.

290. The method of any one of claims 282-288, wherein the subject has not been diagnosed with a coronavirus infection.

291. The method of any one of claims 282-290, wherein the subject has been previously treated for a coronavirus infection.

292. The method of any one of claims 282-291, wherein the coronavirus comprises a coronavirus isolated from bats.

293. The method of claim 292, wherein the coronavirus is SARS-CoV or SARS-CoV-2.

294. The method of any one of claims 282-293, wherein the method comprises preventing or treating COVID-19 or SARS.

295. The method of any one of claims 282-294, wherein the subject has been diagnosed with complications relating to a coronavirus.

296. The method of claim 295, wherein the complication comprises pneumonia, difficulty breathing or shortness of breath, chest pain or chest pressure, acute respiratory failure, acute respiratory distress syndrome, acute cariac injury, secondary infection, acute kidney injury, septic shock, blood clots, multisystem inflammatory syndrome, chronic fatigue, rhabdomyolysis, disseminated intravascular coagulation, and/or acute liver injury.

297. The method of any one of claims 282-296, wherein the subject is vaccinated against a coronavirus.

298. The method of any one of claims 282-296, wherein the subject is not vaccinated against a coronavirus.

299. The method of 202 any one of claims 291-298, wherein the subject has been determined to be resistant or non-responsive to the previous treatment.

300. The method of any one of claims 282-299, wherein the subject is administered an additional therapeutic.

301. The method of claim 300, wherein the additional therapeutic comprises a steroid or an anti-viral therapeutic.

302. The method of claim 301, wherein the additional therapeutic comprises dexamethasone, monoclonal antibodies, remdesivir, Paxlovid, Molnupiravir, convalescent plasma, or combinations thereof.

303. A method for prognosing a patient or for detecting T cell responses in a patient, the method comprising: contacting a biological sample from the patient with the peptide or polypeptide of any one of claims 127-157 or the molecular complex of claim 165.

304. The method of claim 303, wherein the biological sample comprises a blood sample or a fraction thereof.

305. The method of claim 304, wherein the biological sample comprises lymphocytes.

306. The method of claim 305, wherein the biological sample comprises a fractionated sample comprising lymphocytes.

307. The method of any one of claims 303-306, wherein the peptide is linked to a solid support.

308. The method of claim 307, wherein the peptide is conjugated to the solid support or is bound to an antibody that is conjugated to the solid support.

309. The method of claim 307, wherein the solid support comprises a microplate, a bead, a glass surface, a slide, or a cell culture dish.

310. The method of any one of claims 303-309, wherein detecting T cell responses comprises detecting the binding of the peptide to the T cell or TCR.

311. The method of any one of claims 303-310, wherein detecting T cell responses comprises an ELISA, ELISPOT, or a tetramer assay.

312. The method of any one of claims 303-311, wherein the subject has been diagnosed with a coronavirus.

313. The method of any one of claims 303-312, wherein the subject has been diagnosed with complications relating to a coronavirus.

314. The method of claim 313, wherein the complication comprises pneumonia, difficulty breathing or shortness of breath, chest pain or chest pressure, acute respiratory failure, acute respiratory distress syndrome, acute cardiac injury, secondary infection, acute kidney injury, septic shock, blood clots, multisystem inflammatory syndrome, chronic fatigue, rhabdomyolysis, disseminated intravascular coagulation, and/or acute liver injury.

315. The method of any one of claims 303-311, 313, or 314, wherein the subject has not been diagnosed with a coronavirus.

316. The method of any one of claims 303-315, wherein the subject has been vaccinated for coronavirus.

317. The method of any one of claims 312-316, wherein the coronavirus is SARS-CoV or SARS-CoV-2.

318. The method of claim 316 or 317, wherein the method is for determining the efficacy of the vaccine.

319. A method comprising contacting the composition of any one of claims 158-164 with a composition comprising T cells and detecting T cells with bound peptide and/or MHC polypeptide by detecting a detection tag.

320. The method of claim 319, wherein the method further comprises counting the number of T cells bound with peptide and/or MHC.

321. The method of claim 319 or 320, wherein the composition comprising T cells is isolated from a subject.

322. The method of claim 321, wherein the subject is a human subject.

323. The method of claim 321 or 322, wherein the subject has one or more symptoms of a SARS-Cov-2 infection.

324. The method of any one of claims 321-323, wherein the subject does not have any symptoms of a SARS-Cov-2 infection.

325. The method of any one of claims 321-324, wherein the subject has been diagnosed with a SARS-Cov-2 infection.

326. The method of any one of claims 321-324, wherein the subject has not been diagnosed with a SARS-Cov-2 infection.

327. The method of any one of claims 321-326, wherein the subject has been previously treated for a SARS-Cov-2 infection.

328. The method of claim 202, wherein the subject has been determined to be resistant or non-responsive to the previous treatment.

329. The method of any one of claims 319-328, wherein the method further comprises sorting the number of T cells bound with peptide and/or MHC.

330. The method of claim 329, wherein the method further comprises sequencing one or more TCR genes from T cells bound with peptide and/or MHC.

331. The method of claim 330, wherein the method further comprises grouping of lymphocyte interactions by paratope hotspots (GLIPH) analysis.

332. A kit comprising the peptide or polypeptide of any one of claims 127-157 in a container.

333. The kit of claim 332, wherein the peptide is comprised in a pharmaceutical preparation.

334. The kit of claim 333, wherein the pharmaceutical preparation is formulated for parenteral administration or inhalation.

335. The kit of claim 332, wherein the peptide is comprised in a cell culture media.
